(19) United States
(12) Patent Application Publication
(10) Pub. No.: US 2023/0150980 A1
(43) Pub. Date: May 18, 2023

(54) COLLAGEN 1 TRANSLATION INHIBITORS AND METHODS OF USE THEREOF

(71) Applicant: Anima Biotech Inc., Bernardsville, NJ (US)

(72) Inventors: David William SHEPPARD, Ramat Gan (IL); Jason Paul TIERNEY, Ramat Gan (IL); Aviad MANDABI, Ramat Gan (IL); Wolfgang SCHMIDT, Essex (GB); Stefano LEVANTO, Essex (GB); Julie Nicole HAMBLIN, Essex (GB); Richard James BULL, Essex (GB); Iris ALROY, Ramat Gan (IL); Wissam MANSOUR, Ramat Gan (IL); Moty KLEPFISH, Ramat Gan (IL); Yaode WANG, Beijing (CN); Haitang Ll, Beijing (CN); Stephen David PENROSE, Ramat Gan (IL)

(73) Assignee: Anima Biotech Inc., Bernardsville, NJ (US)

(21) Appl. No.: 17/910,838

(22) PCT Filed: Apr. 21, 2021
(86) PCT No.: PCT/US2021/028335

§ 371 Date: Sep. 12, 2022

Related U.S. Application Data
(60) Provisional application No. 63/013,553, filed on Apr. 22, 2020.

(51) Int. Cl. C07D 403/12; C07D 413/12; C07D 401/12; C07D 417/12; C07D 491/048; C07D 471/04; C07D 487/10; C07D 487/04; C07D 487/08; C07D 498/04

(52) U.S. Cl. CPC C07D 403/12; C07D 413/12; C07D 401/12; C07D 417/12; C07D 491/048; C07D 471/04; C07D 487/10; C07D 487/04; C07D 487/08; C07D 498/04

(57) ABSTRACT

The present invention relates to novel Collagen I translation inhibitors, composition and methods of preparation thereof, and uses thereof for treating Fibrosis including lung, liver, kidney, cardiac and dermal fibrosis, IPF, wound healing, scarring and Gingival fibromatosis, Systemic Sclerosis, and alcoholic and non-alcoholic steatohepatitis (NASH).

### FIELD OF THE INVENTION

The present invention relates to novel Collagen 1 translation inhibitors, composition and methods of preparation thereof, and uses thereof for treating Fibrosis including lung, liver, kidney, cardiac and dermal fibrosis, IPF, wound healing, scarring and Gingival fibromatosis, Systemic Sclerosis, and alcoholic and non-alcoholic steatohepatitis (NASH).

### BACKGROUND OF THE INVENTION

The formation of fibrous connective tissue is part of the normal healing process following tissue damage due to injury or inflammation. During this process, activated immune cells including macrophages stimulate the proliferation and activation of fibroblasts, which in turn deposit connective tissue. However, abnormal or excessive production of connective tissue may lead to accumulation of fibrous material such that it interferes with the normal function of the tissue. Fibrotic growth can proliferate and invade healthy surrounding tissue, even after the original injury heals. Such abnormal formation of excessive connective tissue, occurring in a reparative or reactive process, is referred to as fibrosis.

Many agents cause activation of the fibrotic process and are released in response to tissue injury, inflammation and oxidative stress. Regardless of the initiating events, a feature common to all fibrotic diseases is the conversion of tissue resident fibroblast into ECM-producing myofibroblasts that secrete collagen type I. Current programs indirectly target myofibroblast activation and collagen secretion by inhibiting a single fibrosis inducing signal.

Physiologically, fibrosis acts to deposit connective tissue, which can obliterate the architecture and function of the underlying organ or tissue. Defined by the pathological accumulation of extracellular matrix (ECM) proteins, fibrosis results in scarring and thickening of the affected tissue, which interferes with normal organ function. In various conditions, the formation of fibrotic tissue is characterized by the deposition of abnormally large amounts of collagen. The synthesis of collagen is also involved in a number of other pathological conditions. For example, clinical conditions and disorders associated with primary or secondary fibrosis, such as systemic sclerosis, graft-versus host disease (GVHD), pulmonary fibrosis and autoimmune disorders, are distinguished by excessive production of connective tissue, which results in the destruction of normal tissue architecture and function. These diseases can best be interpreted in terms of perturbations in cellular functions, a major manifestation of which, is excessive collagen synthesis and deposition. The role of collagen in fibrosis has prompted attempts to develop drugs that inhibit its accumulation.

Excessive accumulation of collagen is the major pathologic feature in a variety of clinical conditions characterized by tissue fibrosis. These conditions include localized processes, as for example, pulmonary fibrosis and liver cirrhosis, or more generalized processes, like progressive systemic sclerosis. Collagen deposition is a feature of different forms of dermal fibrosis, which in addition to scleroderma, include localized and generalized morphea, keloids, hypertrophic scars, familial cutaneous collagenoma and connective tissue nevi of the collagen type. Recent advances in the understanding of the normal biochemistry of collagen have allowed us to define specific levels of collagen biosynthesis and degradation at which a pharmacologic intervention could lead to reduced collagen deposition in the tissues. Such compounds could potentially provide us with novel means to reduce the excessive collagen accumulation in diseases.

Fibrosis of the liver, also referred to herein as hepatic fibrosis, may be caused by various types of chronic liver injury, especially if an inflammatory component is involved. Self-limited, acute liver injury (e.g., acute viral hepatitis A), even when fulminant, does not necessarily distort the scaffolding architecture and hence does not typically cause fibrosis, despite loss of hepatocytes. However, factors such as chronic alcoholism, malnutrition, hemochromatosis, and exposure to poisons, toxins or drugs, may lead to chronic liver injury and hepatic fibrosis due to exposure to hepatotoxic chemical substances. Hepatic scarring, caused by surgery or other forms of injury associated with mechanical biliary obstruction, may also result in liver fibrosis.

Fibrosis itself is not necessarily symptomatic, however it can lead to the development of portal hypertension, in which scarring distorts blood flow through the liver, or cirrhosis, in which scarring results in disruption of normal hepatic architecture and liver dysfunction. The extent of each of these pathologies determines the clinical manifestation of hepato-fibrotic disorders. For example, congenital hepatic fibrosis affects portal vein branches, largely sparing the parenchyma. The result is portal hypertension with sparing of hepatocellular function.

### Treatment

Attempts to develop anti-fibrotic agents for the treatment of various disorders have been reported. However, treatment of established fibrosis, formed after months or years of chronic or repeated injury, still remains a challenge.

Treatments aimed at reversing the fibrosis are usually too toxic for long-term use (e.g. corticosteroids, penicillamine) or have no proven efficacy (e.g. colchicine).

Many patients do not respond to available treatments for fibrotic disorders, and long-term treatment is limited by toxicity and side effects. Therefore, a need remains for developing therapeutic modalities aimed at reducing fibrosis. The development of safe and effective treatments for established cirrhosis and portal hypertension and for attenuating fibrosis would be highly beneficial.

Attempts to treat idiopathic pulmonary fibrosis (IPF) with a combination of anti-inflammatory drugs (prednisone, azathioprine and N-acetyl-1-cysteine (NAC)), failed to improve outcomes, and instead increased mortality. In 2014, two drugs, pirfenidone, a drug with poorly understood mechanisms, and nintedanib, a tyrosine kinase inhibitor, were approved for the treatment of IPF mainly on the basis of their ability to reduce the decrease in forced vital capacity (FVC) and to slow the pace of disease progression. To date, however, it is unclear whether these drugs improve symptoms such as dyspnoea and cough, or whether their beneficial effect on functional decline translates to increased survival.

The compounds of this invention target activated fibroblasts and collagen over production and can therefore be used for treating fibrosis, including primary or secondary fibrosis, such as systemic sclerosis, graft-versus host disease (GVHD), pulmonary fibrosis and autoimmune disorders, lung fibrosis and idiopathic pulmonary fibrosis (IPF), as well as localized processes, as for example, pulmonary fibrosis and liver cirrhosis, or more generalized processes, like progressive systemic sclerosis. The compounds can be further useful in the treatment of different forms of dermal fibrosis, which in addition to scleroderma, include localized and generalized morphea, keloids, hypertrophic scars, familial cutaneous collagenoma and connective tissue nevi of the collagen type. The compounds can be further useful in the treatment of lung fibrosis and idiopathic pulmonary fibrosis (IPF), as well as hepatic fibrosis, resulting from hepatic scarring, caused by surgery or other forms of injury associated with mechanical biliary obstruction. Such fibrosis can lead to portal hypertension, in which scarring distorts blood flow through the liver, or cirrhosis as well as other hepato-fibrotic disorders including Non-alcoholic steatohepatitis (NASH), and alcoholic steatohepatitis (ASH), non-alcoholic fatty liver disease (NAFLD) and alcoholic fatty liver disease (AFLD), which can be similarly be treated by compounds of the invention.

### SUMMARY OF THE INVENTION

This invention provides a compound or its pharmaceutically acceptable salt, stereoisomer, tautomer, hydrate, N-oxide, reverse amide analog, prodrug, isotopic variants (e.g., deuterated analog), pharmaceutical product or any combination thereof, represented by the structure of formula I-X and by the structures listed in Table 1, as defined herein below. In various embodiments, the compound is Collagen I translation inhibitor.

This invention further provides a pharmaceutical composition comprising a compound or its pharmaceutically acceptable salt, stereoisomer, tautomer, hydrate, N-oxide, prodrug, isotopic variants (e.g., deuterated analog), pharmaceutical product or any combination thereof, represented by the structure of formula I-X and by the structures listed in Table 1, as defined herein below, and a pharmaceutically acceptable carrier.

This invention further provides a method of treating, suppressing, reducing the severity, reducing the risk of developing or inhibiting fibrosis in a subject, comprising administering a compound represented by the structure of formula I-X and by the structures listed in Table 1, as defined herein below, to a subject suffering from fibrosis under conditions effective to treat, suppress, reduce the severity, reduce the risk of developing, or inhibit fibrosis in said subject. In some embodiments, the fibrosis is a systemic fibrotic disease. In some embodiments, the systemic fibrotic disease is systemic sclerosis, multifocal fibrosclerosis (IgG4-associated fibrosis), nephrogenic systemic fibrosis, sclerodermatous graft vs. host disease, or any combination thereof. In some embodiments, the fibrosis is an organ-specific fibrotic disease. In some embodiments, the organ-specific fibrotic disease is lung fibrosis, cardiac fibrosis, kidney fibrosis, pulmonary fibrosis, liver and portal vein fibrosis, radiation-induced fibrosis, bladder fibrosis, intestinal fibrosis, peritoneal sclerosis, diffuse fasciitis, wound healing, scaring, or any combination thereof. In some embodiments, the lung fibrosis is idiopathic pulmonary fibrosis (IPF). In some embodiments, the cardiac fibrosis is hypertension-associated cardiac fibrosis, Post-myocardial infarction, Chagas disease-induced myocardial fibrosis or any combination thereof. In some embodiments, the kidney fibrosis is diabetic and hypertensive nephropathy, urinary tract obstruction-induced kidney fibrosis, inflammatory/autoimmune-induced kidney fibrosis, aristolochic acid nephropathy, polycystic kidney disease, or any combination thereof. In some embodiments, the pulmonary fibrosis is idiopathic pulmonary fibrosis, silica-induced pneumoconiosis (silicosis), asbestos-induced pulmonary fibrosis (asbestosis), chemotherapeutic agent-induced pulmonary fibrosis, or any combination thereof. In some embodiments, the liver and portal vein fibrosis is alcoholic and nonalcoholic liver fibrosis, hepatitis C-induced liver fibrosis, primary biliary cirrhosis, parasite-induced liver fibrosis (schistosomiasis), or any combination thereof. In some embodiments, the diffuse fasciitis is localized scleroderma, keloids, dupuytren's disease, peyronie's disease, myelofibrosis, oral submucous fibrosis, or any combination thereof. In some embodiments, the fibrosis is primary or secondary fibrosis. In some embodiments, the fibrosis is a result of systemic sclerosis, graft-versus host disease (GVHD), pulmonary fibrosis, autoimmune disorder, tissue injury, inflammation, oxidative stress or any combination thereof. In some embodiments, the fibrosis is hepatic fibrosis, lung fibrosis or dermal fibrosis. In some embodiments, the subject has a liver cirrhosis. In some embodiments, the dermal fibrosis is scleroderma. In some embodiments, the dermal fibrosis is a result of a localized or generalized morphea, keloids, hypertrophic scars, familial cutaneous collagenoma, connective tissue nevi of the collagen type, or any combination thereof. In some embodiments, the hepatic fibrosis is a result of hepatic scarring or chronic liver injury. In some embodiments, the chronic liver injury results from alcoholism, malnutrition, hemochromatosis, exposure to poisons, toxins or drugs.

This invention further provides a method of treating, suppressing, reducing the severity, reducing the risk of developing or inhibiting lung fibrosis in a subject, comprising administering a compound represented by the structure of formula I-X and by the structures listed in Table 1, as defined herein below, to a subject suffering from lung fibrosis under conditions effective to treat, suppress, reduce the severity, reduce the risk of developing, or inhibit lung fibrosis in said subject. In some embodiments, the lung fibrosis is idiopathic pulmonary fibrosis (IPF).

This invention further provides a method of treating, suppressing, reducing the severity, reducing the risk of developing or inhibiting idiopathic pulmonary fibrosis (IPF) in a subject, comprising administering a compound represented by the structure of formula I-X and by the structures listed in Table 1, as defined herein below, to a subject suffering from idiopathic pulmonary fibrosis (IPF) under conditions effective to treat, suppress, reduce the severity, reduce the risk of developing, or inhibit idiopathic pulmonary fibrosis (IPF) in said subject.

This invention further provides a method of treating, suppressing, reducing the severity, reducing the risk of developing or inhibiting hepato-fibrotic disorder in a subject, comprising administering a compound represented by the structure of formula I-X and by the structures listed in Table 1, as defined herein below, to a subject suffering from hepato-fibrotic disorder under conditions effective to treat, suppress, reduce the severity, reduce the risk of developing, or inhibit hepato-fibrotic disorder in said subject. In some embodiments, the hepato-fibrotic disorder is a portal hypertension, cirrhosis, congenital hepatic fibrosis or any combination thereof.

This invention further provides a method of treating, suppressing, reducing the severity, reducing the risk of developing or inhibiting cirrhosis in a subject, comprising administering a compound represented by the structure of formula I-X and by the structures listed in Table 1, as defined herein below, to a subject suffering from cirrhosis under conditions effective to treat, suppress, reduce the severity, reduce the risk of developing, or inhibit cirrhosis in said subject. In some embodiments, the cirrhosis is a result of hepatitis or alcoholism.

This invention further provides a method of treating, suppressing, reducing the severity, reducing the risk of developing or inhibiting a alcoholic steatohepatitis (ASH) in a subject, comprising administering a compound represented by the structure of formula I-X and by the structures listed in Table 1, as defined herein below, to a subject suffering from alcoholic steatohepatitis (ASH) under conditions effective to treat, suppress, reduce the severity, reduce the risk of developing, or inhibit the alcoholic steatohepatitis (ASH) in said subject.

This invention further provides a method of treating, suppressing, reducing the severity, reducing the risk of developing or inhibiting a non-alcoholic steatohepatitis (NASH) in a subject, comprising administering a compound represented by the structure of formula I-X and by the structures listed in Table 1, as defined herein below, to a subject suffering from non-alcoholic steatohepatitis (NASH) under conditions effective to treat, suppress, reduce the severity, reduce the risk of developing, or inhibit the non-alcoholic steatohepatitis (NASH) in said subject.

This invention further provides a method of treating, suppressing, reducing the severity, reducing the risk of developing or inhibiting a alcoholic fatty liver disease (AFLD) in a subject, comprising administering a compound represented by the structure of formula I-X and by the structures listed in Table 1, as defined herein below, to a subject suffering from alcoholic fatty liver disease (AFLD) under conditions effective to treat, suppress, reduce the severity, reduce the risk of developing, or inhibit the alcoholic fatty liver disease (AFLD) in said subject.

This invention further provides a method of treating, suppressing, reducing the severity, reducing the risk of developing or inhibiting a non alcoholic fatty liver disease (NAFLD) in a subject, comprising administering a compound represented by the structure of formula I-X and by the structures listed in Table 1, as defined herein below, to a subject suffering from non alcoholic fatty liver disease (NAFLD) under conditions effective to treat, suppress, reduce the severity, reduce the risk of developing, or inhibit the non alcoholic fatty liver disease (NAFLD) in said subject.

This invention further provides a method of treating, suppressing, reducing the severity, reducing the risk of developing or inhibiting an autoimmune disease or disorder in a subject, comprising administering a compound represented by the structure of formula I-X and by the structures listed in Table 1, as defined herein below, to a subject suffering from an autoimmune disease or disorder under conditions effective to treat, suppress, reduce the severity, reduce the risk of developing, or inhibit the autoimmune disease or disorder in said subject.

This invention further provides a method of treating, suppressing, reducing the severity, reducing the risk of developing or inhibiting an autoimmune disease or disorder in a subject, comprising administering a compound represented by the structure of formula I-X and by the structures listed in Table 1, as defined herein below, to a subject suffering from an autoimmune disease or disorder under conditions effective to treat, suppress, reduce the severity, reduce the risk of developing, or inhibit the autoimmune disease or disorder in said subject.

This invention provides a compound or its pharmaceutically acceptable salt, stereoisomer, tautomer, hydrate, N-oxide, reverse amide analog, prodrug, isotopic variants (e.g., deuterated analog), pharmaceutical product or any combination thereof, represented by the structure of formula I-X and by the structures listed in Table 1, as defined herein below. In various embodiments, the compound is Collagen I translation inhibitor.

This invention further provides a pharmaceutical composition comprising a compound or its pharmaceutically acceptable salt, stereoisomer, tautomer, hydrate, N-oxide, prodrug, isotopic variants (e.g., deuterated analog), pharmaceutical product or any combination thereof, represented by the structure of formula I-X and by the structures listed in Table 1, as defined herein below, and a pharmaceutically acceptable carrier.

This invention further provides a method of treating, suppressing, reducing the severity, reducing the risk of developing or inhibiting fibrosis in a subject, comprising administering a compound represented by the structure of formula I-X and by the structures listed in Table 1, as defined herein below, to a subject suffering from fibrosis under conditions effective to treat, suppress, reduce the severity, reduce the risk of developing, or inhibit fibrosis in said subject. In some embodiments, the fibrosis is a systemic fibrotic disease. In some embodiments, the systemic fibrotic disease is systemic sclerosis, multifocal fibrosclerosis (IgG4-associated fibrosis), nephrogenic systemic fibrosis, sclerodermatous graft vs. host disease, or any combination thereof. In some embodiments, the fibrosis is an organ-specific fibrotic disease. In some embodiments, the organ-specific fibrotic disease is lung fibrosis, cardiac fibrosis, kidney fibrosis, pulmonary fibrosis, liver and portal vein fibrosis, radiation-induced fibrosis, bladder fibrosis, intestinal fibrosis, peritoneal sclerosis, diffuse fasciitis, wound healing, scaring, or any combination thereof. In some embodiments, the lung fibrosis is idiopathic pulmonary fibrosis (IPF). In some embodiments, the cardiac fibrosis is hypertension-associated cardiac fibrosis, Post-myocardial infarction, Chagas disease-induced myocardial fibrosis or any combination thereof. In some embodiments, the kidney fibrosis is diabetic and hypertensive nephropathy, urinary tract obstruction-induced kidney fibrosis, inflammatory/autoimmune-induced kidney fibrosis, aristolochic acid nephropathy, polycystic kidney disease, or any combination thereof. In some embodiments, the pulmonary fibrosis is idiopathic pulmonary fibrosis, silica-induced pneumoconiosis (silicosis), asbestos-induced pulmonary fibrosis (asbestosis), chemotherapeutic agent-induced pulmonary fibrosis, or any combination thereof. In some embodiments, the liver and portal vein fibrosis is alcoholic and nonalcoholic liver fibrosis, hepatitis C-induced liver fibrosis, primary biliary cirrhosis, parasite-induced liver fibrosis (schistosomiasis), or any combination thereof. In some embodiments, the diffuse fasciitis is localized scleroderma, keloids, dupuytren's disease, peyronie's disease, myelofibrosis, oral submucous fibrosis, or any combination thereof. In some embodiments, the fibrosis is primary or secondary fibrosis. In some embodiments, the fibrosis is a result of systemic sclerosis, graft-versus host disease (GVHD), pulmonary fibrosis, autoimmune disorder, tissue injury, inflammation, oxidative stress or any combination thereof. In some embodiments, the fibrosis is hepatic fibrosis, lung fibrosis or dermal fibrosis. In some embodiments, the subject has a liver cirrhosis. In some embodiments, the dermal fibrosis is scleroderma. In some embodiments, the dermal fibrosis is a result of a localized or generalized morphea, keloids, hypertrophic scars, familial cutaneous collagenoma, connective tissue nevi of the collagen type, or any combination thereof. In some embodiments, the hepatic fibrosis is a result of hepatic scarring or chronic liver injury. In some embodiments, the chronic liver injury results from alcoholism, malnutrition, hemochromatosis, exposure to poisons, toxins or drugs.

This invention further provides a method of treating, suppressing, reducing the severity, reducing the risk of developing or inhibiting lung fibrosis in a subject, comprising administering a compound represented by the structure of formula I-X and by the structures listed in Table 1, as defined herein below, to a subject suffering from lung fibrosis under conditions effective to treat, suppress, reduce the severity, reduce the risk of developing, or inhibit lung fibrosis in said subject. In some embodiments, the lung fibrosis is idiopathic pulmonary fibrosis (IPF).

This invention further provides a method of treating, suppressing, reducing the severity, reducing the risk of developing or inhibiting idiopathic pulmonary fibrosis (IPF) in a subject, comprising administering a compound represented by the structure of formula I-X and by the structures listed in Table 1, as defined herein below, to a subject suffering from idiopathic pulmonary fibrosis (IPF) under conditions effective to treat, suppress, reduce the severity, reduce the risk of developing, or inhibit idiopathic pulmonary fibrosis (IPF) in said subject.

This invention further provides a method of treating, suppressing, reducing the severity, reducing the risk of developing or inhibiting hepato-fibrotic disorder in a subject, comprising administering a compound represented by the structure of formula I-X and by the structures listed in Table 1, as defined herein below, to a subject suffering from hepato-fibrotic disorder under conditions effective to treat, suppress, reduce the severity, reduce the risk of developing, or inhibit hepato-fibrotic disorder in said subject. In some embodiments, the hepato-fibrotic disorder is a portal hypertension, cirrhosis, congenital hepatic fibrosis or any combination thereof.

This invention further provides a method of treating, suppressing, reducing the severity, reducing the risk of developing or inhibiting cirrhosis in a subject, comprising administering a compound represented by the structure of formula I-X and by the structures listed in Table 1, as defined herein below, to a subject suffering from cirrhosis under conditions effective to treat, suppress, reduce the severity, reduce the risk of developing, or inhibit cirrhosis in said subject. In some embodiments, the cirrhosis is a result of hepatitis or alcoholism.

This invention further provides a method of treating, suppressing, reducing the severity, reducing the risk of developing or inhibiting a alcoholic steatohepatitis (ASH) in a subject, comprising administering a compound represented by the structure of formula I-X and by the structures listed in Table 1, as defined herein below, to a subject suffering from alcoholic steatohepatitis (ASH) under conditions effective to treat, suppress, reduce the severity, reduce the risk of developing, or inhibit the alcoholic steatohepatitis (ASH) in said subject.

This invention further provides a method of treating, suppressing, reducing the severity, reducing the risk of developing or inhibiting a non-alcoholic steatohepatitis (NASH) in a subject, comprising administering a compound represented by the structure of formula I-X and by the structures listed in Table 1, as defined herein below, to a subject suffering from non-alcoholic steatohepatitis (NASH) under conditions effective to treat, suppress, reduce the severity, reduce the risk of developing, or inhibit the non-alcoholic steatohepatitis (NASH) in said subject.

This invention further provides a method of treating, suppressing, reducing the severity, reducing the risk of developing or inhibiting a alcoholic fatty liver disease (AFLD) in a subject, comprising administering a compound represented by the structure of formula I-X and by the structures listed in Table 1, as defined herein below, to a subject suffering from alcoholic fatty liver disease (AFLD) under conditions effective to treat, suppress, reduce the severity, reduce the risk of developing, or inhibit the alcoholic fatty liver disease (AFLD) in said subject.

This invention further provides a method of treating, suppressing, reducing the severity, reducing the risk of developing or inhibiting a non alcoholic fatty liver disease (NAFLD) in a subject, comprising administering a compound represented by the structure of formula I-X and by the structures listed in Table 1, as defined herein below, to a subject suffering from non alcoholic fatty liver disease (NAFLD) under conditions effective to treat, suppress, reduce the severity, reduce the risk of developing, or inhibit the non alcoholic fatty liver disease (NAFLD) in said subject.

This invention further provides a method of treating, suppressing, reducing the severity, reducing the risk of developing or inhibiting an autoimmune disease or disorder in a subject, comprising administering a compound represented by the structure of formula I-X and by the structures listed in Table 1, as defined herein below, to a subject suffering from an autoimmune disease or disorder under conditions effective to treat, suppress, reduce the severity, reduce the risk of developing, or inhibit the autoimmune disease or disorder in said subject.

This invention further provides a method of treating, suppressing, reducing the severity, reducing the risk of developing or inhibiting an autoimmune disease or disorder in a subject, comprising administering a compound represented by the structure of formula I-X and by the structures listed in Table 1, as defined herein below, to a subject suffering from an autoimmune disease or disorder under conditions effective to treat, suppress, reduce the severity, reduce the risk of developing, or inhibit the autoimmune disease or disorder in said subject.

### BRIEF DESCRIPTION OF THE DRAWINGS

The patent of application file contains at least one drawing executed in color. Copies of this patent or patent application publication with color drawing(s) will be provided by the office upon request and payment of the necessary fee.

FIG. 1 demonstrates how Protein synthesis monitoring (PSM) specifically monitors collagen 1 synthesis. The assay system comprises human lung fibroblast cell line, WI-38 cells, which are activated to produce higher levels of collagen. Two tRNAs (di-tRNA) which decode one specific glycine codon and one specific proline codon were transfected with control RNAi or an RNAi directed to Collagen 1. The FRET signal specifically monitors collagen 1 translation, as the FRET signal in collagen 1-targeted siRNA treated cells is inhibited by 90%. In blue, cell nuclei stained with DAPI; In Cyan, FRET signals from tRNA pair which decodes glycine-proline di-codons.

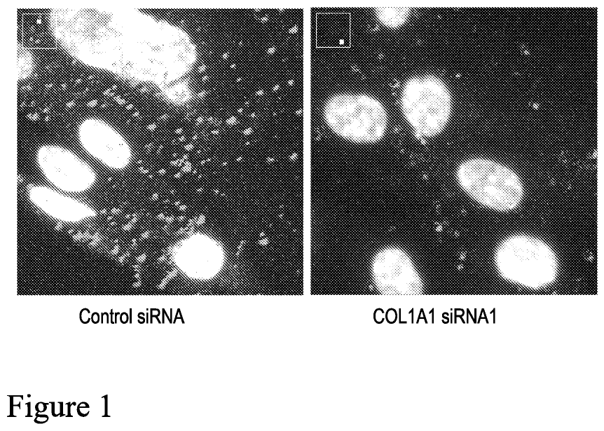

FIG. 2 depicts that hits selectively regulate collagen translation. In the upper panel, the Y-axis depicts normalized values of metabolic labeling in control cells. Only compounds which showed minimal effects on global protein synthesis (±20% of control) and minimal effects on collagen 1 protein accumulation in WI38 cells by di-tRNA Collagen FRET and by Collagen 1 specific immunofluorescence were selected as compounds which selectively regulate collagen synthesis; In the lower panel, Y axis shows the FRET score for the collagen specific di-tRNA (PSM score) and the X-axis shows the normalized immunofluorescence values (relative to control). Compounds that show high PSM score are marked by dot size; compounds that increase collagen content are marked as red, and compounds that decrease collagen content are marked as green.

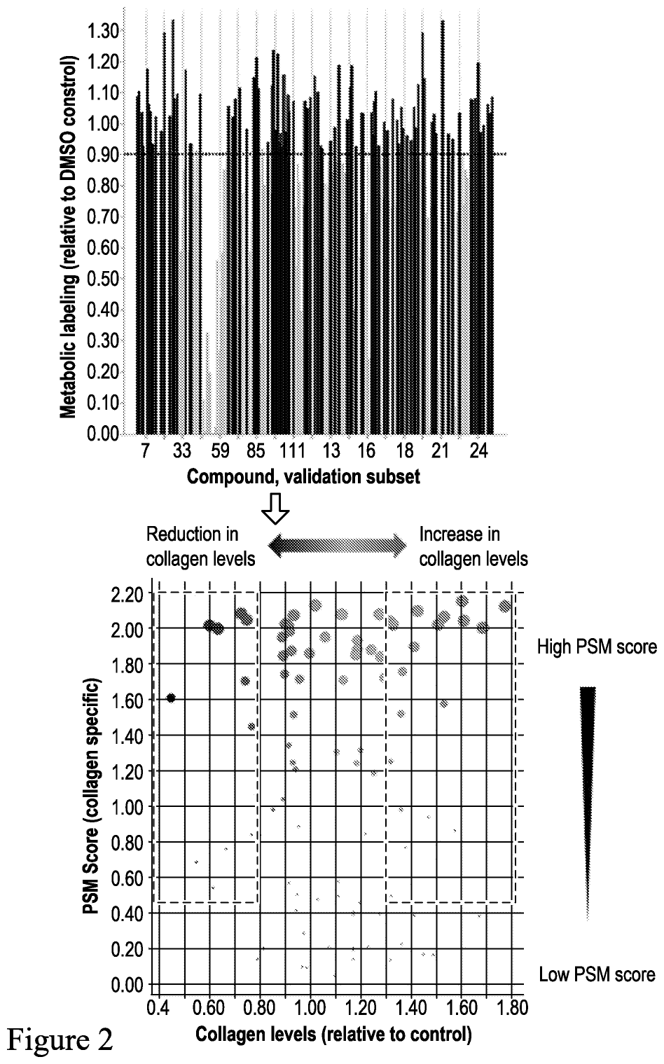

FIG. 3 demonstrates that compounds act at the level of translation. WI-38 Human Lung Fibroblasts, 96 hours incubation with compounds. Immunofluorescence. In blue, cell nuclei stained with DAPI; In green, Collagen protein detected with anti-collagen antibody.

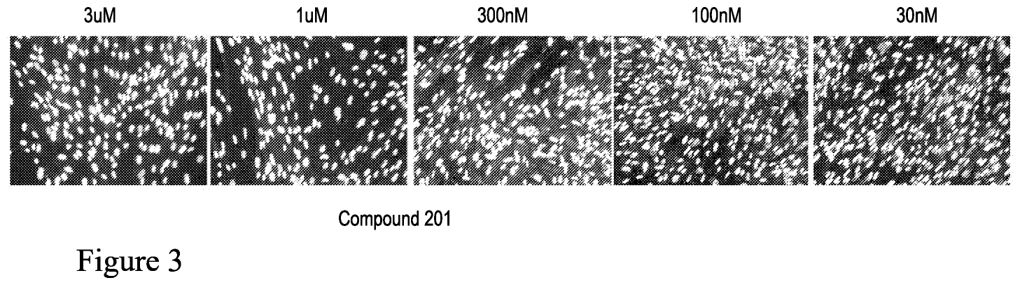

FIG. 4 demonstrates the efficacy and toxicity of compounds 201, 256, and 213. FIG. (4A) pEC50 of efficacy plotted against pEC50 of toxicity. Dashed lines represent ×10 or ×100 window between efficacy and toxicity. Figure (4B) Representative images from compound 213. Images were taken with ×20 objective in Operetta machine (Perkin-Elmer). Green: Collagen type-I; Grey: DAPI.

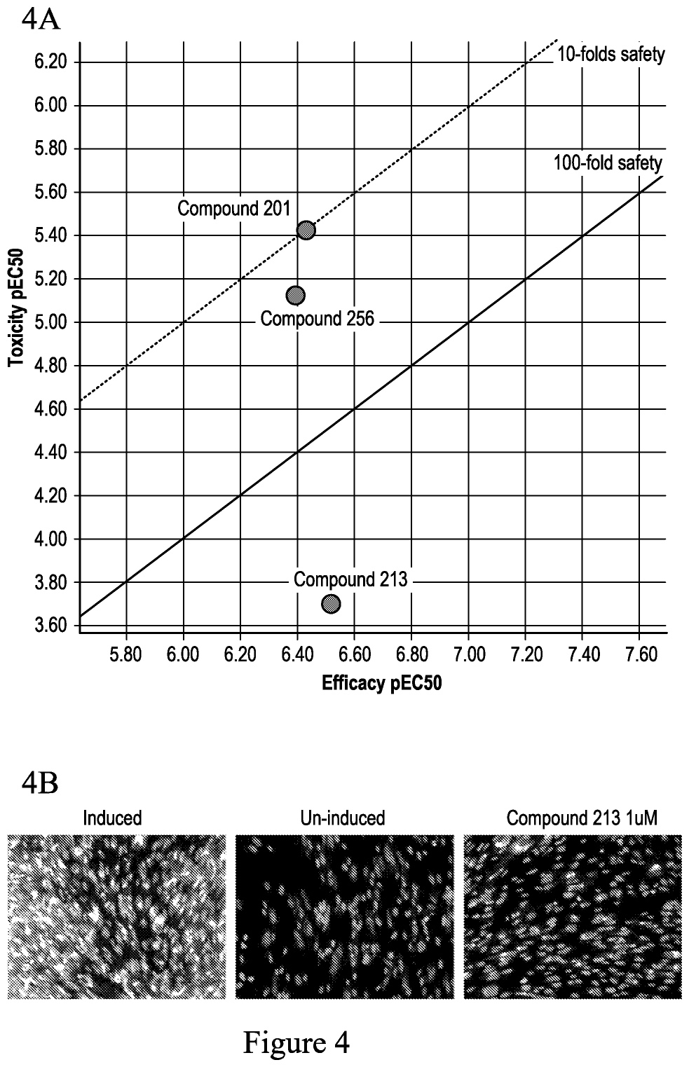

### DETAILED DESCRIPTION OF THE INVENTION

In various embodiments, this invention is directed to a compound represented by the structure of formula (I):

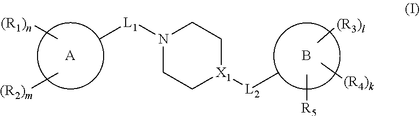

wherein

A and B rings are each independently a single or fused aromatic or heteroaromatic ring system (e.g., benzimidazole, indole, benzothiazole, benzooxazole, imidazopyridin, pyrazolopyridine, pyrrolopyridine, phenyl, pyrimidine, 2-, 3- or 4-pyridine, pyridazine, pyrazine, thiazole, pyrrole, triazole, imidazole, indazole), or a single or fused C₃-C₁₀ cycloalkyl (e.g. cyclohexyl, cyclopentyl) or a single or fused C₃-C₁₀ heterocyclic ring (e.g., piperidine, tetrahydro-2H-pyran); R₁ and R₂ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., pyridine) ring;

R₃, R₄ and R₅ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole, imidazole), substituted or unsubstituted aryl (e.g., phenyl), substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

R₂₀ is represented by the following structure:

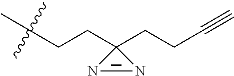 [SMILES: *CCC1(CCC#C)N=N1]

X₁ is N or C—R (e.g., C—H, C—OH);

L₁ is CH₂, CHR, C(R)₂, or C═O;

L₂ is a bond or CH₂, C═O, O or S;

R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]_{q}, [C]_{q}
wherein q is between 2 and 10;

R₁₀ and R₁₁ are each independently H, C₁-C₅ substituted or unsubstituted, linear or branched alkyl (e.g., methyl, ethyl, CH₂—CH₂—O—CH₃), C₁-C₅ linear or branched alkoxy (e.g., O—CH₃), C(O)R, or S(O)₂R;

or R₁₀ and R₁₁ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;

m, n, l and k are each independently an integer between 0 and 4 (e.g., 0, 1 or 2);

or its pharmaceutically acceptable salt, stereoisomer, tautomer, hydrate, N-oxide, prodrug, isotopic variant (e.g., deuterated analog), reverse amide, pharmaceutical product or any combination thereof.

In various embodiments, this invention is directed to a compound represented by the structure of formula I(a):

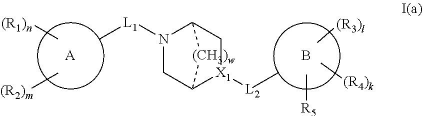

wherein

A and B rings are each independently a single or fused aromatic or heteroaromatic ring system (e.g., benzimidazole, indole, benzothiazole, benzooxazole, imidazopyridin, pyrazolopyridine, pyrrolopyridine, phenyl, pyrimidine, 2-, 3- or 4-pyridine, pyridazine, pyrazine, thiazole, pyrrole, triazole, imidazole, indazole), or a single or fused C₃-C₁₀ cycloalkyl (e.g. cyclohexyl, cyclopentyl) or a single or fused C₃-C₁₀ heterocyclic ring (e.g., piperidine, tetrahydro-2H-pyran);

R₁ and R₂ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., pyridine) ring;

R₃, R₄ and R₅ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole, imidazole), substituted or unsubstituted aryl (e.g., phenyl), substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

R₂₀ is represented by the following structure:

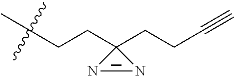 [SMILES: *CCC1(CCC#C)N=N1]

X₁ is N or C—R (e.g., C—H, C—OH);

L₁ is CH₂, CHR, C(R)₂, or C═O;

L₂ is a bond or CH₂, C═O, O or S;

R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 1, 2);

R₉ is [CH]_{q}, [C]_{q}
wherein q is between 2 and 10;

R₁₀ and R₁₁ are each independently H, C₁-C₅ substituted or unsubstituted, linear or branched alkyl (e.g., methyl, ethyl, CH₂—CH₂—O—CH₃), C₁-C₅ linear or branched alkoxy (e.g., O—CH₃), C(O)R, or S(O)₂R;

or R₁₀ and R₁₁ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;

m, n, l and k are each independently an integer between 0 and 4 (e.g., 0, 1 or 2);

w is 0, 1 or 2; wherein if w=0, the bridge on the ring is absent;

or its pharmaceutically acceptable salt, stereoisomer, tautomer, hydrate, N-oxide, prodrug, isotopic variant (e.g., deuterated analog), reverse amide, pharmaceutical product or any combination thereof.

In various embodiments, this invention is directed to a compound represented by the structure of formula II

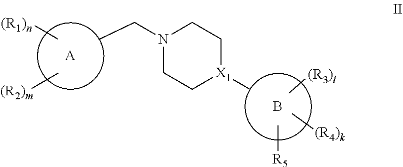

wherein

A and B rings are each independently a single or fused aromatic or heteroaromatic ring system (e.g., benzimidazole, indole, benzothiazole, benzooxazole, imidazopyridin, pyrazolopyridine, pyrrolopyridine, phenyl, pyrimidine, 2-, 3- or 4-pyridine, pyridazine, pyrazine, thiazole, pyrrole, triazole, imidazole, indazole), or a single or fused C₃-C₁₀ cycloalkyl (e.g. cyclohexyl, cyclopentyl) or a single or fused C₃-C₁₀ heterocyclic ring (e.g., piperidine, tetrahydro-2H-pyran);

R₁ and R₂ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., pyridine) ring;

R₃, R₄ and R₅ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole, imidazole), substituted or unsubstituted aryl (e.g., phenyl), substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

R₂₀ is represented by the following structure:

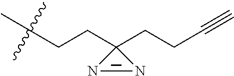 [SMILES: *CCC1(CCC#C)N=N1]

X₁ is N or C—R (e.g., C—H, C—OH);

R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]_{q}, [C]_{q}
wherein q is between 2 and 10;

R₁₀ and R₁₁ are each independently H, C₁-C₅ substituted or unsubstituted, linear or branched alkyl (e.g., methyl, ethyl, CH₂—CH₂—O—CH₃), C₁-C₅ linear or branched alkoxy (e.g., O—CH₃), C(O)R, or S(O)₂R;

or R₁₀ and R₁₁ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;

m, n, l and k are each independently an integer between 0 and 4 (e.g., 0, 1 or 2);

or its pharmaceutically acceptable salt, stereoisomer, tautomer, hydrate, N-oxide, prodrug, isotopic variant (e.g., deuterated analog), reverse amide, pharmaceutical product or any combination thereof.

In various embodiments, this invention is directed to a compound represented by the structure of formula III:

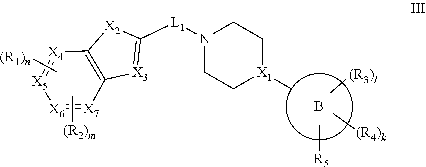

wherein

B ring is a single or fused aromatic or heteroaromatic ring system (e.g., phenyl, pyrimidine, 2-, 3- or 4-pyridine, pyridazine or pyrazine, thiazole, pyrrole, triazole, imidazole, indazole), or a single or fused C₃-C₁₀ cycloalkyl (e.g. cyclohexyl, cyclopentyl) or a single or fused C₃-C₁₀ heterocyclic ring (e.g., piperidine, tetrahydro-2H-pyran);

L₁ is CH₂, CHR, C(R)₂, or C═O;

R₁ and R₂ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈₅ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., pyridine) ring;

R₃, R₄ and R₅ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(Rn), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole, imidazole), substituted or unsubstituted aryl (e.g., phenyl), substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

R₂₀ is represented by the following structure:

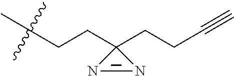 [SMILES: *CCC1(CCC#C)N=N1]

X₁ is N or C(R) (e.g., C—H, C—OH);

X₂ is NH, S, O, N—R (e.g., N—CH₂—CH₂—O—CH₃);

X₃ is N, C(R) (e.g., CH, C—CH₃, C—Cl, C—CN);

X₄, X₅, X₆, and X₇ are each independently C or N;

R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]_{q}, [C]_{q}
wherein q is between 2 and 10;

R₁₀ and R₁₁ are each independently H, C₁-C₅ substituted or unsubstituted, linear or branched alkyl (e.g., methyl, ethyl, CH₂—CH₂—O—CH₃), C₁-C₅ linear or branched alkoxy (e.g., O—CH₃), C(O)R, or S(O)₂R;

or R₁₀ and R₁₁ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;

m, n, l and k are each independently an integer between 0 and 4 (e.g., 0, 1 or 2);

or its pharmaceutically acceptable salt, stereoisomer, tautomer, hydrate, N-oxide, prodrug, isotopic variant (e.g., deuterated analog), reverse amide, pharmaceutical product or any combination thereof.

In various embodiments, if X₃ is N, then X₂ is not NH.

In various embodiments, this invention is directed to a compound represented by the structure of formula IV:

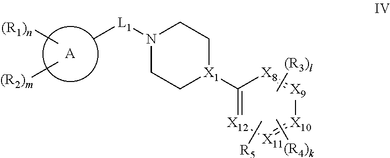

wherein

A ring is a single or fused aromatic or heteroaromatic ring system (e.g., benzimidazole, indole, benzothiazole, benzooxazole, imidazopyridin, pyrazolopyridine, pyrrolopyridine, phenyl, pyrimidine, 2-, 3- or 4-pyridine, pyridazine, or pyrazine), or a single or fused C₃-C₁₀ cycloalkyl, or a single or fused C₃-C₁₀ heterocyclic ring (e.g., piperidine, tetrahydro-2H-pyran);

L₁ is CH₂, CHR, C(R)₂, or C═O;

R₁ and R₂ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., pyridine) ring;

R₃, R₄ and R₅ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole, imidazole), substituted or unsubstituted aryl (e.g., phenyl), substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

R₂₀ is represented by the following structure:

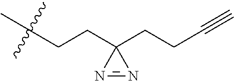 [SMILES: *CCC1(CCC#C)N=N1]

X₁ is N or C(R) (e.g., C—H, C—OH);

X₈, X₉, X₁₀, X₁₁, and X₁₂ are each independently C or N;

R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]_{q}, [C]_{q}
wherein q is between 2 and 10;

R₁₀ and R₁₁ are each independently H, C₁-C₅ substituted or unsubstituted, linear or branched alkyl (e.g., methyl, ethyl, CH₂—CH₂—O—CH₃), C₁-C₅ linear or branched alkoxy (e.g., O—CH₃), C(O)R, or S(O)₂R;

or R₁₀ and R₁₁ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;

m, n, l and k are each independently an integer between 0 and 4 (e.g., 0, 1 or 2);

or its pharmaceutically acceptable salt, stereoisomer, tautomer, hydrate, N-oxide, prodrug, isotopic variant (e.g., deuterated analog), reverse amide, pharmaceutical product or any combination thereof.

In various embodiments, at least one of X₈, X₉, X₁₀, X₁₁, and X₁₂ is N.

In various embodiments, this invention is directed to a compound represented by the structure of formula V:

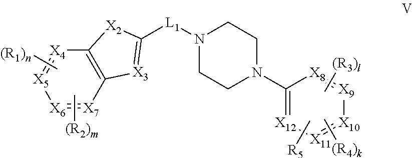

wherein

L₁ is CH₂, CHR, C(R)₂, or C═O;

R₁ and R₂ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., pyridine) ring;

R₃, R₄ and R₅ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole, imidazole), substituted or unsubstituted aryl (e.g., phenyl), substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

R₂₀ is represented by the following structure:

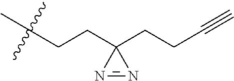 [SMILES: *CCC1(CCC#C)N=N1]

X₁ is N or C(R) (e.g., C—H, C—OH);

X₂ is NH, S, O, N—R (e.g., N—CH₂—CH₂—O—CH₃);

X₃ is N, C(R) (e.g., CH, C—CH₃, C—Cl, C—CN);

X₄, X₅, X₆, and X₇ are each independently C or N;

X₈, X₉, X₁₀, X₁₁, and X₁₂ are each independently C or N;

R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]_{q}, [C]_{q}
wherein q is between 2 and 10;

R₁₀ and R₁₁ are each independently H, C₁-C₅ substituted or unsubstituted, linear or branched alkyl (e.g., methyl, ethyl, CH₂—CH₂—O—CH₃), C₁-C₅ linear or branched alkoxy (e.g., O—CH₃), C(O)R, or S(O)₂R;

or R₁₀ and R₁₁ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;

m, n, l and k are each independently an integer between 0 and 4 (e.g., 0, 1 or 2);

or its pharmaceutically acceptable salt, stereoisomer, tautomer, hydrate, N-oxide, prodrug, isotopic variant (e.g., deuterated analog), reverse amide, pharmaceutical product or any combination thereof.

In various embodiments, if X₃ is N, then X₂ is not NH. In various embodiments, at least one of X₈, X₉, X₁₀, X₁₁, and X₁₂ is N.

In various embodiments, this invention is directed to a compound represented by the structure of formula V(a):

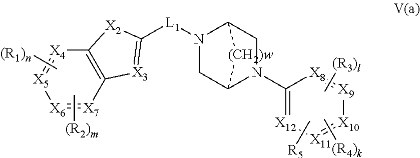

wherein

L₁ is CH₂, CHR, C(R)₂, or C═O;

R₁ and R₂ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., pyridine) ring;

R₃, R₄ and R₅ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole, imidazole), substituted or unsubstituted aryl (e.g., phenyl), substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

R₂₀ is represented by the following structure:

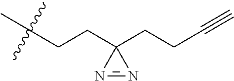 [SMILES: *CCC1(CCC#C)N=N1]

X₁ is N or C(R) (e.g., C—H, C—OH);

X₂ is NH, S, O, N—R (e.g., N—CH₂—CH₂—O—CH₃);

X₃ is N, C(R) (e.g., CH, C—CH₃, C—Cl, C—CN);

X₄, X₅, X₆, and X₇ are each independently C or N;

X₈, X₉, X₁₀, X₁₁, and X₁₂ are each independently C or N;

R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]_{q}, [C]_{q}
wherein q is between 2 and 10;

R₁₀ and R₁₁ are each independently H, C₁-C₅ substituted or unsubstituted, linear or branched alkyl (e.g., methyl, ethyl, CH₂—CH₂—O—CH₃), C₁-C₅ linear or branched alkoxy (e.g., O—CH₃), C(O)R, or S(O)₂R;

or R₁₀ and R₁₁ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;

m, n, 1 and k are each independently an integer between 0 and 4 (e.g., 0, 1 or 2);

w is 0, 1 or 2; wherein if w=0, the bridge on the ring is absent;

or its pharmaceutically acceptable salt, stereoisomer, tautomer, hydrate, N-oxide, prodrug, isotopic variant (e.g., deuterated analog), reverse amide, pharmaceutical product or any combination thereof.

In various embodiments, if X₃ is N, then X₂ is not NH. In various embodiments, at least one of X₈, X₉, X₁₀, X₁₁, and X₁₂ is N.

In various embodiments, this invention is directed to a compound represented by the structure of formula VI:

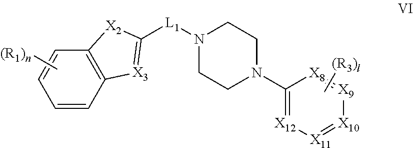

wherein

L₁ is CH₂, CHR, C(R)₂, or C═O;

R₁ is H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl;

R₃ is H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole, imidazole), substituted or unsubstituted aryl (e.g., phenyl), substituted or unsubstituted benzyl;

R₂₀ is represented by the following structure:

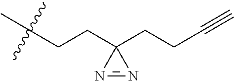 [SMILES: *CCC1(CCC#C)N=N1]

X₂ is NH, S, O, N—R (e.g., N—CH₂—CH₂—O—CH₃);

X₃ is N, C(R) (e.g., CH, C—CH₃, C—Cl, C—CN);

X₈, X₉, X₁₀, X₁₁, and X₁₂ are each independently C or N;

R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]_{q}, [C]_{q}
wherein q is between 2 and 10;

R₁₀ and R₁₁ are each independently H, C₁-C₅ substituted or unsubstituted, linear or branched alkyl (e.g., methyl, ethyl, CH₂—CH₂—O—CH₃), C₁-C₅ linear or branched alkoxy (e.g., O—CH₃), C(O)R, or S(O)₂R;

or R₁₀ and R₁₁ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;

n and 1 are each independently an integer between 0 and 4 (e.g., 0, 1 or 2);

or its pharmaceutically acceptable salt, stereoisomer, tautomer, hydrate, N-oxide, prodrug, isotopic variant (e.g., deuterated analog), reverse amide, pharmaceutical product or any combination thereof.

In various embodiments, if X₃ is N, then X₂ is not NH. In various embodiments, at least one of X₈, X₉, X₁₀, X₁₁, and X₁₂ is N;

In various embodiments, this invention is directed to a compound represented by the structure of formula VII:

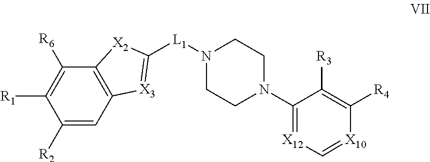

wherein

L₁ is CH₂, CHR, C(R)₂, or C═O;

R₁, R₂, and R₆ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., pyridine) ring;

or R₁ and R₆ are joined together to form a 5 or 6 membered substituted or unsubstituted, aliphatic or aromatic, carbocyclic (e.g., benzene) or heterocyclic (e.g., pyridine) ring;

R₃ and R₄ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole, imidazole), substituted or unsubstituted aryl (e.g., phenyl), substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

R₂₀ is represented by the following structure:

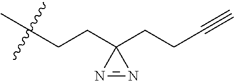 [SMILES: *CCC1(CCC#C)N=N1]

X₂ is NH, S, O, N—R (e.g., N—CH₂—CH₂—O—CH₃);

X₃ is N, C(R) (e.g., CH, C—CH₃, C—Cl, C—CN);

X₁₀ and X₁₂ are each independently C or N;

R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]_{q}, [C]_{q}
wherein q is between 2 and 10;

R₁₀ and R₁₁ are each independently H, C₁-C₅ substituted or unsubstituted, linear or branched alkyl (e.g., methyl, ethyl, CH₂—CH₂—O—CH₃), C₁-C₅ linear or branched alkoxy (e.g., O—CH₃), C(O)R, or S(O)₂R;

or R₁₀ and R₁₁ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;

or its pharmaceutically acceptable salt, stereoisomer, tautomer, hydrate, N-oxide, prodrug, isotopic variant (e.g., deuterated analog), reverse amide, pharmaceutical product or any combination thereof.

In various embodiments, if X₃ is N, then X₂ is not NH. In various embodiments, at least one of X₁₀ and X₁₂ is N;

In various embodiments, this invention is directed to a compound represented by the structure of formula VIII:

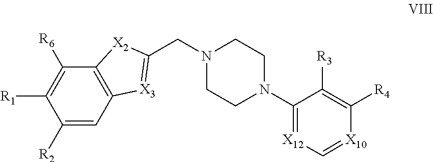

wherein

R₁ is H, O—R₂₀, CF₃, F, Cl, Br, I, OH, SH, —R₈—O—R₁₀ (e.g., CH₂—CH₂—O—CH₃, CH₂—O—CH₂—CH₂—O—CH₃), CN, NO₂, COOH, C₁-C₅ linear or branched C(O)-haloalkyl, NHC(O)—R (e.g., NHCO-Ph), —C(O)NH₂, C(O)NHR, C(O)N(R₁₀)(R₁₁), SO₂R, SO₂N(R₁₀)(R₁₁), C₁-C₅ linear or branched, substituted or unsubstituted alkyl (e.g., methyl, ethyl), C₁-C₅ linear, branched or cyclic haloalkyl (e.g., CHF₂);

R₂ is H, O—R₂₀, CF₃, F, Cl, Br, I, OH, SH, —R₈—O—R₁₀ (e.g., CH₂—CH₂—O—CH₃, CH₂—O—CH₂—CH₂—O—CH₃), CN, NO₂, COOH, C₁-C₅ linear or branched C(O)-haloalkyl, NHC(O)—R (e.g., NHCO-Ph), —C(O)NH₂, C(O)NHR, C(O)N(R₁₀)(R₁₁), SO₂R, SO₂N(R₁₀)(R₁₁), C₁-C₅ linear or branched, substituted or unsubstituted alkyl (e.g., methyl, ethyl), C₁-C₅ linear, branched or cyclic haloalkyl (e.g., CHF₂);

R₆ is H, O—R₂₀, CF₃, F, Cl, Br, I, OH, SH, —R₈—O—R₁₀ (e.g., CH₂—CH₂—O—CH₃, CH₂—O—CH₂—CH₂—O—CH₃), CN, NO₂ or C₁-C₅ linear, branched or cyclic haloalkyl (e.g., CHF₂);

R₃ is H, F, Cl, Br, I, OH, SH, O—R₂₀, CF₃, —R₈—O—R₁₀ (e.g., CH₂—CH₂—O—CH₃, CH₂—O—CH₂—CH₂—O—CH₃), C₁-C₅ linear or branched, substituted or unsubstituted alkyl (e.g., methyl, ethyl), C₁-C₅ linear, branched or cyclic haloalkyl (e.g., CHF₂), substituted or unsubstituted C₃-C₈ cycloalkyl (e.g., cyclopropyl);

R₄ is H, F, Cl, Br, I, OH, SH, O—R₂₀, CF₃, —R₈—O—R₁₀ (e.g., CH₂—CH₂—O—CH₃, CH₂—O—CH₂—CH₂—O—CH₃), C₁-C₅ linear or branched, substituted or unsubstituted alkyl (e.g., methyl, ethyl), C₁-C₅ linear, branched or cyclic haloalkyl (e.g., CHF₂), substituted or unsubstituted C₃-C₈ cycloalkyl (e.g., cyclopropyl);

R₂₀ is represented by the following structure:

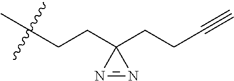 [SMILES: *CCC1(CCC#C)N=N1]

X₂ is NH, S or O;

X₃ is N, C—H, or C—Cl;

X₁₀ and X₁₂ are each independently C or N, wherein at least one of them is N;

R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]ₚ
wherein p is between 1 and 10 (e.g., 2);

R₉ is [CH]_{q}, [C]_{q}
wherein q is between 2 and 10;

R₁₀ and R₁₁ are each independently H, C₁-C₅ substituted or unsubstituted, linear or branched alkyl (e.g., methyl, ethyl, CH₂—CH₂—O—CH₃), C₁-C₅ linear or branched alkoxy (e.g., O—CH₃), C(O)R, or S(O)₂R;

or R₁₀ and R₁₁ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;
wherein if X₃ is N, then X₂ is not NH;
and wherein at least one of X₁₀ and X₁₂ is N;
or its pharmaceutically acceptable salt, stereoisomer, tautomer, hydrate, N-oxide, prodrug, isotopic variant (e.g., deuterated analog), reverse amide, pharmaceutical product or any combination thereof.

In some embodiments, the compounds of formula I-VIII is not 2-((4-(5-ethylpyrimidin-4-yl)piperazin-1-yl)methyl)-5-(trifluoromethyl)-1H-benzo[d]imidazole. In some embodiments, the compound of formula I-VIII is not PF-4708671.

In some embodiments, the compounds of formula I-VIII is not a pharmaceutically acceptable salt, stereoisomer, tautomer, hydrate, N-oxide, prodrug, isotopic variant (e.g., deuterated analog), reverse amide, or a pharmaceutical product of 2-((4-(5-ethylpyrimidin-4-yl)piperazin-1-yl)methyl)-5-(trifluoromethyl)-1H-benzo[d]imidazole.

In some embodiments, if X₃ of formula III, and/or V-VIII is N, then X₂ is not NH.

In some embodiments, if X₃ of formula III, and/or V-VIII is N, then X₂ is O or S. In some embodiments, if X₂ of formula III, and/or V-VIII is NH, then X₃ is CH or C(R).

In some embodiments, at least one of X₁₀ and X₁₂ of formula IV-VIII is N.

In some embodiments, if R₂ of formula I-VIII is CF₃, then R₃ is not ethyl.

In some embodiments, if R₃ of formula I-VIII is ethyl, then R₂ is not CF₃.

In some embodiments, at least one of R₁ and R₂ of formula I-VIII is not H. In some embodiments, R₁, R₂ and R₆ of formula I-VIII are H. In some embodiments, R₁, R₂ or R₆ is CF₃. In some embodiments, R₁, R₂ or R₆ is Cl. In some embodiments, R₁, R₂ or R₆ is CN. In some embodiments, R₁, R₂ or R₆ is NHC(O)Ph.

In some embodiments, at least one of R₃ and R₄ of formula I-VIII is not H. In some embodiments, both R₃ and R₄ are methyls. In some embodiments, both R₃ and R₄ are H. In some embodiments, R₃ is an ethyl.

In some embodiments, R₆ of formula VII and VIII is Cl. In some embodiments, R₆ is H.

In various embodiments, this invention is directed to a compound represented by the structure of formula IX:

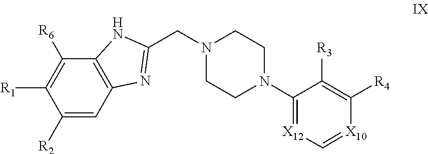

wherein

R₁, R₂ and R₆ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl;

or R₂ and R₁ are joined together to form a 5 or 6 membered substituted or unsubstituted, aliphatic or aromatic, carbocyclic (e.g., benzene) or heterocyclic (e.g., pyridine) ring;

or R₁ and R₆ are joined together to form a 5 or 6 membered substituted or unsubstituted, aliphatic or aromatic, carbocyclic (e.g., benzene) or heterocyclic (e.g., pyridine) ring;

R₃, R₄ and R₅ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole, imidazole), substituted or unsubstituted aryl (e.g., phenyl), substituted or unsubstituted benzyl;

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

X₁₀ and X₁₂ are each independently C or N;

R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or

two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;

R₈ is [CH₂]p
wherein p is between 1 and 10 (e.g., 2);

R₉ is [CH]_{q}, [C]_{q}
wherein q is between 2 and 10;

R₁₀ and R₁₁ are each independently H, C₁-C₅ substituted or unsubstituted, linear or branched alkyl (e.g., methyl, ethyl, CH₂—CH₂—O—CH₃), C₁-C₅ linear or branched alkoxy (e.g., O—CH₃), C(O)R, or S(O)₂R; or R₁₀ and R₁₁ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;
wherein at least one of X₁₀ and X₁₂ is N;
or its pharmaceutically acceptable salt, stereoisomer, tautomer, hydrate, N-oxide, prodrug, isotopic variant (e.g., deuterated analog), reverse amide, pharmaceutical product or any combination thereof.

In some embodiments, if R₃ is ethyl, then R₂ is not CF₃.

In some embodiments, if R₂ is CF₃, then R₃ is not ethyl.

In some embodiments, at least one of R₁, R₂, R₃, R₄ and R₆ is not H.

In some embodiments, at least one of R₁, R₂, and R₆ is not H.

In some embodiments, at least one of R₁ and R₂ is not H. In some embodiments, R₁, R₂ and R₆ are H. In some embodiments, R₁, R₂ or R₆ is CF₃. In some embodiments, R₁, R₂ or R₆ is Cl. In some embodiments, R₁, R₂ or R₆ is CN. In some embodiments, R₁, R₂ or R₆ is NHC(O)Ph.

In some embodiments, at least one of R₃ and R₄ is not H. In some embodiments, both R₃ and R₄ are methyls. In some embodiments, both R₃ and R₄ are H. In some embodiments, R₃ is an ethyl.

In some embodiments, R₆ is Cl. In some embodiments, R₆ is H.

In some embodiments, the compound is not 2-((4-(5-ethylpyrimidin-4-yl)piperazin-1-yl)methyl)-5-(trifluoromethyl)-1H-benzo[d]imidazole. In some embodiments, the compound is not PF-4708671.

In some embodiments, the compound is not a pharmaceutically acceptable salt, stereoisomer, tautomer, hydrate, N-oxide, prodrug, isotopic variant (e.g., deuterated analog), or a pharmaceutical product of 2-((4-(5-ethylpyrimidin-4-yl)piperazin-1-yl)methyl)-5-(trifluoromethyl)-1H-benzo[d]imidazole.

In various embodiments, this invention is directed to a compound represented by the structure of formula X:

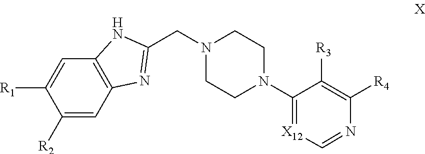

wherein

R₁ and R₂ are each independently H, Cl, F, CHF₂, or CF₃;

R₃ and R₄ are each independently H, Cl, F, CHF₂, CF₃, C₁-C₅ linear or branched, substituted or unsubstituted alkyl (e.g., methyl, ethyl), substituted or unsubstituted C₃-C₈ heterocyclic ring (e.g., imidazole), (wherein substitutions include: F, Cl, Br, I, C₁-C₅ linear or branched alkyl, OH, alkoxy, CF₃, aryl, phenyl, heteroaryl, C₃-C₈ cycloalkyl, halophenyl, (benzyloxy)phenyl, CN, NO₂ or any combination thereof);

or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;

X₁₂ is C or N;

or its pharmaceutically acceptable salt, stereoisomer, tautomer, hydrate, N-oxide, prodrug, isotopic variant (e.g., deuterated analog), reverse amide, pharmaceutical product or any combination thereof.

In some embodiments, if R₃ is ethyl, then R₂ is not CF₃. In some embodiments, if R₃ is ethyl, then R₁ is not CF₃. In some embodiments, if R₃ is ethyl, then R₂ or R₁ is not CF₃.

In some embodiments, if R₂ is CF₃, then R₃ is not ethyl. In some embodiments, if R₁ is CF₃, then R₃ is not ethyl. In some embodiments, if R₁ or R₂ is CF₃, then R₃ is not ethyl.

In some embodiments, at least one of R₁, R₂, R₃ and R₄ is not H.

In some embodiments, at least one of R₁ and R₂ is not H. In some embodiments, both R₁ and R₂ are H. In some embodiments, R₁ or R₂ is CF₃. In some embodiments, R₁ or R₂ is Cl. In some embodiments, R₁ or R₂ is CN. In some embodiments, R₁ or R₂ is NHC(O)Ph.

In some embodiments, at least one of R₃ and R₄ is not H. In some embodiments, both R₃ and R₄ are methyls. In some embodiments, both R₃ and R₄ are H. In some embodiments, R₃ is an ethyl.

In some embodiments, R₆ is Cl. In some embodiments, R₆ is H.

In some embodiments, the compound is not 2-((4-(5-ethylpyrimidin-4-yl)piperazin-1-yl)methyl)-5-(trifluoromethyl)-1H-benzo[d]imidazole. In some embodiments, the compound is not PF-4708671.

In some embodiments, the compound is not a pharmaceutically acceptable salt, stereoisomer, tautomer, hydrate, N-oxide, prodrug, isotopic variant (e.g., deuterated analog), or a pharmaceutical product of 2-((4-(5-ethylpyrimidin-4-yl)piperazin-1-yl)methyl)-5-(trifluoromethyl)-1H-benzo[d]imidazole.

In some embodiments, A of formula I, I(a), II, and/or IV is a phenyl. In other embodiments, A is pyridinyl. In other embodiments, A is 2-pyridinyl. In other embodiments, A is 3-pyridinyl. In other embodiments, A is 4-pyridinyl. In other embodiments, A is pyrimidine. In other embodiments, A is pyridazine. In other embodiments, A is pyrazine. In other embodiments, A is naphthyl. In other embodiments, A is benzothiazolyl. In other embodiments, A is benzimidazolyl. In other embodiments, A is quinolinyl. In other embodiments, A is isoquinolinyl. In other embodiments, A is indolyl. In other embodiments, A is benzoxazole. In other embodiments, A is imidazopyridin. In other embodiments, A is pyrazolopyridine. In other embodiments, A is pyrrolopyridine. In other embodiments, A is tetrahydronaphthyl. In other embodiments, A is indenyl. In other embodiments, A is benzofuran-2(3H)-one. In other embodiments, A is benzo[d][1,3]dioxole. In other embodiments, A is tetrahydrothiophenel, 1-dioxide. In other embodiments, A is thiazole. In others embodiment, A is piperidine. In other embodiments, A is 1-methylpiperidine. In other embodiments, A is imidazole. In other embodiments, A is 1-methylimidazole. In other embodiments, A is thiophene. In other embodiments, A is isoquinoline. In other embodiments, A is 1,3-dihydroisobenzofuran. In other embodiments, A is benzofuran. In other embodiments, A is single or fused C₃-C₁₀ cycloalkyl ring. In other embodiments, A is cyclohexyl.

In some embodiments, B of formula I, I(a) and/or II is a phenyl ring. In other embodiments, B is pyridinyl. In other embodiments, B is 2-pyridinyl. In other embodiments, B is 3-pyridinyl. In other embodiments, B is 4-pyridinyl. In other embodiments, B is pyrimidine. In other embodiments, B is pyridazine. In other embodiments, B is pyrazine. In other embodiments, B is thiazole. In other embodiments, B is imidazole. In other embodiments, B is indazole. In other embodiments, B is pyrrole. In other embodiments, B is triazole. In other embodiments, B is naphthyl. In other embodiments, B is indolyl. In other embodiments, B is benzimidazolyl. In other embodiments, B is benzothiazolyl. In other embodiments, B is quinoxalinyl. In other embodiments, B is tetrahydronaphthyl. In other embodiments, B is quinolinyl. In other embodiments, B is isoquinolinyl. In other embodiments, B is indenyl. In other embodiments, B is naphthalene. In other embodiments, B is tetrahydrothiophenel, 1-dioxide. In other embodiments, B is benzimidazole. In other embodiments, B is piperidine. In other embodiments, B is 1-methylpiperidine. In other embodiments, B is 1-methylimidazole. In other embodiments, B is thiophene. In other embodiments, B is isoquinoline. In other embodiments, B is indole. In other embodiments, B is 1,3-dihydroisobenzofuran. In other embodiments, B is benzofuran. In other embodiments, B is single or fused C₃-C₁₀ cycloalkyl ring. In other embodiments, B is cyclohexyl.

In some embodiments, X₁ of compound of formula I, I(a), II, III and/or IV is N. In other embodiments, X₁ is C—R. In other embodiments, X₁ is C—H. In other embodiments, X₁ is C—OH.

In some embodiments, X₂ of compound of formula III, V, V(a), VI, VII and/or VIII is NH. In other embodiments, X₂ is S. In other embodiments, X₂ is O. In other embodiments, X₂ is N—R. In other embodiments, X₂ is N—CH₂—CH₂—O—CH₃.

In some embodiments, X₃ of compound of formula III, V, V(a), VI, VII and/or VIII is N. In other embodiments, X₃ is C(R). In other embodiments, X₃ is CH. In other embodiments, X₃ is C—CH₃. In other embodiments, X₃ is C—Cl. In other embodiments, X₃ is C—CN.

In various embodiments, if X₃ of compound of formula III, V, V(a), VI, VII and/or VIII is N, then X₂ is not NH.

In some embodiments, X₄ of compound of formula III, V and/or V(a) is C. In other embodiments, X₄ is N.

In some embodiments, X₅ of compound of formula III, V and/or V(a) is C. In other embodiments, X₅ is N.

In some embodiments, X₆ of compound of formula III, V and/or V(a) is C. In other embodiments, X₆ is N.

In some embodiments, X₇ of compound of formula III, V and/or V(a) is C. In other embodiments, X₇ is N.

In some embodiments, X₈ of compound of formula IV, V, V(a) and/or VI is C. In other embodiments, X₈ is N.

In some embodiments, X₉ of compound of formula IV, V, V(a) and/or VI is C. In other embodiments, X₉ is N.

In some embodiments, X₁₀ of compound of formula IV-VIII and/or IX is C. In other embodiments, X₁₀ is N.

In some embodiments, X₁₁ of compound of formula IV, V, V(a) and/or VI is C. In other embodiments, X₁₁ is N.

In some embodiments, X₁₂ of compound of formula IV-VIII, IX, V and/or V(a) is C. In other embodiments, X₁₂ is N.

In some embodiments, at least one of X₄-X₇ is N.

In some embodiments, at least one of X₈-X₁₂ is N. In some embodiments, at least two of X₈-X₁₂ are N.

It is understood that if any of X₄-X₁₂ are N, then any of R₁—R₄ cannot be attached thereto.

In some embodiments, R₁ of formula X is H. In some embodiments, R₁ is Cl. In some embodiments, R₁ is F. In some embodiments, R₁ is CF₃. In some embodiments, R₁ is CHF₂.

In some embodiments, R₁ of formula I-IX is H. In some embodiments, R₁ is C₁-C₅ linear or branched, substituted or unsubstituted alkyl. In other embodiments, R₁ is methyl. In other embodiments, R₁ is ethyl. In other embodiments, R₁ is iso-propyl. In other embodiments, R₁ is t-Bu. In other embodiments, R₁ is iso-butyl. In other embodiments, R₁ is pentyl. In other embodiments, R₁ is propyl. In other embodiments, R₁ is benzyl. In other embodiments, R₁ is in the ortho position. In other embodiments, R₁ is an ortho-methyl.

In other embodiments, R₁ of formula I-IX is F. In other embodiments, R₁ is Cl. In other embodiments, R₁ is Br. In other embodiments, R₁ is —R₈—O—R₁₀. In other embodiments, R₁ is CH₂—CH₂—O—CH₃. In other embodiments, R₁ is CH₂—O—CH₂—CH₂—O—CH₃. In other embodiments, R₁ is —O—R₈—O—R₁₀. In other embodiments, R₁ is O—CH₂—CH₂—O—CH₃. In other embodiments, R₁ is I. In other embodiments, R₁ is R₈—(C₃-C₈ cycloalkyl). In other embodiments, R₁ is CH₂-cyclohexyl. In other embodiments, R₁ is R₈—(C₃-C₈ heterocyclic ring). In other embodiments, R₁ is CH₂-imidazole. In other embodiments, R₁ is CH₂-indazole. In other embodiments, R₁ is CF₃. In other embodiments, R₁ is CN. In other embodiments, R₁ is NH₂. In other embodiments, R₁ is C₁-C₅ linear, branched or cyclic haloalkyl. In other embodiments, R₁ is CHF₂. In other embodiments, R₁ is CF₂CH₂CH₃. In other embodiments, R₁ is CH₂CH₂CF₃. In other embodiments, R₁ is CF₂CH(CH₃)₂. In other embodiments, R₁ is CF(CH₃)—CH(CH₃)₂. In other embodiments, R₁ is OCD₃. In other embodiments, R₁ is NO₂. In other embodiments, R₁ is NH₂. In other embodiments, R₁ is R₈—N(R₁₀)(R₁₁). In other embodiments, R₁ is CH₂—NH₂. In other embodiments, R₁ is CH₂—N(CH₃)₂). In other embodiments, R₁ is R₉—R₈—N(R₁₀)(R₁₁). In other embodiments, R₁ is C═C—CH₂—NH₂. In other embodiments, R₁ is B(OH)₂. In other embodiments, R₁ is NHC(O)—R₁₀. In other embodiments, R₁ is NHC(O)CH₃. In other embodiments, R₁ is NHC(O)—R. In other embodiments, R₁ is NHCO-Ph. In other embodiments, R₁ is NHCO—N(R₁₀)(R₁₁). In other embodiments, R₁ is NHC(O)N(CH₃)₂. In other embodiments, R₁ is COOH. In other embodiments, R₁ is C(O)O—R₁₀. In other embodiments, R₁ is C(O)O—CH(CH₃)₂. In other embodiments, R₁ is C(O)O—CH₃. In other embodiments, R₁ is SO₂N(R₁₀)(R₁₁). In other embodiments, R₁ is SO₂N(CH₃)₂. In other embodiments, R₁ is SO₂NHC(O)CH₃. In other embodiments, R₁ is O—R₂₀. In other embodiments, R₁ is NHSO₂(R₁₀). In other embodiments, R₁ is NHSO₂CH₃. In other embodiments, R₁ is C₁-C₅ linear or branched, substituted or unsubstituted alkyl. In other embodiments, R₁ is methyl. In other embodiments, R₁ is ethyl. In other embodiments, R₁ is iso-propyl. In other embodiments, R₁ is t-Bu. In other embodiments, R₁ is iso-butyl. In other embodiments, R₁ is pentyl. In other embodiments, R₁ is propyl. In other embodiments, R₁ is benzyl. In other embodiments, R₁ is C₁-C₅ linear or branched, substituted or unsubstituted alkenyl. In other embodiments, R₁ is CH═C(Ph)₂. In other embodiments, R₁ is 2-CH₂—C₆H₄—Cl. In other embodiments, R₁ is 3-CH₂—C₆H₄—Cl. In other embodiments, R₁ is 4-CH₂—C₆H₄—Cl. In other embodiments, R₁ is ethyl. In other embodiments, R₁ is iso-propyl. In other embodiments, R₁ is t-Bu. In other embodiments, R₁ is iso-butyl. In other embodiments, R₁ is pentyl. In other embodiments, R₁ is substituted or unsubstituted C₃-C₈ cycloalkyl (e.g., cyclopropyl, cyclopentyl). In other embodiments, R₁ is C₁-C₅ linear, branched or cyclic alkoxy. In other embodiments, R₁ is methoxy. In other embodiments, R₁ is ethoxy. In other embodiments, R₁ is propoxy. In other embodiments, R₁ is isopropoxy. In other embodiments, R₁ is O—CH₂-cyclopropyl. In other embodiments, R₁ is O-cyclobutyl. In other embodiments, R₁ is O-cyclopentyl. In other embodiments, R₁ is O-cyclohexyl. In other embodiments, R₁ is O-1-oxacyclobutyl. In other embodiments, R₁ is O-2-oxacyclobutyl. In other embodiments, R₁ is 1-butoxy. In other embodiments, R₁ is 2-butoxy. In other embodiments, R₁ is O-tBu. In other embodiments, R₁ is C₁-C₅ linear, branched or cyclic alkoxy wherein at least one methylene group (CH₂) in the alkoxy is replaced with an oxygen atom (O). In other embodiments, R₁ is O-1-oxacyclobutyl. In other embodiments, R₁ is O-2-oxacyclobutyl. In other embodiments, R₁ is C₁-C₅ linear or branched haloalkoxy. In other embodiments, R₁ is OCF₃. In other embodiments, R₁ is OCHF₂. In other embodiments, R₁ is substituted or unsubstituted aryl. In other embodiments, R₁ is phenyl. In other embodiments, R₁ is substituted or unsubstituted C₃-C₈ heterocyclic ring. In other embodiments, R₁ is oxazole. In other embodiments, R₁ is methyl substituted oxazole. In other embodiments, R₁ is oxadiazole. In other embodiments, R₁ is methyl substituted oxadiazole. In other embodiments, R₁ is imidazole. In other embodiments, R₁ is methyl substituted imidazole. In other embodiments, R₁ is pyridine. In other embodiments, R₁ is 2-pyridine. In other embodiments, R₁ is 3-pyridine. In other embodiments, R₁ is 4-pyridine. In other embodiments, R₁ is tetrazole. In other embodiments, R₁ is pyrimidine. In other embodiments, R₁ is pyrazine. In other embodiments, R₁ is oxacyclobutane. In other embodiments, R₁ is 1-oxacyclobutane. In other embodiments, R₁ is 2-oxacyclobutane. In other embodiments, R₁ is indole. In other embodiments, R₁ is pyridine oxide. In other embodiments, R₁ is protonated pyridine oxide. In other embodiments, R₁ is deprotonated pyridine oxide. In other embodiments, R₁ is 3-methyl-4H-1,2,4-triazole. In other embodiments, R₁ is 5-methyl-1,2,4-oxadiazole. In other embodiments, R₁ is substituted or unsubstituted aryl. In other embodiments, R₁ is phenyl. In other embodiments, R₁ is bromophenyl. In other embodiments, R₁ is 2-bromophenyl. In other embodiments, R₁ is 3-bromophenyl. In other embodiments, R₁ is 4-bromophenyl. In other embodiments, R₁ is substituted or unsubstituted benzyl. In other embodiments, R₁ is benzyl. In other embodiments, R₁ is R₈—N(R₁₀)(R₁₁). In other embodiments, R₁ is CH₂—NH₂. In other embodiments, substitutions include: C₁-C₅ linear or branched alkyl (e.g. methyl), aryl, phenyl, heteroaryl (e.g., imidazole), and/or C₃-C₈ cycloalkyl, each is a separate embodiment according to this invention.

In some embodiments, R₂ of formula X is H. In some embodiments, R₂ is Cl. In some embodiments, R₂ is F. In some embodiments, R₂ is CF₃. In some embodiments, R₂ is CHF₂.

In some embodiments, R₂ of formula I-IX is H. In some embodiments, R₂ is C₁-C₅ linear or branched, substituted or unsubstituted alkyl. In other embodiments, R₂ is methyl. In other embodiments, R₂ is ethyl. In other embodiments, R₂ is iso-propyl. In other embodiments, R₂ is t-Bu. In other embodiments, R₂ is iso-butyl. In other embodiments, R₂ is pentyl. In other embodiments, R₂ is propyl. In other embodiments, R₂ is benzyl. In other embodiments, R₂ is in the ortho position. In other embodiments, R₂ is an ortho-methyl.

In some embodiments, R₂ of formula I-IX is F. In other embodiments, R₂ is Cl. In other embodiments, R₂ is Br. In other embodiments, R₂ is I. In other embodiments, R₂ is R₈—O—R₁₀. In other embodiments, R₂ is CH₂—CH₂—O—CH₃. In other embodiments, R₂ is CH₂—O—CH₂—CH₂—O—CH₃. In other embodiments, R₂ is —O—R₈—O—R₁₀. In other embodiments, R₂ is O—CH₂—CH₂—O—CH₃. In other embodiments, R₂ is I. In other embodiments, R₂ is R₈—(C₃-C₈ cycloalkyl). In other embodiments, R₂ is CH₂-cyclohexyl. In other embodiments, R₂ is R₈—(C₃-C₈ heterocyclic ring). In other embodiments, R₂ is CH₂-imidazole. In other embodiments, R₂ is CH₂-indazole. In other embodiments, R₂ is CF₃. In other embodiments, R₂ is CN. In other embodiments, R₂ is NH₂. In other embodiments, R₂ is C₁-C₅ linear, branched or cyclic haloalkyl. In other embodiments, R₂ is CHF₂. In other embodiments, R₂ is CF₂CH₂CH₃. In other embodiments, R₂ is CH₂CH₂CF₃. In other embodiments, R₂ is CF₂CH(CH₃)₂. In other embodiments, R₂ is CF(CH₃)—CH(CH₃)₂. In other embodiments, R₂ is OCD₃. In other embodiments, R₂ is NO₂. In other embodiments, R₂ is NH₂. In other embodiments, R₂ is R₈—N(R₁₀)(R₁₁). In other embodiments, R₂ is CH₂—NH₂. In other embodiments, R₂ is CH₂—N(CH₃)₂). In other embodiments, R₂ is R₉—R₈—N(R₁₀)(R₁₁). In other embodiments, R₂ is C═C—CH₂—NH₂. In other embodiments, R₂ is B(OH)₂. In other embodiments, R₂ is NHC(O)—R₁₀. In other embodiments, R₂ is NHC(O)CH₃. In other embodiments, R₂ is NHC(O)—R. In other embodiments, R₂ is NHCO-Ph. In other embodiments, R₂ is NHCO—N(R₁₀)(R₁₁). In other embodiments, R₂ is NHC(O)N(CH₃)₂. In other embodiments, R₂ is COOH. In other embodiments, R₂ is C(O)O—R₁₀. In other embodiments, R₂ is C(O)O—CH(CH₃)₂. In other embodiments, R₂ is C(O)O—CH₃. In other embodiments, R₂ is SO₂N(R₁₀)(R₁₁). In other embodiments, R₂ is SO₂N(CH₃)₂. In other embodiments, R₂ is SO₂NHC(O)CH₃. In other embodiments, R₂ is NHSO₂(R₁₀). In other embodiments, R₂ is NHSO₂CH₃. In other embodiments, R₂ is C₁-C₅ linear or branched, substituted or unsubstituted alkyl. In other embodiments, R₂ is methyl. In other embodiments, R₂ is ethyl. In other embodiments, R₂ is iso-propyl. In other embodiments, R₂ is t-Bu. In other embodiments, R₂ is iso-butyl. In other embodiments, R₂ is pentyl. In other embodiments, R₂ is propyl. In other embodiments, R₂ is benzyl. In other embodiments, R₂ is C₁-C₅ linear or branched, substituted or unsubstituted alkenyl. In other embodiments, R₂ is CH═C(Ph)₂. In other embodiments, R₂ is 2-CH₂—C₆H₄—Cl. In other embodiments, R₂ is 3-CH₂—C₆H₄—Cl. In other embodiments, R₂ is 4-CH₂—C₆H₄—Cl. In other embodiments, R₂ is ethyl. In other embodiments, R₂ is iso-propyl. In other embodiments, R₂ is t-Bu. In other embodiments, R₂ is iso-butyl. In other embodiments, R₂ is pentyl. In other embodiments, R₂ is substituted or unsubstituted C₃-C₈ cycloalkyl (e.g., cyclopropyl, cyclopentyl). In other embodiments, R₂ is C₁-C₅ linear, branched or cyclic alkoxy. In other embodiments, R₂ is methoxy. In other embodiments, R₂ is ethoxy. In other embodiments, R₂ is propoxy. In other embodiments, R₂ is isopropoxy. In other embodiments, R₂ is O—CH₂-cyclopropyl. In other embodiments, R₂ is O-cyclobutyl. In other embodiments, R₂ is O-cyclopentyl. In other embodiments, R₂ is O-cyclohexyl. In other embodiments, R₂ is O-1-oxacyclobutyl. In other embodiments, R₂ is O-2-oxacyclobutyl. In other embodiments, R₂ is 1-butoxy. In other embodiments, R₂ is 2-butoxy. In other embodiments, R₂ is O-tBu. In other embodiments, R₂ is C₁-C₅ linear or branched haloalkoxy. In other embodiments, R₂ is OCF₃. In other embodiments, R₂ is OCHF₂. In other embodiments, R₂ is O—R₂₀. In other embodiments, R₂ is a substituted or unsubstituted aryl. In other embodiments, R₂ is phenyl. In other embodiments, R₂ is substituted or unsubstituted C₃-C₈ heterocyclic ring. In other embodiments, R₂ is oxazole or methyl substituted oxazole. In other embodiments, R₂ is oxadiazole or methyl substituted oxadiazole. In other embodiments, R₂ is imidazole or methyl substituted imidazole. In other embodiments, R₂ is pyridine. In other embodiments, R₂ is 2-pyridine. In other embodiments, R₂ is 3-pyridine. In other embodiments, R₂ is 4-pyridine. In other embodiments, R₂ is tetrazole. In other embodiments, R₂ is pyrimidine. In other embodiments, R₂ is pyrazine. In other embodiments, R₂ is oxacyclobutane. In other embodiments, R₂ is 1-oxacyclobutane. In other embodiments, R₂ is 2-oxacyclobutane. In other embodiments, R₂ is indole. In other embodiments, R₂ is pyridine oxide. In other embodiments, R₂ is protonated pyridine oxide. In other embodiments, R₂ is deprotonated pyridine oxide. In other embodiments, R₂ is 3-methyl-4H-1,2,4-triazole. In other embodiments, R₂ is 5-methyl-1,2,4-oxadiazole. In other embodiments, R₂ is substituted or unsubstituted aryl. In other embodiments, R₂ is phenyl. In other embodiments, R₂ is bromophenyl. In other embodiments, R₂ is 2-bromophenyl. In other embodiments, R₂ is 3-bromophenyl. In other embodiments, R₂ is 4-bromophenyl. In other embodiments, R₂ is substituted or unsubstituted benzyl. In other embodiments, R₂ is benzyl. In other embodiments, R₂ is R₈—N(R₁₀)(R₁₁). In other embodiments, R₂ is CH₂—NH₂. In other embodiments, substitutions include: C₁-C₅ linear or branched alkyl (e.g. methyl), aryl, phenyl, heteroaryl (e.g., imidazole), and/or C₃-C₈ cycloalkyl, each is a separate embodiment according to this invention.

In some embodiments, R₁ and R₂ of formula, I-IX are joined together to form a pyrrol ring. In some embodiments, R₁ and R₂ are joined together to form a benzene ring. In some embodiments, R₁ and R₂ are joined together to form a pyridine ring. In some embodiments, R₁ and R₂ are joined together to form a [1,3]dioxole ring. In some embodiments, R₁ and R₂ are joined together to form a furanone ring (e.g., furan-2(3H)-one).

In some embodiments, R₃ of formula X is H. In other embodiments, R₃ is C₁-C₅ linear or branched, substituted or unsubstituted alkyl. In other embodiments, R₃ is methyl. In other embodiments, R₃ is ethyl. In other embodiments, R₃ is F. In other embodiments, R₃ is Cl. In other embodiments, R₃ is CF₃. In other embodiments, R₃ is CHF₂.

In some embodiments, R₃ of formula I-IX is H. In other embodiments, R₃ is Cl. In other embodiments, R₃ is I. In other embodiments, R₃ is F. In other embodiments, R₃ is Br. In other embodiments, R₃ is CF₃. In other embodiments, R₃ is CHF₂. In other embodiments, R₃ is CN. In other embodiments, R₃ is OH. In other embodiments, R₃ is CD₃. In other embodiments, R₃ is OCD₃. In other embodiments, R₃ is R₈—OH. In other embodiments, R₃ is CH₂—OH. In other embodiments, R₃ is —R₈—O—R₁₀. In other embodiments, R₃ is CH₂—O—CH₂—CH₂—O—CH₃. In other embodiments, R₃ is CH₂—O—CH₃. In other embodiments, R₃ is R₈—N(R₁₀)(R₁₁). In other embodiments, R₃ is CH₂—NH₂. In other embodiments, R₃ is CH₂—N(CH₃)₂. In other embodiments, R₃ is COOH. In other embodiments, R₃ is C(O)O—R₁₀. In other embodiments, R₃ is C(O)O—CH₂CH₃. In other embodiments, R₃ is R₈—C(O)—R₁₀. In other embodiments, R₃ is CH₂C(O)CH₃. In other embodiments, R₃ is C(O)—R₁₀. In other embodiments, R₃ is C(O)—H. In other embodiments, R₃ is C(O)—CH₃. In other embodiments, R₃ is C(O)—CH₂CH₃. In other embodiments, R₃ is C(O)—CH₂CH₂CH₃. In other embodiments, R₃ is C₁-C₅ linear or branched C(O)-haloalkyl. In other embodiments, R₃ is C(O)—CF₃. In other embodiments, R₃ is C(O)N(R₁₀)(R₁₁). In other embodiments, R₃ is C(O)N(CH₃)₂). In other embodiments, R₃ is SO₂N(R₁₀)(R₁₁). In other embodiments, R₃ is SO₂N(CH₃)₂. In other embodiments, R₃ is O—R₂₀. In other embodiments, R₃ is C₁-C₅ linear or branched, substituted or unsubstituted alkyl. In other embodiments, R₃ is methyl. In other embodiments, R₃ is ethyl. In other embodiments, R₃ is propyl. In other embodiments, R₃ is iso-propyl. In other embodiments, R₃ is t-Bu. In other embodiments, R₃ is iso-butyl. In other embodiments, R₃ is pentyl. In other embodiments, R₃ is C(OH)(CH₃)(Ph). In other embodiments, R₃ is C₁-C₅ linear, branched or cyclic haloalkyl. In other embodiments, R₃ is CF₂CH₃. In other embodiments, R₃ is CF₂-cyclobutyl. In other embodiments, R₃ is CH₂CF₃. In other embodiments, R₃ is CF₂CH₂CH₃. In other embodiments, R₃ is CF₃. In other embodiments, R₃ is CF₂CH₂CH₃. In other embodiments, R₃ is CH₂CH₂CF₃. In other embodiments, R₃ is CF₂CH(CH₃)₂. In other embodiments, R₃ is CF(CH₃)—CH(CH₃)₂. In other embodiments, R₃ is C₁-C₅ linear, branched or cyclic alkoxy. In other embodiments, R₃ is methoxy. In other embodiments, R₃ is isopropoxy. In other embodiments, R₃ is substituted or unsubstituted C₃-C₈ cycloalkyl. In other embodiments, R₃ is cyclopropyl. In other embodiments, R₃ is cyclopentyl. In other embodiments, R₃ is substituted or unsubstituted C₃-C₈ heterocyclic ring. In other embodiments, R₃ is pyrazole. In other embodiments, R₃ is thiazole. In other embodiments, R₃ is thiophene. In other embodiments, R₃ is oxazole. In other embodiments, R₃ is isoxazole. In other embodiments, R₃ is imidazole. In other embodiments, R₃ is furane. In other embodiments, R₃ is triazole. In other embodiments, R₃ is pyridine. In other embodiments, R₃ is 2-pyridine. In other embodiments, R₃ is 3-pyridine. In other embodiments, R₃ is 4-pyridine. In other embodiments, R₃ is pyrimidine. In other embodiments, R₃ is pyrazine. In other embodiments, R₃ is oxacyclobutane. In other embodiments, R₃ is 1-oxacyclobutane. In other embodiments, R₃ is 2-oxacyclobutane. In other embodiments, R₃ is indole. In other embodiments, R₃ is 3-methyl-4H-1,2,4-triazole. In other embodiments, R₃ is 5-methyl-1,2,4-oxadiazole. In other embodiments, R₃ is substituted or unsubstituted aryl. In other embodiments, R₃ is phenyl. In other embodiments, R₃ is CH(CF₃)(NH—R₁₀).

In some embodiments, R₄ of formula X is H. In other embodiments, R₄ is C₁-C₅ linear or branched, substituted or unsubstituted alkyl. In other embodiments, R₄ is methyl. In other embodiments, R₄ is ethyl. In other embodiments, R₄ is F. In other embodiments, R₄ is Cl. In other embodiments, R₄ is CF₃. In other embodiments, R₄ is CHF₂. In other embodiments, R₄ is CN. In some embodiments, R₄ of formula I-V, and/or VII-IX is H. In other embodiments, R₄ is Cl. In other embodiments, R₄ is I. In other embodiments, R₄ is F. In other embodiments, R₄ is Br. In other embodiments, R₄ is CF₃. In other embodiments, R₄ is CHF₂. In other embodiments, R₄ is OH. In other embodiments, R₄ is CD₃. In other embodiments, R₄ is OCD₃. In other embodiments, R₄ is R₈—OH. In other embodiments, R₄ is CH₂—OH. In other embodiments, R₄ is —R₈—O—R₁₀. In other embodiments, R₄ is CH₂—O—CH₂—CH₂—O—CH₃. In other embodiments, R₄ is CH₂—O—CH₃. In other embodiments, R₄ is R₈—N(R₁₀)(R₁₁). In other embodiments, R₄ is CH₂—NH₂. In other embodiments, R₄ is CH₂—N(CH₃)₂. In other embodiments, R₄ is COOH. In other embodiments, R₄ is C(O)O—R₁₀. In other embodiments, R₄ is C(O)O—CH₂CH₃. In other embodiments, R₄ is R₈—C(O)—R₁₀. In other embodiments, R₄ is CH₂C(O)CH₃. In other embodiments, R₄ is C(O)—R₁₀. In other embodiments, R₄ is C(O)—H. In other embodiments, R₄ is C(O)—CH₃. In other embodiments, R₄ is C(O)—CH₂CH₃. In other embodiments, R₄ is C(O)—CH₂CH₂CH₃. In other embodiments, R₄ is C₁-C₅ linear or branched C(O)-haloalkyl. In other embodiments, R₄ is C(O)—CF₃. In other embodiments, R₄ is C(O)N(R₁₀)(R₁₁). In other embodiments, R₄ is C(O)N(CH₃)₂). In other embodiments, R₄ is SO₂N(R₁₀)(R₁₁). In other embodiments, R₄ is SO₂N(CH₃)₂. In other embodiments, R₄ is O—R₂₀. In other embodiments, R₄ is C₁-C₅ linear or branched, substituted or unsubstituted alkyl. In other embodiments, R₄ is methyl. In other embodiments, R₄ is C(OH)(CH₃)(Ph). In other embodiments, R₄ is ethyl. In other embodiments, R₄ is propyl. In other embodiments, R₄ is iso-propyl. In other embodiments, R₄ is t-Bu. In other embodiments, R₄ is iso-butyl. In other embodiments, R₄ is pentyl. In other embodiments, R₄ is C₁-C₅ linear, branched or cyclic haloalkyl. In other embodiments, R₃ is CF₂CH₃. In other embodiments, R₃ is CF₂-cyclobutyl. In other embodiments, R₄ is CH₂CF₃. In other embodiments, R₄ is CF₂CH₂CH₃. In other embodiments, R₄ is CF₃. In other embodiments, R₄ is CF₂CH₂CH₃. In other embodiments, R₄ is CH₂CH₂CF₃. In other embodiments, R₄ is CF₂CH(CH₃)₂. In other embodiments, R₄ is CF(CH₃)—CH(CH₃)₂. In other embodiments, R₄ is C₁-C₅ linear, branched or cyclic alkoxy. In other embodiments, R₄ is methoxy. In other embodiments, R₄ is isopropoxy. In other embodiments, R₄ is substituted or unsubstituted C₃-C₈ cycloalkyl. In other embodiments, R₄ is cyclopropyl. In other embodiments, R₄ is cyclopentyl. In other embodiments, R₄ is substituted or unsubstituted C₃-C₈ heterocyclic ring. In other embodiments, R₄ is pyrazole. In other embodiments, R₄ is thiazole. In other embodiments, R₄ is thiophene. In other embodiments, R₄ is oxazole. In other embodiments, R₄ is isoxazole. In other embodiments, R₄ is imidazole. In other embodiments, R₄ is furane. In other embodiments, R₄ is triazole. In other embodiments, R₄ is pyridine. In other embodiments, R₄ is 2-pyridine. In other embodiments, R₄ is 3-pyridine. In other embodiments, R₄ is 4-pyridine. In other embodiments, R₄ is pyrimidine. In other embodiments, R₄ is pyrazine. In other embodiments, R₄ is oxacyclobutane. In other embodiments, R₄ is 1-oxacyclobutane. In other embodiments, R₄ is 2-oxacyclobutane. In other embodiments, R₄ is indole. In other embodiments, R₄ is 3-methyl-4H-1,2,4-triazole. In other embodiments, R₄ is 5-methyl-1,2,4-oxadiazole. In other embodiments, R₄ is substituted or unsubstituted aryl. In other embodiments, R₄ is phenyl. In other embodiments, R₄ is CH(CF₃)(NH—R₁₀).

In some embodiments, R₅ of formula I, I(a), II, III, IV, V and V(a) is H. In other embodiments, R₅ is Cl. In other embodiments, R₅ is I. In other embodiments, R₅ is F. In other embodiments, R₅ is Br. In other embodiments, R₅ is OH. In other embodiments, R₅ is CD₃. In other embodiments, R₅ is OCD₃. In other embodiments, R₅ is R₈—OH. In other embodiments, R₅ is CH₂—OH. In other embodiments, R₅ is —R₈—O—R₁₀. In other embodiments, R₅ is CH₂—O—CH₃. In other embodiments, R₅ is R₈—N(R₁₀)(R₁₁). In other embodiments, R₅ is CH₂—NH₂. In other embodiments, R₅ is CH₂—N(CH₃)₂. In other embodiments, R₅ is COOH. In other embodiments, R₅ is C(O)O—R₁₀. In other embodiments, R₅ is C(O)O—CH₂CH₃. In other embodiments, R₅ is R₈—C(O)—R₁₀. In other embodiments, R₅ is CH₂C(O)CH₃. In other embodiments, R₅ is C(O)—R₁₀. In other embodiments, R₅ is C(O)—CH₃. In other embodiments, R₄ is C(O)—CH₂CH₃. In other embodiments, R₅ is C(O)—CH₂CH₂CH₃. In other embodiments, R₅ is C₁-C₅ linear or branched C(O)-haloalkyl. In other embodiments, R₅ is C(O)—CF₃. In other embodiments, R₅ is C(O)N(R₁₀)(R₁₁). In other embodiments, R₅ is C(O)N(CH₃)₂). In other embodiments, R₅ is SO₂N(R₁₀)(R₁₁). In other embodiments, R₅ is SO₂N(CH₃)₂. In other embodiments, R₅ is O—R₂₀. In other embodiments, R₄ is C₁-C₅ linear or branched, substituted or unsubstituted alkyl. In other embodiments, R₅ is methyl. In other embodiments, R₅ is C(OH)(CH₃)(Ph). In other embodiments, R₅ is ethyl. In other embodiments, R₅ is propyl. In other embodiments, R₅ is iso-propyl. In other embodiments, R₅ is t-Bu. In other embodiments, R₅ is iso-butyl. In other embodiments, R₅ is pentyl. In other embodiments, R₅ is C₁-C₅ linear, branched or cyclic haloalkyl. In other embodiments, R₅ is CF₂CH₃. In other embodiments, R₅ is CF₂-cyclobutyl. In other embodiments, R₅ is CH₂CF₃. In other embodiments, R₅ is CHF₂. In other embodiments, R₅ is CF₂CH₂CH₃. In other embodiments, R₅ is CF₃. In other embodiments, R₄ is CF₂CH₂CH₃. In other embodiments, R₅ is CH₂CH₂CF₃. In other embodiments, R₅ is CF₂CH(CH₃)₂. In other embodiments, R₅ is CF(CH₃)—CH(CH₃)₂. In other embodiments, R₅ is C₁-C₅ linear, branched or cyclic alkoxy. In other embodiments, R₅ is methoxy. In other embodiments, R₅ is isopropoxy. In other embodiments, R₅ is substituted or unsubstituted C₃-C₈ cycloalkyl. In other embodiments, R₅ is cyclopropyl. In other embodiments, R₅ is cyclopentyl. In other embodiments, R₅ is substituted or unsubstituted C₃-C₈ heterocyclic ring. In other embodiments, R₅ is pyrazole. In other embodiments, R₅ is thiazole. In other embodiments, R₅ is thiophene. In other embodiments, R₅ is oxazole. In other embodiments, R₅ is isoxazole. In other embodiments, R₅ is imidazole. In other embodiments, R₅ is furane. In other embodiments, R₅ is triazole. In other embodiments, R₅ is pyridine. In other embodiments, R₅ is 2-pyridine. In other embodiments, R₄ is 3-pyridine. In other embodiments, R₅ is 4-pyridine. In other embodiments, R₅ is pyrimidine. In other embodiments, R₅ is pyrazine. In other embodiments, R₅ is oxacyclobutane. In other embodiments, R₅ is 1-oxacyclobutane. In other embodiments, R₅ is 2-oxacyclobutane. In other embodiments, R₅ is indole. In other embodiments, R₅ is 3-methyl-4H-1,2,4-triazole. In other embodiments, R₅ is 5-methyl-1,2,4-oxadiazole. In other embodiments, R₅ is substituted or unsubstituted aryl. In other embodiments, R₅ is phenyl. In other embodiments, R₅ is CH(CF₃)(NH—R₁₀).

In some embodiments, R₃ and R₄ of formula I, I(a), II, III, IV, V and V(a) are joined together to form a 5 or 6 membered substituted or unsubstituted, aliphatic ring. In some embodiments, R₃ and R₄ are joined together to form a cyclopentene. In some embodiments, R₃ and R₄ are joined together to form an aromatic carbocyclic ring. In some embodiments, R₃ and R₄ are joined together to form a benzene. In some embodiments, R₃ and R₄ are joined together to form an aromatic heterocyclic ring. In some embodiments, R₃ and R₄ are joined together to form a thiophene. In some embodiments, R₃ and R₄ are joined together to form a furane. In some embodiments, R₃ and R₄ are joined together to form a pyrrol. In some embodiments, R₃ and R₄ are joined together to form a pyrazole ring. a [1,3]dioxole ring. In some embodiments, R₃ and R₄ are joined together to form a furanone ring (e.g., furan-2(3H)-one). In some embodiments, R₃ and R₄ are joined together to form a cyclopentene ring. In some embodiments, R₃ and R₄ are joined together to form an imidazole ring.

In some embodiments, L₁ of formula I, I(a) and/or III-VII is CH₂. In other embodiments, L₁ is C═O. In other embodiments, L₁ is CHR. In other embodiments, L₁ is C(R)₂. In other embodiments, R is H, F, C₁-C₅ linear or branched, substituted or unsubstituted alkyl, methyl; each represents a separate embodiment according to this invention. In other embodiments, two geminal R substitutions are joined together to form a 3-6 membered substituted or unsubstituted, aliphatic ring. In other embodiments, two geminal R substitutions are joined together to form a cyclopropyl ring.

In some embodiments, L₂ of formula I and/or I(a) is a bond. In other embodiments, L₂ is CH₂. In other embodiments, L₂ is C═O. In other embodiments, L₂ is O. In other embodiments, L₂ is S.

In some embodiments, R of formula I-IX is H. In other embodiments, R is OH. In other embodiments, R is F. In other embodiments, R is Cl. In other embodiments, R is Br. In other embodiments, R is I. In other embodiments, R is CN. In other embodiments, R is CF₃. In other embodiments, R is NO₂. In other embodiments, R is C₁-C₅ linear or branched, substituted or unsubstituted alkyl. In other embodiments, R is methyl. In other embodiments, R is ethyl. In other embodiments, R is C₁-C₅ linear or branched alkoxy. In other embodiments, R is —R₈—O—R₁₀. In other embodiments, R is CH₂—CH₂—O—CH₃. In other embodiments, R is C₁-C₅ linear or branched haloalkyl. In other embodiments, R is CF₃. In other embodiments, R is CF₂CH₃. In other embodiments, R is CH₂CF₃. In other embodiments, R is CF₂CH₂CH₃. In other embodiments, R is CH₂CH₂CF₃. In other embodiments, R is CF₂CH(CH₃)₂. In other embodiments, R is CF(CH₃)—CH(CH₃)₂. In other embodiments, R is R₈-aryl. In other embodiments, R is CH₂-Ph. In other embodiments, R is substituted or unsubstituted aryl. In other embodiments, R is phenyl. In other embodiments, R is substituted or unsubstituted heteroaryl. In other embodiments, R is pyridine. In other embodiments, R is 2, 3, or 4-pyridine. In other embodiments, two geminal R substitutions are joined together to form a 3-6 membered substituted or unsubstituted, aliphatic or aromatic, carbocyclic or heterocyclic ring. In other embodiments, two geminal R substitutions are joined together to form a 3-6 membered aliphatic ring. In other embodiments, two geminal R substitutions are joined together to form a cyclopropyl ring. In other embodiments, R may be further substituted with at least one substitution selected from: F, Cl, Br, I, OH, SH, C₁-C₅ linear or branched alkyl, OH, alkoxy, N(R)₂, CF₃, phenyl, halophenyl, (benzyloxy)phenyl, CN, NO₂; each represents a separate embodiment according to this invention.

In some embodiments, R₈ of formula I-IX is CH₂. In other embodiments, R₈ is CH₂CH₂. In other embodiments, R₈ is CH₂CH₂CH₂.

In some embodiments, p of formula I-IX is 1. In other embodiments, p is 2. In other embodiments, p is 3.

In some embodiments, R₉ of formula I-IX is C═C.

In some embodiments, q of formula I-IX is 2.

In some embodiments, R₁₀ of formula I-IX is C₁-C₅ substituted or unsubstituted linear or branched alkyl. In other embodiments, R₁₀ is H. In other embodiments, R₁₀ is CH₃. In other embodiments, R₁₀ is CH₂CH₃. In other embodiments, R₁₀ is CH₂CH₂CH₃. In other embodiments, R₁₀ is CH₂—CH₂—O—CH₃. In other embodiments, R₁₀ is C₁-C₅ linear or branched alkoxy. In other embodiments, R₁₀ is O—CH₃.

In some embodiments, R₁₁ of formula I-IX is C₁-C₅ linear or branched alkyl. In other embodiments, R₁₀ is H. In other embodiments, R₁₁ is CH₃.

In some embodiments, R₁₀ and R₁₁ of formula I-IX are joined to form a substituted or unsubstituted C₃-C₈ heterocyclic ring. In other embodiments, R₁₀ and R₁₁ are joined to form a piperazine ring. In other embodiments, R₁₀ and R₁₁ are joined to form a piperidine ring. In some embodiments, substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof; each represents a separate embodiment according to this invention.

In some embodiments, m of formula I-V is 0. In some embodiments, m is 1. In some embodiments, m is 2.

In some embodiments, n of formula I-VI is 0. In other embodiments, n is 1. In other embodiments, n is 2.

In some embodiments, k of formula I-V is 0. In other embodiments, k is 1. In other embodiments, k is 2.

In some embodiments, 1 of formula I-VI is 0. In other embodiments, 1 is 1. In other embodiments, 1 is 2.

In some embodiments, w of formula I(a) and/or V(a) is 0 and the bridging moiety is absent. In other embodiments, w is 1. In other embodiments, w is 2.

In various embodiments, this invention is directed to the compounds presented in Table 1, pharmaceutical compositions and/or method of use thereof:

TABLE 1

| Com- | |
|---|---|
| pound | |
| Number | Compound Structure |
| 200 | 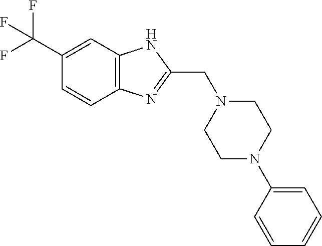 [SMILES: FC(F)(F)c1ccc2nc(CN3CCN(c4ccccc4)CC3)[nH]c2c1] |
| 201 | 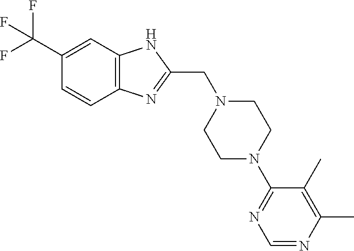 [SMILES: Cc1ncnc(N2CCN(Cc3nc4ccc(C(F)(F)F)cc4[nH]3)CC2)c1C] |
| 202 | 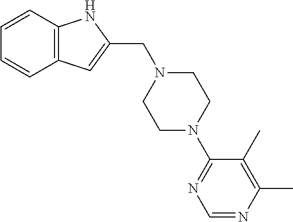 [SMILES: Cc1ncnc(N2CCN(Cc3cc4ccccc4[nH]3)CC2)c1C] |
| 203 | 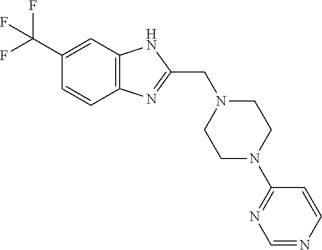 [SMILES: FC(F)(F)c1ccc2nc(CN3CCN(c4ccncn4)CC3)[nH]c2c1] |
| 204 | 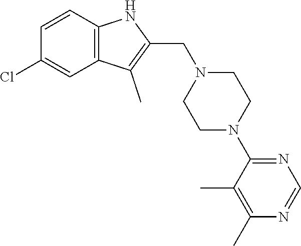 [SMILES: Cc1ncnc(N2CCN(Cc3[nH]c4ccc(Cl)cc4c3C)CC2)c1C] |
| 205 | 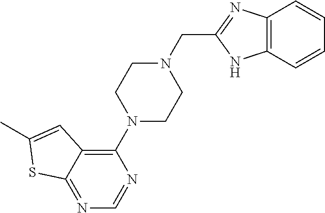 [SMILES: Cc1cc2c(N3CCN(Cc4nc5ccccc5[nH]4)CC3)ncnc2s1] |
| 207 | 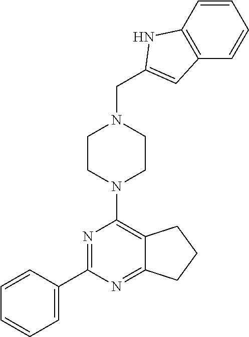 [SMILES: c1ccc(-c2nc3c(c(N4CCN(Cc5cc6ccccc6[nH]5)CC4)n2)CCC3)cc1] |
| 208 | 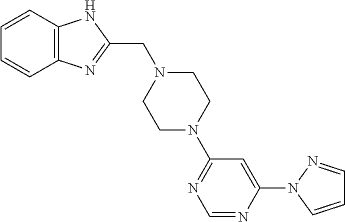 [SMILES: c1ccc2[nH]c(CN3CCN(c4cc(-n5cccn5)ncn4)CC3)nc2c1] |
| 209 | 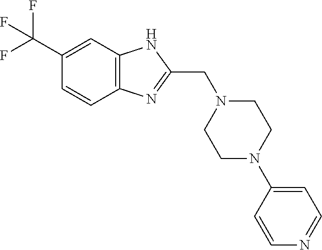 [SMILES: FC(F)(F)c1ccc2nc(CN3CCN(c4ccncc4)CC3)[nH]c2c1] |
| 210 | 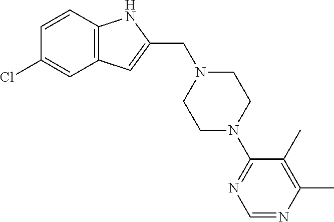 [SMILES: Cc1ncnc(N2CCN(Cc3cc4cc(Cl)ccc4[nH]3)CC2)c1C] |
| 211 | 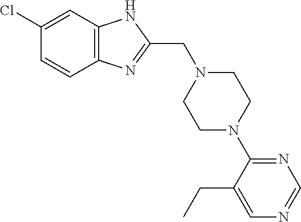 [SMILES: CCc1cncnc1N1CCN(Cc2nc3ccc(Cl)cc3[nH]2)CC1] |
| 212 | 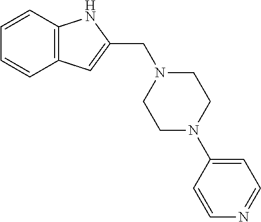 [SMILES: c1ccc2[nH]c(CN3CCN(c4ccncc4)CC3)cc2c1] |
| 213 | 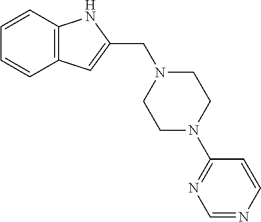 [SMILES: c1ccc2[nH]c(CN3CCN(c4ccncn4)CC3)cc2c1] |
| 214 | 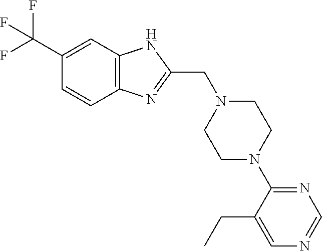 [SMILES: CCc1cncnc1N1CCN(Cc2nc3ccc(C(F)(F)F)cc3[nH]2)CC1] |
| 215 | 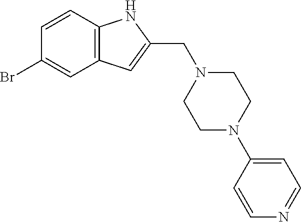 [SMILES: Brc1ccc2[nH]c(CN3CCN(c4ccncc4)CC3)cc2c1] |
| 216 | 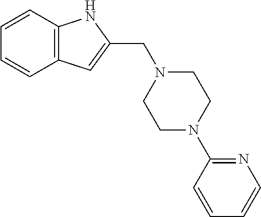 [SMILES: c1ccc(N2CCN(Cc3cc4ccccc4[nH]3)CC2)nc1] |
| 217 | 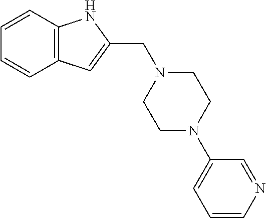 [SMILES: c1cncc(N2CCN(Cc3cc4ccccc4[nH]3)CC2)c1] |
| 218 | 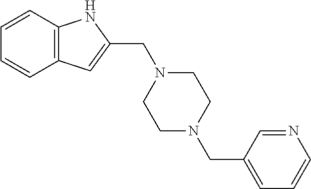 [SMILES: c1cncc(CN2CCN(Cc3cc4ccccc4[nH]3)CC2)c1] |
| 219 | 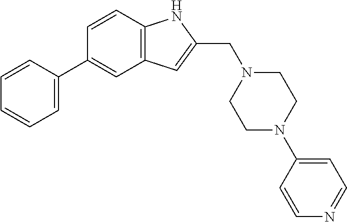 [SMILES: c1ccc(-c2ccc3[nH]c(CN4CCN(c5ccncc5)CC4)cc3c2)cc1] |
| 220 | 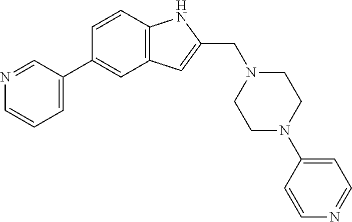 [SMILES: c1cncc(-c2ccc3[nH]c(CN4CCN(c5ccncc5)CC4)cc3c2)c1] |
| 221 | 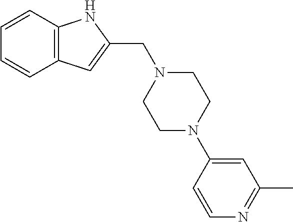 [SMILES: Cc1cc(N2CCN(Cc3cc4ccccc4[nH]3)CC2)ccn1] |
| 222 | 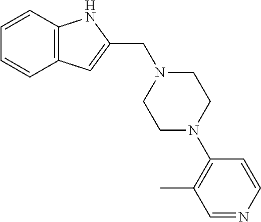 [SMILES: Cc1cnccc1N1CCN(Cc2cc3ccccc3[nH]2)CC1] |
| 223 | 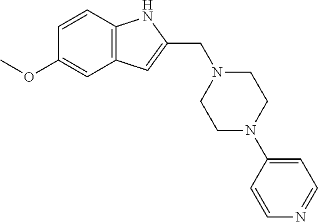 [SMILES: COc1ccc2[nH]c(CN3CCN(c4ccncc4)CC3)cc2c1] |
| 224 | 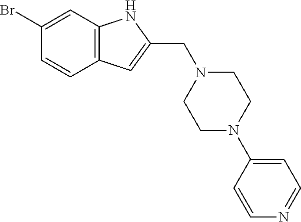 [SMILES: Brc1ccc2cc(CN3CCN(c4ccncc4)CC3)[nH]c2c1] |
| 225 | 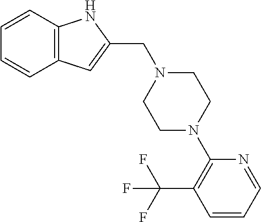 [SMILES: FC(F)(F)c1cccnc1N1CCN(Cc2cc3ccccc3[nH]2)CC1] |
| 226 | 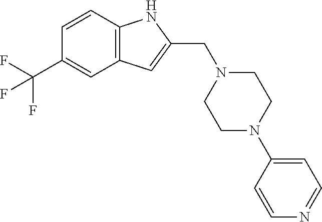 [SMILES: FC(F)(F)c1ccc2[nH]c(CN3CCN(c4ccncc4)CC3)cc2c1] |
| 227 | 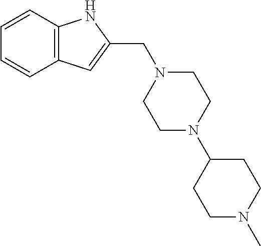 [SMILES: CN1CCC(N2CCN(Cc3cc4ccccc4[nH]3)CC2)CC1] |
| 228 | 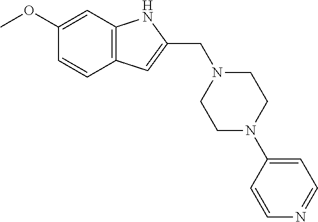 [SMILES: COc1ccc2cc(CN3CCN(c4ccncc4)CC3)[nH]c2c1] |
| 229 | 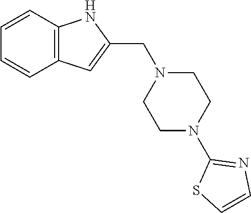 [SMILES: c1ccc2[nH]c(CN3CCN(c4nccs4)CC3)cc2c1] |
| 230 | 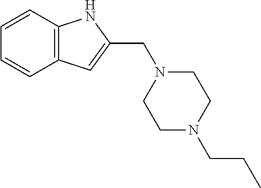 [SMILES: CCCN1CCN(Cc2cc3ccccc3[nH]2)CC1] |
| 231 | 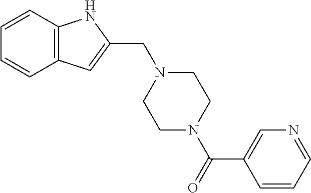 [SMILES: O=C(c1cccnc1)N1CCN(Cc2cc3ccccc3[nH]2)CC1] |
| 232 | 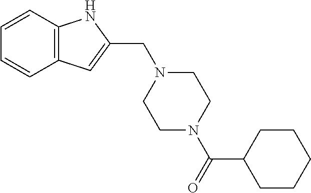 [SMILES: O=C(C1CCCCC1)N1CCN(Cc2cc3ccccc3[nH]2)CC1] |
| 233 | 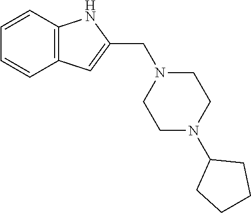 [SMILES: c1ccc2[nH]c(CN3CCN(C4CCCC4)CC3)cc2c1] |
| 234 | 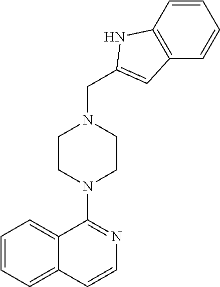 [SMILES: c1ccc2[nH]c(CN3CCN(c4nccc5ccccc45)CC3)cc2c1] |
| 235 | 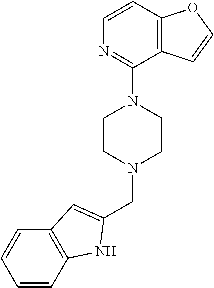 [SMILES: c1ccc2[nH]c(CN3CCN(c4nccc5occc45)CC3)cc2c1] |
| 236 | 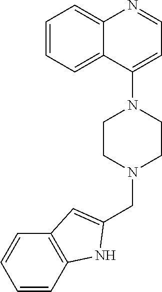 [SMILES: c1ccc2[nH]c(CN3CCN(c4ccnc5ccccc45)CC3)cc2c1] |
| 237 | 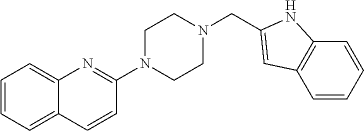 [SMILES: c1ccc2nc(N3CCN(Cc4cc5ccccc5[nH]4)CC3)ccc2c1] |
| 238 | 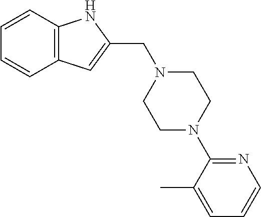 [SMILES: Cc1cccnc1N1CCN(Cc2cc3ccccc3[nH]2)CC1] |
| 239 | 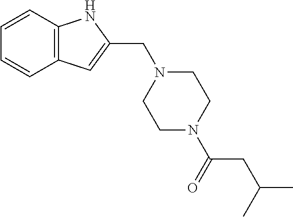 [SMILES: CC(C)CC(=O)N1CCN(Cc2cc3ccccc3[nH]2)CC1] |
| 240 | 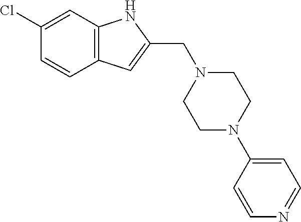 [SMILES: Clc1ccc2cc(CN3CCN(c4ccncc4)CC3)[nH]c2c1] |
| 241 | 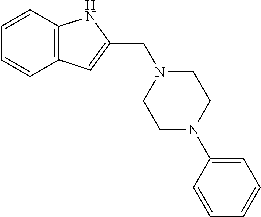 [SMILES: c1ccc(N2CCN(Cc3cc4ccccc4[nH]3)CC2)cc1] |
| 242 | 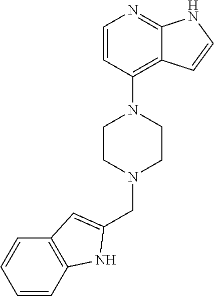 [SMILES: c1ccc2[nH]c(CN3CCN(c4ccnc5[nH]ccc45)CC3)cc2c1] |
| 243 | 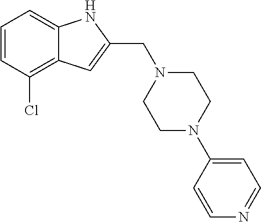 [SMILES: Clc1cccc2[nH]c(CN3CCN(c4ccncc4)CC3)cc12] |
| 244 | 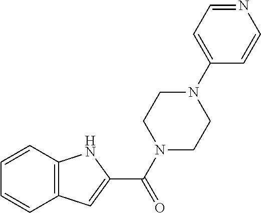 [SMILES: O=C(c1cc2ccccc2[nH]1)N1CCN(c2ccncc2)CC1] |
| 245 | 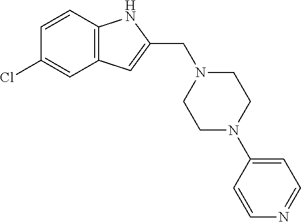 [SMILES: Clc1ccc2[nH]c(CN3CCN(c4ccncc4)CC3)cc2c1] |
| 246 | 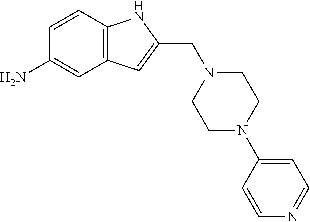 [SMILES: Nc1ccc2[nH]c(CN3CCN(c4ccncc4)CC3)cc2c1] |
| 247 | 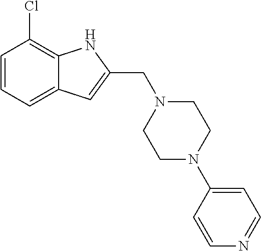 [SMILES: Clc1cccc2cc(CN3CCN(c4ccncc4)CC3)[nH]c12] |
| 248 | 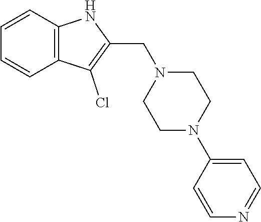 [SMILES: Clc1c(CN2CCN(c3ccncc3)CC2)[nH]c2ccccc12] |
| 249 | 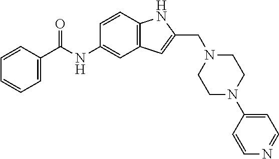 [SMILES: O=C(Nc1ccc2[nH]c(CN3CCN(c4ccncc4)CC3)cc2c1)c1ccccc1] |
| 250 | 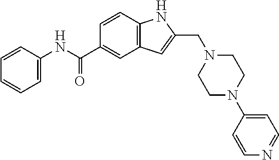 [SMILES: O=C(Nc1ccccc1)c1ccc2[nH]c(CN3CCN(c4ccncc4)CC3)cc2c1] |
| 251 | 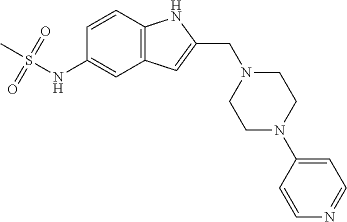 [SMILES: CS(=O)(=O)Nc1ccc2[nH]c(CN3CCN(c4ccncc4)CC3)cc2c1] |
| 252 | 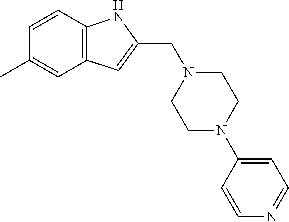 [SMILES: Cc1ccc2[nH]c(CN3CCN(c4ccncc4)CC3)cc2c1] |
| 253 | 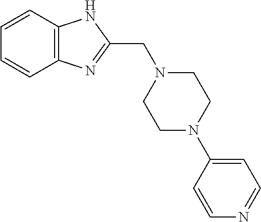 [SMILES: c1ccc2[nH]c(CN3CCN(c4ccncc4)CC3)nc2c1] |
| 254 | 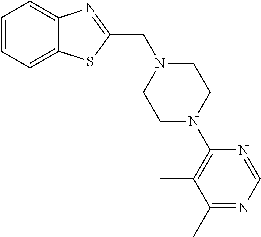 [SMILES: Cc1ncnc(N2CCN(Cc3nc4ccccc4s3)CC2)c1C] |
| 255 | 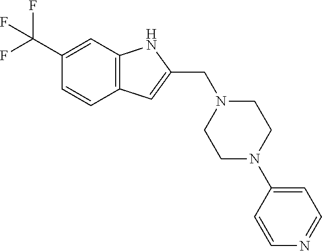 [SMILES: FC(F)(F)c1ccc2cc(CN3CCN(c4ccncc4)CC3)[nH]c2c1] |
| 256 | 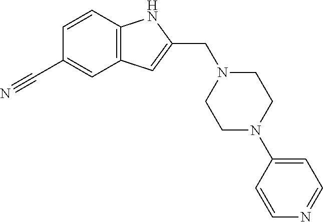 [SMILES: N#Cc1ccc2[nH]c(CN3CCN(c4ccncc4)CC3)cc2c1] |
| 257 | 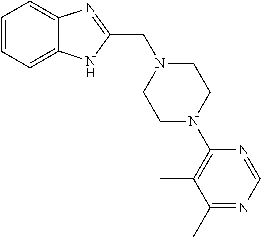 [SMILES: Cc1ncnc(N2CCN(Cc3nc4ccccc4[nH]3)CC2)c1C] |
| 258 | 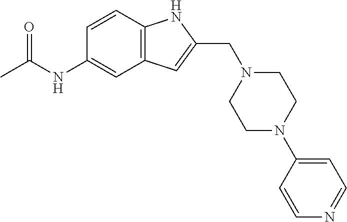 [SMILES: CC(=O)Nc1ccc2[nH]c(CN3CCN(c4ccncc4)CC3)cc2c1] |
| 259 | 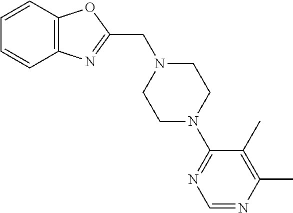 [SMILES: Cc1ncnc(N2CCN(Cc3nc4ccccc4o3)CC2)c1C] |
| 260 | 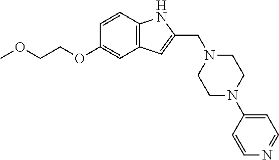 [SMILES: COCCOc1ccc2[nH]c(CN3CCN(c4ccncc4)CC3)cc2c1] |
| 261 | 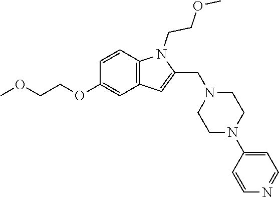 [SMILES: COCCOc1ccc2c(c1)cc(CN1CCN(c3ccncc3)CC1)n2CCOC] |
| 262 | 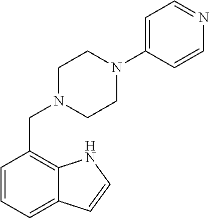 [SMILES: c1cc(CN2CCN(c3ccncc3)CC2)c2[nH]ccc2c1] |
| 263 | 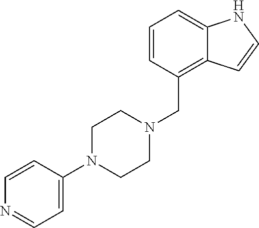 [SMILES: c1cc(CN2CCN(c3ccncc3)CC2)c2cc[nH]c2c1] |
| 264 | 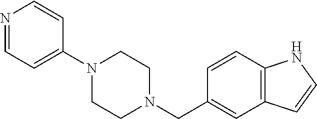 [SMILES: c1cc(N2CCN(Cc3ccc4[nH]ccc4c3)CC2)ccn1] |
| 265 | 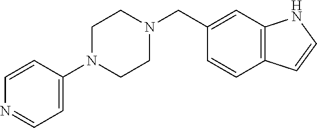 [SMILES: c1cc(N2CCN(Cc3ccc4cc[nH]c4c3)CC2)ccn1] |
| 266 | 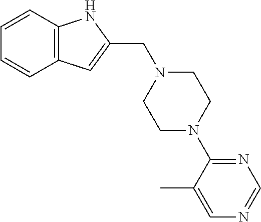 [SMILES: Cc1cncnc1N1CCN(Cc2cc3ccccc3[nH]2)CC1] |
| 267 | 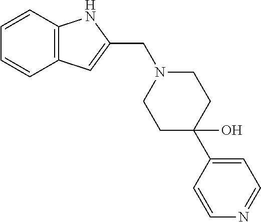 [SMILES: OC1(c2ccncc2)CCN(Cc2cc3ccccc3[nH]2)CC1] |
| 268 | 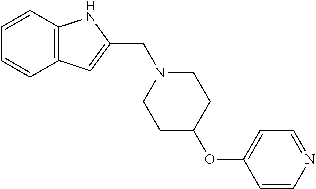 [SMILES: c1ccc2[nH]c(CN3CCC(Oc4ccncc4)CC3)cc2c1] |
| 269 | 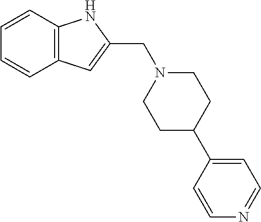 [SMILES: c1ccc2[nH]c(CN3CCC(c4ccncc4)CC3)cc2c1] |
| 270 | 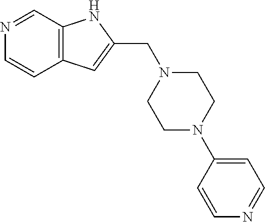 [SMILES: c1cc(N2CCN(Cc3cc4ccncc4[nH]3)CC2)ccn1] |
| 271 | 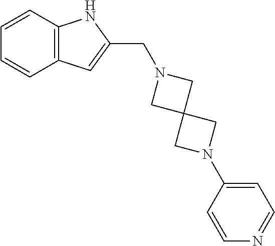 [SMILES: c1ccc2[nH]c(CN3CC4(C3)CN(c3ccncc3)C4)cc2c1] |
| 272 | 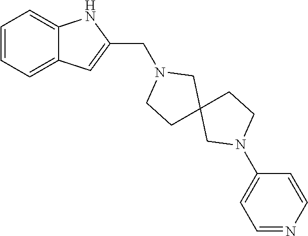 [SMILES: c1ccc2[nH]c(CN3CCC4(CCN(c5ccncc5)C4)C3)cc2c1] |
| 273 | 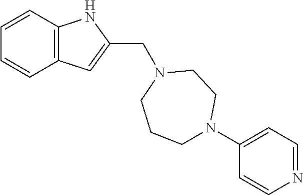 [SMILES: c1ccc2[nH]c(CN3CCCN(c4ccncc4)CC3)cc2c1] |
| 274 | 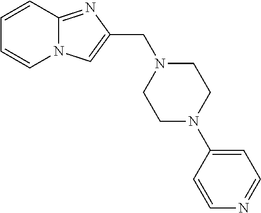 [SMILES: c1ccn2cc(CN3CCN(c4ccncc4)CC3)nc2c1] |
| 275 | 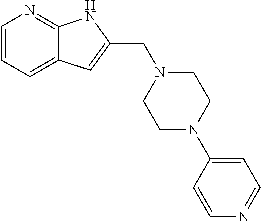 [SMILES: c1cnc2[nH]c(CN3CCN(c4ccncc4)CC3)cc2c1] |
| 276 | 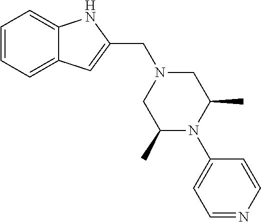 [SMILES: C[C@@H]1CN(Cc2cc3ccccc3[nH]2)C[C@H](C)N1c1ccncc1] |
| 277 | 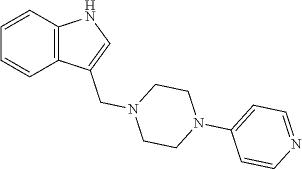 [SMILES: c1ccc2c(CN3CCN(c4ccncc4)CC3)c[nH]c2c1] |
| 278 | 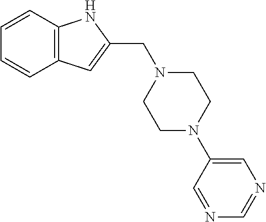 [SMILES: c1ccc2[nH]c(CN3CCN(c4cncnc4)CC3)cc2c1] |
| 279 | 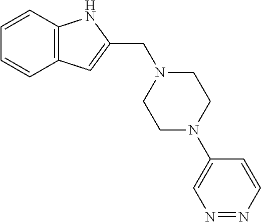 [SMILES: c1ccc2[nH]c(CN3CCN(c4ccnnc4)CC3)cc2c1] |
| 280 | 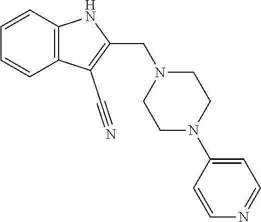 [SMILES: N#Cc1c(CN2CCN(c3ccncc3)CC2)[nH]c2ccccc12] |
| 281 | 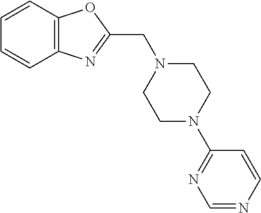 [SMILES: c1ccc2oc(CN3CCN(c4ccncn4)CC3)nc2c1] |
| 282 | 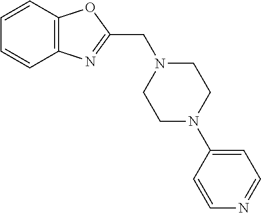 [SMILES: c1ccc2oc(CN3CCN(c4ccncc4)CC3)nc2c1] |
| 283 | 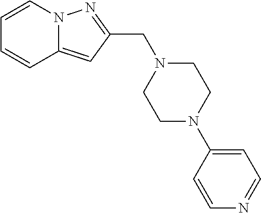 [SMILES: c1ccn2nc(CN3CCN(c4ccncc4)CC3)cc2c1] |
| 284 | 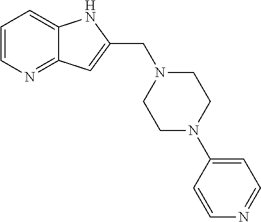 [SMILES: c1cnc2cc(CN3CCN(c4ccncc4)CC3)[nH]c2c1] |
| 285 | 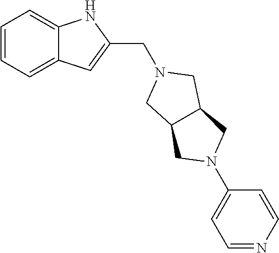 [SMILES: c1ccc2[nH]c(CN3C[C@@H]4CN(c5ccncc5)C[C@@H]4C3)cc2c1] |
| 286 | 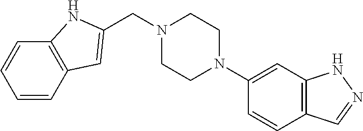 [SMILES: c1ccc2[nH]c(CN3CCN(c4ccc5cn[nH]c5c4)CC3)cc2c1] |
| 287 | 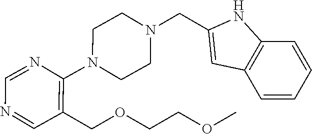 [SMILES: COCCOCc1cncnc1N1CCN(Cc2cc3ccccc3[nH]2)CC1] |
| 288 | 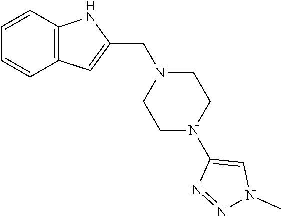 [SMILES: Cn1cc(N2CCN(Cc3cc4ccccc4[nH]3)CC2)nn1] |
| 289 | 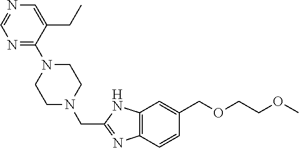 [SMILES: CCc1cncnc1N1CCN(Cc2nc3ccc(COCCOC)cc3[nH]2)CC1] |
| 290 | 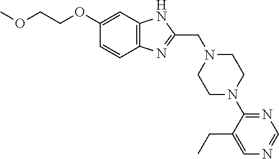 [SMILES: CCc1cncnc1N1CCN(Cc2nc3ccc(OCCOC)cc3[nH]2)CC1] |
| 291 | 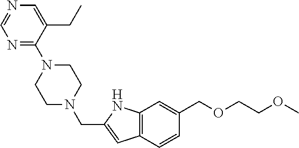 [SMILES: CCc1cncnc1N1CCN(Cc2cc3ccc(COCCOC)cc3[nH]2)CC1] |
| 292 | 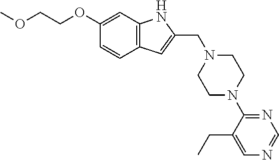 [SMILES: CCc1cncnc1N1CCN(Cc2cc3ccc(OCCOC)cc3[nH]2)CC1] |
| 293 | 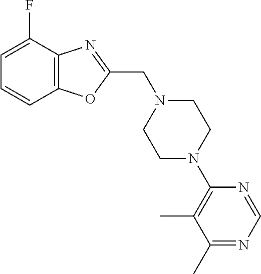 [SMILES: Cc1ncnc(N2CCN(Cc3nc4c(F)cccc4o3)CC2)c1C] |
| 294 | 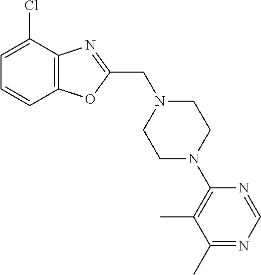 [SMILES: Cc1ncnc(N2CCN(Cc3nc4c(Cl)cccc4o3)CC2)c1C] |
| 295 | 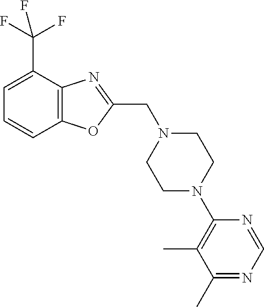 [SMILES: Cc1ncnc(N2CCN(Cc3nc4c(C(F)(F)F)cccc4o3)CC2)c1C] |
| 296 | 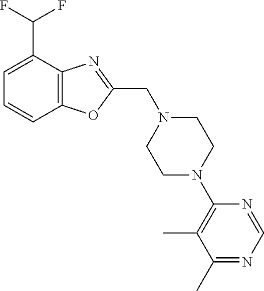 [SMILES: Cc1ncnc(N2CCN(Cc3nc4c(C(F)F)cccc4o3)CC2)c1C] |
| 297 | 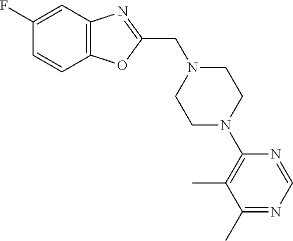 [SMILES: Cc1ncnc(N2CCN(Cc3nc4cc(F)ccc4o3)CC2)c1C] |
| 298 | 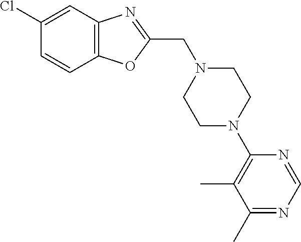 [SMILES: Cc1ncnc(N2CCN(Cc3nc4cc(Cl)ccc4o3)CC2)c1C] |
| 299 | 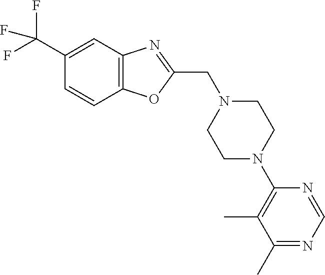 [SMILES: Cc1ncnc(N2CCN(Cc3nc4cc(C(F)(F)F)ccc4o3)CC2)c1C] |
| 300 | 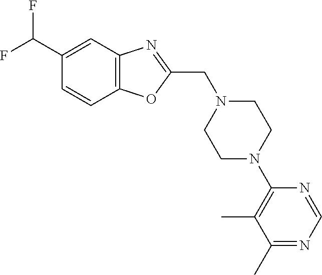 [SMILES: Cc1ncnc(N2CCN(Cc3nc4cc(C(F)F)ccc4o3)CC2)c1C] |
| 301 | 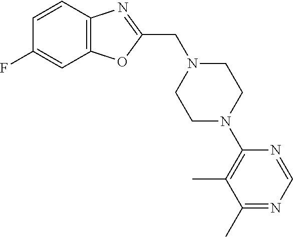 [SMILES: Cc1ncnc(N2CCN(Cc3nc4ccc(F)cc4o3)CC2)c1C] |
| 302 | 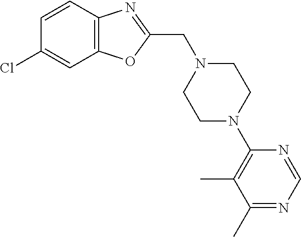 [SMILES: Cc1ncnc(N2CCN(Cc3nc4ccc(Cl)cc4o3)CC2)c1C] |
| 303 | 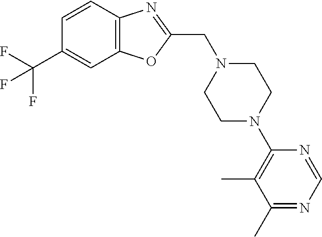 [SMILES: Cc1ncnc(N2CCN(Cc3nc4ccc(C(F)(F)F)cc4o3)CC2)c1C] |
| 304 | 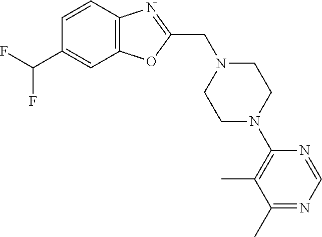 [SMILES: Cc1ncnc(N2CCN(Cc3nc4ccc(C(F)F)cc4o3)CC2)c1C] |
| 305 | 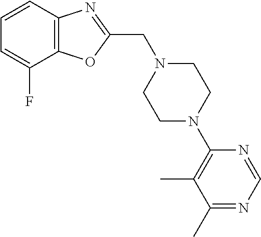 [SMILES: Cc1ncnc(N2CCN(Cc3nc4cccc(F)c4o3)CC2)c1C] |
| 306 | 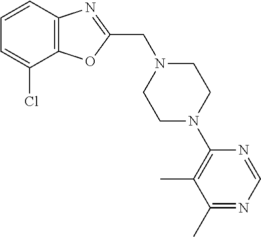 [SMILES: Cc1ncnc(N2CCN(Cc3nc4cccc(Cl)c4o3)CC2)c1C] |
| 307 | 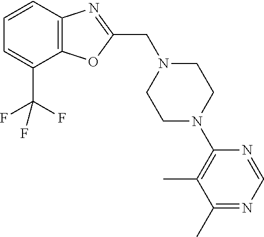 [SMILES: Cc1ncnc(N2CCN(Cc3nc4cccc(C(F)(F)F)c4o3)CC2)c1C] |
| 308 | 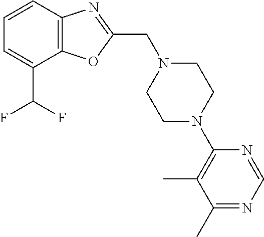 [SMILES: Cc1ncnc(N2CCN(Cc3nc4cccc(C(F)F)c4o3)CC2)c1C] |
| 309 | 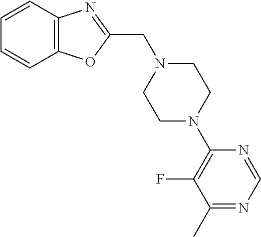 [SMILES: Cc1ncnc(N2CCN(Cc3nc4ccccc4o3)CC2)c1F] |
| 310 | 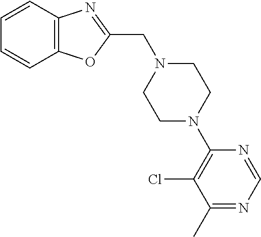 [SMILES: Cc1ncnc(N2CCN(Cc3nc4ccccc4o3)CC2)c1Cl] |
| 311 | 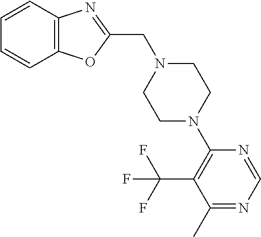 [SMILES: Cc1ncnc(N2CCN(Cc3nc4ccccc4o3)CC2)c1C(F)(F)F] |
| 312 | 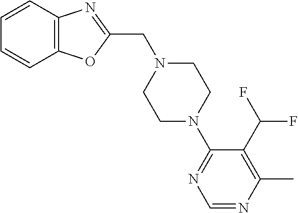 [SMILES: Cc1ncnc(N2CCN(Cc3nc4ccccc4o3)CC2)c1C(F)F] |
| 313 | 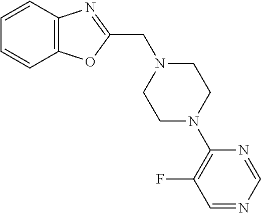 [SMILES: Fc1cncnc1N1CCN(Cc2nc3ccccc3o2)CC1] |
| 314 | 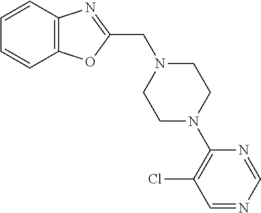 [SMILES: Clc1cncnc1N1CCN(Cc2nc3ccccc3o2)CC1] |
| 315 | 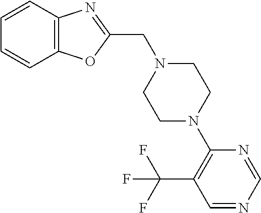 [SMILES: FC(F)(F)c1cncnc1N1CCN(Cc2nc3ccccc3o2)CC1] |
| 316 | 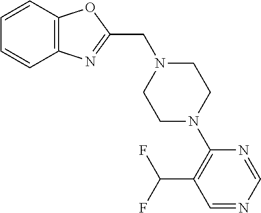 [SMILES: FC(F)c1cncnc1N1CCN(Cc2nc3ccccc3o2)CC1] |
| 317 | 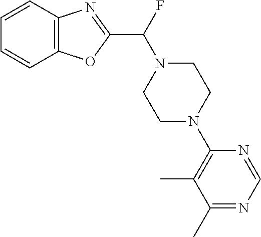 [SMILES: Cc1ncnc(N2CCN(C(F)c3nc4ccccc4o3)CC2)c1C] |
| 318 | 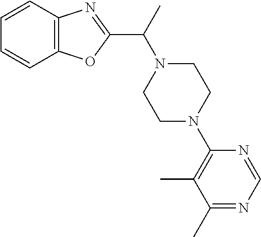 [SMILES: Cc1ncnc(N2CCN(C(C)c3nc4ccccc4o3)CC2)c1C] |
| 320 | 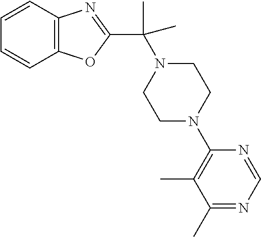 [SMILES: Cc1ncnc(N2CCN(C(C)(C)c3nc4ccccc4o3)CC2)c1C] |
| 321 | 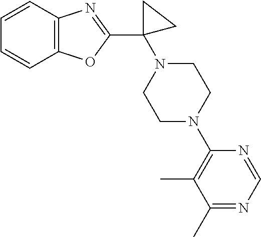 [SMILES: Cc1ncnc(N2CCN(C3(c4nc5ccccc5o4)CC3)CC2)c1C] |
| 322 | 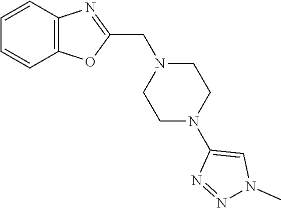 [SMILES: Cn1cc(N2CCN(Cc3nc4ccccc4o3)CC2)nn1] |
| 323 | 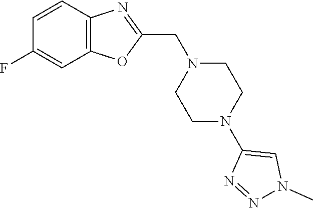 [SMILES: Cn1cc(N2CCN(Cc3nc4ccc(F)cc4o3)CC2)nn1] |
| 324 | 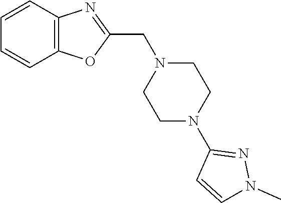 [SMILES: Cn1ccc(N2CCN(Cc3nc4ccccc4o3)CC2)n1] |
| 325 | 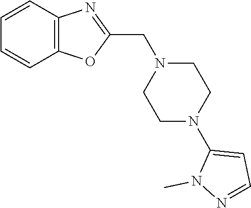 [SMILES: Cn1nccc1N1CCN(Cc2nc3ccccc3o2)CC1] |
| 326 | 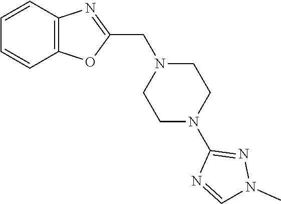 [SMILES: Cn1cnc(N2CCN(Cc3nc4ccccc4o3)CC2)n1] |
| 327 | 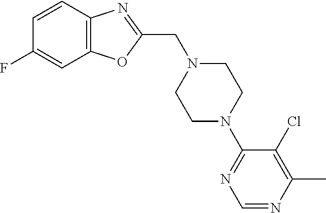 [SMILES: Cc1ncnc(N2CCN(Cc3nc4ccc(F)cc4o3)CC2)c1Cl] |
| 328 | 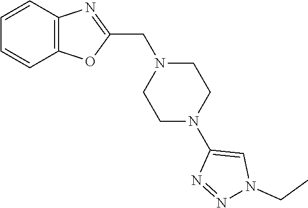 [SMILES: CCn1cc(N2CCN(Cc3nc4ccccc4o3)CC2)nn1] |
| 329 | 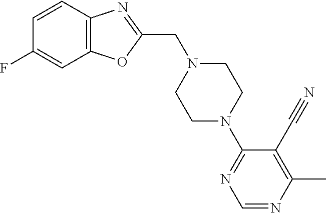 [SMILES: Cc1ncnc(N2CCN(Cc3nc4ccc(F)cc4o3)CC2)c1C#N] |
| 330 | 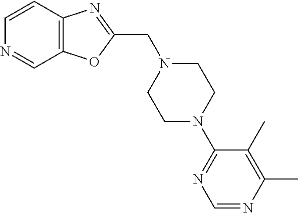 [SMILES: Cc1ncnc(N2CCN(Cc3nc4ccncc4o3)CC2)c1C] |
| 331 | 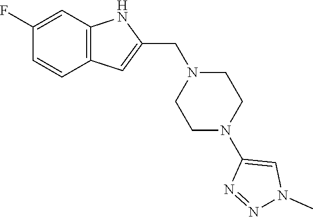 [SMILES: Cn1cc(N2CCN(Cc3cc4ccc(F)cc4[nH]3)CC2)nn1] |
| 332 | 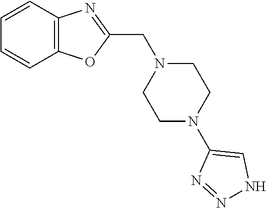 [SMILES: c1ccc2oc(CN3CCN(c4c[nH]nn4)CC3)nc2c1] |
| 333 | 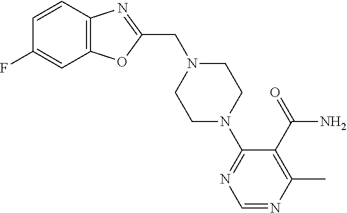 [SMILES: Cc1ncnc(N2CCN(Cc3nc4ccc(F)cc4o3)CC2)c1C(N)=O] |
| 334 | 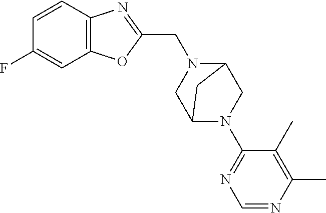 [SMILES: Cc1ncnc(N2CC3CC2CN3Cc2nc3ccc(F)cc3o2)c1C] |
| 336 | 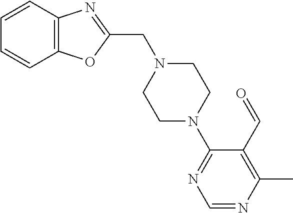 [SMILES: Cc1ncnc(N2CCN(Cc3nc4ccccc4o3)CC2)c1C=O] |
| 337 | 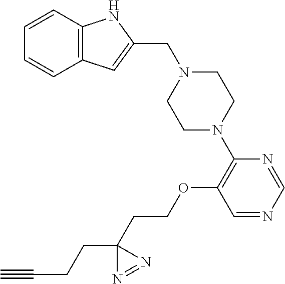 [SMILES: C#CCCC1(CCOc2cncnc2N2CCN(Cc3cc4ccccc4[nH]3)CC2)N=N1] |
| 338 | 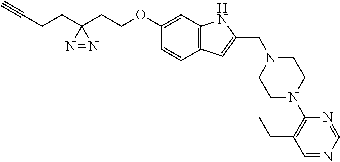 [SMILES: C#CCCC1(CCOc2ccc3cc(CN4CCN(c5ncncc5CC)CC4)[nH]c3c2)N=N1] |
| 339 | 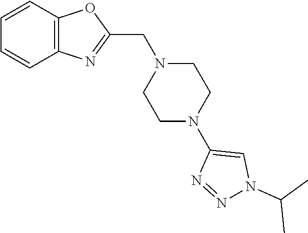 [SMILES: CC(C)n1cc(N2CCN(Cc3nc4ccccc4o3)CC2)nn1] |
| 340 | 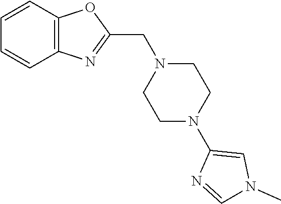 [SMILES: Cn1cnc(N2CCN(Cc3nc4ccccc4o3)CC2)c1] |

It is well understood that in structures presented in this invention wherein the carbon atom has less than 4 bonds, H atoms are present to complete the valence of the carbon. It is well understood that in structures presented in this invention wherein the nitrogen atom has less than 3 bonds, H atoms are present to complete the valence of the nitrogen.

In some embodiments, this invention is directed to the compounds listed hereinabove, pharmaceutical compositions and/or method of use thereof, wherein the compound is pharmaceutically acceptable salt, stereoisomer, tautomer, hydrate, N-oxide, prodrug, isotopic variant (deuterated analog), pharmaceutical product or any combination thereof. In some embodiments, the compounds are Collagen I translation inhibitors.

In various embodiments, the A ring of formula I, I(a), II, and/or IV is phenyl, naphthyl, pyridinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, isoquinoline, pyrazolyl, pyrrolyl, furanyl, thiophene-yl, isoquinolinyl, indolyl, 1H-indole, isoindolyl, naphthyl, anthracenyl, benzimidazolyl, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, cinnolinyl, phthalazinyl, quinolinyl, isoquinolinyl, 2,3-dihydroindenyl, indenyl, tetrahydronaphthyl, 3,4-dihydro-2H-benzo[b][1,4]dioxepine, benzo[d][1,3]dioxole, acridinyl, benzofuranyl, 1-benzofuran, isobenzofuranyl, benzofuran-2(3H)-one, benzothiophenyl, benzoxadiazole, benzo[c][1,2,5]oxadiazolyl, benzo[c]thiophenyl, benzodioxolyl, benzo[d][1,3]dioxole, thiadiazolyl, [1,3]oxazolo[4,5-b]pyridine, oxadiaziolyl, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]thiazole, thieno[3,2-d]pyrimidin-4(3H)-one, 4-oxo-4H-thieno[3,2-d][1,3]thiazin, imidazopyridin, imidazo[1,2-a]pyridine, 1H-imidazo[4,5-b]pyridine, 1H-imidazo[4,5-c]pyridine, 3H-imidazo[4,5-c]pyridine, pyrazolopyridine, pyrazolo[1,5-a]pyridine, imidazo[1,2-a]pyrazine, imidazo[1,2-a]pyrimidine, 1H-pyrrolo[2,3-b]pyridine, pyrido[2,3-b]pyrazine, pyrido[2,3-b]pyrazin-3(4H)-one, 4H-thieno[3,2-b]pyrrole, quinoxalin-2(1H)-one, pyrrolopyridine, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine; each represents a separate embodiment according to this invention; or A is C₃-C₈ cycloalkyl (e.g. cyclohexyl, cyclopentyl) or C₃-C₈ heterocyclic ring including but not limited to: tetrahydropyran, piperidine, 1-methylpiperidine, tetrahydrothiophene 1,1-dioxide, 1-(piperidin-1-yl)ethanone or morpholine; each represents a separate embodiment according to this invention. In some embodiments, A is a C₃-C₈ heterocyclic ring.

In various embodiments, the B ring of formula I, I(a), II, III, and/or IV is phenyl, naphthyl, pyridinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, isoquinoline, pyrazolyl, pyrrolyl, triazolyl, furanyl, thiophene-yl, isoquinolinyl, indolyl, 1H-indole, isoindolyl, naphthyl, anthracenyl, benzimidazolyl, 2,3-dihydro-1H-benzo[d]imidazolyl, tetrahydronaphthyl 3,4-dihydro-2H-benzo[b][1,4]dioxepine, benzofuran-2(3H)-one, benzo[d][1,3]dioxole, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, cinnolinyl, phthalazinyl, quinolinyl, isoquinolinyl, acridinyl, benzofuranyl, 1-benzofuran, isobenzofuranyl, benzothiophenyl, benzoxadiazole, benzo[c][1,2,5]oxadiazolyl, benzo[c]thiophenyl, benzodioxolyl, thiadiazolyl, [1,3]oxazolo[4,5-b]pyridine, oxadiaziolyl, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]thiazole, thieno[3,2-d]pyrimidin-4(3H)-one, 4-oxo-4H-thieno[3,2-d][1,3]thiazin, imidazo[1,2-a]pyridine, 1H-imidazo[4,5-b]pyridine, 3H-imidazo[4,5-b]pyridine, 3H-imidazo[4,5-c]pyridine, pyrazolo[1,5-a]pyridine, imidazo[1,2-a]pyrazine, imidazo[1,2-a]pyrimidine, pyrido[2,3-b]pyrazin or pyrido[2,3-b]pyrazin-3(4H)-one, 4H-thieno[3,2-b]pyrrole, quinoxalin-2(1H)-one, 1,2,3,4-tetrahydroquinoxaline, 1-(pyridin-1(2H)-yl)ethanone, 1H-pyrrolo[2,3-b]pyridine, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine, C₃-C₈ cycloalkyl, or C₃-C₈ heterocyclic ring including but not limited to: tetrahydropyran, piperidine, 1-methylpiperidine, tetrahydrothiophene 1,1-dioxide, 1-(piperidin-1-yl)ethanone or morpholine; each represents a separate embodiment according to this invention. In some embodiments, B is a C₃-C₈ heterocyclic ring. In some embodiments, B is pyrimidine.

In various embodiments, compound of formula I, I(a), II, III, IV, V, V(a) and/or VI is substituted by R₁, R₂, R₃, R₄ and R₅. Single substituents can be present at the ortho, meta, or para positions.

In various embodiments, R₁ of formula I-X and/or R₂ of formula I-IX are each independently H.

In various embodiments, R₁ of formula I, I(a), II, III, IV, V, V(a) and/or VI and/or R₂ of formula I, I(a), II, III, IV, V and/or V(a) are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph, NHCO—CH₃), NHC(O)—R₁₀ (e.g., NHCO—CH₃), NHCO—N(R₁₀)(R₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl, CHF₂, C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl; each represents a separate embodiment according to this invention. In some embodiments, substitutions include at least one of: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂; each represents a separate embodiment according to this invention.

In some embodiments, R₁ and R₂ are joined together to form a 5 or 6 membered substituted or unsubstituted, aliphatic or aromatic, carbocyclic or heterocyclic ring. In some embodiments, R₁ and R₂ are joined together to form a 5 or 6 membered heterocyclic ring. In some embodiments, R₁ and R₂ are joined together to form a pyrrol ring. In some embodiments, R₁ and R₂ are joined together to form a [1,3]dioxole ring. In some embodiments, R₁ and R₂ are joined together to form a furan-2(3H)-one ring. In some embodiments, R₁ and R₂ are joined together to form a benzene ring. In some embodiments, R₁ and R₂ are joined together to form a pyridine ring. In some embodiments, R₁ and R₂ are joined together to form a morpholine ring. In some embodiments, R₁ and R₂ are joined together to form a piperazine ring. In some embodiments, R₁ and R₂ are joined together to form an imidazole ring. In some embodiments, R₁ and R₂ are joined together to form a pyrrole ring. In some embodiments, R₁ and R₂ are joined together to form a cyclohexene ring. In some embodiments, R₁ and R₂ are joined together to form a pyrazine ring.

In various embodiments, compound of formula I, I(a), II, III, IV, V, V(a) and/or VI is substituted by R₃ and/or R₄. Single substituents can be present at the ortho, meta, or para positions.

In various embodiments, R₃ of formula I-IX; R₄ of formula I-V(a) and/or VII-IX; and/or R₅ of formula I-V(a) are each independently H, F, Cl, Br, I, OH, SH, R₈—OH, R₈—SH, —R₈—O—R₁₀, CH₂—O—CH₂—CH₂—O—CH₃, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl, CHF₂, C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole, imidazole), substituted or unsubstituted aryl (e.g., phenyl), substituted or unsubstituted benzyl. In various embodiments, R₃, R₄ or R₅ may each independently be further substituted with at least one substitution selected from: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂; each represents a separate embodiment of this invention.

In some embodiments, R₃ and R₄ are joined together to form a 5 or 6 membered substituted or unsubstituted, aliphatic or aromatic, carbocyclic or heterocyclic ring. In some embodiments, R₃ and R₄ are joined together to form a 5 or 6 membered carbocyclic ring. In some embodiments, R₃ and R₄ are joined together to form a 5 or 6 membered heterocyclic ring. In some embodiments, R₃ and R₄ are joined together to form a dioxole ring. [1,3]dioxole ring. In some embodiments, R₃ and R₄ are joined together to form a dihydrofuran-2(3H)-one ring. In some embodiments, R₃ and R₄ are joined together to form a furan-2(3H)-one ring. In some embodiments, R₃ and R₄ are joined together to form a benzene ring. In some embodiments, R₃ and R₄ are joined together to form an imidazole ring. In some embodiments, R₃ and R₄ are joined together to form a pyridine ring. In some embodiments, R₃ and R₄ are joined together to form a thiophene ring. In some embodiments, R₃ and R₄ are joined together to form a furane ring. In some embodiments, R₃ and R₄ are joined together to form a pyrrole ring. In some embodiments, R₃ and R₄ are joined together to form a pyrazole ring. In some embodiments, R₃ and R₄ are joined together to form a cyclohexene ring. In some embodiments, R₃ and R₄ are joined together to form a cyclopentene ring. In some embodiments, R₄ and R₃ are joined together to form a dioxepine ring.

In various embodiments, n of compound of formula I, I(a), II, III, IV, V, V(a) and/or VI is 0. In some embodiments, n is 0 or 1. In some embodiments, n is between 1 and 3. In some embodiments, n is between 1 and 4. In some embodiments, n is between 0 and 2. In some embodiments, n is between 0 and 3. In some embodiments, n is between 0 and 4. In some embodiments, n is 1. In some embodiments, n is 2. In some embodiments, n is 3. In some embodiments, n is 4.

In various embodiments, m of compound of formula I, I(a), II, III, IV, V and/or V(a) is 0. In some embodiments, m is 0 or 1. In some embodiments, m is between 1 and 3. In some embodiments, m is between 1 and 4. In some embodiments, m is between 0 and 2. In some embodiments, m is between 0 and 3. In some embodiments, m is between 0 and 4. In some embodiments, m is 1. In some embodiments, m is 2. In some embodiments, m is 3. In some embodiments, m is 4.

In various embodiments, 1 of compound of formula I, I(a), II, III, IV, V, V(a) and/or VI is 0. In some embodiments, 1 is 0 or 1. In some embodiments, 1 is between 1 and 3. In some embodiments, 1 is between 1 and 4. In some embodiments, 1 is 1 or 2. In some embodiments, 1 is between 0 and 3. In some embodiments, 1 is between 0 and 4. In some embodiments, 1 is 1. In some embodiments, 1 is 2. In some embodiments, 1 is 3. In some embodiments, 1 is 4.

In various embodiments, k of compound of formula I, I(a), II, III, IV, V and/or V(a) is 0. In some embodiments, k is 0 or 1. In some embodiments, k is between 1 and 3. In some embodiments, k is between 1 and 4. In some embodiments, k is between 0 and 2. In some embodiments, k is between 0 and 3. In some embodiments, k is between 0 and 4. In some embodiments, k is 1. In some embodiments, k is 2. In some embodiments, k is 3. In some embodiments, k is 4.

It is understood that for heterocyclic rings, n, m, 1 and/or k are limited to the number of available positions for substitution, i.e. to the number of CH or NH groups minus one. Accordingly, if A and/or B rings are, for example, furanyl, thiophenyl or pyrrolyl, n, m, 1 and k are between 0 and 2; and if A and/or B rings are, for example, oxazolyl, imidazolyl or thiazolyl, n, m, 1 and k are either 0 or 1; and if A and/or B rings are, for example, oxadiazolyl or thiadiazolyl, n, m, 1 and k are 0.

In various embodiments, R₈ of compound of formula I-IX is CH₂. In some embodiments, R₈ is CH₂CH₂. In some embodiments, R₈ is CH₂CH₂CH₂. In some embodiments, R₈ is CH₂CH₂CH₂CH₂.

In various embodiments, p of compound of formula I-IX is 1. In some embodiments, p is 2. In some embodiments, p is 3. In some embodiments, p is 4. In some embodiments, p is 5. In some embodiments, p is between 1 and 3. In some embodiments, p is between 1 and 5. In some embodiments, p is between 1 and 10.

In some embodiments, R₉ of compound of formula I-IX is C≡C. In some embodiments, R₉ is C≡C—C≡C. In some embodiments, R₉ is CH═CH. In some embodiments, R₉ is CH═CH—CH═CH.

In some embodiments, q of compound of formula I-IX is 2. In some embodiments, q is 4. In some embodiments, q is 6. In some embodiments, q is 8. In some embodiments, q is between 2 and 6.

In various embodiments, R₁₀ of compound of formula I-IX is H. In some embodiments, R₁₀ is substituted or unsubstituted C₁-C₅ linear or branched alkyl. In some embodiments, R₁₀ is methyl. In some embodiments, R₁₀ is ethyl. In some embodiments, R₁₀ is propyl. In some embodiments, R₁₀ is isopropyl. In some embodiments, R₁₀ is butyl. In some embodiments, R₁₀ is isobutyl. In some embodiments, R₁₀ is t-butyl. In some embodiments, R₁₀ is cyclopropyl. In some embodiments, R₁₀ is pentyl. In some embodiments, R₁₀ is isopentyl. In some embodiments, R₁₀ is neopentyl. In some embodiments, R₁₀ is benzyl. In some embodiments, R₁₀ is CH₂—CH₂—O—CH₃. In some embodiments, R₁₀ is C₁-C₅ linear or branched alkoxy. In some embodiments, R₁₀ is O—CH₃. In some embodiments, R₁₀ is C(O)R. In some embodiments, R₁₀ is S(O)₂R.

In various embodiments, R₁₁ of compound of formula I-IX is H. In some embodiments, R₁₁ is C₁-C₅ linear or branched alkyl. In some embodiments, R₁₁ is methyl. In some embodiments, R₁₁ is ethyl. In some embodiments, R₁₀ is propyl. In some embodiments, R₁₁ is isopropyl. In some embodiments, R₁₁ is butyl. In some embodiments, R₁₁ is isobutyl. In some embodiments, R₁₁ is t-butyl. In some embodiments, R₁₁ is cyclopropyl. In some embodiments, R₁₁ is pentyl. In some embodiments, R₁₁ is isopentyl. In some embodiments, R₁ is neopentyl. In some embodiments, R₁₁ is benzyl. In some embodiments, R₁₁ is C(O)R. In some embodiments, R₁₁ is S(O)₂R.

In some embodiments, R₁₀ and R₁₁ of formula I-IX are joined to form a substituted or unsubstituted C₃-C₈ heterocyclic ring. In other embodiments, R₁₀ and R₁₁ are joined to form a piperazine ring. In other embodiments, R₁₀ and R₁₁ are joined to form a piperidine ring. In some embodiments, substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof; each represents a separate embodiment according to this invention.

In some embodiments, R of formula I-IX is H. In other embodiments, R is OH. In other embodiments, R is F. In other embodiments, R is Cl. In other embodiments, R is Br. In other embodiments, R is I. In other embodiments, R is CN. In other embodiments, R is CF₃. In other embodiments, R is NO₂. In other embodiments, R is C₁-C₅ linear or branched, substituted or unsubstituted alkyl. In other embodiments, R is methyl. In other embodiments, R is ethyl. In other embodiments, R is C₁-C₅ linear or branched alkoxy. In other embodiments, R is —R₈—O—R₁₀. In other embodiments, R is CH₂—CH₂—O—CH₃. In other embodiments, R is C₁-C₅ linear or branched haloalkyl. In other embodiments, R is CF₃. In other embodiments, R is CF₂CH₃. In other embodiments, R is CH₂CF₃. In other embodiments, R is CF₂CH₂CH₃. In other embodiments, R is CH₂CH₂CF₃. In other embodiments, R is CF₂CH(CH₃)₂. In other embodiments, R is CF(CH₃)—CH(CH₃)₂. In other embodiments, R is R₈-aryl. In other embodiments, R is CH₂-Ph. In other embodiments, R is substituted or unsubstituted aryl. In other embodiments, R is phenyl. In other embodiments, R is substituted or unsubstituted heteroaryl. In other embodiments, R is pyridine. In other embodiments, R is 2, 3, or 4-pyridine. In other embodiments, two geminal R substitutions are joined together to form a 3-6 membered substituted or unsubstituted, aliphatic or aromatic, carbocyclic or heterocyclic ring. In other embodiments, two geminal R substitutions are joined together to form a 3-6 membered aliphatic ring. In other embodiments, two geminal R substitutions are joined together to form a cyclopropyl ring. In other embodiments, substitutions include at least one of: F, Cl, Br, I, OH, SH, C₁-C₅ linear or branched alkyl, OH, alkoxy, N(R)₂, CF₃, phenyl, halophenyl, (benzyloxy)phenyl, CN, NO₂; each represents a separate embodiment according to this invention.

In some embodiments, L₁ of formula I, I(a) and III-VII is CH₂. In other embodiments, L₁ is C═O. In other embodiments, L₁ is CHR. In other embodiments, L₁ is C(R)₂. In other embodiments, R is H, F, C₁-C₅ linear or branched, substituted or unsubstituted alkyl, methyl; each represents a separate embodiment according to this invention. In other embodiments, two geminal R substitutions are joined together to form a 3-6 membered substituted or unsubstituted, aliphatic ring. In other embodiments, two geminal R substitutions are joined together to form a cyclopropyl ring.

In some embodiments, L₂ of formula I and/or I(a) is a bond. In other embodiments, L₂ is CH₂. In other embodiments, L₂ is C═O. In other embodiments, L₂ is O. In other embodiments, L₂ is S.

In some embodiments, X₁ of compound of formula I, I(a) II, III and/or IV is N. In other embodiments, X₁ is C—R. In other embodiments, X₁ is C—H. In other embodiments, X₁ is C—OH.

In some embodiments, X₂ of compound of formula III and/or V-VIII is NH. In other embodiments, X₂ is S. In other embodiments, X₂ is O. In other embodiments, X₂ is N—R. In other embodiments, X₂ is N—CH₂—CH₂—O—CH₃.

In some embodiments, X₃ of compound of formula III and/or V-VIII is N. In other embodiments, X₃ is C(R). In other embodiments, X₃ is CH. In other embodiments, X₃ is C—CH₃. In other embodiments, X₃ is C—Cl. In other embodiments, X₃ is C—CN.

In some embodiments, X₄ of compound of formula III, V and/or V(a) is C. In other embodiments, X₄ is N.

In some embodiments, X₅ of compound of formula III, V and/or V(a) is C. In other embodiments, X₅ is N.

In some embodiments, X₆ of compound of formula III, V and/or V(a) is C. In other embodiments, X₆ is N.

In some embodiments, X₇ of compound of formula III, V and/or V(a) is C. In other embodiments, X₇ is N.

In some embodiments, X₈ of compound of formula IV, V, V(a) and/or VI is C. In other embodiments, X₈ is N.

In some embodiments, X₉ of compound of formula IV, V, V(a) and/or VI is C. In other embodiments, X₉ is N.

In some embodiments, X₁₀ of compound of formula IV, V-VIII and/or IX is C. In other embodiments, X₁₀ is N.

In some embodiments, X₁₁ of compound of formula IV, V, V(a) and/or VI is C. In other embodiments, X₁₁ is N.

In some embodiments, X₁₂ of compound of formula IV, V-VIII and/or IX is C. In other embodiments, X₁₂ is N.

In some embodiments, at least one of X₄-X₇ is N.

In some embodiments, at least one of X₈-X₁₂ is N. In some embodiments, at least two of X₈-X₁₂ are N. In some embodiments, at least one of X₁₀ and X₁₂ is N.

As used herein, “single or fused aromatic or heteroaromatic ring systems” can be any such ring, including but not limited to phenyl, naphthyl, pyridinyl, (2-, 3-, and 4-pyridinyl), quinolinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, pyrazolyl, pyrrolyl, furanyl, thiophene-yl, quinolinyl, isoquinolinyl, 2,3-dihydroindenyl, indenyl, tetrahydronaphthyl, 3,4-dihydro-2H-benzo[b][1,4]dioxepine benzodioxolyl, benzo[d][1,3]dioxole, tetrahydronaphthyl, indolyl, 1H-indole, isoindolyl, anthracenyl, benzimidazolyl, 2,3-dihydro-1H-benzo[d]imidazolyl, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, 1,2,3,4-tetrahydroquinoxaline, 1-(pyridin-1(2H)-yl)ethanone, cinnolinyl, phthalazinyl, quinolinyl, isoquinolinyl, acridinyl, benzofuranyl, 1-benzofuran, isobenzofuranyl, benzofuran-2(3H)-one, benzothiophenyl, benzoxadiazole, benzo[c][1,2,5]oxadiazolyl, benzo[c]thiophenyl, benzodioxolyl, thiadiazolyl, [1,3]oxazolo[4,5-b]pyridine, oxadiaziolyl, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]thiazole, thieno[3,2-d]pyrimidin-4(3H)-one, 4-oxo-4H-thieno[3,2-d][1,3]thiazin, imidazo[1,2-a]pyridine, 1H-imidazo[4,5-b]pyridine, 1H-imidazo[4,5-c]pyridine, 3H-imidazo[4,5-c]pyridine, pyrazolo[1,5-a]pyridine, imidazo[1,2-a]pyrazine, imidazo[1,2-a]pyrimidine, 1H-pyrrolo[2,3-b]pyridine, pyrido[2,3-b]pyrazine, pyrido[2,3-b]pyrazin-3(4H)-one, 4H-thieno[3,2-b]pyrrole, quinoxalin-2(1H)-one, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine, 3-methyl-4H-1,2,4-triazole, 5-methyl-1,2,4-oxadiazole, etc.

As used herein, the term “alkyl” can be any straight- or branched-chain alkyl group containing up to about 30 carbons unless otherwise specified. In various embodiments, an alkyl includes C₁-C₅ carbons. In some embodiments, an alkyl includes C₁-C₆ carbons. In some embodiments, an alkyl includes C₁-C₅ carbons. In some embodiments, an alkyl includes C₁-C₁₀ carbons. In some embodiments, an alkyl is a C₁-C₁₂ carbons. In some embodiments, an alkyl is a C₁-C₂₀ carbons. In some embodiments, branched alkyl is an alkyl substituted by alkyl side chains of 1 to 5 carbons. In various embodiments, the alkyl group may be unsubstituted. In some embodiments, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof.

The alkyl group can be a sole substituent, or it can be a component of a larger substituent, such as in an alkoxy, alkoxyalkyl, haloalkyl, arylalkyl, alkylamino, dialkylamino, alkylamido, alkylurea, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylamido, methyl-urea, ethyl-urea, propyl-urea, 2, 3, or 4-CH₂—C₆H₄—Cl, C(OH)(CH₃)(Ph), etc.

As used herein, the term “aryl” refers to any aromatic ring that is directly bonded to another group and can be either substituted or unsubstituted. The aryl group can be a sole substituent, or the aryl group can be a component of a larger substituent, such as in an arylalkyl, arylamino, arylamido, etc. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, indolyl, phenylmethyl, phenylethyl, phenylamino, phenylamido, 3-methyl-4H-1,2,4-triazolyl, 5-methyl-1,2,4-oxadiazolyl, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, phenyl, halophenyl, (benzyloxy)phenyl, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O— alkyl, C(O)H, —C(O)NH₂ or any combination thereof.

As used herein, the term “alkoxy” refers to an ether group substituted by an alkyl group as defined above. Alkoxy refers both to linear and to branched alkoxy groups. Nonlimiting examples of alkoxy groups are methoxy, ethoxy, propoxy, iso-propoxy, tert-butoxy.

As used herein, the term “aminoalkyl” refers to an amine group substituted by an alkyl group as defined above. Aminoalkyl refers to monoalkylamine, dialkylamine or trialkylamine. Nonlimiting examples of aminoalkyl groups are —N(Me)₂, —NHMe, —NH₃.

A “haloalkyl” group refers, in some embodiments, to an alkyl group as defined above, which is substituted by one or more halogen atoms, e.g. by F, Cl, Br or I. The term “haloalkyl” include but is not limited to fluoroalkyl, i.e., to an alkyl group bearing at least one fluorine atom. Nonlimiting examples of haloalkyl groups are CF₃, CF₂CF₃, CF₂CH₃, CH₂CF₃, CF₂CH₂CH₃, CH₂CH₂CF₃, CF₂CH(CH₃)₂ and CF(CH₃)—CH(CH₃)₂.

A “halophenyl” group refers, in some embodiments, to a phenyl substitutent which is substituted by one or more halogen atoms, e.g. by F, Cl, Br or I. In one embodiment, the halophenyl is 4-chlorophenyl.

An “alkoxyalkyl” group refers, in some embodiments, to an alkyl group as defined above, which is substituted by alkoxy group as defined above, e.g. by methoxy, ethoxy, propoxy, i-propoxy, t-butoxy etc. Nonlimiting examples of alkoxyalkyl groups are —CH₂—O—CH₃, —CH₂—O—CH(CH₃)₂, —CH₂—O—C(CH₃)₃, —CH₂—CH₂—O—CH₃, —CH₂—CH₂—O—CH(CH₃)₂, —CH₂—CH₂—O—C(CH₃)₃.

A “cycloalkyl” or “carbocyclic” group refers, in various embodiments, to a ring structure comprising carbon atoms as ring atoms, which may be either saturated or unsaturated, substituted or unsubstituted, single or fused. In some embodiments the cycloalkyl is a 3-10 membered ring. In some embodiments the cycloalkyl is a 3-12 membered ring. In some embodiments the cycloalkyl is a 6 membered ring. In some embodiments the cycloalkyl is a 5-7 membered ring. In some embodiments the cycloalkyl is a 3-8 membered ring. In some embodiments, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof. In some embodiments, the cycloalkyl ring may be fused to another saturated or unsaturated cycloalkyl or heterocyclic 3-8 membered ring. In some embodiments, the cycloalkyl ring is a saturated ring. In some embodiments, the cycloalkyl ring is an unsaturated ring. Non limiting examples of a cycloalkyl group comprise cyclohexyl, cyclohexenyl, cyclopropyl, cyclopropenyl, cyclopentyl, cyclopentenyl, cyclobutyl, cyclobutenyl, cycloctyl, cycloctadienyl (COD), cycloctaene (COE) etc.

A “heterocycle” or “heterocyclic” group refers, in various embodiments, to a ring structure comprising in addition to carbon atoms, sulfur, oxygen, nitrogen or any combination thereof, as part of the ring. A “heteroaromatic ring” refers in various embodiments, to an aromatic ring structure comprising in addition to carbon atoms, sulfur, oxygen, nitrogen or any combination thereof, as part of the ring. In some embodiments the heterocycle or heteroaromatic ring is a 3-10 membered ring. In some embodiments the heterocycle or heteroaromatic ring is a 3-12 membered ring. In some embodiments the heterocycle or heteroaromatic ring is a 6 membered ring. In some embodiments the heterocycle or heteroaromatic ring is a 5-7 membered ring. In some embodiments the heterocycle or heteroaromatic ring is a 3-8 membered ring. In some embodiments, the heterocycle group or heteroaromatic ring may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof. In some embodiments, the heterocycle ring or heteroaromatic ring may be fused to another saturated or unsaturated cycloalkyl or heterocyclic 3-8 membered ring. In some embodiments, the heterocyclic ring is a saturated ring. In some embodiments, the heterocyclic ring is an unsaturated ring. Non limiting examples of a heterocyclic ring or heteroaromatic ring systems comprise pyridine, piperidine, morpholine, piperazine, thiophene, pyrrole, benzodioxole, benzofuran-2(3H)-one, benzo[d][1,3]dioxole, indole, oxazole, isoxazole, imidazole and 1-methylimidazole, furane, triazole, pyrimidine, pyrazine, oxacyclobutane (1 or 2-oxacyclobutane), naphthalene, tetrahydrothiophene 1,1-dioxide, thiazole, benzimidazole, piperidine, 1-methylpiperidine, isoquinoline, 1,3-dihydroisobenzofuran, benzofuran, 3-methyl-4H-1,2,4-triazole, 5-methyl-1,2,4-oxadiazole, or indole.

In various embodiments, this invention provides a compound of this invention or its isomer, metabolite, pharmaceutically acceptable salt, pharmaceutical product, tautomer, hydrate, N-oxide, prodrug, isotopic variant (deuterated analog), polymorph, or crystal or combinations thereof. In various embodiments, this invention provides an isomer of the compound of this invention. In some embodiments, this invention provides a metabolite of the compound of this invention. In some embodiments, this invention provides a pharmaceutically acceptable salt of the compound of this invention. In some embodiments, this invention provides a pharmaceutical product of the compound of this invention. In some embodiments, this invention provides a tautomer of the compound of this invention. In some embodiments, this invention provides a hydrate of the compound of this invention. In some embodiments, this invention provides an N-oxide of the compound of this invention. In some embodiments, this invention provides a prodrug of the compound of this invention. In some embodiments, this invention provides an isotopic variant (including but not limited to deuterated analog) of the compound of this invention. In some embodiments, this invention provides a PROTAC (Proteolysis targeting chimera) of the compound of this invention. In some embodiments, this invention provides a polymorph of the compound of this invention. In some embodiments, this invention provides a crystal of the compound of this invention. In some embodiments, this invention provides composition comprising a compound of this invention, as described herein, or, In some embodiments, a combination of an isomer, metabolite, pharmaceutically acceptable salt, pharmaceutical product, tautomer, hydrate, N-oxide, prodrug, isotopic variant (deuterated analog), polymorph, or crystal of the compound of this invention.

In various embodiments, the term “isomer” includes, but is not limited to, stereoisomers and analogs, structural isomers and analogs, conformational isomers and analogs, and the like. In some embodiments, the isomer is an optical isomer. In some embodiments, the isomer is a stereoisomer.

In various embodiments, this invention encompasses the use of various stereoisomers of the compounds of the invention. It will be appreciated by those skilled in the art that the compounds of the present invention may contain at least one chiral center. Accordingly, the compounds used in the methods of the present invention may exist in, and be isolated in, optically-active or racemic forms. Accordingly, the compounds according to this invention may exist as optically-active isomers (enantiomers or diastereomers, including but not limited to: the (R), (S), (R)(R), (R)(S), (S)(S), (S)(R), (R)(R)(R), (R)(R)(S), (R)(S)(R), (S)(R)(R), (R)(S)(S), (S)(R)(S), (S)(S)(R) or (S)(S)(S) isomers); as racemic mixtures, or as enantiomerically enriched mixtures. Some compounds may also exhibit polymorphism. It is to be understood that the present invention encompasses any racemic, optically-active, polymorphic, or stereroisomeric form, or mixtures thereof, which form possesses properties useful in the treatment of the various conditions described herein.

It is well known in the art how to prepare optically-active forms (for example, by resolution of the racemic form by recrystallization techniques, by synthesis from optically-active starting materials, by chiral synthesis, or by chromatographic separation using a chiral stationary phase).

The compounds of the present invention can also be present in the form of a racemic mixture, containing substantially equivalent amounts of stereoisomers. In some embodiments, the compounds of the present invention can be prepared or otherwise isolated, using known procedures, to obtain a stereoisomer substantially free of its corresponding stereoisomer (i.e., substantially pure). By substantially pure, it is intended that a stereoisomer is at least about 95% pure, more preferably at least about 98% pure, most preferably at least about 99% pure.

Compounds of the present invention can also be in the form of a hydrate, which means that the compound further includes a stoichiometric or non-stoichiometric amount of water bound by non-covalent intermolecular forces.

As used herein, when some chemical functional group (e.g. alkyl or aryl) is said to be “substituted”, it is herein defined that one or more substitutions are possible.

Compounds of the present invention may exist in the form of one or more of the possible tautomers and depending on the conditions it may be possible to separate some or all of the tautomers into individual and distinct entities. It is to be understood that all of the possible tautomers, including all additional enol and keto tautomers and/or isomers are hereby covered. For example, the following tautomers, but not limited to these, are included: Tautomerization of the imidazole ring:

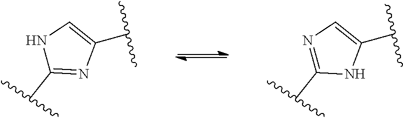 [SMILES: *c1c[nH]c(*)n1 ; *c1cnc(*)[nH]1]

Tautomerization of the pyrazolone ring:

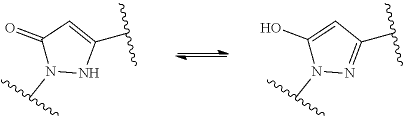 [SMILES: *c1cc(=O)n(*)[nH]1 ; *c1cc(O)n(*)n1]

The invention includes “pharmaceutically acceptable salts” of the compounds of this invention, which may be produced, by reaction of a compound of this invention with an acid or base. Certain compounds, particularly those possessing acid or basic groups, can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically-acceptable salts of amines of compounds of this invention may be prepared from an inorganic acid or from an organic acid. In various embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In various embodiments, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In some embodiments, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In various embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

### Pharmaceutical Composition

Another aspect of the present invention relates to a pharmaceutical composition including a pharmaceutically acceptable carrier and a compound according to the aspects of the present invention. The pharmaceutical composition can contain one or more of the above-identified compounds of the present invention. Typically, the pharmaceutical composition of the present invention will include a compound of the present invention or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Treatment regimen for the administration of the compounds of the present invention can also be determined readily by those with ordinary skill in art. That is, the frequency of administration and size of the dose can be established by routine optimization, preferably while minimizing any side effects.

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In some embodiments, these compounds are tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, these active compounds can be incorporated with excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 mg and 800 mg of active compound.

The active compounds of the present invention may be orally administered, for example, with an inert diluent, or with an assimilable edible carrier, or they can be enclosed in hard or soft shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

In various embodiments, the compounds of this invention are administered in combination with an agent treating fibrosis. In some embodiment, the agent treating lung fibrosis is at least one selected from: pirfenidone and Nintedanib. Other examples of agents which can be useful in treating lung fibrosis including IPF, in combination with compound of the invention, include but are not limited to: Pioglitazone, Tralokinumab, Lebrikizumab, FG-3019, Simtuzumab, STX-100, BMS-986020, R₁ tuximab, Carbon Monoxide, Azithromycin, and Cotrimoxazole. In various embodiments, the compounds of this invention are administered in combination with an agent treating NASH.

When administering the compounds of the present invention, they can be administered systemically or, alternatively, they can be administered directly to a specific site where fibrosis is present. Thus, administering can be accomplished in any manner effective for delivering the compounds or the pharmaceutical compositions to the fibrotic cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

### Biological Activity

In various embodiments, the invention provides compounds and compositions, including any embodiment described herein, for use in any of the methods of this invention. In various embodiments, use of a compound of this invention or a composition comprising the same, will have utility in inhibiting, suppressing, enhancing or stimulating a desired response in a subject, as will be understood by one skilled in the art. In some embodiments, the compositions may further comprise additional active ingredients, whose activity is useful for the particular application for which the compound of this invention is being administered.

The invention relates to the treatment, inhibition and reduction of fibrosis, including lung and hepatic fibrosis. More specifically, embodiments of the invention provide compositions and methods useful for the treatment and inhibition of fibrotic disorders, lung fibrosis, Idiotypic pulmonary fibrosis (IPF), hepato-fibrotic conditions associated with Non-Alcoholic Fatty Liver Disease (NAFLD) and Non-Alcoholic Steatohepatitis (NASH), employing the use of a compound according to this invention or a pharmaceutically acceptable salt thereof. In another embodiment, the human subject is afflicted with lung fibrosis. In another embodiment, the human subject is afflicted with Idiotypic pulmonary fibrosis (IPF). In another embodiment, the human subject is afflicted with Non-Alcoholic Fatty Liver Disease (NAFLD). In another embodiment, the human subject is afflicted with Non-Alcoholic Steatohepatitis (NASH). In another embodiment, the human subject is not afflicted with Non-Alcoholic Steatohepatitis (NASH).

In various conditions, the formation of fibrotic tissue is characterized by the deposition of abnormally large amounts of collagen. The synthesis of collagen is also involved in a number of other pathological conditions. For example, clinical conditions and disorders associated with primary or secondary fibrosis, such as systemic sclerosis, graft-versus host disease (GVHD), pulmonary fibrosis and autoimmune disorders, are distinguished by excessive production of connective tissue, which results in the destruction of normal tissue architecture and function. These diseases can best be interpreted in terms of perturbations in cellular functions, a major manifestation of which is excessive collagen synthesis and deposition. The role of collagen in fibrosis has prompted attempts to develop drugs that inhibit its accumulation.

Excessive accumulation of collagen is the major pathologic feature in a variety of clinical conditions characterized by tissue fibrosis. These conditions include localized processes, as for example, pulmonary fibrosis and liver cirrhosis, or more generalized processes, like progressive systemic sclerosis. Collagen deposition is a feature of different forms of dermal fibrosis, which in addition to scleroderma, include localized and generalized morphea, keloids, hypertrophic scars, familial cutaneous collagenoma and connective tissue nevi of the collagen type. Recent advances in the understanding of the normal biochemistry of collagen have allowed us to define specific levels of collagen biosynthesis and degradation at which a pharmacologic intervention could lead to reduced collagen deposition in the tissues. Such compounds could potentially provide us with novel means to reduce the excessive collagen accumulation in diseases.

Accordingly, in various embodiments, this invention is directed to a method of treating, suppressing, reducing the severity, reducing the risk of developing or inhibiting fibrosis in a subject, comprising administering a compound according to this invention, to a subject suffering from fibrosis under conditions effective to treat, suppress, reduce the severity, reduce the risk of developing, or inhibit fibrosis in said subject. In some embodiments, the fibrosis is systemic. In some embodiments, the fibrosis is organ specific. In some embodiments, the fibrosis is a result of wound healing. In some embodiments, the fibrosis is a result of scarring. In some embodiments, the fibrosis is primary or secondary fibrosis. In some embodiments, the fibrosis is a result of systemic sclerosis, progressive systemic sclerosis, graft-versus host disease (GVHD), pulmonary fibrosis, autoimmune disorders, or any combination thereof; each represents a separate embodiment according to this invention. In another embodiment, the human subject is afflicted with lung fibrosis. In another embodiment, the human subject is afflicted with Idiotypic pulmonary fibrosis (IPF). In some embodiments, the fibrosis is pulmonary fibrosis. In some embodiments, the subject has a liver cirrhosis. In some embodiments, the fibrosis is hepatic fibrosis, lung fibrosis or dermal fibrosis. In some embodiments, the dermal fibrosis is scleroderma. In some embodiments, the dermal fibrosis is a result of a localized or generalized morphea, keloids, hypertrophic scars, familial cutaneous collagenoma, connective tissue nevi of the collagen type, or any combination thereof; each represents a separate embodiment according to this invention. In some embodiments, the fibrosis results from tissue injury, inflammation, oxidative stress or any combination thereof; each represents a separate embodiment according to this invention. In some embodiments, the fibrosis is gingival fibromatosis. In some embodiments, the compound is a Collagen I translation inhibitor. In some embodiments, the compound is any one of the compounds listed in Table 1; each compound represents a separate embodiment according to this invention.

Human fibrotic diseases constitute a major health problem worldwide owing to the large number of affected individuals, the incomplete knowledge of the fibrotic process pathogenesis, the marked heterogeneity in their etiology and clinical manifestations, the absence of appropriate and fully validated biomarkers, and, most importantly, the current void of effective disease-modifying therapeutic agents. The fibrotic disorders encompass a wide spectrum of clinical entities including systemic fibrotic diseases such as systemic sclerosis (SSc), sclerodermatous graft vs. host disease, and nephrogenic systemic fibrosis, as well as numerous organ-specific disorders including radiation-induced fibrosis and cardiac, pulmonary, lung, liver, and kidney fibrosis. Although their causative mechanisms are quite diverse and, in several instances have remained elusive, these diseases share the common feature of an uncontrolled and progressive accumulation of fibrotic tissue in affected organs causing their dysfunction and ultimate failure. Despite the remarkable heterogeneity in the etiologic mechanisms responsible for the development of fibrotic diseases and in their clinical manifestations, numerous studies have identified activated myofibroblasts as the common cellular element ultimately responsible for the replacement of normal tissues with nonfunctional fibrotic tissue.

In various embodiments, this invention is directed to a method of treating, suppressing, reducing the severity, reducing the risk of developing or inhibiting systemic fibrotic disease in a subject, comprising administering a compound according to this invention, to a subject suffering from a systemic fibrotic disease under conditions effective to treat, suppress, reduce the severity, reduce the risk of developing, or inhibit the systemic fibrotic disease in said subject. In some embodiments, the systemic fibrotic disease is systemic sclerosis. In some embodiments, the systemic fibrotic disease is multifocal fibrosclerosis (IgG4-associated fibrosis). In some embodiments, the systemic fibrotic disease is nephrogenic systemic fibrosis. In some embodiments, the systemic fibrotic disease is sclerodermatous graft vs. host disease.

In various embodiments, this invention is directed to a method of treating, suppressing, reducing the severity, reducing the risk of developing or inhibiting an organ-specific fibrotic disease in a subject, comprising administering a compound according to this invention, to a subject suffering from an organ-specific fibrotic disease under conditions effective to treat, suppress, reduce the severity, reduce the risk of developing, or inhibit the organ-specific fibrotic disease in said subject.

In some embodiments, the organ-specific fibrotic disease is lung fibrosis. In some embodiments, the organ-specific fibrotic disease is Idiotypic pulmonary fibrosis (IPF).

In some embodiments, the organ-specific fibrotic disease is cardiac fibrosis. In some embodiments, the cardiac fibrosis is hypertension-associated cardiac fibrosis. In some embodiments, the cardiac fibrosis is post-myocardial infarction. In some embodiments, the cardiac fibrosis is chagas disease-induced myocardial fibrosis.

In some embodiments, the organ-specific fibrotic disease is kidney fibrosis. In some embodiments, the kidney fibrosis is diabetic and hypertensive nephropathy. In some embodiments, the kidney fibrosis is urinary tract obstruction-induced kidney fibrosis. In some embodiments, the kidney fibrosis is inflammatory/autoimmune-induced kidney fibrosis. In some embodiments, the kidney fibrosis is aristolochic acid nephropathy. In some embodiments, the kidney fibrosis is polycystic kidney disease.

In various embodiments, this invention is directed to a method of treating, suppressing, reducing the severity, reducing the risk of developing or inhibiting cardiac fibrosis in a subject, comprising administering a compound of this invention, to a subject suffering from cardiac fibrosis under conditions effective to treat, suppress, reduce the severity, reduce the risk of developing, or inhibit cardiac fibrosis in said subject. In some embodiments, the compound is a Collagen I translation inhibitor. In some embodiments, the compound is any one of the compounds listed in Table 1; each compound represents a separate embodiment according to this invention.

In some embodiments, the organ-specific fibrotic disease is pulmonary fibrosis. In some embodiments, the pulmonary fibrosis is idiopathic pulmonary fibrosis. In some embodiments, the pulmonary fibrosis is silica-induced pneumoconiosis (silicosis). In some embodiments, the pulmonary fibrosis is asbestos-induced pulmonary fibrosis (asbestosis). In some embodiments, the pulmonary fibrosis is chemotherapeutic agent-induced pulmonary fibrosis.

In some embodiments, the organ-specific fibrotic disease is liver and portal vein fibrosis. In some embodiments, the liver and portal vein fibrosis is alcoholic and nonalcoholic liver fibrosis. In some embodiments, the liver and portal vein fibrosis is hepatitis C-induced liver fibrosis. In some embodiments, the liver and portal vein fibrosis is primary biliary cirrhosis. In some embodiments, the liver and portal vein fibrosis is parasite-induced liver fibrosis (schistosomiasis).

In some embodiments, the organ-specific fibrotic disease is radiation-induced fibrosis (various organs). In some embodiments, the organ-specific fibrotic disease is bladder fibrosis. In some embodiments, the organ-specific fibrotic disease is intestinal fibrosis. In some embodiments, the organ-specific fibrotic disease is peritoneal sclerosis.

In some embodiments, the organ-specific fibrotic disease is diffuse fasciitis. In some embodiments, the diffuse fasciitis is localized scleroderma, keloids. In some embodiments, the diffuse fasciitis is dupuytren's disease. In some embodiments, the diffuse fasciitis is peyronie's disease. In some embodiments, the diffuse fasciitis is myelofibrosis. In some embodiments, the diffuse fasciitis is oral submucous fibrosis.

In some embodiments, the organ-specific fibrotic disease is a result of wound healing. In some embodiments, the organ-specific fibrotic disease is a result of scarring.

Fibrosis of the liver, also referred to herein as hepatic fibrosis, may be caused by various types of chronic liver injury, especially if an inflammatory component is involved. Self-limited, acute liver injury (e.g., acute viral hepatitis A), even when fulminant, does not necessarily distort the scaffolding architecture and hence does not typically cause fibrosis, despite loss of hepatocytes. However, factors such as chronic alcoholism, malnutrition, hemochromatosis, and exposure to poisons, toxins or drugs, may lead to chronic liver injury and hepatic fibrosis due to exposure to hepatotoxic chemical substances. Hepatic scarring, caused by surgery or other forms of injury associated with mechanical biliary obstruction, may also result in liver fibrosis.

In various embodiments, this invention is directed to a method of treating, suppressing, reducing the severity, reducing the risk of developing or inhibiting hepatic fibrosis in a subject, comprising administering a compound of this invention, to a subject suffering from hepatic fibrosis under conditions effective to treat, suppress, reduce the severity, reduce the risk of developing, or inhibit hepatic fibrosis in said subject. In some embodiments, the hepatic fibrosis results from hepatic scarring. In some embodiments, the hepatic fibrosis results from chronic liver injury. In some embodiments, the chronic liver injury results from chronic alcoholism, malnutrition, hemochromatosis, exposure to poisons, toxins or drugs; each represents a separate embodiment according to this invention. In some embodiments, the subject has a liver cirrhosis. In some embodiments, the compound is a Collagen I translation inhibitor. In some embodiments, the compound is any one of the compounds listed in Table 1; each compound represents a separate embodiment according to this invention.

Fibrosis itself is not necessarily symptomatic, however it can lead to the development of portal hypertension, in which scarring distorts blood flow through the liver, or cirrhosis, in which scarring results in disruption of normal hepatic architecture and liver dysfunction. The extent of each of these pathologies determines the clinical manifestation of hepato-fibrotic disorders. For example, congenital hepatic fibrosis affects portal vein branches, largely sparing the parenchyma. The result is portal hypertension with sparing of hepatocellular function.

In various embodiments, this invention is directed to a method of treating, suppressing, reducing the severity, reducing the risk of developing or inhibiting an hepato-fibrotic disorder in a subject, comprising administering a compound of this invention, to a subject suffering from hepato-fibrotic disorder under conditions effective to treat, suppress, reduce the severity, reduce the risk of developing, or inhibit the hepato-fibrotic disorder in said subject. In some embodiments, the hepato-fibrotic disorder is: portal hypertension, cirrhosis, congenital hepatic fibrosis or any combination thereof; each represents a separate embodiment according to this invention. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting portal hypertension in a subject, comprising administering a compound of this invention, to a subject suffering from portal hypertension under conditions effective to treat, suppress, reduce the severity, reduce the risk of developing, or inhibit portal hypertension in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting cirrhosis in a subject, comprising administering a compound of this invention, to a subject suffering from cirrhosis under conditions effective to treat, suppress, reduce the severity, reduce the risk of developing, or inhibit cirrhosis in said subject. In some embodiments, the cirrhosis is a result of hepatitis. In some embodiments, the cirrhosis is a result of alcoholism. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting human alcoholism in a subject, comprising administering a compound of this invention, to a subject suffering from alcoholism under conditions effective to treat, suppress, reduce the severity, reduce the risk of developing, or inhibit alcoholism in said subject. In some embodiments, the compound is a Collagen I translation inhibitor. In some embodiments, the compound is any one of the compounds listed in Table 1; each compound represents a separate embodiment according to this invention.

Non-alcoholic steatohepatitis (NASH) and alcoholic steatohepatitis (ASH) have a similar pathogenesis and histopathology but a different etiology and epidemiology. NASH and ASH are advanced stages of non-alcoholic fatty liver disease (NAFLD) and alcoholic fatty liver disease (AFLD). NAFLD is characterized by excessive fat accumulation in the liver (steatosis), without any other evident causes of chronic liver diseases (viral, autoimmune, genetic, etc.), and with an alcohol consumption Z 20-30 g/day. On the contrary, AFLD is defined as the presence of steatosis and alcohol consumption>20-30 g/day.

In various embodiments, this invention is directed to a method of treating, suppressing, reducing the severity, reducing the risk of developing or inhibiting Non-alcoholic steatohepatitis (NASH) in a subject, comprising administering a compound of this invention, to a subject suffering from Non-alcoholic steatohepatitis (NASH) under conditions effective to treat, suppress, reduce the severity, reduce the risk of developing, or inhibit Non-alcoholic steatohepatitis (NASH) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting alcoholic steatohepatitis (ASH) in a subject, comprising administering a compound of this invention, to a subject suffering from alcoholic steatohepatitis (ASH) under conditions effective to treat, suppress, reduce the severity, reduce the risk of developing, or inhibit alcoholic steatohepatitis (ASH) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting non-alcoholic fatty liver disease (NAFLD) in a subject, comprising administering a compound of this invention, to a subject suffering from non-alcoholic fatty liver disease (NAFLD) under conditions effective to treat, suppress, reduce the severity, reduce the risk of developing, or inhibit non-alcoholic fatty liver disease (NAFLD) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting alcoholic fatty liver disease (AFLD) in a subject, comprising administering a compound of this invention, to a subject suffering from alcoholic fatty liver disease (AFLD) under conditions effective to treat, suppress, reduce the severity, reduce the risk of developing, or inhibit alcoholic fatty liver disease (AFLD) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting lung fibrosis in a subject, comprising administering a compound of this invention, to a subject suffering from lung fibrosis under conditions effective to treat, suppress, reduce the severity, reduce the risk of developing, or inhibit lung fibrosis in said subject. In some embodiments, the compound is a Collagen I translation inhibitor. In some embodiments, the compound is any one of the compounds listed in Table 1; each compound represents a separate embodiment according to this invention.

Idiopathic pulmonary fibrosis (IPF) is an aging-associated recalcitrant lung disease with historically limited therapeutic options. The recent approval of two drugs, pirfenidone and nintedanib, by the United States Food and Drug Administration (FDA) in 2014 has heralded a new era in its management. Both drugs demonstrated efficacy in Phase III clinical trials by retarding the rate of progression of IPF; neither drug appears to be able to completely arrest disease progression. Advances in the understanding of IPF pathobiology have led to an unprecedented expansion in the number of potential therapeutic targets. Drugs targeting several of these are under investigation in various stages of clinical development.

In various embodiments, this invention is directed to a method of treating, suppressing, reducing the severity, reducing the risk of developing or inhibiting idiopathic pulmonary fibrosis (IPF) in a subject, comprising administering a compound of this invention, to a subject suffering from idiopathic pulmonary fibrosis (IPF) under conditions effective to treat, suppress, reduce the severity, reduce the risk of developing, or inhibit idiopathic pulmonary fibrosis (IPF) in said subject. In some embodiments, the compound is a Collagen I translation inhibitor. In some embodiments, the compound is any one of the compounds listed in Table 1; each compound represents a separate embodiment according to this invention. In some embodiments, the compound is administered in combination with an agent treating IPF. In some embodiments, the compound is administered in combination with pirfenidone, nintedanib, or combination thereof; each represents a separate embodiment according to this invention.

In various embodiments, this invention is directed to a method of treating, suppressing, reducing the severity, reducing the risk of developing or inhibiting dermal fibrosis in a subject, comprising administering a compound of this invention, to a subject suffering from dermal fibrosis under conditions effective to treat, suppress, reduce the severity, reduce the risk of developing, or inhibit dermal fibrosis in said subject. In some embodiments, the dermal fibrosis is scleroderma. In some embodiments, the dermal fibrosis is a result of a localized or generalized morphea, keloids, hypertrophic scars, familial cutaneous collagenoma, connective tissue nevi of the collagen type, or any combination thereof; each represents a separate embodiment according to this invention. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting scleroderma in a subject, comprising administering a compound of this invention, to a subject suffering from scleroderma under conditions effective to treat, suppress, reduce the severity, reduce the risk of developing, or inhibit scleroderma in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of inhibiting Collagen I (Col I) over production in a subject, comprising administering a compound of this invention, to a subject suffering from Collagen I (Col I) over production under conditions effective to inhibit Collagen I (Col I) over production in said subject. In some embodiments, the compound is a Collagen I translation inhibitor. In some embodiments, the compound is any one of the compounds listed in Table 1; each compound represents a separate embodiment according to this invention.

In some embodiments, this invention is directed to a method of treating, suppressing, reducing the severity, reducing the risk of developing or inhibiting an autoimmune disease or disorder in a subject, comprising administering a compound of this invention, to a subject suffering from an autoimmune disease or disorder under conditions effective to treat, suppress, reduce the severity, reduce the risk of developing, or inhibit the autoimmune disease or disorder in said subject. In some embodiments, the compound is a Collagen I translation inhibitor. In some embodiments, the compound is any one of the compounds listed in Table 1; each compound represents a separate embodiment according to this invention.

As used herein, subject or patient refers to any mammalian patient, including without limitation, humans and other primates, dogs, cats, horses, cows, sheep, pigs, rats, mice, and other rodents. In various embodiments, the subject is male. In some embodiments, the subject is female. In some embodiments, while the methods as described herein may be useful for treating either males or females.

The following examples are presented in order to more fully illustrate the preferred embodiments of the invention. They should in no way, however, be construed as limiting the broad scope of the invention.

### EXAMPLES

### Example 1

### Synthetic Details for Compounds of the Invention (Schemes 1-32)

### General Methods

All reagents were commercial grade and were used as received without further purification, unless otherwise specified. Reagent grade solvents were used in all cases, unless otherwise specified. Thin layer chromatography was carried out using pre-coated silica gel F-254 plates (thickness 0.25 mm). ¹H-NMR and ¹⁹F-NMR spectra were recorded on a Bruker Bruker Avance 400 MHz or Avance III 400 MHz spectrometer. The chemical shifts are expressed in ppm using the residual solvent as internal standard. Splitting patterns are designated as s (singlet), d (doublet), dd (doublet of doublets), t (triplet), dt (doublet of triplets), q (quartet), m (multiplet) and br s (broad singlet).

### Abbreviations

### ACN Acetonitrile

AcOH Acetic acid
amphos Bis(di-tert-butyl(4-dimethylaminophenyl)phosphine
Boc tert-Butyloxycarbonyl
BuLi n-butyllithium
t-BuLi tert-butyllithium
DAST Diethylaminosulfur trifluoride
DBU 1,8-Diazabicyclo[5.4.0]undec-7-ene
dppb 1,4-Bis(diphenylphosphino)butane
dppf 1,1′-Bis(diphenylphosphino)ferrocene

### DCM Dichloromethane

DIBAL-H Diisobutylaluminum hydride

### DIPEA N,N-Diisopropylethylamine

### DMF N,N-Dimethylformamide

### DMA Dimethylacetamide

### DME 1,2-Dimethoxyethane

### DMSO Dimethylsulfoxide

HATU [O-(7-Azabenzotriazol-1-yl)-N,N,N′,N′-tetramethyluronium-hexafluorphosphat]
HPLC High performance liquid chromatography
MsCl Methanesulfonyl chloride

### NBS N-Bromosuccinimide

NMP N-Methyl-2-pyrrolidone
PPA Polyphosphoric acid
rt Room temperature

### SEM 2-(Trimethylsilyl)ethoxymethyl

T3P Propylphosphonic anhydride
TBAF Tetrabutylammonium fluoride
TBTU 2-(1H-Benzotriazole-1-yl)-1,1,3,3-tetramethylaminium tetrafluoroborate
TCFH N,N,N,N-tetramethylchloroformamidinium hexafluorophosphate

### THF Tetrahydrofuran

### TMSCF₃ Trimethyl(trifluoromethyl)silane

TMS-OTf Trimethylsilyl trifluoromethanesulfonate

### General Synthesis of Compounds of the Invention

### RHS Piperazine Head-Group Modifications

The original general synthesis towards RHS-modified compounds (Compound 212 analogues, see Table 1 for structures) is shown in Scheme 1 and Scheme 2.

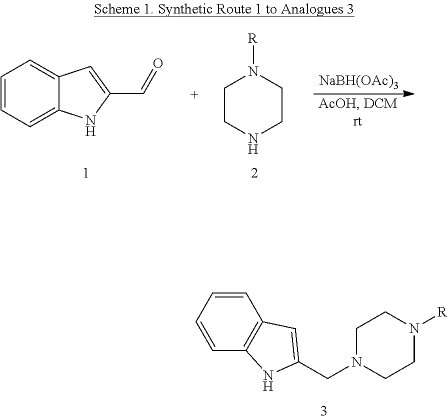

Compound 212 analogues 3 were synthesized via reductive amination chemistry (Route 1) reacting 1H-indole-2-carbaldehyde 1 and the corresponding substituted piperazines 2 in the presence of sodium triacetoxyborohydride and acetic acid in DCM (Scheme 1).

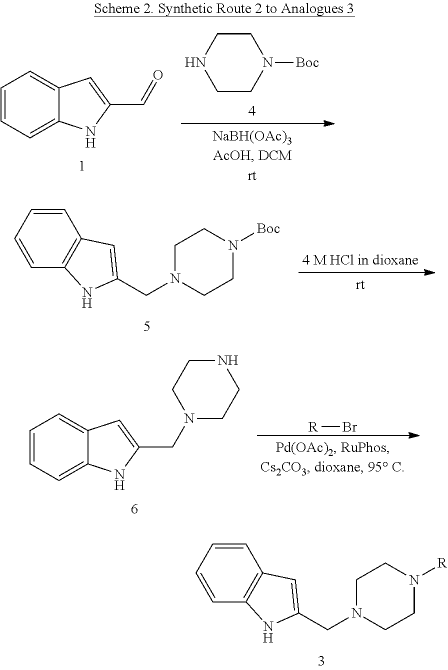

Route 2 (Scheme 2) uses Buchwald chemistry to synthesize compound 212 analogues 3 from (hetero)aryl halides and amine intermediate 6. Amine intermediate 6 was prepared in two steps from commercial 1H-indole-2-carbaldehyde 1 and N-Boc-piperazine 4 via N-Boc intermediate 5. Following the reductive amination first step, the N-Boc intermediate 5 was deprotected under acidic conditions to afford the amine intermediate 6 after generation of the free base via SCX ion exchange chromatography. The final Buchwald chemistry step used various (hetero)aryl halides in the presence of Pd(II) acetate, RuPhos and cesium carbonate in dioxane at 95° C.

### LHS Modifications

The synthesis of 5-carbon-linked LHS modified analogues 11 is outlined in Scheme 3.

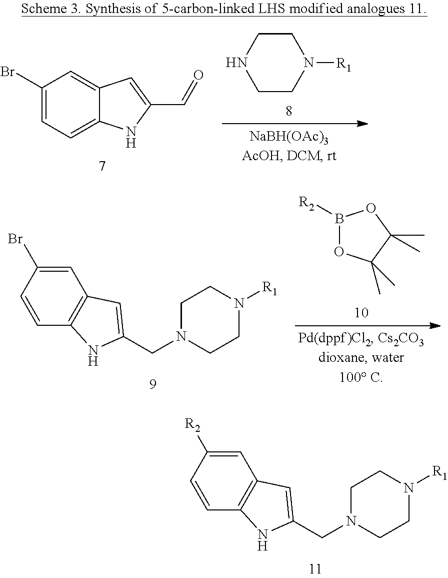

The synthesis to the 5-carbon-linked LHS modified analogues 11 involved initial reductive amination of commercial 5-bromo-1H-indole-2-carbaldehyde 7 with R₁-substituted piperazines 8 using similar conditions of sodium triacetoxyborohydride and acetic acid in DCM. The resulting 5-bromoindole intermediate 9 was subsequently used in a final Suzuki reaction step employing boronic esters 10 to afford the final compound analogues 11 in moderate yields.

The synthesis of of 5-nitrogen-linked LHS modified analogues 17a and 17b is shown in Scheme 4.

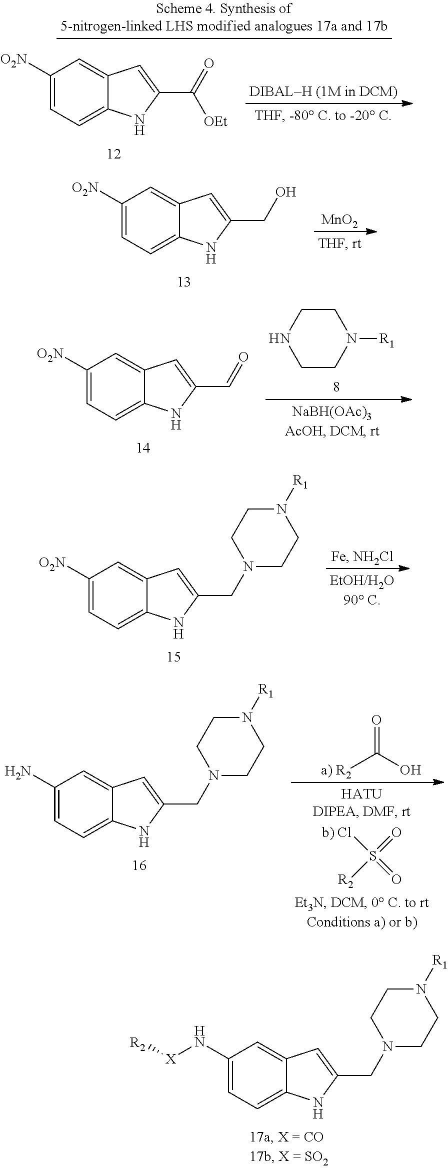

The synthesis started with chemoselective reduction of the ester function of commercial ethyl 5-nitro-1H-indole-2-carboxylate 12 using a solution of diisobutylaluminum hydride in DCM. The resulting alcohol 13 was then oxidized to the aldehyde 14, using manganese(IV) oxide in THF. Subsequent reductive amination of substituted piperazines 8 with the aldehyde intermediate 14 afforded the 5-nitroindole intermediates 15. Reduction of the nitro moiety of these intermediates 15 employing mild conditions of iron powder in the presence of ammonium chloride gave the resulting key 5-aminoindole intermediates 16. The final step involved either amidation of the 5-aminoindole intermediates 15 using carboxylic acids with HATU coupling conditions (conditions a) or sulfonylation of the 5-aminoindole intermediates 15, using the corresponding sulfonyl chloride (conditions b) to afford final compound analogues 17a and 17b respectively (Scheme 4).

The synthesis of reversed 5-indole amide analogues 22 is displayed in Scheme 5.

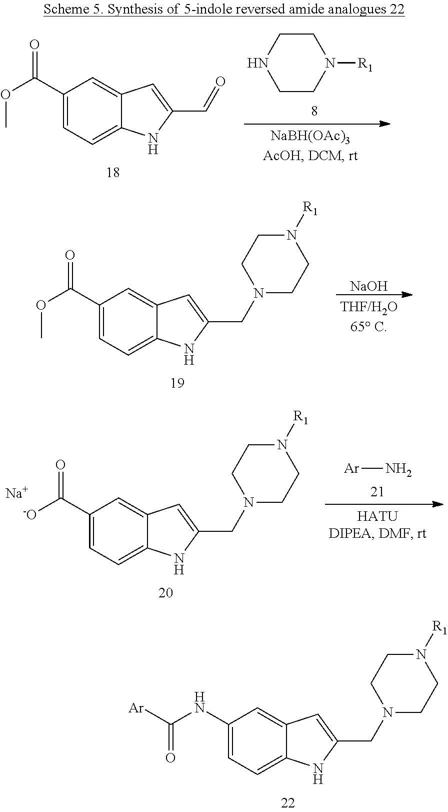

The synthesis of the reversed amine analogues 22 started with reductive amination of substituted piperazines 8 with commercial methyl 2-formyl-1H-indole-5-carboxylate 18 to afford 5-methyl ester indole intermediates 19. Hydrolysis of the ester moiety of the intermediates 19 using sodium hydroxide gave the resulting carboxylic acid intermediates 20, which were isolated as its sodium salt. Amidation of the resulting carboxylic acid sodium salt 20 intermediates with anilines 21 using HATU amide coupling conditions gave the reversed 5-indole amide analogues 22 in good yields.

The synthesis of LHS-modified variable substituted indole scaffold analogues 26 with small R₁ substituents is outlined in Scheme 6.

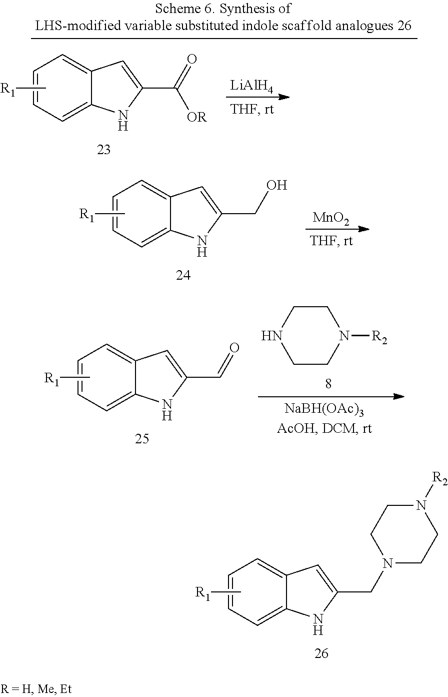

The synthesis started from commercial esters or carboxylic acids 23 bearing R₁ substituents at various indole positions. The commercial esters or carboxylic acids starting materials 23 were converted to aldehyde intermediates 25 in a two-step sequence via the corresponding primary alcohol intermediates 24. The first step of the sequence involved reduction of the carboxylic acid/ester moiety with lithium aluminium hydride in THF. The second step of the sequence was oxidation of primary alcohol intermediates 24 to the aldehyde intermediates 25 using manganese(IV) oxide. The key aldehyde intermediates 25 were then converted to the target analogues 26 employing reductive amination reaction conditions with substituted piperazines 8.

The synthesis of 5-methoxyethoxy indole analogues 31 is displayed in Scheme 7.

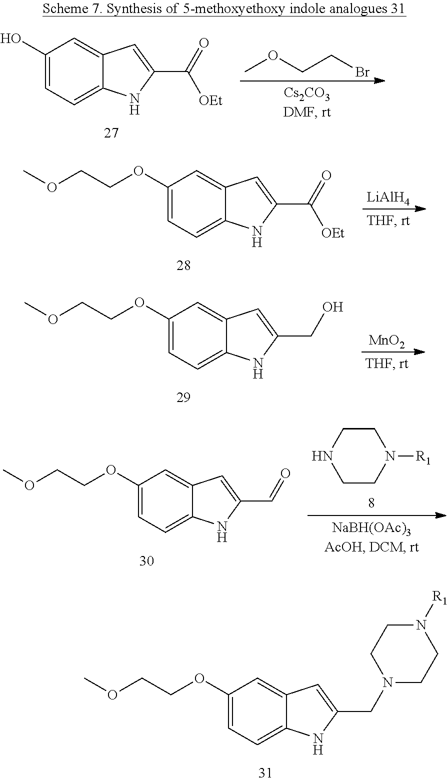

The synthesis of the 5-methoxyethoxy indole analogues 31 commenced with O-alkylation of commercial ethyl 5-hydroxy-1H-indole-2-carboxylate 27, using 1-bromo-2-methoxyethane and cesium carbonate to provide intermediate 28. Intermediate 28 was then converted to the aldehyde intermediate 30 in a two-step reduction (LiAlH₄)/oxidation (MnO₂) sequence similar to Scheme 6. The final step to the analogues 31 involved reductive amination of aldehyde intermediate 30 with substituted piperazines 8, using similar conditions described previously.

The 3-cyanoindole analogues 36 were synthesised as shown in Scheme 8.

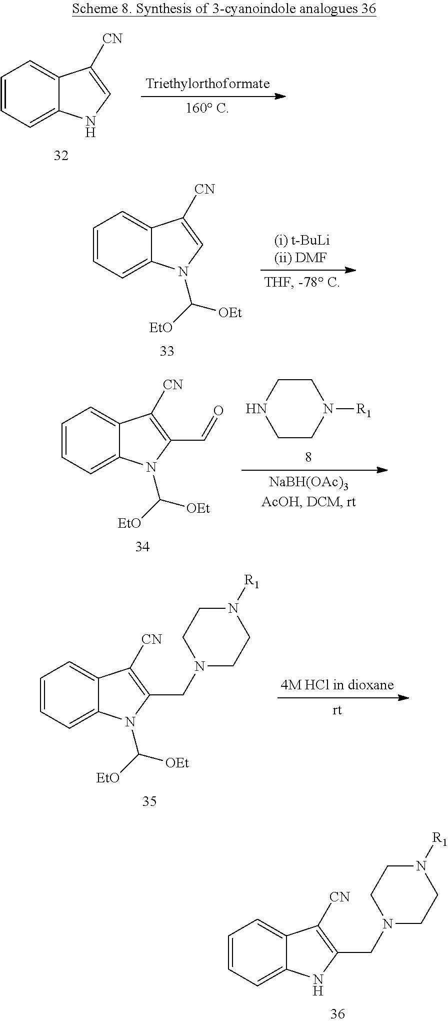

The synthesis of the 3-cyanoindole analogues 36 started with N-protection of commercial 3-cyanoindole 32 by heating in a high-pressure tube at 160° C. in the presence of neat triethylorthoformate. The resulting N-protected indole acetal 33 was formylated via deprotonation (t-BuLi) at position 2 of the indole and subsequent quenching of the anion with DMF. The intermediate aldehyde 34 was taken forward without purification to the reductive amination reaction with substituted piperazines 8. Previously used conditions of sodium triacetoxyborohydride and acetic acid in DCM were used to afford intermediates 35. The target 3-cyanoindole analogues 36 were then obtained in a final step, by acidic N-deprotection of the acetal moiety of intermediates 35.

### Linker Modifications

The synthesis of amide-linked analogues 38 is displayed in Scheme 9.

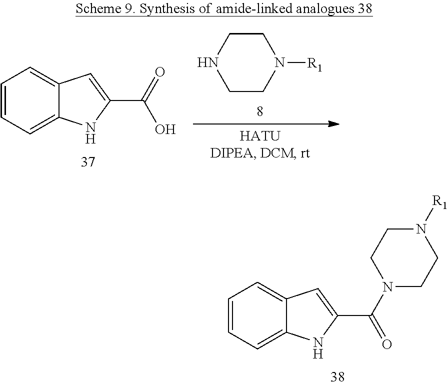

Amide-linked analogues 38 were synthesized in a single step via HATU amide coupling of commercial indole-2-carboxylic acid 37 with substituted piperazines 8.

The general synthetic approach towards linker-modified analogues 43 is illustrated in Scheme 10.

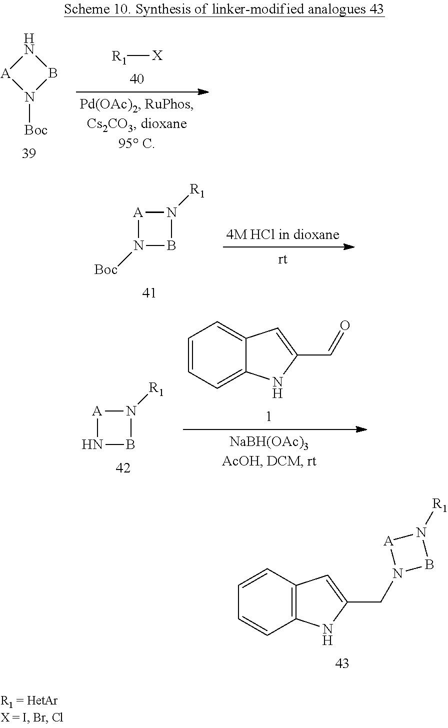

Amine precursors of type 42 (monocyclic, fused bicyclic or spirocyclic) were either available commercially or prepared via a two-step sequence. N-Boc-protected amines 39 were reacted with heteroaryl halides 40 in the presence of palladium(II) acetate, RuPhos and cesium carbonate providing the N-Boc-protected amine intermediates 41. These N-Boc-protected amine intermediates 41 were then N-deprotected under acidic conditions to afford the key amine intermediates 42. Final reductive amination step of amine intermediates 42 with 1H-indole-2-carbaldehyde 1 afforded the linker-modified analogues 43 in moderate to good yields.

### Scaffold Modifications

Compound analogues 47 with one additional nitrogen atom introduced at various positions to the indole core were prepared following the general synthetic route outlined in Scheme 11.

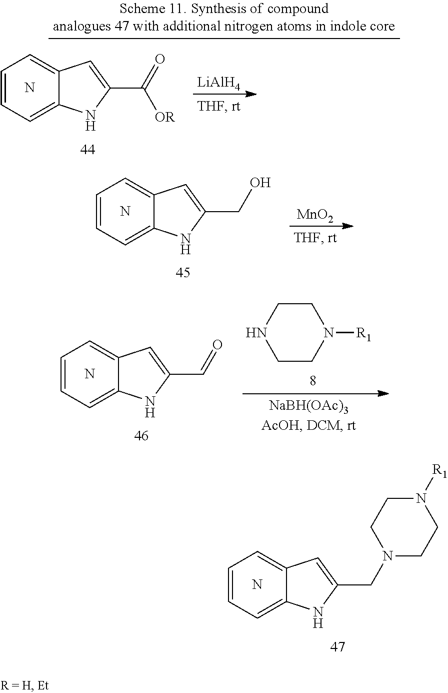

The synthesis of the compound analogues 47 started with reduction of the carboxylic acids or esters 44 to the resulting primary alcohols 45, using lithium aluminium hydride in THF. The resulting primary alcohol intermediates 45 were oxidised to the aldehydes 46, by treatment with manganese(IV) oxide in THF. The final scaffold-modified compound analogues 47 were obtained by reductive amination of aldehyde intermediates 46 with substituted piperazines 8 using previously described conditions.

The synthesis of compound analogues 49 with varying indole attachment points of the heteroaryl piperazinemethyl head-group is displayed in Scheme 12.

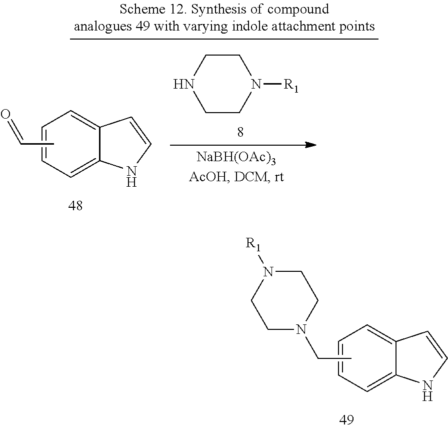

All indole attachment points modified compound analogues 49 were prepared by reductive amination of aldehyde intermediates 48 with substituted piperazines 8, using sodium triacetoxyborohydride and acetic acid in DCM.

The synthesis of scaffold-modified analogues of compound 202 (see Table 1 for structures) is shown in Scheme 13.

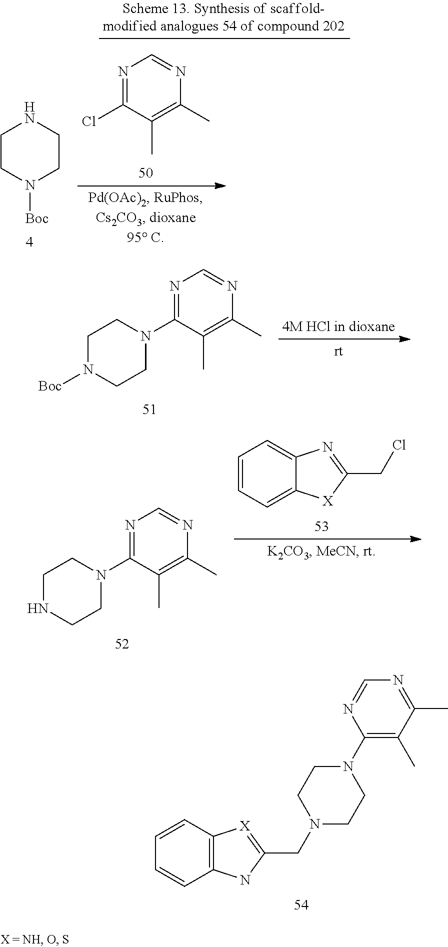

The amine precursor 52 was first constructed via a two-step sequence involving a Buchwald amination of commercial chloropyrimidine 50 with N-Boc-piperazine 4 and subsequent N-Boc-deprotection of the resulting N-Boc piperazine intermediate 51 under acidic conditions to give the hydrochloride salt. The key nucleophilic piperazine intermediate 52 as the hydrochloride salt was then reacted with several chloromethyl heterocycles 53, in the presence of potassium carbonate in acetonitrile to deliver the target scaffold-modified compound analogues 54.

The synthesis of RHS-modified triazole analogues 58 is described in Scheme 14.

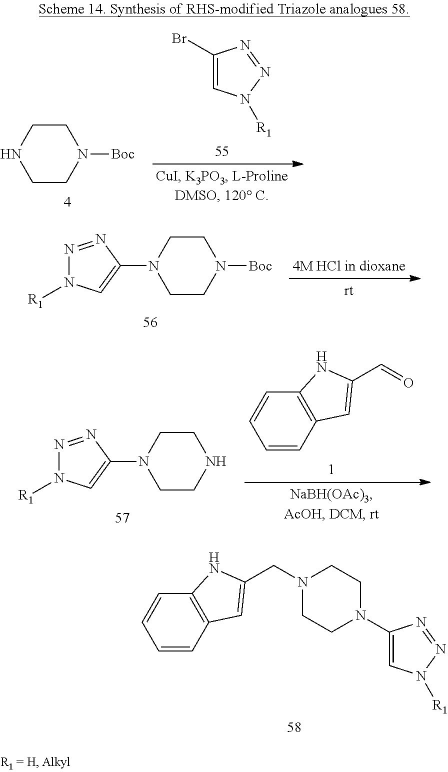

Triazole piperazine amine intermediates 57 were synthesized in two steps from N-Boc piperazine 4. The first step involved amination of the bromotriazoles 55 with N-Boc piperazine 4 using copper (I) iodide/L-Proline/potassium phosphate tribasic reaction conditions in DMSO at 120° C. The second step involved acid mediated N-Boc deprotection of the N-Boc triazole piperazine intermediates 56 to afford the amine intermediates 57, which were generated as the free base. Reductive amination of these amine intermediates 57 with 1H-indole-2-carbaldehyde 1 afforded the final triazole piperazine analogues 58, employing the previous used conditions of sodium triacetoxyborohydride and acetic acid in DCM.

The synthesis of 2-((4-(5-((2-methoxyethoxy)methyl)pyrimidin-4-yl)piperazin-1-yl)methyl)-1H-indole 287 is described in Scheme 15.

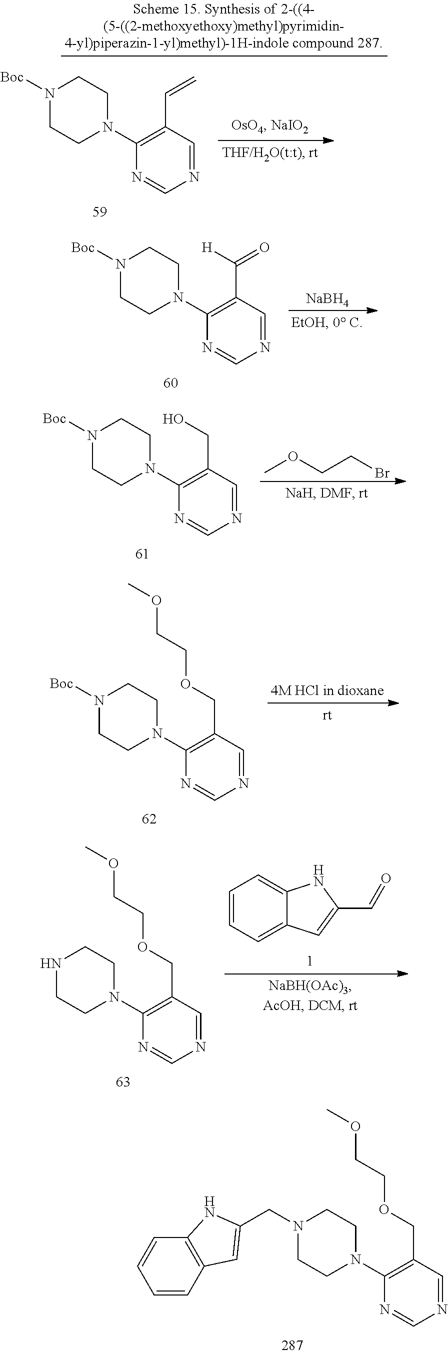

Amine intermediate 63 was synthesized in 4 steps starting from intermediate 59. Oxidative cleavage of the vinyl group of intermediate 59 gave aldehyde intermediate 60, using osmium tetroxide and sodium periodate in aqueous THF. Reduction of the aldehyde moiety of 60 using sodium borohydride afforded the primary alcohol 61, which was O-alkylated using sodium hydride and 1-bromo-2-methoxyethane in DMF to furnish the N-Boc piperazine intermediate 62. N-Boc deprotection of the N-Boc piperazine intermediate 62 using a solution of hydrogen chloride in dioxane gave the amine piperazine intermediate 63, which was generated as the free base. Final reductive amination of amine intermediate 63 with 1H-indole-2-carbaldehyde 1 afforded the final compound 287, using typical conditions of sodium triacetoxyborohydride and acetic acid in DCM.

The synthesis of 5-ethyl-4-(piperazin-1-yl)pyrimidine intermediate 68 in four steps is displayed in Scheme 16.

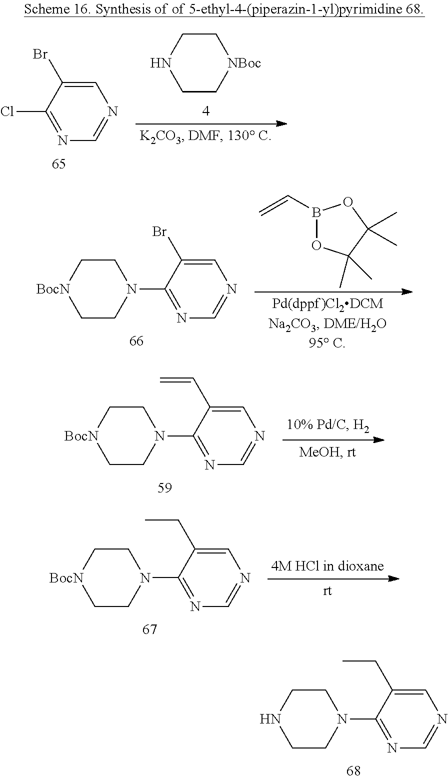

Aromatic substitution of 5-bromo-4-chloropyrimidine 65 with N-Boc piperazine 4 gave intermediate 66. The 5-bromo substituent was substituted by a vinyl group via Suzuki coupling using 4,4,5,5-tetramethyl-2-vinyl-1,3,2-dioxaborolane to afford vinyl pyrimidine intermediate 59. The vinyl moiety of intermediate 59 was rated to an ethyl group, by palladium catalyzed hydrogenation to give ethyl pyrimidine intermediate 67. N-Boc deprotection of the N-Boc piperazine intermediate 67 under acidic conditions afforded the amine piperazine intermediate 68, which was generated as the free base.

The synthesis of 2-((4-(5-ethylpyrimidin-4-yl)piperazin-1-yl)methyl)-6-((2-methoxyethoxy)methyl)-1H-benzo[d]imidazole compound 289 in seven steps is outlined in Scheme 17, which uses amine intermediate 68 earlier synthesized in Scheme 16.

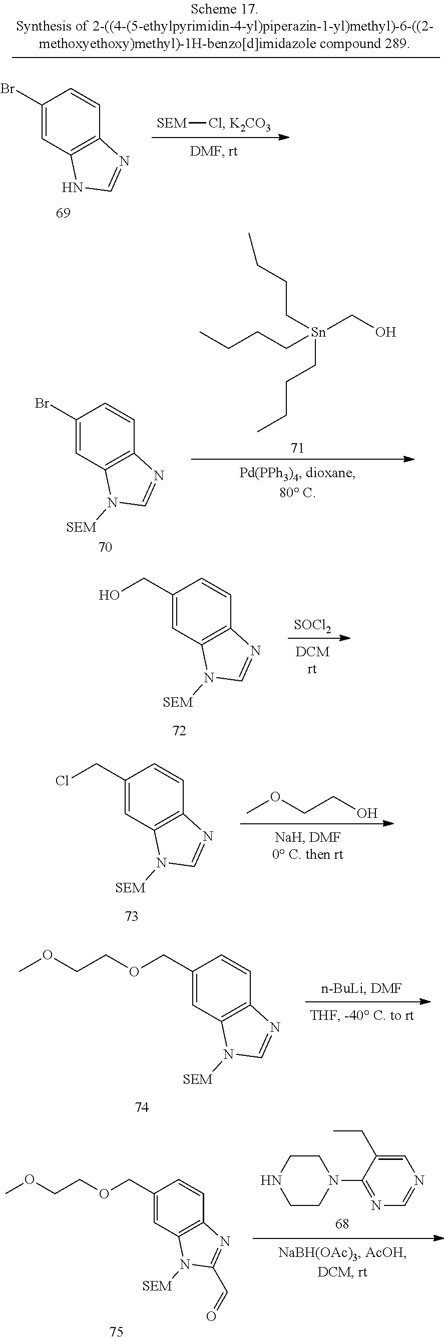

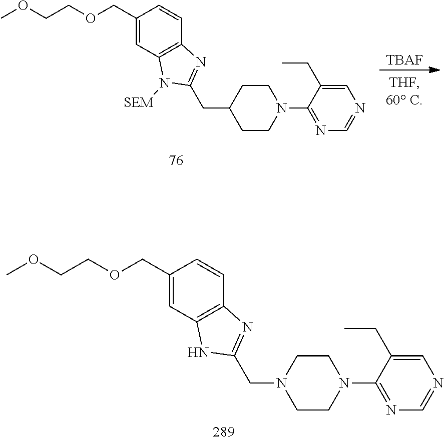 [SMILES: CCc1cncnc1N1CCC(Cc2nc3ccc(COCCOC)cc3n2COCC[Si](C)(C)C)CC1 ; "TBAF" ; CCc1cncnc1N1CCN(Cc2nc3ccc(COCCOC)cc3[nH]2)CC1]

N-SEM benzimidazole protected aldehyde intermediate 75 was synthesized in 5 steps from commercially available 5-bromo-3H-1,3-benzodiazole 69, which included N-SEM protection of the benzimidazole (step 1), hydroxymethyl substituent introduction via a Stille coupling (step 2), conversion of the benzylic alcohol to a a benzylic chloride (step 3), base-mediated O-alkylation with 2-methoxyethan-1-ol (step 4) and formylation of intermediate 74 using n-Buli and DMF (step 5). Reductive amination of the N-SEM benzimidazole protected aldehyde intermediate 75 with amine intermediate 68 using conditions previously described, followed by subsequent TBAF mediated N-SEM group deprotection afforded the final target compound 289.

The synthesis of 2-((4-(5-ethylpyrimidin-4-yl)piperazin-1-yl)methyl)-6-(2-methoxyethoxy)-1H-benzo[d]imidazole compound 290 in five steps is outlined in Scheme 18, which uses amine intermediate 68 earlier described in Scheme 16.

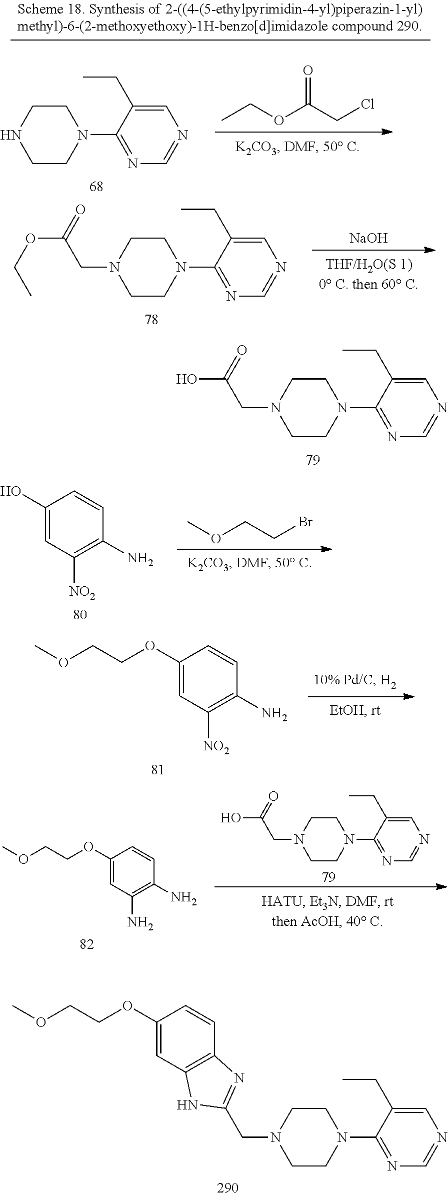

Carboxylic acid intermediate 79 was synthesized in two steps from amine intermediate 68. Step 1 involved alkylation of amine intermediate 68 with ethyl 2-chloroacetate followed by step 2, hydrolysis of the ethyl ester group of intermediate 78. Intermediate 82—4-(2-methoxyethoxy)benzene-1,2-diamine was readily prepared from commercially available 4-amino-3-nitrophenol 80 in two steps. Base-mediated O-alkylation of 4-amino-3-nitrophenol 80 with 1-bromo-2-methoxyethane gave intermediate 81, which was reduced to the diamine intermediate 82 by palladium catalyzed hydrogenation. Finally, amide formation of diamine intermediate 82 and carboxylic acid intermediate 79 using HATU coupling conditions, followed by acetic acid mediated cyclization afforded the desired benzimidazole compound 290.

The synthesis of 2-((4-(5-ethylpyrimidin-4-yl)piperazin-1-yl)methyl)-6-((2-methoxyethoxy)methyl)-1H-indole compound 291 in six steps is outlined in Scheme 19.

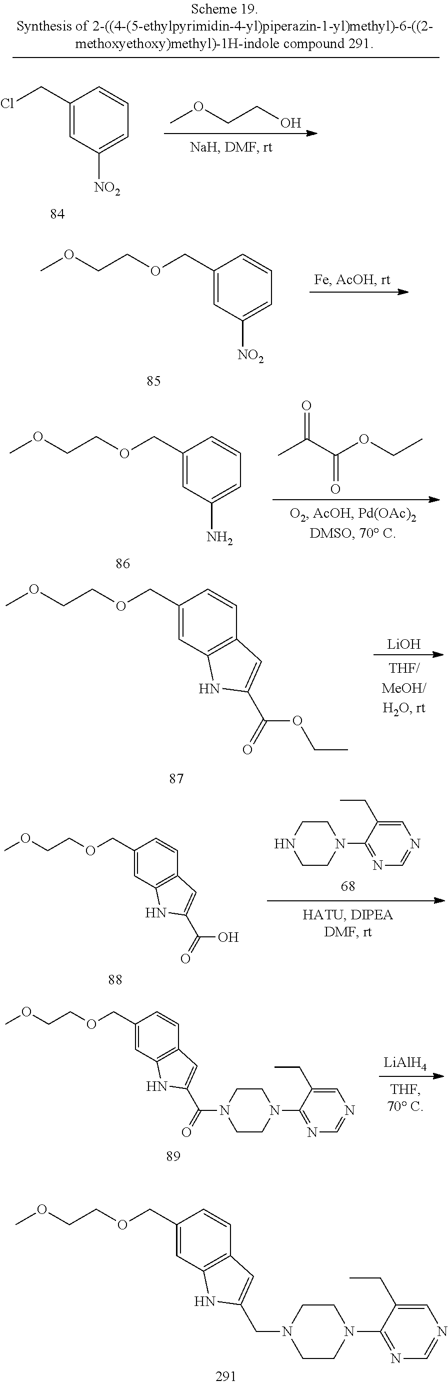

Indole ethyl ester intermediate 87 was synthesized in 3 steps from commercially available 1-(chloromethyl)-3-nitrobenzene 84. 2-Methoxyethanol was O-alkylated with 1-(chloromethyl)-3-nitrobenzene 84, using sodium hydride in DMF. The resulting nitrophenyl intermediate 85 was reduced to the aniline intermediate 86, using iron in acetic acid. Aniline intermediate 86 was then subjected to aerobic cross-dehydrogenative coupling conditions, using palladium (II) acetate, acetic acid and ethyl 2-oxopropanoate in DMSO to afford indole ethyl ester intermediate 87. Hydrolysis of the resulting indole ethyl ester intermediate 87 to the carboxylic acid 88 followed by amide formation with amine intermediate 68 gave the indole amide intermediate 89. Finally, reduction of the indole amide intermediate 89 with lithium aluminium hydride enabled synthesis of the desired indole piperazine amine compound 291.

The synthesis of 2-((4-(5-ethylpyrimidin-4-yl)piperazin-1-yl)methyl)-6-(2-methoxyethoxy)-1H-indole 292 in three steps is outlined in Scheme 20.

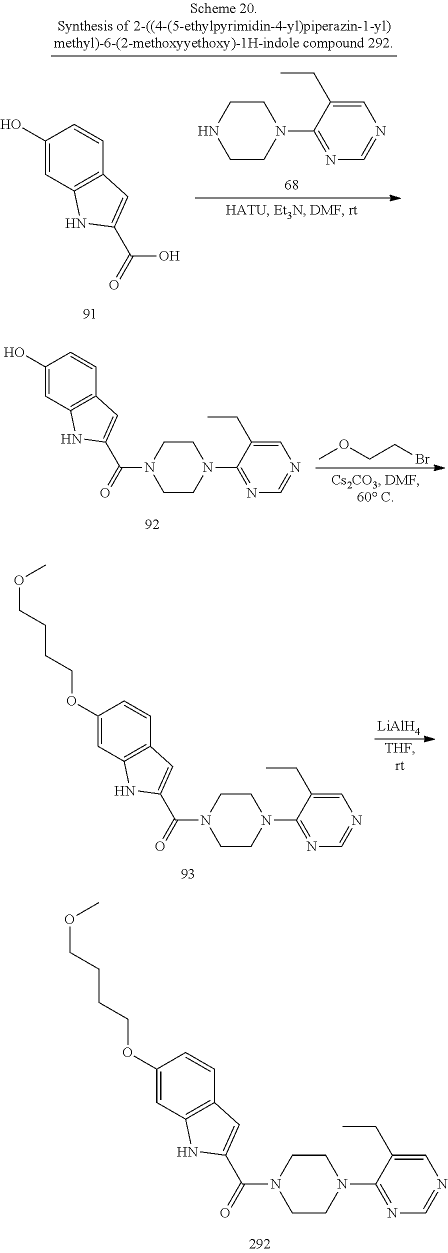

Amide intermediate 92 was synthesized from commercially available 6-hydroxy-1H-indole-2-carboxylic acid 91 and amine intermediate 68, using HATU coupling conditions. The phenol moiety of the amide intermediate 92 was then O-alkylated with 1-bromo-2-methoxyethane under basic conditions to afford O-alkylated intermediate 93. The final step of the synthesis involved reduction of the indole amide intermediate 93 with lithium aluminium hydride gave the desired indole piperazine amine compound 292.

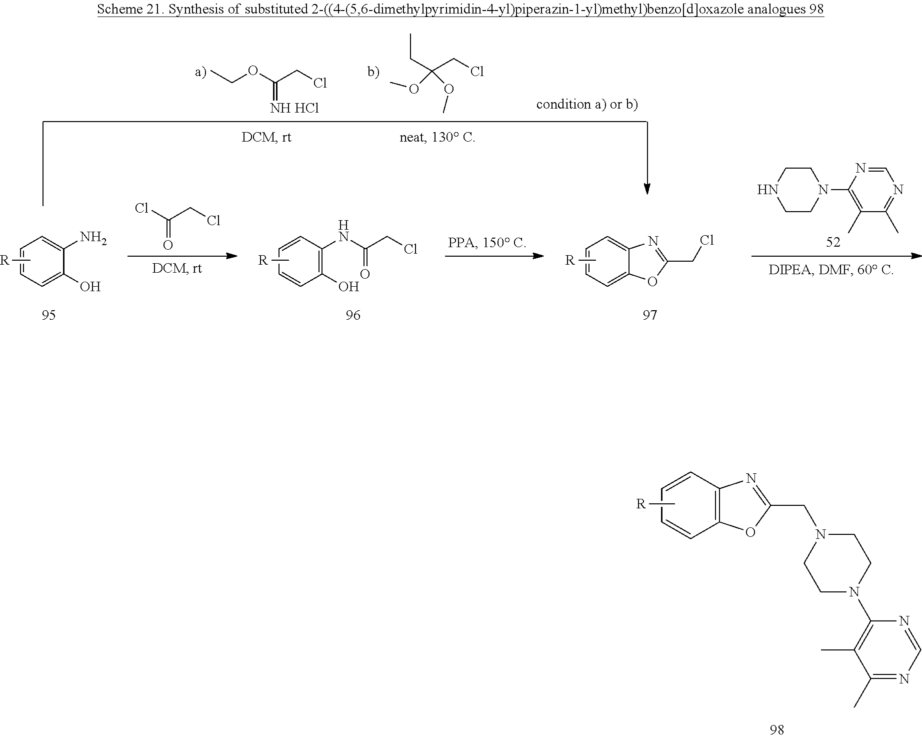

The first step involves an amide coupling reaction with substituted 2-aminophenol 95 and chloroacetyl chloride, to give substituted phenol intermediate 96. Intermediate 96 undergoes intramolecular cyclization in polyphosphoric acid at elevated temperature to afford 2-chloromethylbenzoxazole intermediate 97. Intermediate 97 undergoes nucleophilic substitution with piperazine intermediate 52 in a mixture of DMF and DIPEA, at elevated temperature to yield final compounds 98.

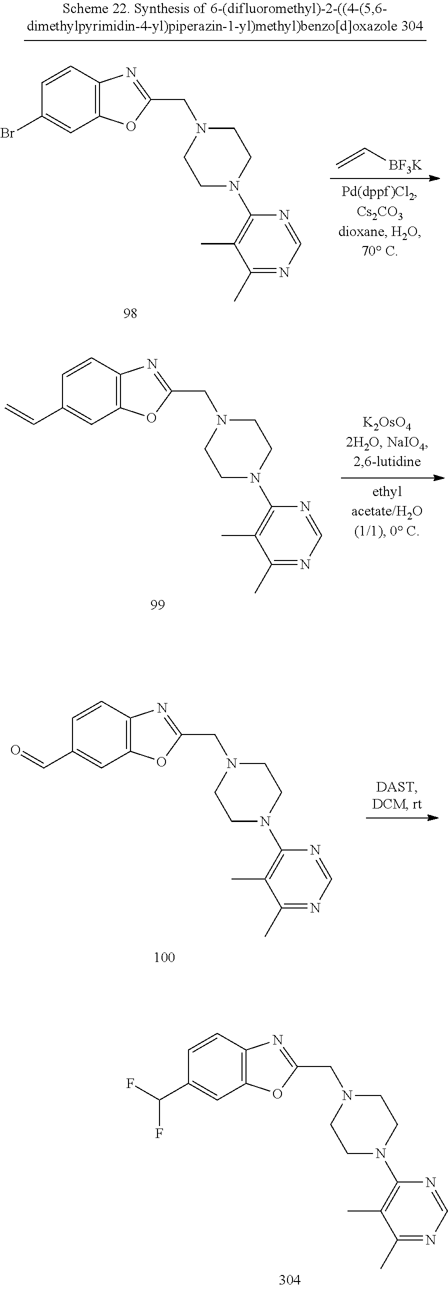

Synthesis of vinyl benzoxazole intermediate 99 was completed using palladium catalysed Suzuki cross-coupling using potassium vinyltrifluoroborate and 6-bromo-2-((4-(5,6-dimethylpyrimidin-4-yl)piperazin-1-yl)methyl)benzo[d]oxazole 98. Vinyl intermediate 99 was then oxidized using potassium osmate(VI) dihydrate to give aldehyde intermediate 100. Intermediate 100 was fluorinated using DAST in DCM to yield final compound 304.

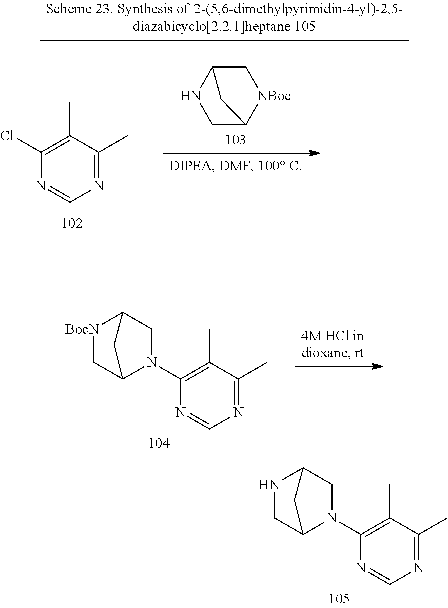

Initial displacement of 4-chloro-5,6-dimethylpyrimidine 102 with N-Boc-2,5-diazabicyclo[2.2.1]heptane 103 in a mixture of DIPEA and DMF gave N-Boc 2,5-diazabicyclo[2.2.1]heptane intermediate 104. N-Boc deprotection was carried out using a solution of 4 M HCl in dioxane yielded intermediate 105.

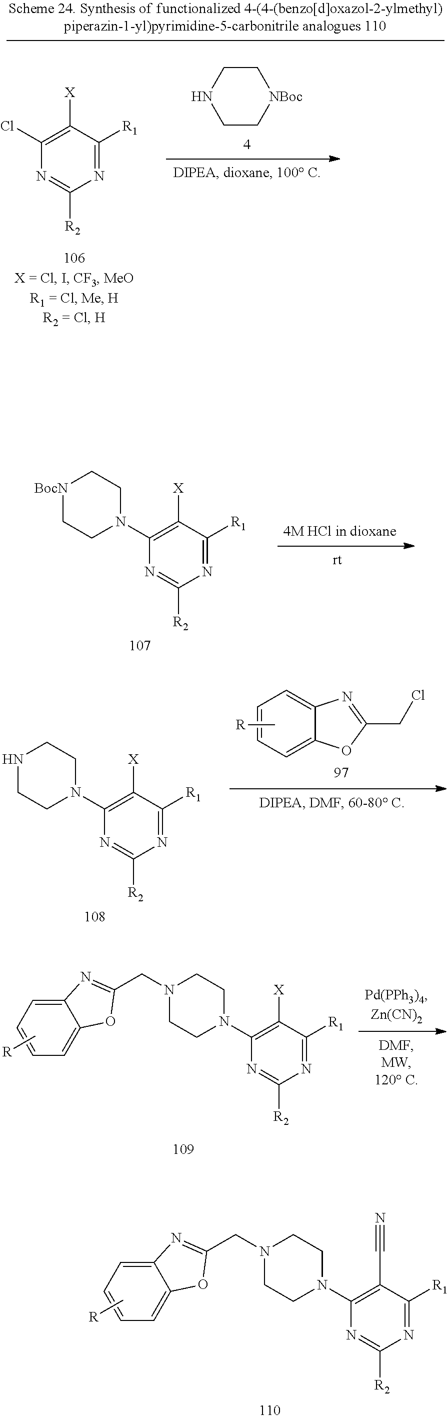

Initial displacement of substituted 4-chloropyrimidine analogues 106 and N-Boc protected piperazine 4 in dioxane at elevated temperature yields functionalised N-Boc piperazine intermediates 107. Acid mediated deprotection of the N-Boc protecting group is achieved with a solution of 4 M HCl in dioxane, affording piperazine intermediate 108. Intermediate 108 is alkylated with substituted 2-(chloromethyl)benzo[d]oxazoles 97 in DMF and DIPEA at elevated temperature to give intermediate 109. Intermediate 109 undergoes microwave assisted palladium catalysed cyanation using tetrakis(triphenylphosphine) palladium and zinc cyanide in DMF at elevated temperature to give final target 110.

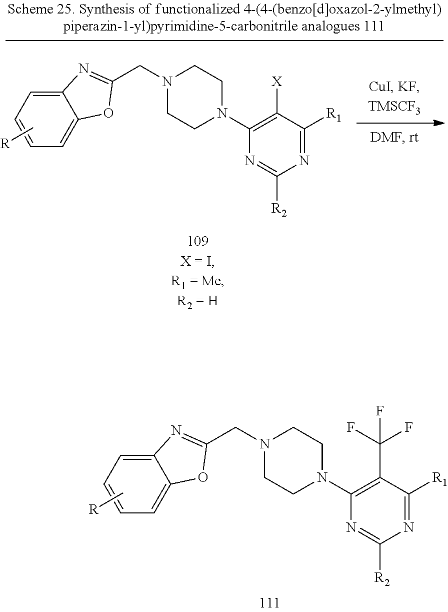

Halide conversion to introduce a trifluoromethyl group is carried out by reacting intermediate 109 with trifluoromethyltrimethylsilane in the presence of copper iodide and potassium fluoride in DMF, to give final compound 111.

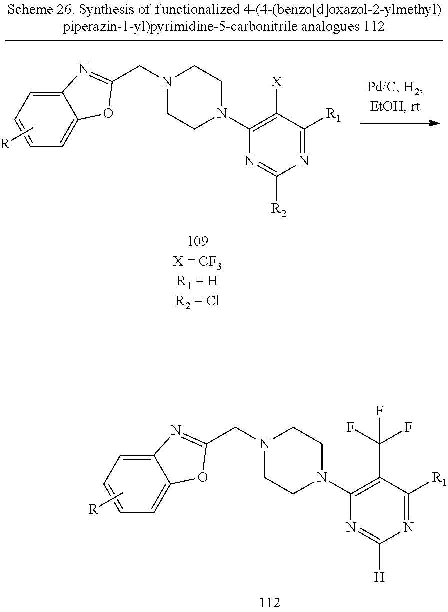

Reduction of halogenated intermediate 109 under palladium catalysed hydrogenation conditions was achieved, to give final compound 112.

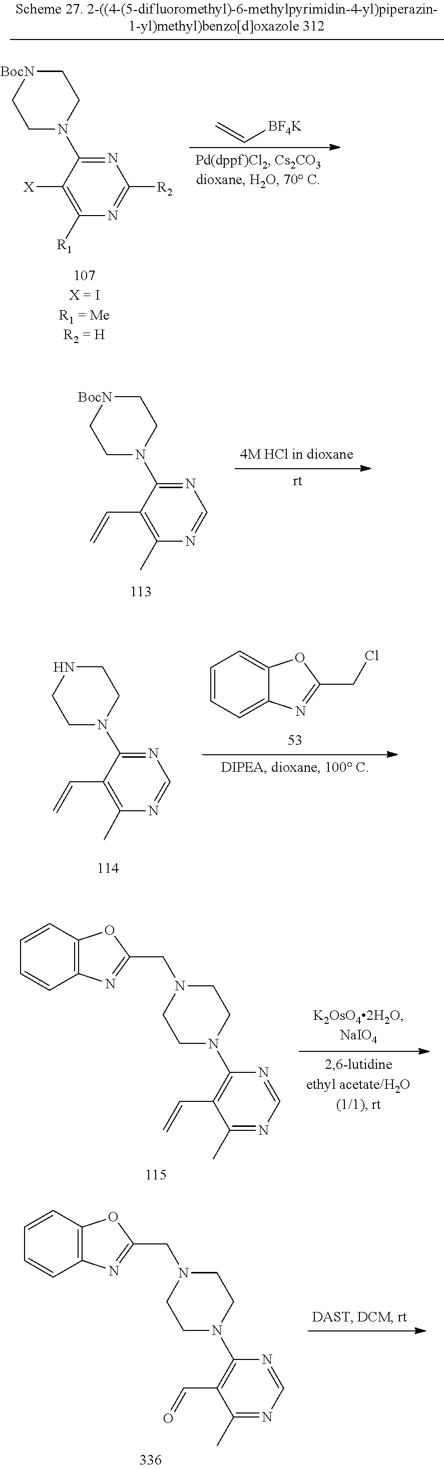

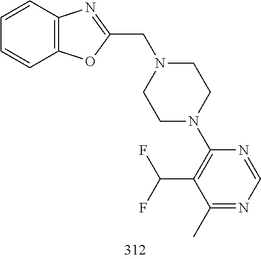 [SMILES: Cc1ncnc(N2CCN(Cc3nc4ccccc4o3)CC2)c1C(F)F]

Synthesis of vinyl pyrimidine intermediate 113 is completed using palladium catalysed Suzuki cross-coupling using potassium vinyltrifluoroborate and tert-butyl 4-(5-iodo-6-methylpyrimidin-4-yl)piperazine-1-carboxylate 107. The N-Boc protecting group is removed under acidic conditions using a solution of HCl in dioxane to give intermediate 114, as the hydrochloride salt. Substitution reaction between intermediate 114 and 2-(chloromethyl)benzo[d]oxazole 53 in DMF and DIPEA gives intermediate 115. Vinyl intermediate 115 is oxidazied using potassium osmate(VI) dihydrate to afford aldehyde intermediate 336. Intermediate 336 is subjected to fluorination with DAST to give 2-((4-(5-(difluoromethyl)-6-methylpyrimidin-4-yl)piperazin-1-yl)methyl)benzo[d]oxazole 312

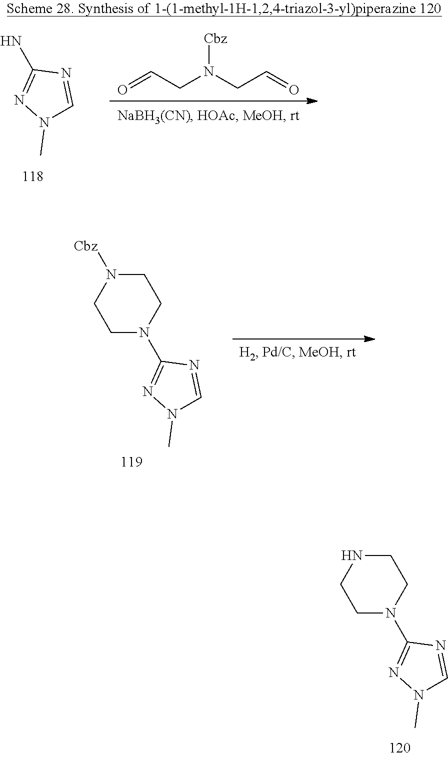

Synthesis of Cbz protected intermediate 119 is completed via reductive amination reaction of 1-methyl-1H-1,2,4-triazol-3-ylamine 118 with phenylmethyl N,N-bis(2-oxoethyl)carbamate in the presence of sodium cyanoborohydride and acetic acid. The N-Cbz intermediate 119 is deprotected using palladium catalysed hydrogenation in methanol to yield compound 120.

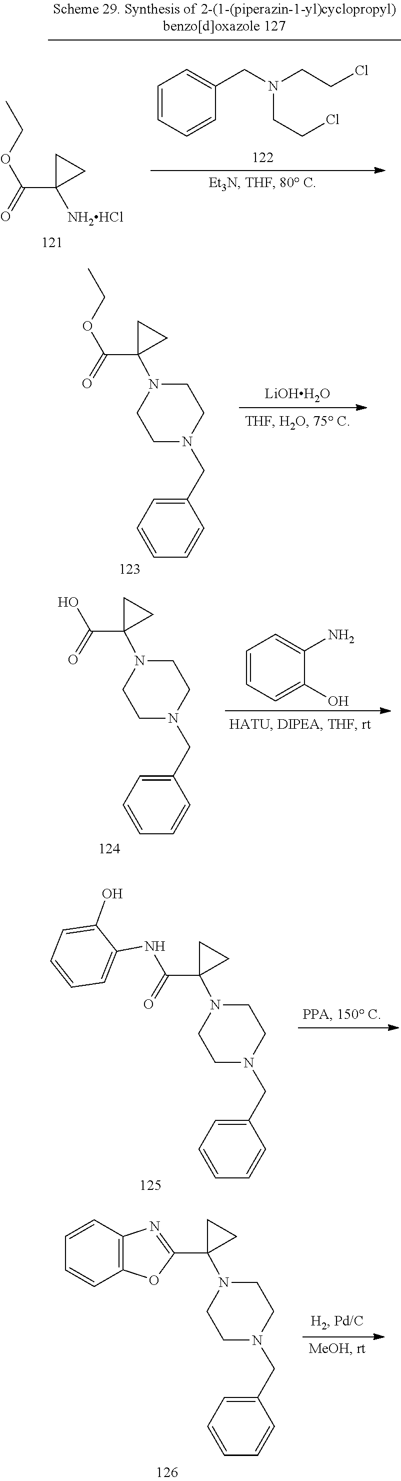

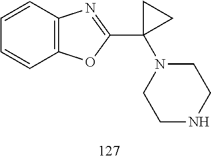 [SMILES: c1ccc2oc(C3(N4CCNCC4)CC3)nc2c1]

Synthesis of ethyl ester intermediate 123 is completed via alkylation of ethyl 1-aminocyclopropanecarboxylate hydrochloride 121 with N-benzyl-2-chloro-N-(2-chloroethyl)ethanamine 122 in THF and triethylamine. Intermediate 123 is hydrolysed with aqueous lithium hydroxide in THF to give carboxylic acid intermediate 124. Intermediate 124 is subjected to HATU mediated amide coupling conditions with 2-aminophenol to give amidophenol intermediate 125. Intermediate 125 undergoes intramolecular cyclization in polyphosphoric acid to give benyl protected benzoxazole intermediate 126. Deprotection of intermediate 126 is achieved using palladium catalysed hydrogenation in methanol to give intermediate 127.

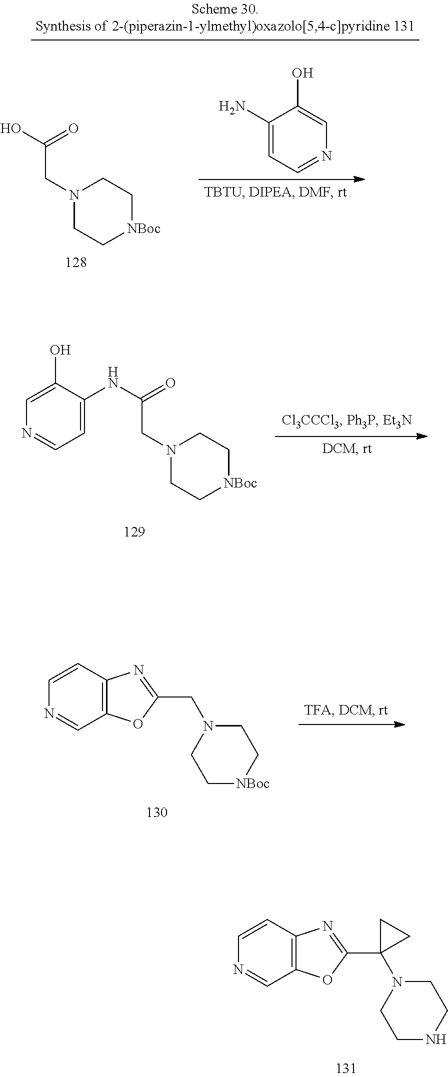 [SMILES: CC(C)(C)OC(=O)N1CCN(CC(=O)O)CC1 ; Nc1ccncc1O ; CC(C)(C)OC(=O)N1CCN(CC(=O)Nc2ccncc2O)CC1 ; CC(C)(C)OC(=O)N1CCN(Cc2nc3ccncc3o2)CC1 ; c1cc2nc(C3(N4CCNCC4)CC3)oc2cn1]

Synthesis of amide intermediate 129 is completed via TBTU mediated coupling between acid 128 and 4-amino-3-pyridinol in a mixture of DIPEA and DMF. Intermediate 129 is subjected to intramolecular cyclisation using triphenylphosphine, hexachloroethane and triethylamine in DCM to give oxazolo[5,4-c]pyridine intermediate 130. Intermediate 130 is deprotected under acidic conditions using a solution of 4 M HCl in dioxane to give intermediate 131.

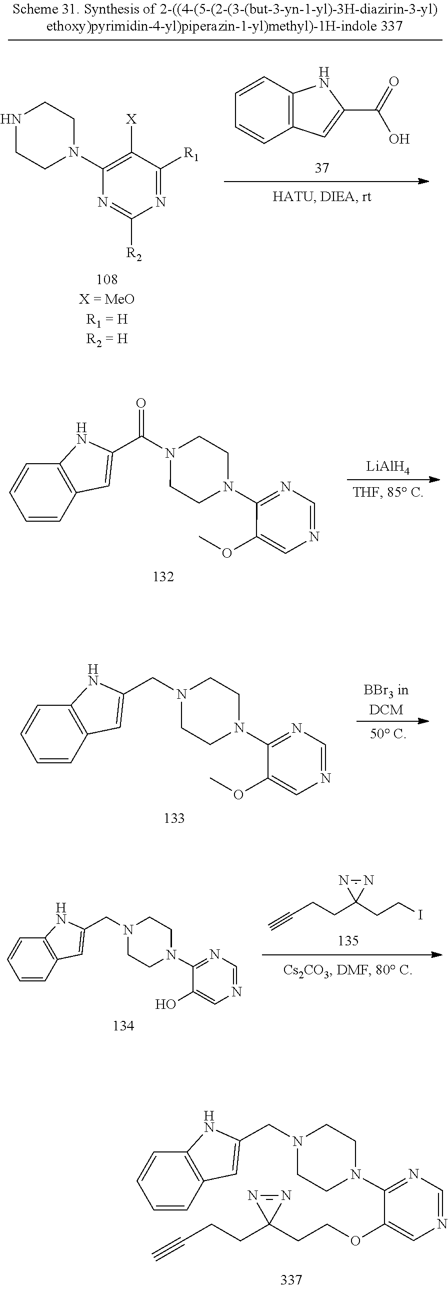

Synthesis of amide intermediate 132 is completed via HATU mediated coupling reaction between 1H-indole-2-carboxylic acid 37 and analogues of intermediate 108. Amide intermediate 132 is reduced to the corresponding amine using lithium aluminium hydride in THF to give intermediate 133. Demethylation of intermediate 133 is achieved using boron tribromide in DCM at elevated temperature to yield intermediate 134. Base mediated alkylation of intermediate 134 with 3-(but-3-yn-1-yl)-3-(2-iodoethyl)-3H-diazirine 135 in DMF gives final compound 337.

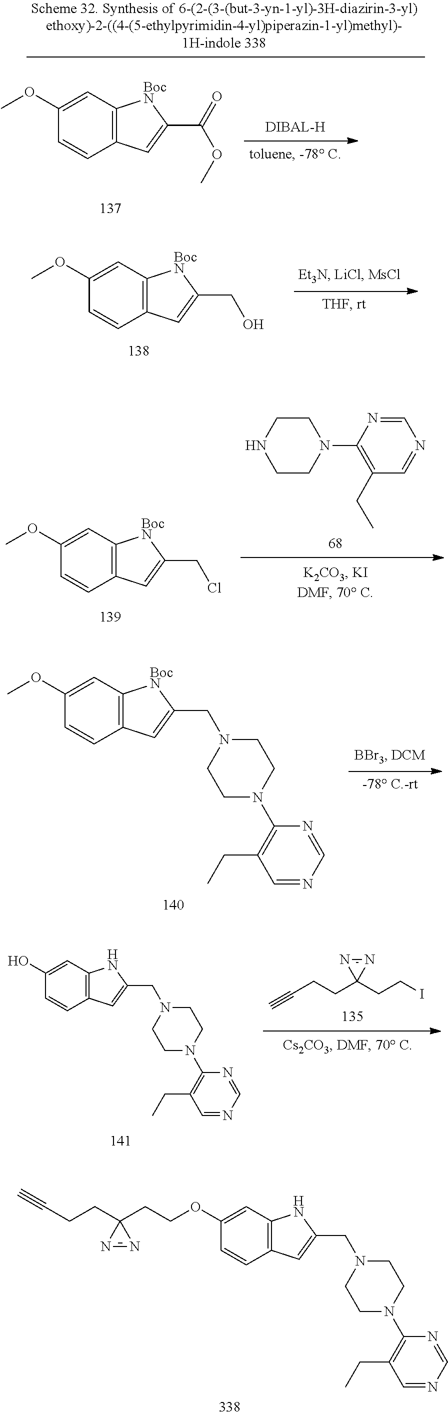

Synthesis of alcohol intermediate 138 is completed via DIBAL-H reduction at −78° C. to give alcohol intermediate 138. Intermediate 138 is chlorinated using lithium chloride, methanesulfonyl chloride and triethylamine in THF to give tert-butyl 2-(chloromethyl)-6-methoxy-1H-indole-1-carboxylate 139. Intermediate 139 and piperazine 68 are coupled in the present of potassium carbonate and potassium iodide in DMF at elevated temperature to give intermediate 140. Intermediate 140 is subjected to cocurrent deprotection with boron tribromide in DCM at elevated temperature to give intermediate 141. Base mediated alkylation of intermediate 141 with 3-(but-3-yn-1-yl)-3-(2-iodoethyl)-3H-diazirine 135 in DMF gives final compound 338.

### Detailed Synthesis of Intermediates of Compounds of the Invention

### Synthesis of Tert-Butyl 4-((6-(trifluoromethyl)-1H-benzo[d]imidazol-2-yl)methyl)piperazine-1-carboxylate

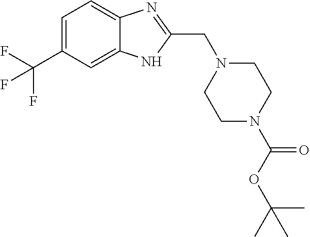 [SMILES: CC(C)(C)OC(=O)N1CCN(Cc2nc3ccc(C(F)(F)F)cc3[nH]2)CC1]

2-(Chloromethyl)-6-(trifluoromethyl)-1H-benzo[d]imidazole (1 g, 4.26 mmol) was dissolved in anhydrous DMF (10 mL) and tert-butyl piperazine-1-carboxylate (873 mg, 4.69 mmol) was added followed by DIPEA (1.5 mL, 8.52 mmol). The reaction mixture was stirred at 80° C. for 18 hours. After cooling to room temperature, the reaction mixture was partitioned between ethyl acetate (100 mL) and water (100 mL) and the aqueous layer was extracted with ethyl acetate (2×50 mL). The combined organic extracts were dried (MgSO₄), filtered and evaporated. The residue was purified by column chromatography on silica gel (0-5% MeOH in DCM) to provide tert-butyl 4-((6-(trifluoromethyl)-1H-benzo[d]imidazol-2-yl)methyl)piperazine-1-carboxylate as an orange solid.

Yield 1.6 g (97%). ¹H NMR (400 MHz, DMSO) δ 12.78 (br s, 1H), 7.87 (s, 1H), 7.76-7.70 (m, 1H), 7.48 (d, J=8.2 Hz, 1H), 3.81 (s, 2H), 3.38 (m, 4H), 2.44 (dd, J=5.0, 5.0 Hz, 4H), 1.39 (s, 9H).

### Synthesis of 2-(piperazin-1-ylmethyl)-6-(trifluoromethyl)-1H-benzo[d]imidazole

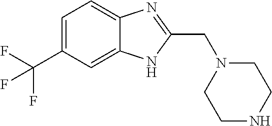 [SMILES: FC(F)(F)c1ccc2nc(CN3CCNCC3)[nH]c2c1]

tert-Butyl 4-((6-(trifluoromethyl)-1H-benzo[d]imidazol-2-yl)methyl)piperazine-1-carboxylate (1.6 g, 4.16 mmol) was treated with a solution of HCl in dioxane (4 M, 4.3 mL, 17.2 mmol) at room temperature for 18 hours. The solvent was removed in vacuo to furnish 2-(piperazin-1-ylmethyl)-6-(trifluoromethyl)-1H-benzo[d]imidazole dihydrochloride as a light brown solid. This material (980 mg, 2.74 mmol) was dissolved in MeOH (10 mL), loaded onto an SCX-2 cartridge (20 g, 0.6 mmol/g loading), washed with MeOH and eluted with ammonia/MeOH (2 M) to give 2-(piperazin-1-ylmethyl)-6-(trifluoromethyl)-1H-benzo[d]imidazole as a light brown solid.

Yield 475 mg (61%). ¹H NMR (400 MHz, DMSO) δ 7.90 (m, 1H), 7.73 (d, J=7.1 Hz, 1H), 7.52 (d, J=8.3 Hz, 1H), 3.78 (s, 2H), 2.78 (dd, J=4.5, 4.5 Hz, 4H), 2.47-2.40 (m, 4H).

### Synthesis of Tert-Butyl 4-((6-chloro-1H-benzo[d]imidazol-2-yl)methyl)piperazine-1-carboxylate

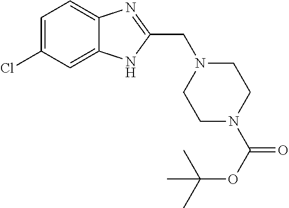 [SMILES: CC(C)(C)OC(=O)N1CCN(Cc2nc3ccc(Cl)cc3[nH]2)CC1]

6-Chloro-2-(chloromethyl)-1H-benzo[d]imidazole (300 mg, 1.49 mmol) and tert-butyl piperazine-1-carboxylate (306 mg, 1.64 mmol) were dissolved in anhydrous acetonitrile (4 mL), potassium carbonate (1.03 g, 7.46 mmol) was added and the reaction was stirred at room temperature for 18 hours. The reaction mixture was partioned between ethyl acetate (50 mL) and brine (30 mL) and the aqueous layer was extracted with ethyl acetate (2×30 mL). The combined organic extracts were dried (MgSO₄), filtered and evaporated to provide tert-butyl 4-((6-chloro-1H-benzo[d]imidazol-2-yl)methyl)piperazine-1-carboxylate as an orange solid.

Yield 525 mg (quant). ¹H NMR (400 MHz, DMSO) δ 12.52 (br s, 1H), 7.59 (m, 1H), 7.49 (m, 1H), 7.21-7.16 (m, 1H), 3.75 (s, 2H), 3.23 (dd, J=5.0, 5.0 Hz, 2H), 2.64-2.60 (m, 2H), 2.43 (dd, J=5.0, 5.0 Hz, 4H), 1.40 (s, 9H).

### Synthesis of 6-chloro-2-(piperazin-1-ylmethyl)-1H-benzo[d]imidazole

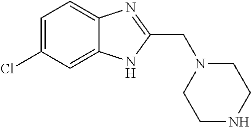 [SMILES: Clc1ccc2nc(CN3CCNCC3)[nH]c2c1]

Compound 6-chloro-2-(piperazin-1-ylmethyl)-1H-benzo[d]imidazole was prepared following a procedure similar to that described for the synthesis of 2-(piperazin-1-ylmethyl)-6-(trifluoromethyl)-1H-benzo[d]imidazole, and was isolated as a beige solid.

Yield 355 mg (95%). ¹H NMR (400 MHz, DMSO) δ 7.56 (m, 2H), 7.21 (d, J=8.3 Hz, 1H), 3.72 (s, 2H), 2.76 (m, 4H), 2.46-2.39 (m, 4H).

### Synthesis of Tert-Butyl 4-((1H-indol-2-yl)methyl)piperazine-1-carboxylate

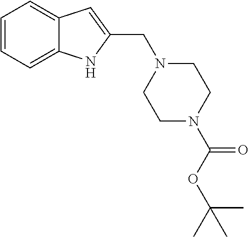 [SMILES: CC(C)(C)OC(=O)N1CCN(Cc2cc3ccccc3[nH]2)CC1]

To a solution of 1H-indole-2-carbaldehyde (250 mg, 1.72 mmol) and tert-butyl piperazine-1-carboxylate (353 mg, 1.89 mmol) in anhydrous DCM (5 mL) were added sequentially sodium triacetoxyborohydride (913 mg, 4.31 mmol) and acetic acid (10 μL, 0.17 mmol), and the reaction mixture was stirred at room temperature for 18 hours. The reaction mixture was diluted with DCM (30 mL) and partitioned with a saturated solution of NaHCO₃ (30 mL). The organic phase was dried (MgSO₄), filtered and evaporated. The residue was purified by column chromatography on silica gel (0-50% ethyl acetate in cyclohexane) to furnish tert-butyl 4-((1H-indol-2-yl)methyl)piperazine-1-carboxylate as a light brown solid

Yield 456 mg (84%). ¹H NMR (400 MHz, DMSO) δ 11.00 (br s, 1H), 7.45 (d, J=7.8 Hz, 1H), 7.33 (d, J=8.0 Hz, 1H), 7.03 (dd, J=7.0, 7.0 Hz, 1H), 6.95 (dd, J=7.2, 7.2 Hz, 1H), 6.28 (d, J=1.4 Hz, 1H), 3.63 (s, 2H), 2.37 (dd, J=5.0, 5.0 Hz, 4H), 1.40 (s, 9H). 4 protons obstructed by solvent/water peak.

### Synthesis of 2-(piperazin-1-ylmethyl)-1H-indole

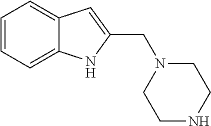 [SMILES: c1ccc2[nH]c(CN3CCNCC3)cc2c1]

Compound 2-(piperazin-1-ylmethyl)-1H-indole was prepared following a procedure similar to that described for the synthesis of 2-(piperazin-1-ylmethyl)-6-(trifluoromethyl)-1H-benzo[d]imidazole, and was isolated as a brown solid.

Yield 260 mg (60%). ¹H NMR (400 MHz, DMSO) δ 11.02-10.95 (br s, 1H), 7.45 (d, J=7.8 Hz, 1H), 7.33-7.27 (m, 1H), 7.05-7.00 (m, 1H), 6.96-6.89 (m, 1H), 6.27 (d, J=1.3 Hz, 1H), 3.58 (s, 2H), 2.75 (dd, J=4.8, 4.8 Hz, 4H), 2.40-2.33 (m, 4H).

### Synthesis of Tert-Butyl 4-((5-chloro-1H-indol-2-yl)methyl)piperazine-1-carboxylate

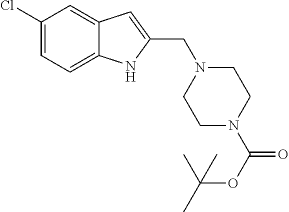 [SMILES: CC(C)(C)OC(=O)N1CCN(Cc2cc3cc(Cl)ccc3[nH]2)CC1]

Compound tert-butyl 4-((5-chloro-1H-indol-2-yl)methyl)piperazine-1-carboxylate was prepared from 5-chloro-1H-indole-2-carbaldehyde following a procedure similar to that described for the synthesis of tert-butyl 4-((1H-indol-2-yl)methyl)piperazine-1-carboxylate, and was isolated as a yellow solid.

Yield 781 mg (quant). ¹H NMR (400 MHz, DMSO) δ 11.22 (s, 1H), 7.50 (d, J=2.1 Hz, 1H), 7.33 (d, J=8.7 Hz, 1H), 7.03 (dd, J=2.1, 8.5 Hz, 1H), 6.29 (d, J=1.3 Hz, 1H), 3.63 (s, 2H), 3.34 (m, 4H), 2.36 (dd, J=5.0, 5.0 Hz, 4H), 1.40 (s, 9H).

### Synthesis of 5-chloro-2-(piperazin-1-ylmethyl)-1H-indole

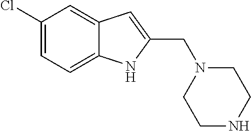 [SMILES: Clc1ccc2[nH]c(CN3CCNCC3)cc2c1]

Compound 5-chloro-2-(piperazin-1-ylmethyl)-1H-indole was prepared following a procedure similar to that described for the synthesis of 2-(piperazin-1-ylmethyl)-6-(trifluoromethyl)-1H-benzo[d]imidazole, and was isolated as a brown solid.

Yield 488 mg (87%). ¹H NMR (400 MHz, DMSO) δ 11.21 (br s, 1H), 7.52 (d, J=1.8 Hz, 1H), 7.36 (d, J=8.6 Hz, 1H), 7.06 (dd, J=2.0, 8.6 Hz, 1H), 6.31 (s, 1H), 3.60 (s, 2H), 2.74 (dd, J=4.5, 4.5 Hz, 4H), 2.41-2.33 (m, 4H), 2.19 (br s, 1H).

### Synthesis of 5-bromo-2-[[4-(4-pyridyl)piperazin-1-yl]methyl]-1H-indole (Compound 215)

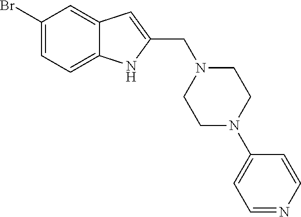 [SMILES: Brc1ccc2[nH]c(CN3CCN(c4ccncc4)CC3)cc2c1]

To a solution of 5-bromo-1H-indole-2-carbaldehyde (1 g, 4.46 mmol) and 1-(4-pyridyl)piperazine (801 mg, 4.91 mmol) in anhydrous DCM (25 mL) were added sequentially sodium triacetoxyborohydride (2.36 g, 11.16 mmol) and acetic acid (26 μL, 0.446 mmol), and the reaction mixture was stirred at room temperature for 2 hours. The reaction mixture was diluted with DCM (30 mL) and washed with a saturated aqueous solution of NaHCO₃ (30 mL) and brine (10 mL). The organic phase was dried (Na₂SO₄), filtered and evaporated. The residue was purified by column chromatography on silica gel (0-10% 10% ammonia/methanol in methanol in DCM) to afford 5-bromo-2-[[4-(4-pyridyl)piperazin-1-yl]methyl]-1H-indole as an off-white solid.

Yield 743 mg (45%). ¹H NMR (400 MHz, DMSO) δ 11.28 (s, 1H), 8.16 (d, J=6.5 Hz, 2H), 7.65 (d, J=1.9 Hz, 1H), 7.30 (d, J=8.5 Hz, 1H), 7.15 (dd, J=1.9, 8.6 Hz, 1H), 6.81 (d, J=6.7 Hz, 2H), 6.32 (d, J=1.3 Hz, 1H), 3.68 (s, 2H). 8 protons obscured by water and DMSO peaks. m/z: [ESI⁺] 371 (M+H)⁺, (C₁₈H₁₉BrN₄).

### Synthesis of Tert-Butyl 4-((1H-indol-2-yl)methyl)piperazine-1-carboxylate

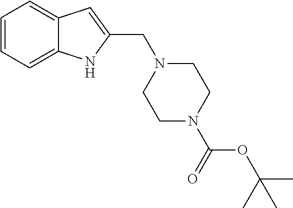 [SMILES: CC(C)(C)OC(=O)N1CCN(Cc2cc3ccccc3[nH]2)CC1]

To a solution of 1H-indole-2-carbaldehyde (1.0 g, 6.89 mmol) and tert-butyl piperazine-1-carboxylate (1.41 g, 7.58 mmol) in anhydrous DCM (25 mL) were added sequentially sodium triacetoxyborohydride (3.65 g, 17.2 mmol) and acetic acid (39 μL, 0.69 mmol), and the reaction mixture was stirred at room temperature for 2 hours. The reaction mixture was diluted with DCM (30 mL) and washed with a saturated aqueous solution of NaHCO₃ (30 mL) and brine (10 mL). The organic phase was dried (Na₂SO₄), filtered and evaporated to furnish tert-butyl 4-((1H-indol-2-yl)methyl)piperazine-1-carboxylate as an off-white solid.

Yield 2.03 g (94%). ¹H NMR (400 MHz, DMSO) δ 11.00 (br s, 1H), 7.45 (d, J=8.0 Hz, 1H), 7.33 (d, J=8.0 Hz, 1H), 7.03 (dd, J=7.0, 7.0 Hz, 1H), 6.95 (dd, J=7.2, 7.2 Hz, 1H), 6.28 (d, J=1.4 Hz, 1H), 3.63 (s, 2H), 2.37 (dd, J=5.0, 5.0 Hz, 4H), 1.40 (s, 9H). 4 protons obstructed by solvent/water peak.

### Synthesis of 2-(piperazin-1-ylmethyl)-1H-indole

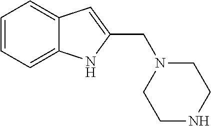 [SMILES: c1ccc2[nH]c(CN3CCNCC3)cc2c1]

To a solution of tert-butyl 4-((1H-indol-2-yl)methyl)piperazine-1-carboxylate (2.03 g, 6.45 mmol) in dioxane (4 mL) was added a solution of HCl in dioxane (4 M, 10 mL, 40 mmol) and the reaction mixture was stirred at room temperature for 2 hours. Additional HCl in dioxane (4 M, 10 mL, 40 mmol) was added and the reaction was stirred at room temperature for 18 hours. The solvent was removed in vacuo to furnish a residue that was dissolved in MeOH (10 mL), loaded onto an SCX-2 cartridge (20 g, 0.6 mmol/g loading), washed with MeOH and eluted with ammonia/MeOH (1 M) to give 2-(piperazin-1-ylmethyl)-1H-indole as a light brown solid.

Yield 1.35 g (97%). ¹H NMR (400 MHz, DMSO) δ 10.98 (br s, 1H), 7.45 (d, J=7.8 Hz, 1H), 7.30 (d, J=7.8 Hz, 1H), 7.02 (dd, J=7.1, 7.1 Hz, 1H), 6.92 (dd, J=7.1, 7.1 Hz, 1H), 6.27 (d, J=1.3 Hz, 1H), 3.58 (s, 2H), 2.75 (dd, J=4.8, 4.8 Hz, 4H), 2.40-2.33 (m, 4H). NH proton obscured under residual water peak.

### Synthesis of 4-bromo-1-((2-(trimethylsilyl)ethoxy)methyl)-1H-pyrrolo[2,3-b]pyridine

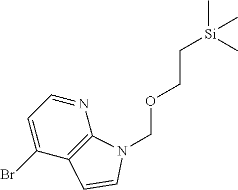 [SMILES: C[Si](C)(C)CCOCn1ccc2c(Br)ccnc21]

To a solution of 4-bromo-1H-pyrrolo[2,3-b]pyridine (100 mg, 0.508 mmol) in anhydrous DMF (2 mL) at 0° C. was added sodium hydride (60%, 30 mg, 0.761 mmol), and the mixture was stirred for 10 minutes at this temperature. 2-(Trimethylsilyl)ethoxymethyl chloride (0.11 mL, 0.609 mmol) was then added and the reaction was allowed to warm to room temperature and stirred for 2 hours. The reaction mixture was carefully quenched by dropwise addition of water (0.1 mL) and partitioned between ethyl acetate (20 mL) and water (20 mL). The layers were separated, and the organic phase was washed with water (10 mL), water/brine 1:1 (10 mL) and brine (10 mL). The organic layer was dried (Na₂SO₄), filtered and evaporated and the residue was purified by column chromatography on silica gel (0-20% ethyl acetate in cyclohexane) to furnish 4-bromo-1-((2-(trimethylsilyl)ethoxy)methyl)-1H-pyrrolo[2,3-b]pyridine as a colourless oil.

Yield 111 mg (69%). ¹H NMR (400 MHz, DMSO) δ 8.27 (d, J=5.1 Hz, 1H), 7.90 (d, J=3.6 Hz, 1H), 7.54 (d, J=5.1 Hz, 1H), 6.63 (d, J=3.6 Hz, 1H), 5.74 (s, 2H), 3.64-3.59 (m, 2H), 0.94-0.90 (m, 2H), 0.00 (s, 9H).

### Synthesis of 4-(4-((1H-indol-2-yl)methyl)piperazin-1-yl)-1-((2-(trimethylsilyl)ethoxy)-methyl)-1H-pyrrolo[2,3-b]pyridine

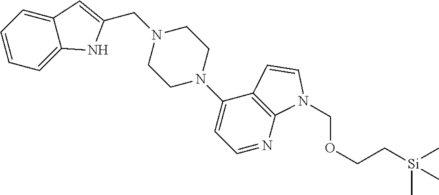 [SMILES: C[Si](C)(C)CCOCn1ccc2c(N3CCN(Cc4cc5ccccc5[nH]4)CC3)ccnc21]

To a degassed suspension of 2-(piperazin-1-ylmethyl)-1H-indole (70 mg, 0.325 mmol), 4-bromo-1-((2-(trimethylsilyl)ethoxy)methyl)-1H-pyrrolo[2,3-b]pyridine (117 mg, 0.358 mmol) and cesium carbonate (212 mg, 0.65 mmol) in anhydrous dioxane (3 mL) was added palladium(II) acetate (7.3 mg, 0.033 mmol) and RuPhos (30 mg, 0.065 mmol). The mixture was sparged for 10 minutes with nitrogen and heated at 95° C. for 3 hours in a sealed tube. After cooling to room temperature, the reaction mixture was partitioned between ethyl acetate (20 mL) and water (20 mL). The aqueous layer was extracted with ethyl acetate (2×15 mL) and the combined organic extracts were washed with brine (10 mL), dried (Na₂SO₄), filtered and concentrated. The residue was purified by column chromatography on silica gel (0-8% methanol in DCM) to give 4-(4-((1H-indol-2-yl)methyl)piperazin-1-yl)-1-((2-(trimethylsilyl)ethoxy)-methyl)-1H-pyrrolo[2,3-b]pyridine as a brown glass.

Yield 144 mg (96%). ¹H NMR (400 MHz, DMSO) δ 11.14 (s, 1H), 8.09 (d, J=5.5 Hz, 1H), 7.55 (d, J=8.0 Hz, 1H), 7.49 (d, J=3.8 Hz, 1H), 7.43 (d, J=8.0 Hz, 1H), 7.12 (dd, J=7.0, 7.0 Hz, 1H), 7.04 (dd, J=7.2, 7.2 Hz, 1H), 6.65 (d, J=3.6 Hz, 1H), 6.58 (d, J=5.6 Hz, 1H), 6.41 (d, J=1.3 Hz, 1H), 5.64 (s, 2H), 3.80 (s, 2H), 3.60-3.50 (m, 6H), 2.72 (dd, J=4.6, 4.6 Hz, 4H), 0.93-0.88 (m, 2H), 0.00 (s, 9H).

### Synthesis of 6-bromo-2-[[4-(4-pyridyl)piperazin-1-yl]methyl]-1H-indole (Compound 224)

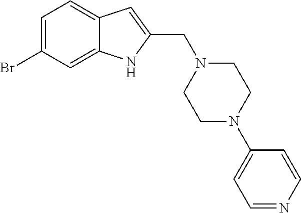 [SMILES: Brc1ccc2cc(CN3CCN(c4ccncc4)CC3)[nH]c2c1]

Compound 6-bromo-2-[[4-(4-pyridyl)piperazin-1-yl]methyl]-1H-indole was prepared from 5-bromo-1H-indole-2-carbaldehyde following a similar procedure to that described for the synthesis of 5-bromo-2-[[4-(4-pyridyl)piperazin-1-yl]methyl]-1H-indole, and was isolated as a brown solid.

Yield 644 mg (39%). ¹H NMR (400 MHz, DMSO) δ 11.20 (s, 1H), 8.20 (br s, 2H), 7.51 (d, J=1.3 Hz, 1H), 7.43 (d, J=8.4 Hz, 1H), 7.09 (dd, J=1.9, 8.4 Hz, 1H), 6.87 (br s, 2H), 6.35 (d, J=1.3 Hz, 1H), 3.67 (s, 2H), 3.35-3.29 (m, 4H), 2.55-2.52 (m, 4H). m/z: [ESI⁺] 371 (M+H)⁺, (C₁₈H₁₉BrN₄), Rₜ=2.41 (98.2%).

### Synthesis of (5-(trifluoromethyl)-1H-indol-2-yl)methanol

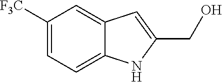 [SMILES: OCc1cc2cc(C(F)(F)F)ccc2[nH]1]

To a solution of 5-(trifluoromethyl)-1H-indole-2-carboxylic acid (800 mg, 3.49 mmol) in THF (5 mL) at 0° C. was added a 1 M solution of lithium aluminium hydride in THF (3.8 mL, 3.84 mmol) dropwise and the mixture was stirred at room temperature for 2 hrs, then heated to 65° C. and stirred for 2 hr. After cooling to 0° C., the mixture was quenched with saturated aqueous Rochelle salt solution (approx. 20 mL) and extracted with ethyl acetate. The organic layer was washed with brine (20 mL), dried (Na₂SO₄), filtered and concentrated to afford crude (5-(trifluoromethyl)-1H-indol-2-yl)methanol as a yellow solid. The material was taken onto the next step without further purification.

Yield 661 mg (88%). m/z: [ESI⁺] 214 (M−H)⁺.

### Synthesis of 5-(trifluoromethyl)-1H-indole-2-carbaldehyde

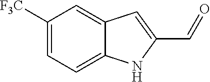 [SMILES: O=Cc1cc2cc(C(F)(F)F)ccc2[nH]1]

To a solution of (5-(trifluoromethyl)-1H-indol-2-yl)methanol (751 mg, 3.49 mmol) in THF (25 mL) was added MnO₂ (3034 mg, 34.9 mmol) and the mixture was stirred at room temperature for 18 hours. The mixture was then diluted with ethyl acetate and filtered through a pad of celite to afford 5-(trifluoromethyl)-1H-indole-2-carbaldehyde as a yellow solid.

Yield 541 mg (73%, over 2 steps). ¹H NMR (400 MHz, DMSO) δ 12.40 (s, 1H), 9.95 (s, 1H), 8.23 (s, 1H), 7.66 (d, J=8.9 Hz, 1H), 7.62 (dd, J=1.5, 8.9 Hz, 1H), 7.58 (d, J=1.5 Hz, 1H).

### Synthesis of (6-methoxy-1H-indol-2-yl)methanol

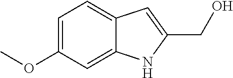

Compound (6-methoxy-1H-indol-2-yl)methanol was prepared from 6-methoxy-1H-indole-2-carboxylic acid following a similar procedure to that described for the synthesis of (5-(trifluoromethyl)-1H-indol-2-yl)methanol, and was isolated as an off-white solid. The intermediate was taken onto the next step without further purification.

Yield 1.13 g. m/z: [ESI⁺] 178 (M+H)⁺.

### Synthesis of 6-methoxy-1H-indole-2-carbaldehyde

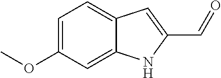

Compound 6-methoxy-1H-indole-2-carbaldehyde was prepared from (6-methoxy-1H-indol-2-yl)methanol following a similar procedure to that described for the synthesis of 5-(trifluoromethyl)-1H-indole-2-carbaldehyde, and was isolated as a brown solid.

Yield 369 mg (51%, over 2 steps). ¹H NMR (400 MHz, DMSO) δ 11.79 (s, 1H), 9.72 (s, 1H), 7.63 (d, J=8.8 Hz, 1H), 7.33 (dd, J=0.8, 2.1 Hz, 1H), 6.86 (d, J=2.3 Hz, 1H), 6.78 (dd, J=2.3, 8.8 Hz, 1H), 3.82 (s, 3H).

### Synthesis of (6-(trifluoromethyl)-1H-indol-2-yl)methanol

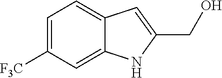 [SMILES: OCc1cc2ccc(C(F)(F)F)cc2[nH]1]

Compound (6-(trifluoromethyl)-1H-indol-2-yl)methanol was prepared from 6-(trifluoromethyl)-1H-indole-2-carboxylic acid following a similar procedure to that described for the synthesis of (5-(trifluoromethyl)-1H-indol-2-yl)methanol, and was isolated as a brown oil.

Yield 391 mg (83%). ¹H NMR (400 MHz, DMSO) δ 11.47 (s, 1H), 7.66 (d, J=7.4 Hz, 2H), 7.25 (d, J=8.4 Hz, 1H), 6.42 (d, J=1.1 Hz, 1H), 5.43 (t, J=5.5 Hz, 1H), 4.67 (d, J=5.5 Hz, 2H).

### Synthesis of 6-(trifluoromethyl)-1H-indole-2-carbaldehyde

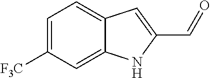 [SMILES: O=Cc1cc2ccc(C(F)(F)F)cc2[nH]1]

Compound 6-(trifluoromethyl)-1H-indole-2-carbaldehyde was prepared from (6-(trifluoromethyl)-1H-indol-2-yl)methanol following a similar procedure to that described for the synthesis of 5-(trifluoromethyl)-1H-indole-2-carbaldehyde, and was isolated as a brown solid. The intermediate was taken onto the next step without further purification.

Yield 244 mg (63%). m/z: [ESI⁺] 212 (M−H)⁺.

### Synthesis of (5-nitro-1H-indol-2-yl)methanol

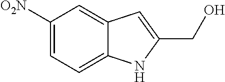 [SMILES: O=[N+]([O-])c1ccc2[nH]c(CO)cc2c1]

To a solution of ethyl 5-nitro-1H-indole-2-carboxylate (1300 mg, 5.5 mmol) in THF (44 mL) at −60° C. was added dropwise a 1 M solution of diisobutylaluminium hydride in DCM (17 mL, 16.6 mmol) and the mixture was stirred at −60° C. for 4 hours and then at −20° C. for 1 hour. The mixture was quenched at 0° C. with saturated aqueous Rochelle salt solution (approx. 20 mL) and extracted with ethyl acetate (3×50 mL). The combined organics were washed with brine (20 mL), dried (Na₂SO₄), filtered and concentrated.to afford (5-nitro-1H-indol-2-yl)methanol as a brown solid.

Yield 1068 mg (quant). ¹H NMR (400 MHz, DMSO) δ 11.82 (s, 1H), 8.50 (d, J=2.3 Hz, 1H), 7.97 (dd, J=2.3, 9.0 Hz, 1H), 7.49 (d, J=9.0 Hz, 1H), 6.58 (s, 1H), 5.46 (t, J=5.3 Hz, 1H), 4.66 (d, J=5.3 Hz, 2H).

### Synthesis of 5-nitro-1H-indole-2-carbaldehyde

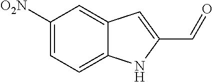 [SMILES: O=Cc1cc2cc([N+](=O)[O-])ccc2[nH]1]

Compound 5-nitro-1H-indole-2-carbaldehyde was prepared from (5-nitro-1H-indol-2-yl)methanol following a similar procedure to that described for the synthesis of 5-(trifluoromethyl)-1H-indole-2-carbaldehyde, and was isolated as a brown solid.

Yield 893 mg (84%). ¹H NMR (400 MHz, DMSO) δ 12.68 (s, 1H), 9.98 (s, 1H), 8.85 (s, 1H), 8.20 (d, J=9.0 Hz, 1H), 7.70 (s, 1H), 7.63 (d, J=9.0 Hz, 1H).

### Synthesis of 5-nitro-2-((4-(pyridin-4-yl)piperazin-1-yl)methyl)-1H-indole

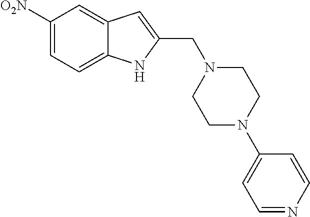 [SMILES: O=[N+]([O-])c1ccc2[nH]c(CN3CCN(c4ccncc4)CC3)cc2c1]

Compound 5-nitro-2-((4-(pyridin-4-yl)piperazin-1-yl)methyl)-1H-indole was prepared from 5-nitro-1H-indole-2-carbaldehyde following a similar procedure to that described for the synthesis of 5-bromo-2-[[4-(4-pyridyl)piperazin-1-yl]methyl]-1H-indole, and was isolated as a brown solid.

Yield 1053 mg (67%). ¹H NMR (400 MHz, DMSO) δ 11.90 (s, 1H), 8.56 (d, J=2.3 Hz, 1H), 8.21 (d, J=6.5 Hz, 2H), 8.03 (dd, J=2.3, 9.0 Hz, 1H), 7.55 (d, J=9.0 Hz, 1H), 6.86 (d, J=6.5 Hz, 2H), 6.69 (s, 1H), 3.79 (s, 2H), 3.43-3.39 (m, 4H), 2.63-2.58 (m, 4H).

### Synthesis of (7-chloro-1H-indol-2-yl)methanol

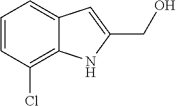 [SMILES: OCc1cc2cccc(Cl)c2[nH]1]

Compound (7-chloro-1H-indol-2-yl)methanol was prepared from 7-chloro-1H-indole-2-carboxylic acid following a similar procedure to that described for the synthesis of (5-(trifluoromethyl)-1H-indol-2-yl)methanol, except it was further purified by column chromatography on silica gel (0-100% ethyl acetate in cyclohexane) to afford (7-chloro-1H-indol-2-yl)methanol as a brown oil.

Yield 264 mg (57%). ¹H NMR (400 MHz, DMSO) δ 11.24 (s, 1H), 7.46 (d, J=7.9 Hz, 1H), 7.12 (d, J=7.9 Hz, 1H), 6.97 (dd, J=7.7, 7.7 Hz, 1H), 6.42 (d, J=1.8 Hz, 1H), 5.19 (t, J=5.8 Hz, 1H), 4.63 (d, J=5.8 Hz, 2H).

### Synthesis of 7-chloro-1H-indole-2-carbaldehyde

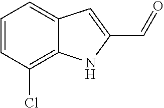 [SMILES: O=Cc1cc2cccc(Cl)c2[nH]1]

Compound 7-chloro-1H-indole-2-carbaldehyde was prepared from (7-chloro-1H-indol-2-yl)methanol following a similar procedure to that described for the synthesis of 5-(trifluoromethyl)-1H-indole-2-carbaldehyde, and was isolated as a brown solid.

Yield 222 mg (86%). ¹H NMR (400 MHz, DMSO) δ 12.34 (s, 1H), 9.94 (s, 1H), 7.76 (d, J=7.8 Hz, 1H), 7.50 (d, J=2.0 Hz, 1H), 7.45 (d, J=7.8 Hz, 1H), 7.15 (dd, J=7.8, 7.8 Hz, 1H).

### Synthesis of Methyl 2-((4-(pyridin-4-yl)piperazin-1-yl)methyl)-1H-indole-5-carboxylate

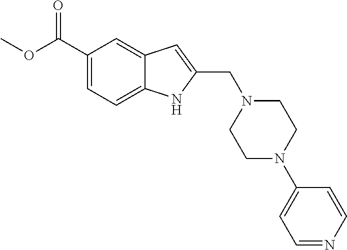 [SMILES: COC(=O)c1ccc2[nH]c(CN3CCN(c4ccncc4)CC3)cc2c1]

Compound methyl 2-((4-(pyridin-4-yl)piperazin-1-yl)methyl)-1H-indole-5-carboxylate was prepared from methyl 2-formyl-1H-indole-5-carboxylate and 1-(pyridin-4-yl)piperazine following a similar procedure to that described for the synthesis of 5-bromo-2-[[4-(4-pyridyl)piperazin-1-yl]methyl]-1H-indole, except it was purified by SCX-2 ion exchange chromatography (2 g, 0.6 mmol/g loading, washed with MeOH and eluted with 1 N ammonia/MeOH), and was isolated as a brown solid.

Yield 342 mg (79%). ¹H NMR (400 MHz, DMSO) δ 11.49 (s, 1H), 8.20 (d, J=1.1 Hz, 1H), 8.16 (d, J=6.5 Hz, 2H), 7.70 (dd, J=1.6, 8.5 Hz, 1H), 7.42 (d, J=8.5 Hz, 1H), 6.82 (dd, J=1.6, 6.5 Hz, 2H), 6.49 (d, J=1.1 Hz, 1H), 3.85 (s, 3H), 3.71 (s, 2H), 3.36-3.32 (m, 4H), 2.58-2.52 (m, 4H).

### Synthesis of 2-((4-(pyridin-4-yl)piperazin-1-yl)methyl)-1H-indole-5-carboxylic Acid

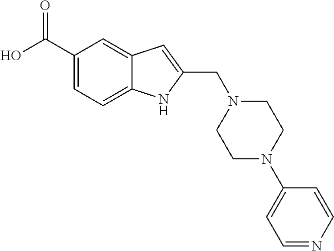 [SMILES: O=C(O)c1ccc2[nH]c(CN3CCN(c4ccncc4)CC3)cc2c1]

To a suspension of methyl 2-((4-(pyridin-4-yl)piperazin-1-yl)methyl)-1H-indole-5-carboxylate (100 mg, 0.28 mmol) in THF (3 mL) was added a solution of NaOH (11 mg, 0.28 mmol) in water (1 mL) and the reaction was stirred at room temperature for 2 hours and then at 65° C. for 4 days. The reaction mixture was concentrated, re-dissolved in MeOH/water/DMSO and purified by SAX-2 ion exchange chromatography (1 g, 0.6 mmol/g loading, washed with MeOH and eluted with 50% AcOH/MeOH) and then SCX-2 ion exchange chromatography (2 g, 0.6 mmol/g loading, washed with MeOH and eluted with 1 N ammonia/MeOH), to afford 2-((4-(pyridin-4-yl)piperazin-1-yl)methyl)-1H-indole-5-carboxylic acid as a brown solid.

Yield 34 mg (33%). ¹H NMR (400 MHz, DMSO) δ 11.42 (s, 1H), 8.17-8.15 (m, 3H), 7.69 (dd, J=1.6, 8.5 Hz, 1H), 7.39 (d, J=8.5 Hz, 1H), 6.82 (d, J=6.7 Hz, 2H), 6.47 (d, J=1.1 Hz, 1H), 3.70 (s, 2H), 3.39-3.31 (m, 4H), 2.58-2.53 (m, 4H). Acid OH proton hidden.

### Synthesis of 2-(hydroxymethyl)-1H-indole-5-carbonitrile

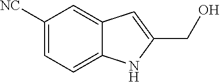 [SMILES: N#Cc1ccc2[nH]c(CO)cc2c1]

Compound 2-(hydroxymethyl)-1H-indole-5-carbonitrile was prepared from 5-cyano-1H-indole-2-carboxylic acid following a similar procedure to that described for the synthesis of (5-(trifluoromethyl)-1H-indol-2-yl)methanol, and was isolated as a yellow solid. The intermediate was aken onto the next step without further purification.

Yield 727 mg. m/z: [ESI⁺] 171 (M−H)⁺.

### Synthesis of 2-formyl-1H-indole-5-carbonitrile

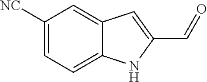 [SMILES: N#Cc1ccc2[nH]c(C=O)cc2c1]

Compound 6-methoxy-1H-indole-2-carbaldehyde was prepared from (6-methoxy-1H-indol-2-yl)methanol following a similar procedure to that described for the synthesis of 5-(trifluoromethyl)-1H-indole-2-carbaldehyde, and was isolated as a yellow solid.

Yield 600 mg (76%, over 2 steps). ¹H NMR (400 MHz, DMSO) δ 12.50 (s, 1H), 9.95 (s, 1H), 8.39 (dd, J=0.8, 1.5 Hz, 1H), 7.67 (dd, J=1.5, 8.6 Hz, 1H), 7.62 (d, J=8.6 Hz, 1H), 7.56 (s, 1H).

### Synthesis of Eethyl 5-(2-methoxyethoxy)-1H-indole-2-carboxylate (I) and ethyl 5-(2-methoxyethoxy)-1-(2-methoxyethyl)-1H-indole-2-carboxylate (II)

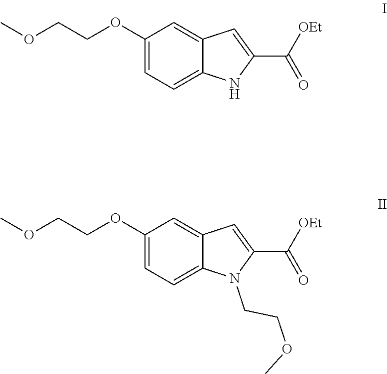 [SMILES: CCOC(=O)c1cc2cc(OCCOC)ccc2[nH]1 ; CCOC(=O)c1cc2cc(OCCOC)ccc2n1CCOC]

To a solution of ethyl 5-hydroxy-1H-indole-2-carboxylate (200 mg, 0.98 mmol) and cesium carbonate (953 mg, 2.92 mmol) in anhydrous DMF (2 mL) was added at room temperature 1-bromo-2-methoxyethane (110 μL, 1.17 mmol) and the mixture was stirred at room temperature for 18 hours. The reaction mixture was partitioned between ethyl acetate (20 mL) and water (10 mL). The layers were separated, and the organic phase was washed with water (10 mL), water/brine 1:1 (10 mL) and brine (10 mL) successively. The organic layer was dried (Na₂SO₄), filtered and evaporated and the residue was purified by column chromatography on silica gel (0-100% ethyl acetate in cyclohexane) to furnish ethyl 5-(2-methoxyethoxy)-1H-indole-2-carboxylate and ethyl 5-(2-methoxyethoxy)-1-(2-methoxyethyl)-1H-indole-2-carboxylate as a white solid.

Yield 141 mg. I: m/z: [ESI⁺] 264 (M+H)⁺ (68% pure). II: m/z: [ESI⁺] 322 (M+H)⁺ (30% pure).

### Synthesis of (5-(2-methoxyethoxy)-1H-indol-2-yl)methanol (I) and (5-(2-methoxyethoxy)-1-(2-methoxyethyl)-1H-indol-2-yl)methanol (II)

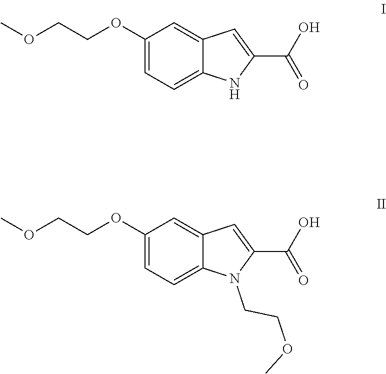 [SMILES: COCCOc1ccc2[nH]c(C(=O)O)cc2c1 ; COCCOc1ccc2c(c1)cc(C(=O)O)n2CCOC]

Compounds (5-(2-methoxyethoxy)-1H-indol-2-yl)methanol and (5-(2-methoxyethoxy)-1-(2-methoxyethyl)-1H-indol-2-yl)methanol were prepared from ethyl 5-(2-methoxyethoxy)-1H-indole-2-carboxylate and ethyl 5-(2-methoxyethoxy)-1-(2-methoxyethyl)-1H-indole-2-carboxylate following a similar procedure to that described for the synthesis of (5-(trifluoromethyl)-1H-indol-2-yl)methanol, and were isolated as a brown oil. The mixture was taken onto the next step without further purification.

Yield 120 mg. I: m/z: [ESI⁺] 222 (M+H)⁺ (61% pure). II: m/z: [ESI⁺] 280 (M+H)⁺ (34% pure).

### Synthesis of 5-(2-methoxyethoxy)-1H-indole-2-carbaldehyde (I) and 5-(2-methoxyethoxy)-1-(2-methoxyethyl)-1H-indole-2-carbaldehyde (II)

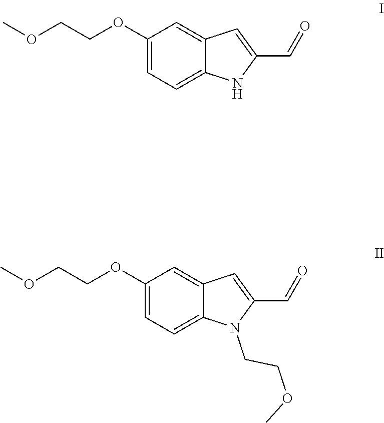 [SMILES: COCCOc1ccc2[nH]c(C=O)cc2c1 ; COCCOc1ccc2c(c1)cc(C=O)n2CCOC]

Compounds 5-(2-methoxyethoxy)-1H-indole-2-carbaldehyde and 5-(2-methoxyethoxy)-1-(2-methoxyethyl)-1H-indole-2-carbaldehyde were prepared from (5-(2-methoxyethoxy)-1H-indol-2-yl)methanol and (5-(2-methoxyethoxy)-1-(2-methoxyethyl)-1H-indol-2-yl)methanol following a similar procedure to that described for the synthesis of 5-(trifluoromethyl)-1H-indole-2-carbaldehyde, and were isolated as a brown solid. The mixture was taken onto the next step without further purification.

Yield 120 mg. I: m/z: [ESI] 218 (M−H)⁻ (66% pure). II: m/z: [ESI⁺] 278 (M+H)⁺ (29% pure).

### Synthesis of 1-(diethoxymethyl)-1H-indole-3-carbonitrile

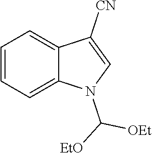 [SMILES: CCOC(OCC)n1cc(C#N)c2ccccc21]

A suspension of 1H-indole-3-carbonitrile (1000 mg, 7.03 mmol) in triethyl orthoformate (11.7 mL, 70.34 mmol) was heated to 160° C. in a high-pressure tube and stirred for 3 days. The reaction mixture was evaporated to dryness and the residue was purified by column chromatography on silica gel (0-20% ethyl acetate in iso-hexane) to afford 1-(diethoxymethyl)-1H-indole-3-carbonitrile as a colourless oil.

Yield 1806 mg (quant.). ¹H NMR (400 MHz, DMSO) δ 8.41 (s, 1H), 7.80 (d, J=7.7 Hz, 1H), 7.68 (d, J=7.7 Hz, 1H), 7.38 (dd, J=7.5, 7.5 Hz, 1H), 7.33 (dd, J=7.5, 7.5 Hz, 1H), 6.59 (s, 1H), 3.62 (q, J=7.0 Hz, 4H), 1.17 (t, J=7.0 Hz, 6H).

### Synthesis of 1-(diethoxymethyl)-2-formyl-1H-indole-3-carbonitrile

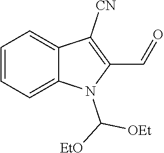 [SMILES: CCOC(OCC)n1c(C=O)c(C#N)c2ccccc21]

To a solution of 1-(diethoxymethyl)-1H-indole-3-carbonitrile (200 mg, 0.82 mmol) in THF (6 mL) at −78° C. was added a 1.7 M tert-butyllithium in pentane solution (0.53 mL, 0.90 mmol) and the mixture was allowed to warm to −10° C. and stirred for 30 minutes. Then the mixture was cooled to −78° C. and DMF (0.57 mL, 7.32 mmol) was added and the mixture was allowed to warm to −10° C. and stirred for 2 hours. The reaction mixture was quenched at 0° C. with saturated aqueous of NaHCO₃ (10 mL) and extracted with Et₂O (2×10 mL). The combined organic extracts were dried (Na₂SO₄), filtered and evaporated to afford 1-(diethoxymethyl)-2-formyl-1H-indole-3-carbonitrile as a yellow oil. The intermediate was taken onto the next step without further purification.

Yield 185 mg (crude).

### Synthesis of 1-(diethoxymethyl)-2-((4-(pyridin-4-yl)piperazin-1-yl)methyl)-1H-indole-3-carbonitrile

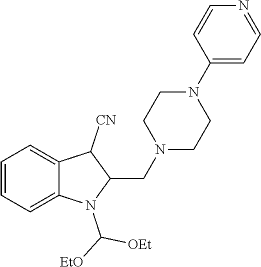 [SMILES: CCOC(OCC)N1c2ccccc2C(C#N)C1CN1CCN(c2ccncc2)CC1]

Compound 1-(diethoxymethyl)-2-((4-(pyridin-4-yl)piperazin-1-yl)methyl)-1H-indole-3-carbonitrile was prepared from 1-(diethoxymethyl)-2-formyl-1H-indole-3-carbonitrile following a similar procedure to that described for the synthesis of 5-bromo-2-[[4-(4-pyridyl)piperazin-1-yl]methyl]-1H-indole and was isolated as a yellow oil. Taken onto the next step without further purification.

Yield 180 mg (crude). m/z: [ESI⁺] 420 (M+H)⁺.

### Synthesis of Tert-Butyl 6-(pyridin-4-yl)-2,6-diazaspiro[3.3]heptane-2-carboxylate

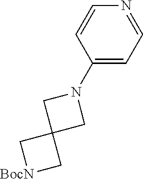 [SMILES: CC(C)(C)OC(=O)N1CC2(C1)CN(c1ccncc1)C2]

To a degassed suspension of tert-butyl 2,6-diazaspiro[3.3]heptane-2-carboxylate (106 mg, 0.54 mmol), 4-iodopyridine (100 mg, 0.49 mmol) and cesium carbonate (318 mg, 0.98 mmol) in anhydrous dioxane (4 mL) was added palladium(II) acetate (11 mg, 0.05 mmol) and RuPhos (46 mg, 0.10 mmol). The mixture was sparged for 10 minutes with nitrogen and heated at 95° C. for 3 hours in a sealed tube. After cooling to room temperature, the reaction mixture was partitioned between ethyl acetate (20 mL) and water (20 mL). The aqueous layer was extracted with ethyl acetate (2×15 mL) and the combined organic extracts were washed with brine (10 mL), dried (Na₂SO₄), filtered and concentrated. The residue was purified by column chromatography on silica gel (0-10% 1 N ammonia/methanol in DCM) to give tert-butyl 6-(pyridin-4-yl)-2,6-diazaspiro[3.3]heptane-2-carboxylate as a brown gum.

Yield 195 mg (97%). ¹H NMR (400 MHz, DMSO) δ 8.13 (dd, J=1.6, 4.7 Hz, 2H), 6.34 (dd, J=1.6, 4.7 Hz, 2H), 4.04 (s, 8H), 1.39 (s, 9H).

### Synthesis of 2-(pyridin-4-yl)-2,6-diazaspiro[3.3]heptane

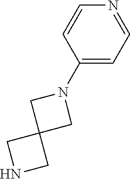 [SMILES: c1cc(N2CC3(CNC3)C2)ccn1]

To a solution of tert-butyl 6-(pyridin-4-yl)-2,6-diazaspiro[3.3]heptane-2-carboxylate (195 mg, 0.71 mmol) in anhydrous DCM (3 mL) was added TFA (0.5 mL, 6.53 mmol) and the mixture was stirred at room temperature for 2 hours. The reaction mixture was concentrated and purified by SCX-2 ion exchange chromatography (2 g, 0.6 mmol/g loading, washed with MeOH and eluted with 1 N ammonia/MeOH), and was isolated as an off-white solid.

Yield 67 mg (54%). ¹H NMR (400 MHz, DMSO) δ 8.11 (dd, J=1.6, 4.7 Hz, 2H), 6.33 (dd, J=1.6, 4.7 Hz, 2H), 3.97 (s, 4H), 3.61 (s, 4H). NH proton obscured under residual water peak.

### Synthesis of Tert-Butyl 7-(pyridin-4-yl)-2,7-diazaspiro[4.4]nonane-2-carboxylate

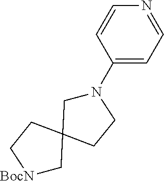 [SMILES: CC(C)(C)OC(=O)N1CCC2(CCN(c3ccncc3)C2)C1]

Compound tert-butyl 7-(pyridin-4-yl)-2,7-diazaspiro[4.4]nonane-2-carboxylate was prepared from tertbutyl 2,7-diazaspiro[4.4]nonane-2-carboxylate and 4-iodopyridine following a similar procedure to that described for the synthesis of tert-butyl 6-(pyridin-4-yl)-2,6-diazaspiro[3.3]heptane-2-carboxylate, and was isolated as a yellow gum.

Yield 235 mg (79%). ¹H NMR (400 MHz, DMSO) δ 8.09 (dd, J=1.6, 4.9 Hz, 2H), 6.45 (dd, J=1.6, 4.9 Hz, 2H), 3.41-3.31 (m, 4H), 3.27-3.22 (m, 4H), 1.99-1.94 (m, 2H), 1.89-1.83 (m, 2H), 1.41 (d, J=5.6 Hz, 9H).

### Synthesis of 2-(pyridin-4-yl)-2,7-diazaspiro[4.4]nonane

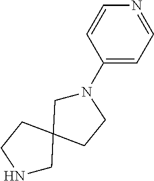 [SMILES: c1cc(N2CCC3(CCNC3)C2)ccn1]

Compound 2-(pyridin-4-yl)-2,7-diazaspiro[4.4]nonane was prepared from tert-butyl 7-(pyridin-4-yl)-2,7-diazaspiro[4.4]nonane-2-carboxylate following a similar procedure to that described for the synthesis of 2-(piperazin-1-ylmethyl)-1H-indole, and was isolated as a colourless oil.

Yield 100 mg (65%). ¹H NMR (400 MHz, DMSO) δ 8.08 (dd, J=1.5, 4.9 Hz, 2H), 6.42 (dd, J=1.5, 4.9 Hz, 2H), 4.11 (br s, 1H), 3.32-3.14 (m, 4H), 2.86 (t, J=7.1 Hz, 2H), 2.67 (s, 2H), 1.98-1.89 (m, 2H), 1.72-1.66 (m, 2H).

### Synthesis of Tert-Butyl (3R,5S)-3,5-dimethyl-4-(pyridin-4-yl)piperazine-1-carboxylate

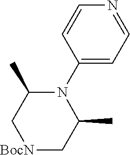 [SMILES: C[C@@H]1CN(C(=O)OC(C)(C)C)C[C@H](C)N1c1ccncc1]

Compound tert-butyl (3R,5S)-3,5-dimethyl-4-(pyridin-4-yl)piperazine-1-carboxylate was prepared from tert-butyl (3R,5S)-3,5-dimethylpiperazine-1-carboxylate and 4-iodopyridine following a similar procedure to that described for the synthesis of tert-butyl 6-(pyridin-4-yl)-2,6-diazaspiro[3.3]heptane-2-carboxylate, and was isolated as a brown glass.

Yield 39 mg (14%). m/z: [ESI⁺] 292 (M+H)⁺

### Synthesis of (2R,6S)-2,6-dimethyl-1-(pyridin-4-yl)piperazine Dihydrochloride

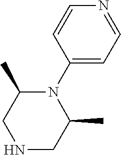

To a solution of tert-butyl (3R,5S)-3,5-dimethyl-4-(pyridin-4-yl)piperazine-1-carboxylate (39 mg, 0.13 mmol) in dioxane (1 mL) was added a solution of HCl in dioxane (4 M, 2 mL, 8 mmol) and the reaction mixture was stirred at room temperature for 3 hours. The reaction mixture was concentrated to afford (2R,6S)-2,6-dimethyl-1-(pyridin-4-yl)piperazine dihydrochloride as an off white solid. Taken onto the next step without further purification.

Yield 35 mg (quant.). m/z: [ESI⁺] 192 (M+H)⁺.

### Synthesis of Tert-Butyl (3aR,6aS)-5-(pyridin-4-yl)hexahydropyrrolo[3,4-c]pyrrole-2(1H)-carboxylate

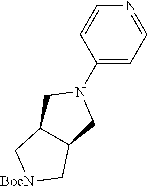 [SMILES: CC(C)(C)OC(=O)N1C[C@@H]2CN(c3ccncc3)C[C@@H]2C1]

Compound tert-butyl (3aR,6aS)-5-(pyridin-4-yl)hexahydropyrrolo[3,4-c]pyrrole-2(1H)-carboxylate was prepared from tert-butyl (3aR,6aS)-hexahydropyrrolo[3,4-c]pyrrole-2(1H)-carboxylate and 4-iodopyridine following a similar procedure to that described for the synthesis of tert-butyl 6-(pyridin-4-yl)-2,6-diazaspiro[3.3]heptane-2-carboxylate, and was isolated as a yellow oil.

Yield 281 mg (quant.). ¹H NMR (400 MHz, DMSO) δ 8.10 (dd, J=1.6, 4.9 Hz, 2H), 6.45 (dd, J=1.6, 4.9 Hz, 2H), 3.58-3.50 (m, 4H), 3.20-3.16 (m, 4H), 3.04-3.00 (m, 2H), 1.40 (s, 9H).

### Synthesis of (3aR,6aS)-2-(pyridin-4-yl)octahydropyrrolo[3,4-c]pyrrole

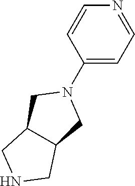 [SMILES: c1cc(N2C[C@H]3CNC[C@H]3C2)ccn1]

Compound (3aR,6aS)-2-(pyridin-4-yl)octahydropyrrolo[3,4-c]pyrrole was prepared from tert-butyl (3aR,6aS)-5-(pyridin-4-yl)hexahydropyrrolo[3,4-c]pyrrole-2(1H)-carboxylate following a similar procedure to that described for the synthesis of 2-(piperazin-1-ylmethyl)-1H-indole, and was isolated as an off-white solid.

Yield 128 mg (70%). ¹H NMR (400 MHz, DMSO) δ 8.10 (dd, J=1.6, 4.8 Hz, 2H), 6.47 (dd, J=1.6, 4.8 Hz, 2H), 3.49 (dd, J=8.1, 10.4 Hz, 2H), 3.08 (dd, J=3.7, 10.4 Hz, 2H), 2.93 (dd, J=8.1, 10.9 Hz, 2H), 2.89-2.81 (m, 2H), 2.64 (dd, J=2.9, 10.9 Hz, 2H). NH proton obscured under residual water peak.

### Synthesis of Tert-Butyl 4-(5,6-dimethylpyrimidin-4-yl)piperazine-1-carboxylate

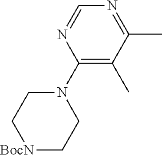 [SMILES: Cc1ncnc(N2CCN(C(=O)OC(C)(C)C)CC2)c1C]

Compound tert-butyl 4-(5,6-dimethylpyrimidin-4-yl)piperazine-1-carboxylate was prepared from tert-butyl piperazine-1-carboxylate and 4-chloro-5,6-dimethylpyrimidine following a similar procedure to that described for the synthesis of tert-butyl 6-(pyridin-4-yl)-2,6-diazaspiro[3.3]heptane-2-carboxylate, and was isolated as an off-white solid.

Yield 459 mg (40%). ¹H NMR (400 MHz, CDCl₃) δ 8.54 (s, 1H), 3.57-3.53 (m, 4H), 3.27-3.23 (m, 4H), 2.42 (s, 3H), 2.16 (s, 3H), 1.48 (s, 9H).

### Synthesis of 4,5-dimethyl-6-(piperazin-1-yl)pyrimidine Hydrochloride

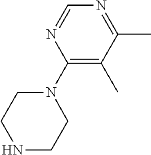

Compound 4,5-dimethyl-6-(piperazin-1-yl)pyrimidine hydrochloride was prepared from tert-butyl 4-(5,6-dimethylpyrimidin-4-yl)piperazine-1-carboxylate following a similar procedure to that described for the synthesis of 2-(piperazin-1-ylmethyl)-1H-indole, except that it was not purified by SCX-2 ion exchange chromatography and was isolated as an off-white solid. The intermediate was taken as the hydrochloride salt onto the next step without further purification.

Yield 511 mg (crude). m/z: [ESI⁺] 193 (M+H)⁺.

### Synthesis of Pyrazolo[1,5-a]pyridin-2-ylmethanol

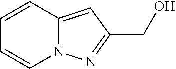 [SMILES: OCc1cc2ccccn2n1]

Compound pyrazolo[1,5-a]pyridin-2-ylmethanol was prepared from pyrazolo[1,5-a]pyridine-2-carboxylic acid following a similar procedure to that described for the synthesis of (5-(trifluoromethyl)-1H-indol-2-yl)methanol, and was isolated as a brown oil. The intermediate was taken onto the next step without further purification.

Yield 338 mg (crude). m/z: [ESI⁺] 149 (M+H)⁺.

### Synthesis of pyrazolo[1,5-a]pyridine-2-carbaldehyde

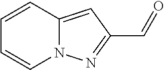 [SMILES: O=Cc1cc2ccccn2n1]

Compound pyrazolo[1,5-a]pyridine-2-carbaldehyde was prepared from pyrazolo[1,5-a]pyridin-2-ylmethanol following a similar procedure to that described for the synthesis of 5-(trifluoromethyl)-1H-indole-2-carbaldehyde, and was isolated as a yellow solid.

Yield 208 mg (crude). m/z: [ESI⁺] 147 (M+H)⁺.

### Synthesis of (1H-pyrrolo[2,3-b]pyridin-2-yl)methanol

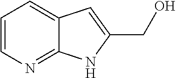 [SMILES: OCc1cc2cccnc2[nH]1]

Compound (1H-pyrrolo[2,3-b]pyridin-2-yl)methanol was prepared from 1H-pyrrolo[2,3-b]pyridine-2-carboxylic acid following a similar procedure to that described for the synthesis of (5-(trifluoromethyl)-1H-indol-2-yl)methanol, and was isolated as a brown solid. The intermediate was taken onto the next step without further purification.

Yield 248 mg (crude). m/z: [ESI⁺] 149 (M+H)⁺.

### Synthesis of 1H-pyrrolo[2,3-b]pyridine-2-carbaldehyde

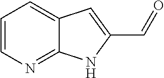 [SMILES: O=Cc1cc2cccnc2[nH]1]

Compound 1H-pyrrolo[2,3-b]pyridine-2-carbaldehyde was prepared from (1H-pyrrolo[2,3-b]pyridin-2-yl)methanol following a similar procedure to that described for the synthesis of 5-(trifluoromethyl)-1H-indole-2-carbaldehyde, and was isolated as a yellow solid.

Yield 168 mg (crude). m/z: [ESI⁺] 147 (M+H)⁺.

### Synthesis of (1H-pyrrolo[2,3-c]pyridin-2-yl)methanol

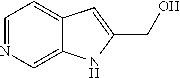 [SMILES: OCc1cc2ccncc2[nH]1]

Compound (1H-pyrrolo[2,3-c]pyridin-2-yl)methanol was prepared from 1H-pyrrolo[2,3-c]pyridine-2-carboxylic acid following a similar procedure to that described for the synthesis of (5-(trifluoromethyl)-1H-indol-2-yl)methanol, and was isolated as a brown solid. The intermediate was taken onto the next step without further purification.

Yield 321 mg (crude). m/z: [ESI⁺] 149 (M+H)⁺.

### Synthesis of 1H-pyrrolo[2,3-c]pyridine-2-carbaldehyde

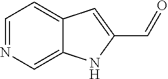 [SMILES: O=Cc1cc2ccncc2[nH]1]

Compound 1H-pyrrolo[2,3-c]pyridine-2-carbaldehyde was prepared from (1H-pyrrolo[2,3-c]pyridin-2-yl)methanol following a similar procedure to that described for the synthesis of 5-(trifluoromethyl)-1H-indole-2-carbaldehyde, and was isolated as a yellow solid.

Yield 214 mg (68%). m/z: [ESI⁺] 147 (M+H)⁺.

### Synthesis of (1H-pyrrolo[3,2-b]pyridin-2-yl)methanol

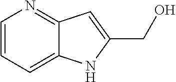 [SMILES: OCc1cc2ncccc2[nH]1]

Compound (1H-pyrrolo[3,2-b]pyridin-2-yl)methanol was prepared from 1H-pyrrolo[3,2-b]pyridine-2-carboxylic acid following a similar procedure to that described for the synthesis of (5-(trifluoromethyl)-1H-indol-2-yl)methanol, and was isolated as a yellow solid. The intermediate was taken onto the next step without further purification.

Yield 186 mg (crude). m/z: [ESI⁺] 149 (M+H)⁺.

### Synthesis of 1H-pyrrolo[3,2-b]pyridine-2-carbaldehyde

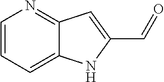 [SMILES: O=Cc1cc2ncccc2[nH]1]

Compound 1H-pyrrolo[3,2-b]pyridine-2-carbaldehyde was prepared from (1H-pyrrolo[3,2-b]pyridin-2-yl)methanol following a similar procedure to that described for the synthesis of 5-(trifluoromethyl)-1H-indole-2-carbaldehyde, and was isolated as a yellow solid.

Yield 164 mg (90%). m/z: [ESI⁺] 147 (M+H)⁺.

### Synthesis of Tert-Butyl 4-(5-formylpyrimidin-4-yl)piperazine-1-carboxylate

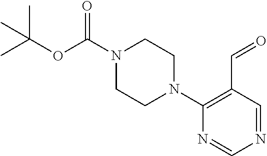 [SMILES: CC(C)(C)OC(=O)N1CCN(c2ncncc2C=O)CC1]

To a solution of tert-butyl 4-(5-ethenylpyrimidin-4-yl)piperazine-1-carboxylate (2.00 g, 6.89 mmol) in THF (20 mL) were added water (20 mL), osmium tetroxide (35 mg, 0.138 mmol) at room temperature under nitrogen atmosphere. The resulting mixture was stirred for 2 minutes at room temperature under a nitrogen atmosphere. To the above mixture was added sodium metaperiodate (2.95 g, 13.79 mmol) in portions over 2 minutes at room temperature. The resulting mixture was stirred for additional 16 h at room temperature. The resulting solution was diluted with water (300 mL) and extracted with ethyl acetate (3×200 mL). The combined organic layers were dried over anhydrous Na₂SO₄. After filtration, the filtrate was concentrated under reduced pressure. The residue was purified by reverse phase flash chromatography with the following conditions: Column: Spherical C18, 20-40 um, 330 g; Mobile Phase A: water; Mobile Phase B: ACN; How rate: 80 m/min; Gradient: 45% B-65% B in 20 min; Detector: UV 254/220 nm. The fractions containing the desired product were collected and concentrated under reduced pressure to afford tert-butyl 4-(5-formylpyrimidin-4-yl)piperazine-1-carboxylate as a brown solid.

Yield 1.10 g (55%). ¹H NMR (400 MHz, DMSO) δ 9.84 (s, 1H), 8.80 (s, 1H), 8.66 (s, 1H), 3.63 (t, J=5.2 Hz, 4H), 3.47 (t, J=5.2 Hz, 4H), 1.43 (s, 9H). m/z: [ESI⁺] 293 (M+H)⁺.

### Synthesis of Tert-Butyl 4-(5-(hydroxymethyl)pyrimidin-4-yl)piperazine-1-carboxylate

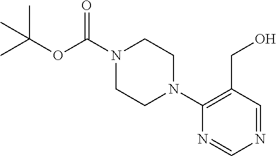 [SMILES: CC(C)(C)OC(=O)N1CCN(c2ncncc2CO)CC1]

To a solution of tert-butyl 4-(5-formylpyrimidin-4-yl)piperazine-1-carboxylate (270 mg, 0.924 mmol) in ethanol (10 mL) was added sodium borohydride (52 mg, 1.374 mmol) at 0° C. under a nitrogen atmosphere. The resulting mixture was stirred for 30 min at 0° C. under a nitrogen atmosphere. The resulting mixture was quenched with acetic acid (0.15 mL, 2.623 mmol). The resulting mixture was concentrated under reduced pressure. The residue was purified by reverse phase flash chromatography with the following conditions: Column: Spherical C18, 20-40 μm, 330 g; Mobile Phase A: water (plus 10 mM NH₄HCO₃); Mobile Phase B: ACN; Flow rate: 80 mL/min; Gradient: 35% B-55% B in 20 min; Detector: UV 254/220 nm. The fractions containing the desired product were collected and concentrated under reduced pressure to afford tert-butyl 4-(5-(hydroxymethyl)pyrimidin-4-yl)piperazine-1-carboxylate as a brown oil.

Yield 200 mg (74%). ¹H NMR (400 MHz, DMSO) δ 8.53 (s, 1H), 8.30 (s, 1H), 5.44 (t, J=5.2 Hz, 1H), 4.43 (d, J=5.2 Hz, 2H), 3.55 (t, J=4.8 Hz, 4H), 3.43 (t, J=4.8 Hz, 4H), 1.43 (s, 9H). m/z: [ESI⁺]295 (M+H)⁺.

### Synthesis of Tert-Butyl 4-(5-((2-methoxyethoxy)methyl)pyrimidin-4-yl)piperazine-1-carboxylate

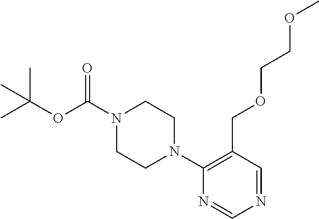 [SMILES: COCCOCc1cncnc1N1CCN(C(=O)OC(C)(C)C)CC1]

To a solution of tert-butyl 4-[5-(hydroxymethyl)pyrimidin-4-yl]piperazine-1-carboxylate (0.73 g, 2.48 mmol) in anhydrous DMF (10 mL) was added sodium hydride (0.15 g, 3.75 mmol, 60% w/w dispersed into mineral oil) at 0° C. under a nitrogen atmosphere. After stirring for additional 30 min, 2-bromoethyl methyl ether (0.35 mL, 3.72 mmol) was added dropwise over 5 min. The resulting mixture was stirred for additional 2 h at ambient temperature. The reaction was quenched with water (100 mL) and extracted with ethyl acetate (3×100 mL). The combined organic layers were dried over anhydrous sodium sulfate. After filtration, the filtrate was concentrated under reduced pressure. The residue was purified by reverse phase flash chromatography with the following conditions: Column: Spherical C18, 20-40 μm, 330 g; Mobile Phase A: water (plus 10 mM NH₄HCO₃); Mobile Phase B: ACN; Flow rate: 80 mL/min; Gradient: 40% B-60% B in 20 min; Detector: UV 254/220 nm. The fractions containing the desired product were collected and concentrated under reduced pressure to afford tert-butyl 4-[5-[(2-methoxyethoxy)methyl]pyrimidin-4-yl]piperazine-1-carboxylate as a brown oil.

Yield 0.70 g (80%). ¹H NMR (400 MHz, DMSO) δ 8.54 (s, 1H), 8.26 (s, 1H), 4.41 (s, 2H), 3.60-3.55 (m, 6H), 3.48 (t, J=4.4 Hz, 2H), 3.42 (t, J=5.0 Hz, 4H), 3.26 (s, 3H), 1.43 (s, 9H). m/z: [ESI⁺]353 (M+H)⁺.

### Synthesis of 5-((2-methoxyethoxy)methyl)-4-(piperazin-1-yl)pyrimidine

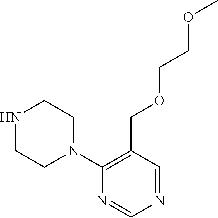 [SMILES: COCCOCc1cncnc1N1CCNCC1]

tert-Butyl 4-[5-[(2-methoxyethoxy)methyl]pyrimidin-4-yl]piperazine-1-carboxylate (0.70 g, 1.99 mmol) was treated with a 4 M solution of HCl in dioxane (15 mL, 60.00 mmol) for 1 h at room temperature under a nitrogen atmosphere. The resulting mixture was concentrated under reduced pressure. The residue was purified by reverse phase flash chromatography with the following conditions: Column: Spherical C18, 20-40 μm, 330 g; Mobile Phase A: water (plus 10 mM NH₄HCO₃); Mobile Phase B: ACN; How rate: 80 mL/min; Gradient: 10% B-30% B in 20 min; Detector: UV 254/220 nm. The fractions containing desired product were collected and concentrated under reduced pressure to afford 5-[(2-methoxyethoxy)methyl]-4-(piperazin-1-yl)pyrimidine as a brown oil.

Yield 0.40 g (80%). ¹H NMR (400 MHz, DMSO) δ 8.50 (s, 1H), 8.21 (s, 1H), 4.28 (s, 2H), 3.58-3.42 (m, 8H), 3.22 (s, 3H), 2.76 (t, J=4.8 Hz, 4H), NH not visible—under water peak. m/z: [ESI⁺]253 (M+H)⁺.

### Synthesis of Tert-Butyl 4-(1-methyl-1H-1,2,3-triazol-4-yl)piperazine-1-carboxylate

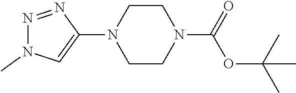 [SMILES: Cn1cc(N2CCN(C(=O)OC(C)(C)C)CC2)nn1]

To a solution of 4-bromo-1-methyl-1,2,3-triazole (3.00 g, 18.52 mmol) in DMSO (45 mL) were added tert-butyl piperazine-1-carboxylate (4.14 g, 22.23 mmol), copper(I) iodide (1.06 g, 5.57 mmol), potassium phosphate tribasic (11.80 g, 55.59 mmol) and L-proline (1.28 g, 11.12 mmol) at room temperature under a nitrogen atmosphere. The resulting mixture was stirred at 120° C. for 16 h under a nitrogen atmosphere. After cooling down to ambient temperature, the resulting mixture was filtered and the collected filter cake was washed with acetonitrile (2×10 mL). The combined washings and filtrate were concentrated under reduced pressure. The residue was purified by reverse phase flash chromatography with the following conditions: Column: Spherical C18, 20-40 um, 330 g; Mobile Phase A: water (plus 10 mM NH₄HCO₃); Mobile Phase B: ACN; How rate: 80 mL/min; Gradient: 35% B-55% B in 20 min; Detector: 254/220 nm. The fractions containing the desired product were collected and concentrated under reduced pressure to afford tert-butyl 4-(1-methyl-1,2,3-triazol-4-yl)piperazine-1-carboxylate as an off-white solid.

Yield 0.16 g (3%). ¹H NMR (400 MHz, DMSO) δ 7.46 (s, 1H), 3.93 (s, 3H), 3.44 (t, J=4.8 Hz, 4H), 3.01 (t, J=4.8 Hz, 4H), 1.41 (s, 9H). m/z: [ESI⁺] 268 (M+H)⁺.

### Synthesis of 1-(1-methyl-1H-1,2,3-triazol-4-yl)piperazine

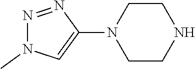

tert-Butyl 4-(1-methyl-1,2,3-triazol-4-yl)piperazine-1-carboxylate (159 mg, 0.561 mmol) was treated with a 4 M solution of HCl in dioxane (10 mL, 40.00 mmol) for 1 h at room temperature under a nitrogen atmosphere. The resulting mixture was concentrated under reduced pressure and the residue was purified by reverse phase flash chromatography with the following conditions: Column: Spherical C18, 20-40 m, 330 g; Mobile Phase A: water (plus 10 mM NH₄HCO₃); Mobile Phase B: ACN; How rate: 80 mL/min; Gradient: 20% B-40% B in 20 min; Detector: 254/220 nm. The fractions containing the desired product were collected and concentrated under reduced pressure to afford 1-(1-methyl-1,2,3-triazol-4-yl)piperazine as a yellow solid.

Yield 25 mg (25%). ¹H NMR (400 MHz, DMSO) δ 7.41 (s, 1H), 3.93 (s, 3H), 3.05 (t, J=5.2 Hz, 4H), 2.85 (t, J=5.2 Hz, 4H), NH not visible—under water peak. m/z: [ESI⁺] 168 (M+H)⁺.

### Synthesis of Tert-Butyl 4-(5-bromopyrimidin-4-yl)piperazine-1-carboxylate

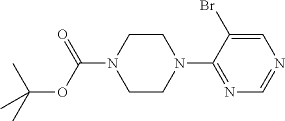 [SMILES: CC(C)(C)OC(=O)N1CCN(c2ncncc2Br)CC1]

To a solution of tert-butyl piperazine-1-carboxylate (2.00 g, 10.74 mmol) and 5-bromo-4-chloropyrimidine (2.08 g, 10.75 mmol) in DMF (30 mL) was added potassium carbonate (1.48 g, 10.71 mmol) at room temperature under a nitrogen atmosphere. The resulting mixture was stirred for 16 h at 130° C. After cooling down to room temperature, the resulting mixture was filtered. The collected filtered cake was washed with ethyl acetate (3×30 mL). The combined washings and filtrate were concentrated under reduced pressure. The residue was purified by reverse phase flash chromatography with the following conditions: column, C18 silica gel; Mobile Phase A: water (plus 10 mM NH₄HCO₃); Mobile Phase B: ACN; How rate: 80 mL/min; Gradient: 60% B-80% B in 20 min; Detector: UV 254/220 nm. The fractions containing the desired product were collected and concentrated under reduced pressure to afford tert-butyl 4-(5-bromopyrimidin-4-yl)piperazine-1-carboxylate as a yellow solid.

Yield 3.20 g (87%). ¹H NMR (400 MHz, DMSO) δ 8.63 (s, 1H), 8.57 (s, 1H), 3.60 (t, J=5.2 Hz, 4H), 3.46 (t, J=5.2 Hz, 4H), 1.43 (s, 9H). m/z: [ESI⁺] 343, 345 (M+H)⁺.

### Synthesis of Tert-Butyl 4-(5-ethenylpyrimidin-4-yl)piperazine-1-carboxylate

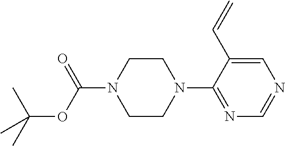 [SMILES: C=Cc1cncnc1N1CCN(C(=O)OC(C)(C)C)CC1]

To a solution of tert-butyl 4-(5-bromopyrimidin-4-yl)piperazine-1-carboxylate (3.00 g, 8.74 mmol) in DME (30 mL) and water (9 mL) were added 2-ethenyl-4,4,5,5-tetramethyl-1,3,2-dioxaborolane (1.48 g, 9.61 mmol), sodium carbonate (1.85 g, 17.45 mmol) and [1,1′-bis(diphenylphosphino)ferrocene]dichloropalladium(II), complex with dichloromethane (0.36 g, 0.44 mmol) at room temperature under an argon atmosphere. The resulting mixture was stirred for 16 h at 95° C. under an argon atmosphere. After cooling down to room temperature, the resulting mixture was filtered and the collected filter cake was washed with ethyl acetate (3×50 mL). The combined washings and filtrate were concentrated under reduced pressure. The residue was purified by silica gel column chromatography, eluted with 1%-90% ethyl acetate in petroleum ether to afford tert-butyl 4-(5-ethenylpyrimidin-4-yl)piperazine-1-carboxylate as a brown oil.

Yield 2.00 g (79%). ¹H NMR (400 MHz, CDCl₃) δ 8.64 (s, 1H), 8.36 (s, 1H), 6.62-6.50 (m, 1H), 5.69 (dd, J=1.1, 17.6 Hz, 1H), 5.38 (dd, J=1.0, 11.0 Hz, 1H), 3.56-3.52 (m, 8H), 1.50 (s, 9H). m/z: [ESI⁺] 291 (M+H)⁺.

### Synthesis of Tert-Butyl 4-(5-ethylpyrimidin-4-yl)piperazine-1-carboxylate

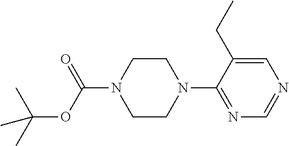 [SMILES: CCc1cncnc1N1CCN(C(=O)OC(C)(C)C)CC1]

To a stirred solution of tert-butyl 4-(5-ethenylpyrimidin-4-yl)piperazine-1-carboxylate (4.00 g, 13.77 mmol) in methanol (30 mL) was added 10% wt. palladium on carbon (600 mg). Following degassing of the stirred mixture, the mixture was stirred for 18 h at room temperature under a hydrogen atmosphere with a balloon. The resulting mixture was filtered through a Celite pad and washed with ethyl acetate (3×30 mL). The combined washings and filtrate were concentrated under reduced pressure. The residue was purified by silica gel column chromatography, eluted with 33% EtOAc in petroleum ether to afford tert-butyl 4-(5-ethylpyrimidin-4-yl)piperazine-1-carboxylate as a brown solid.

Yield 3.80 g (94%). ¹H NMR (400 MHz, CDCl₃) δ 8.64 (s, 1H), 8.28 (s, 1H), 3.57 (t, J=4.8 Hz, 4H), 3.41 (t, J=4.8 Hz, 4H), 2.62 (q, J=7.5 Hz, 2H), 1.50 (s, 9H), 1.30 (t, J=7.5 Hz, 3H). m/z: [ESI⁺] 293 (M+H)⁺.

### Synthesis of 5-ethyl-4-(piperazin-1-yl)pyrimidine

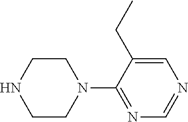

To a solution of tert-butyl 4-(5-ethylpyrimidin-4-yl)piperazine-1-carboxylate (3.80 g, 12.99 mmol) in THF (20 mL) was added a 4 M solution of HCl in dioxane (5 mL, 20.00 mmol). The resulting mixture was stirred for 2 h at room temperature under a nitrogen atmosphere. The resulting mixture was concentrated under reduced pressure. The residue was purified by reverse phase flash chromatography with the following conditions: column, C18, 20-40 m, 330 g; Mobile Phase A: water (plus 10 mM NH₄HCO₃); Mobile Phase B: ACN; Flow rate: 80 mL/min; Gradient: 30% B-50% B in 20 min; Detector: UV 254/220 nm. The fractions containing the desired product were collected and concentrated under reduced pressure to afford 5-ethyl-4-(piperazin-1-yl)pyrimidine as a brown solid.

Yield 2.20 g (88%). ¹H NMR (400 MHz, DMSO) δ 8.58 (s, 1H), 8.32 (s, 1H), 3.48 (t, J=4.4 Hz, 4H), 3.10 (t, J=4.4 Hz, 4H), 2.60 (q, J=7.5 Hz, 2H), 1.20 (t, J=7.5 Hz, 3H), NH not visible—under water peak. m/z: [ESI⁺] 193 (M+H)⁺.

### Synthesis of 6-bromo-1-[[2-(trimethylsilyl)ethoxy]methyl]-1,3-benzodiazole

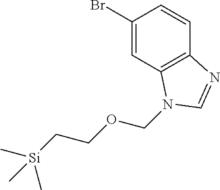 [SMILES: C[Si](C)(C)CCOCn1cnc2ccc(Br)cc21]

To a solution of 5-bromo-3H-1,3-benzodiazole (5.00 g, 25.38 mmol) in DMF (60 mL) were added potassium carbonate (7.10 g, 51.37 mmol) and 2-(trimethylsilyl)ethoxymethyl chloride (6.80 g, 40.79 mmol) at room temperature under a nitrogen atmosphere. After stirring for 16 h, the resulting mixture was filtered and the filtrate was concentrated under reduced pressure. The residue was purified by reverse phase flash chromatography with the following conditions: Column: Spherical C18, 20-40 m, 330 g; Mobile Phase A: water (plus 10 mM NH₄HCO₃); Mobile Phase B: ACN; How rate: 80 mL/min; Gradient: 55% B-75% B in 20 min; Detector: UV 254/220 nm. The fractions containing the desired product were collected and concentrated under reduced pressure to afford 6-bromo-1-[[2-(trimethylsilyl)ethoxy]methyl]-1,3-benzodiazole as a brown oil.

Yield 3.40 g (41%). ¹H NMR (400 MHz, CDCl₃) δ 7.99-7.97 (m, 1.5H), 7.73-7.68 (m, 1H), 7.48-7.42 (m, 1.5H), 5.54 (s, 1H), 5.52 (s, 1H), 3.57-3.49 (m, 2H), 0.95-0.90 (m, 2H), −0.031 (s, 4.5H), −0.039 (s, 4.5H). (mixture of two regio-isomers, ratio=ca. 1:1). m/z: [ESI⁺] 327, 329 (M+H)⁺.

### Synthesis of (3-[[2-(trimethylsilyl)ethoxy]methyl]-1,3-benzodiazol-5-yl)methanol

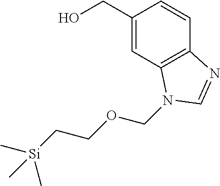 [SMILES: C[Si](C)(C)CCOCn1cnc2ccc(CO)cc21]

To a solution of 6-bromo-1-[[2-(trimethylsilyl)ethoxy]methyl]-1,3-benzodiazole (4.00 g, 12.22 mmol) in degassed anhydrous dioxane (40 mL) were added (tributylstannyl)methanol (7.90 g, 24.60 mmol) and tetrakis(triphenylphosphine)palladium(0) (1.40 g, 1.21 mmol) at room temperature under an argon atmosphere. The resulting mixture was stirred at 80° C. for 16 h. After cooling down to room temperature, the resulting mixture was concentrated under reduced pressure. The residue was purified by reverse phase flash chromatography with the following conditions: Column: Spherical C18, 20-40 m, 330 g; Mobile Phase A: water (plus 10 mM NH₄HCO₃); Mobile Phase B: ACN; How rate: 80 mL/min; Gradient: 40% B-60% B in 20 min; Detector: UV 254/220 nm. The fractions containing the desired product were collected and concentrated under reduced pressure to afford (3-[[2-(trimethylsilyl)ethoxy]methyl]-1,3-benzodiazol-5-yl)methanol as a brown oil.

Yield 0.90 g (27%). ¹H NMR (400 MHz, DMSO) δ 9.13 (s, 1H), 7.80 (s, 1H), 7.75 (d, J=8.4 Hz, 1H), 7.43 (d, J=8.4 Hz, 1H), 5.79 (s, 2H), 4.67 (s, 2H), 3.57 (t, J=3.2 Hz, 2H), 0.89 (t, J=3.2 Hz, 2H), −0.064 (s, 9H), OH proton not visible—under water peak. m/z: [ESI⁺] 279 (M+H)⁺.

### Synthesis of 6-(chloromethyl)-1-((2-(trimethylsilyl)ethoxy)methyl)-1H-benzo[d]imidazole

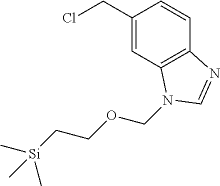 [SMILES: C[Si](C)(C)CCOCn1cnc2ccc(CCl)cc21]

A solution of (3-[[2-(trimethylsilyl)ethoxy]methyl]-1,3-benzodiazol-5-yl)methanol (0.90 g, 3.23 mmol) in DCM (20 mL) was treated with thionyl chloride (2.30 mL, 31.67 mmol) at room temperature for 1.5 h under an argon atmosphere. The reaction was quenched with saturated aqueous NaHCO₃ (20 mL) and the resulting mixture was extracted with DCM (3×20 mL). The combined organic layers were dried over anhydrous Na₂SO₄. After filtration, the filtrate was concentrated under reduced pressure afford the crude product as a brown oil, which was used in the next step directly without further purification.

Yield 0.80 g (crude). m/z: [ESI⁺] 297, 299 (M+H)⁺.

### Synthesis of 6-[(2-methoxyethoxy)methyl]-1-[[2-(trimethylsilyl)ethoxy]methyl]-1,3-benzodiazole

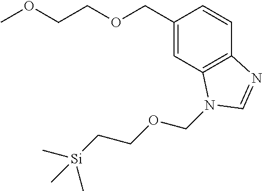 [SMILES: COCCOCc1ccc2ncn(COCC[Si](C)(C)C)c2c1]

A solution of 2-methoxyethanol (0.51 g, 6.70 mmol) in DMF (10 mL) was treated with sodium hydride (0.23 g, 5.75 mmol, 60% wt dispersed into mineral oil) at 0° C. for 30 min followed by the addition of the above 6-(chloromethyl)-1-[[2-(trimethylsilyl)ethoxy]methyl]-1,3-benzodiazole (0.80 g, crude). The resulting solution was stirred for additional 16 h at ambient temperature. The reaction was quenched by saturated aqueous NH₄Cl (15 mL). The resulting mixture was extracted with ethyl acetate (3×10 mL). The combined organic layers were dried over anhydrous Na₂SO₄. After filtration, the filtrate was concentrated under reduced pressure. The residue was purified by reverse phase flash chromatography with the following conditions: Column: Spherical C18, 20-40 μm, 330 g; Mobile Phase A: water (plus 10 mM NH₄HCO₃); Mobile Phase B: ACN; Flow rate: 80 mL/min; Gradient: 30% B-50% B in 20 min; Detector: UV 254/220 nm. The fractions containing the desired product were collected and concentrated under reduced pressure to afford 6-[(2-methoxyethoxy)methyl]-1-[[2-(trimethylsilyl)ethoxy]methyl]-1,3-benzodiazole as a brown oil.

Yield 0.23 g (21% over 2 steps). ¹H NMR (400 MHz, CDCl₃) δ 8.02 (s, 1H), 7.80 (d, J=8.4 Hz, 1H), 7.56 (d, J=1.6 Hz, 1H), 7.33 (dd, J=1.6, 8.4 Hz, 1H), 5.56 (s, 2H), 4.74 (s, 2H), 3.70-3.57 (m, 4H), 3.51 (t, J=4.4 Hz, 2H), 3.42 (s, 3H), 0.97 (t, J=4.4 Hz, 2H), −0.03 (s, 9H). m/z: [ESI⁺] 337 (M+H)⁺.

### Synthesis of 6-[(2-methoxyethoxy)methyl]-1-[[2-(trimethylsilyl)ethoxy]methyl]-1,3-benzodiazole-2-carbaldehyde

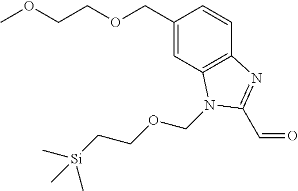 [SMILES: COCCOCc1ccc2nc(C=O)n(COCC[Si](C)(C)C)c2c1]

A 1.6 M solution of n-butyllithium in THF (1.80 mL, 2.88 mmol) was added dropwise over 40 minutes to a solution of 6-[(2-methoxyethoxy)methyl]-1-[[2-(trimethylsilyl)ethoxy]methyl]-1,3-benzodiazole (0.47 g, 1.40 mmol) in THF (30 mL) at −40° C. under an argon atmosphere. DMF (0.60 mL, 7.75 mmol) was then added dropwise to the stirred reaction mixture at −40° C. over 5 minutes. After stirring for an additional 1 h at room temperature, the reaction was quenched by saturated aqueous NH₄Cl (10 mL) at room temperature. The resulting mixture was extracted with ethyl acetate (3×10 mL). The combined organic extracts were dried over anhydrous Na₂SO₄. After filtration, the filtrate was concentrated under reduced pressure to afford the crude product as a brown oil which was used in the next step directly without further purification.

Yield 0.37 g (crude). m/z: [ESI⁺] 365 (M+H)⁺.

### Synthesis of 2-[[4-(5-ethylpyrimidin-4-yl)piperazin-1-yl]methyl]-6-[(2-methoxyethoxy)methyl]-1-[[2-(trimethylsilyl)ethoxy]methyl]-1,3-benzodiazole

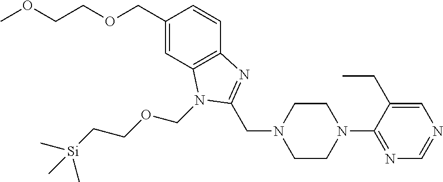 [SMILES: CCc1cncnc1N1CCN(Cc2nc3ccc(COCCOC)cc3n2COCC[Si](C)(C)C)CC1]

To a solution of the crude 6-[(2-methoxyethoxy)methyl]-1-[[2-(trimethylsilyl)ethoxy]methyl]-1,3-benzodiazole-2-carbaldehyde (0.37 g) in DCM (15 mL) were added 5-ethyl-4-(piperazin-1-yl)pyrimidine (0.19 g, 0.99 mmol), sodium triacetoxyborohydride (0.43 g, 2.03 mmol) and acetic acid (0.01 mL, 0.173 mmol) at room temperature under an argon atmosphere. After stirring for 1 h at room temperature, the reaction was quenched with saturated aqueous NH₄Cl solution (5 mL) at 0° C. The resulting mixture was concentrated under reduced pressure. The residue was purified by reverse phase flash chromatography with the following conditions: Column: Spherical C18, 20-40 μm, 330 g; Mobile Phase A: water (plus 10 mM NH₄HCO₃); Mobile Phase B: ACN; How rate: 80 mL/min; Gradient: 60% B-80% B in 20 min; Detector: UV 254/220 nm. The fractions containing the desired product were collected and concentrated under reduced pressure to afford 2-((4-(5-ethylpyrimidin-4-yl)piperazin-1-yl)methyl)-6-((2-methoxyethoxy)methyl)-1-((2-(trimethylsilyl)ethoxy)methyl)-1H-benzo[d]imidazole as a brown oil.

Yield 0.23 g (31% over 2 steps). ¹H NMR (400 MHz, DMSO) δ 8.52 (s, 1H), 8.25 (s, 1H), 7.63-7.51 (m, 2H), 7.19 (d, J=8.8 Hz, 1H), 5.74 (s, 2H), 4.59 (s, 2H), 3.86 (s, 2H), 3.63-3.53 (m, 4H), 3.49 (t, J=5.8 Hz, 2H), 3.40-3.30 (m, 4H), 3.26 (s, 3H), 2.65-2.56 (m, 6H), 1.19 (t, J=7.5 Hz, 3H), 0.93 (t, J=4.4 Hz, 2H), −0.08 (s, 9H). m/z: [ESI⁺] 541 (M+H)⁺.

### Synthesis of Ethyl 2-[4-(5-ethylpyrimidin-4-yl)piperazin-1-yl]acetate

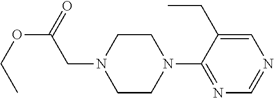 [SMILES: CCOC(=O)CN1CCN(c2ncncc2CC)CC1]

To a solution of 5-ethyl-4-(piperazin-1-yl)pyrimidine (0.50 g, 2.60 mmol) in DMF (10 mL) were added potassium carbonate (0.72 g, 5.21 mmol) and ethyl chloroacetate (0.31 mL, 2.90 mmol) at room temperature. The resulting mixture was stirred at 50° C. for 16 h. After cooling down to room temperature, the resulting mixture was filtered. The filtered cake was washed with DMF (3×2 mL). The combined washings and filtrate were concentrated under reduced pressure. The residue was purified by reverse phase flash chromatography with the following conditions: Column: Spherical C18, 20-40 m, 330 g; Mobile Phase A: water (plus 10 mM NH₄HCO₃); Mobile Phase B: ACN; How rate: 80 mL/min; Gradient: 25% B-45% B in 20 min; Detector: UV 254/220 nm. The fractions containing the desired product were collected and concentrated under reduced pressure to afford ethyl 2-[4-(5-ethylpyrimidin-4-yl)piperazin-1-yl]acetate as a yellow oil.

Yield 0.51 g (70%). ¹H NMR (400 MHz, CDCl₃) δ 8.62 (d, J=0.8 Hz, 1H), 8.23 (d, J=0.8 Hz, 1H), 4.24 (q, J=7.6 Hz, 2H), 3.50 (t, J=4.4 Hz, 4H), 3.32 (s, 2H), 2.73 (t, J=4.4 Hz, 4H), 2.55 (q, J=7.5 Hz, 2H), 1.42-1.21 (m, 6H). m/z: [ESI⁺] 279 (M+H)⁺.

### Synthesis of [4-(5-ethylpyrimidin-4-yl)piperazin-1-yl]acetic Acid

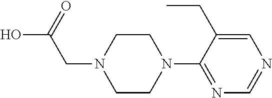 [SMILES: CCc1cncnc1N1CCN(CC(=O)O)CC1]

To a solution of ethyl 2-[4-(5-ethylpyrimidin-4-yl)piperazin-1-yl]acetate (0.51 g, 1.83 mmol) in THF (10 mL) were added water (2 mL) and sodium hydroxide (0.73 g, 18.25 mmol) at 0° C. The resulting mixture was stirred for additional 1 h at 60° C. After cooling down to room temperature, the resulting mixture was neutralized with acetic acid (1.10 mL, 19.25 mmol) and concentrated under reduced pressure. The residue was purified by reverse phase flash chromatography with the following conditions: Column: Spherical C18, 20-40 μm, 330 g; Mobile Phase A: water (plus 10 mM NH₄HCO₃); Mobile Phase B: ACN; How rate: 80 mL/min; Gradient: 10% B-30% B in 20 min; Detector: UV 254/220 nm. The fractions containing the desired product were collected and concentrated under reduced pressure to give [4-(5-ethylpyrimidin-4-yl)piperazin-1-yl]acetic acid as a yellow oil.

Yield 0.31 g (68%). 1H NMR (400 MHz, DMSO) δ 8.98 (br s, 1H), 8.68 (s, 1H), 8.36 (s, 1H), 4.06 (s, 2H), 3.80 (t, J=4.4 Hz, 4H), 3.31 (t, J=4.4 Hz, 4H), 2.64 (q, J=7.5 Hz, 2H), 1.20 (t, J=7.5 Hz, 3H). m/z: [ESI⁺] 251 (M+H)⁺.

### Synthesis of 4-(2-methoxyethoxy)-2-nitroaniline

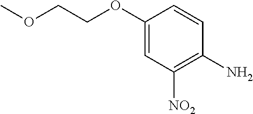

To a solution of 4-amino-3-nitrophenol (4.75 g, 30.81 mmol) in DMF (50 mL) were added potassium carbonate (8.52 g, 61.65 mmol) and 2-bromoethyl methyl ether (3.20 mL, 34.05 mmol) at room temperature under a nitrogen atmosphere. The resulting mixture was stirred for 2 h at 50° C. under a nitrogen atmosphere. After cooling down to room temperature, the resulting mixture was filtered. The filtered cake was washed with ethyl acetate (3×20 mL). The filtrate was concentrated under reduced pressure. The residue was purified by reverse phase flash chromatography with the following conditions: column, C18 20-40 μm, 330 g; Mobile Phase A: water (plus 10 mM NH₄HCO₃); Mobile Phase B: ACN; Gradient: 20%-40% of gradient B in 25 min; Detector: UV 254/220 nm. The fractions containing the desired product were collected and concentrated under reduced pressure to afford 4-(2-methoxyethoxy)-2-nitroaniline as a light yellow solid.

Yield 1.99 g (30%). 1H NMR (400 MHz, DMSO) δ 7.38 (d, J=3.0 Hz, 1H), 7.26 (br s, 2H), 7.18 (dd, J=3.0, 9.2 Hz, 1H), 7.00 (d, J=9.3 Hz, 1H), 4.05 (t, J=4.8 Hz, 2H), 3.63 (t, J=4.8 Hz, 2H), 3.30 (s, 3H). m/z: [ESI⁺] 213 (M+H)⁺.

### Synthesis of 4-(2-methoxyethoxy)benzene-1,2-diamine

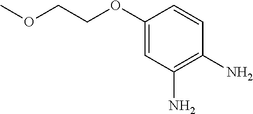

To a solution of 4-(2-methoxyethoxy)-2-nitroaniline (4.50 g, 21.21 mmol) in ethanol (200 mL) was added 10% wt. palladium on carbon (1.10 g). Following degassing of the stirred mixture, the mixture was stirred at room temperature for 4 h under a hydrogen atmosphere (30 psi). The resulting mixture was filtered through a Celite pad and washed with ethanol (3×50 mL). The combined washings and filtrate were concentrated under reduced pressure. The residue was purified by reverse phase flash chromatography with the following conditions: Column: Spherical C18, 20-40 μm, 330 g; Mobile Phase A: water (plus 10 mM NH₄HCO₃); Mobile Phase B: ACN; How rate: 80 mL/min; Gradient: 20% B-40% B in 20 min; Detector: UV 254/220 nm. The fractions containing the desired product were collected and concentrated under reduced pressure to afford 4-(2-methoxyethoxy)benzene-1,2-diamine as a brown oil.

Yield 3.00 g (78%). ¹H NMR (400 MHz, DMSO) δ 6.40 (d, J=8.4 Hz, 1H), 6.16 (d, J=2.8 Hz, 1H), 5.98 (dd, J=2.8, 8.4 Hz, 1H), 4.48 (br s, 2H), 4.05 (br s, 2H), 3.88 (t, J=5.2 Hz, 2H), 3.58 (t, J=5.2 Hz, 2H), 3.31 (s, 3H). m/z: [ESI⁺] 183 (M+H)⁺.

### Synthesis of 1-[(2-methoxyethoxy)methyl]-3-nitrobenzene

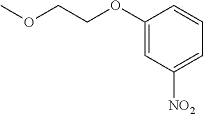

A solution of 2-methoxyethanol (6.90 mL, 87.49 mmol) in DMF (60 mL) was treated with sodium hydride (2.30 g, 57.50 mmol, 60% w/w dispersion in mineral oil) for 30 mins at 0° C. under a nitrogen atmosphere. To the above mixture was added 1-(chloromethyl)-3-nitrobenzene (5.00 g, 29.14 mmol) in portions over 5 mins at 0° C. The resulting mixture was stirred for additional 1.5 h at room temperature. The reaction was quenched with water (200 mL). The resulting mixture was extracted with ethyl acetate (3×500 mL). The combined organic extracts were dried over anhydrous Na₂SO₄. After filtration, the filtrate was concentrated under reduced pressure. The residue was purified by silica gel column chromatography, eluted with 1%-40% ethyl acetate in petroleum ether to afford 1-[(2-methoxyethoxy)methyl]-3-nitrobenzene as a brown oil.

Yield 2.00 g (33%). ¹H NMR (400 MHz, CDCl₃) δ 8.25 (d, J=2.0 Hz, 1H), 8.18-8.15 (m, 1H), 7.72-7.70 (m, 1H), 7.57-7.53 (m, 1H), 4.69 (s, 2H), 3.70 (t, J=4.4 Hz, 2H), 3.63 (t, J=4.4 Hz, 2H), 3.43 (s, 3H). m/z: [ESI⁺] 229 (M+NH₄)⁺.

### Synthesis of 3-[(2-methoxyethoxy)methyl]aniline

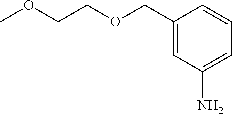

To a solution of 1-[(2-methoxyethoxy)methyl]-3-nitrobenzene (2.00 g, 9.47 mmol) in acetic acid (20 mL) was added iron powder (5.00 g, 89.53 mmol) in portions at 0° C. under a nitrogen atmosphere. The resulting mixture was stirred for 1 h at room temperature under a nitrogen atmosphere. The resulting mixture was filtered through a Celite pad and washed with ethyl acetate (3×100 mL). The combined washings and filtrate were concentrated under reduced pressure. The residue was purified by reverse phase flash chromatography with the following conditions: Column: Spherical C18, 20-40 μm, 330 g; Mobile Phase A: water (plus 10 mM NH₄HCO3); Mobile Phase B: ACN; How rate: 80 mL/min; Gradient: 30% B-50% B in 20 min; Detector: UV 254/220 nm. The fractions containing the desired product were collected and concentrated under reduced pressure to afford 3-[(2-methoxyethoxy)methyl]aniline as a brown oil.

Yield 1.50 g (88%). 1H NMR (400 MHz, DMSO) δ 6.97-6.93 (m, 1H), 6.54-6.52 (m, 1H), 6.50-6.40 (m, 2H), 5.03 (br s, 2H), 4.32 (s, 2H), 3.51 (t, J=4.8 Hz, 2H), 3.46 (t, J=4.8 Hz, 2H), 3.25 (s, 3H). m/z: [ESI⁺] 182 (M+H)⁺.

### Synthesis of Ethyl 6-[(2-methoxyethoxy)methyl]-1H-indole-2-carboxylate

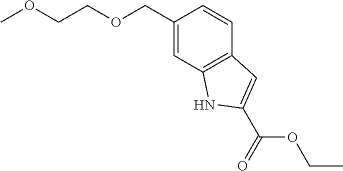 [SMILES: CCOC(=O)c1cc2ccc(COCCOC)cc2[nH]1]

To a solution of 3-[(2-methoxyethoxy)methyl]aniline (1.00 g, 5.52 mmol) in DMSO (28 mL) were added palladium(II)acetate (0.12 g, 0.53 mmol), 4 Å molecular sieves (1.11 g), ethyl pyruvate (1.20 mL, 10.80 mmol) and acetic acid (1.30 mL, 22.71 mmol) at room temperature under an oxygen atmosphere. The resulting mixture was stirred for 16 h at 70° C. under an oxygen atmosphere. The resulting mixture was cooled down to room temperature and diluted with ethyl acetate (50 mL). The resulting mixture was filtered through a Celite pad and washed with ethyl acetate (3×30 mL). The combined washings and filtrate were concentrated under reduced pressure. The residue was purified by reverse phase flash chromatography with the following conditions: Column: Spherical C18, 20-40 m, 330 g; Mobile Phase A: water (plus 10 mM NH₄HCO₃); Mobile Phase B: ACN; How rate: 80 mL/min; Gradient: 60% B-80% B in 20 min; Detector: UV 254/220 nm. The fractions containing the desired product were collected and concentrated under reduced pressure to afford ethyl 6-[(2-methoxyethoxy)methyl]-1H-indole-2-carboxylate as a brown solid.

Yield 1.10 g (72%). ¹H NMR (400 MHz, DMSO) δ 11.88 (br s, 1H), 7.62 (d, J=8.4 Hz, 1H), 7.40 (d, J=1.2 Hz, 1H), 7.13 (s, 1H), 7.04 (dd, J=1.2, 8.4 Hz, 1H), 4.58 (s, 2H), 4.35 (q, J=7.2 Hz, 2H), 3.60 (t, J=4.4 Hz, 2H), 3.48 (t, J=4.4 Hz, 2H), 3.27 (s, 3H), 1.35 (t, J=7.2 Hz, 3H). m/z: [ESI⁺] 278 (M+H)⁺.

### Synthesis of 6-[(2-methoxyethoxy)methyl]-1H-indole-2-carboxylic Acid

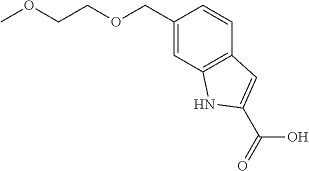 [SMILES: COCCOCc1ccc2cc(C(=O)O)[nH]c2c1]

To a stirred solution of ethyl 6-[(2-methoxyethoxy)methyl]-1H-indole-2-carboxylate (1.10 g, 3.97 mmol) in a solvent mixture of methanol (12 mL), THF (6 mL) and water (2 mL) was added lithium hydroxide (0.76 g, 31.73 mmol) in portions at 0° C. The resulting mixture was stirred for 2 h at room temperature. The resulting mixture was concentrated under reduced pressure. The residue was purified by reverse phase flash chromatography with the following conditions: Column: Spherical C18, 20-40 m, 330 g; Mobile Phase A: water (plus 10 mM NH₄HCO₃); Mobile Phase B: ACN; Flow rate: 80 mL/min; Gradient: 10% B-30% B in 20 min; Detector: UV 254/220 nm. The fractions containing the desired product were collected and concentrated under reduced pressure to afford 6-[(2-methoxyethoxy)methyl]-1H-indole-2-carboxylic acid as a brown solid.

Yield 0.50 g (51%). ¹H NMR (400 MHz, DMSO) δ 11.65 (br s, 1H), 7.51 (s, 1H), 7.46 (d, J=8.4 Hz, 1H), 6.91 (dd, J=1.6, 8.4 Hz, 1H), 6.66 (d, J=1.6 Hz, 1H), 4.52 (s, 2H), 3.63-3.44 (m, 4H), 3.26 (s, 3H). OH acid proton hidden. m/z: [ESI⁺] 267 (M+NH₄)⁺.

### Synthesis of 2-[4-(5-ethylpyrimidin-4-yl)piperazine-1-carbonyl]-6-[(2-methoxyethoxy)methyl]-1H-indole

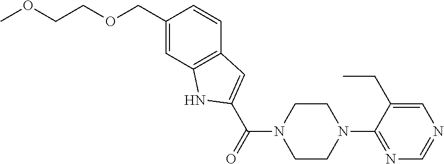 [SMILES: CCc1cncnc1N1CCN(C(=O)c2cc3ccc(COCCOC)cc3[nH]2)CC1]

To a solution of 5-ethyl-4-(piperazin-1-yl)pyrimidine (0.35 g, 1.82 mmol) in DMF (10 mL) were added 6-[(2-methoxyethoxy)methyl]-1H-indole-2-carboxylic acid (0.49 g, 1.97 mmol), HATU (1.03 g, 2.71 mmol) and DIPEA (0.94 mL, 5.69 mmol) at room temperature under a nitrogen atmosphere. The resulting mixture was stirred for 1 h at room temperature under a nitrogen atmosphere. The resulting mixture was purified by reverse phase flash chromatography with the following conditions: Column: Spherical C18, 20-40 μm, 330 g; Mobile Phase A: water (plus 10 mM NH₄HCO₃); Mobile Phase B: ACN; Flow rate: 80 mL/min; Gradient: 45% B-65% B in 20 min; Detector: UV 254/220 nm. The fractions containing the desired product were collected and concentrated under reduced pressure to afford (4-(5-ethylpyrimidin-4-yl)piperazin-1-yl)(6-((2-methoxyethoxy)methyl)-1H-indol-2-yl)methanone as an off-white solid.

Yield 0.66 g (86%). ¹H NMR (400 MHz, DMSO) δ 11.63 (br s, 1H), 8.57 (s, 1H), 8.31 (s, 1H), 7.58 (d, J=8.4 Hz, 1H), 7.40 (s, 1H), 7.02 (dd, J=1.6, 8.4 Hz, 1H), 6.86 (d, J=1.6 Hz, 1H), 4.57 (s, 2H), 3.94-3.91 (m, 4H), 3.63-3.47 (m, 8H), 3.27 (s, 3H), 2.65 (q, J=7.6 Hz, 2H), 1.22 (t, J=7.6 Hz, 3H). m/z: [ESI⁺] 424 (M+H)⁺.

### Synthesis of 2-[4-(5-ethylpyrimidin-4-yl)piperazine-1-carbonyl]-1H-indol-6-ol

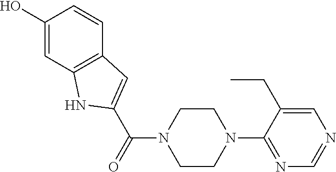 [SMILES: CCc1cncnc1N1CCN(C(=O)c2cc3ccc(O)cc3[nH]2)CC1]

To a solution of 6-hydroxy-1H-indole-2-carboxylic acid (1.11 g, 6.27 mmol) in DMF (20 mL) were added HATU (3.00 g, 7.89 mmol), DIPEA (1.71 mL, 10.35 mmol) and 5-ethyl-4-(piperazin-1-yl)pyrimidine (1.00 g, 5.20 mmol) at room temperature under an argon atmosphere. The resulting mixture was stirred for 3 h at room temperature. The resulting mixture was filtered and the collected filter cake was washed with ethyl acetate (3×20 mL). The combined washings and filtrate were concentrated under reduced pressure. The residue was purified by reverse flash chromatography with the following conditions: Column: Spherical C18, 20-40 m, 330 g; Mobile Phase A: water (plus 10 mM NH₄HCO₃); Mobile Phase B: ACN; Flow rate: 80 mL/min; Gradient: 30% B-50% B in 20 min; Detector: UV 254/220 nm. The fractions containing the desired product were collected and concentrated under reduced pressure to afford 2-[4-(5-ethylpyrimidin-4-yl)piperazine-1-carbonyl]-1H-indol-6-ol as a brown solid.

Yield 0.88 g (48%). ¹H NMR (400 MHz, DMSO) δ 11.19 (br s, 1H), 9.20 (br s, 1H), 8.56 (s, 1H), 8.30 (s, 1H), 7.39 (d, J=8.4 Hz, 1H), 6.81-6.68 (m, 2H), 6.63 (d, J=1.6 Hz, 1H), 3.95-3.89 (m, 4H), 3.54-3.44 (m, 4H), 2.70 (q, J=7.6 Hz, 2H), 1.22 (t, J=7.6 Hz, 3H). m/z: [ESI⁺] 352 (M+H)⁺.

### Synthesis of 2-[4-(5-ethylpyrimidin-4-yl)piperazine-1-carbonyl]-6-(2-methoxyethoxy)-1-(2-methoxyethyl)indole

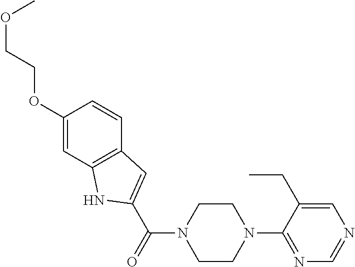 [SMILES: CCc1cncnc1N1CCN(C(=O)c2cc3ccc(OCCOC)cc3[nH]2)CC1]

To a solution of 2-[4-(5-ethylpyrimidin-4-yl)piperazine-1-carbonyl]-1H-indol-6-ol (200 mg, 0.569 mmol) in DMF (15 mL) were added 2-bromoethyl methyl ether (96 mg, 0.691 mmol), cesium carbonate (185 mg, 0.568 mmol) and potassium iodide (95 mg, 0.572 mmol) at room temperature. The resulting mixture was sealed and stirred at 60° C. for 16 h under a nitrogen atmosphere. After cooling down to room temperature, the resulting mixture was filtered, and the collected filter cake was washed with ethyl acetate (3×20 mL). The combined washings and filtrate were concentrated under reduced pressure. The residue was purified by reverse phase flash chromatography with the following conditions: Column: Spherical C18, 20-40 m, 330 g; Mobile Phase A: water (plus 10 mM NH₄HCO₃); Mobile Phase B: ACN; How rate: 80 mL/min; Gradient: 30% B-50% B in 20 min; Detector: UV 254/220 nm. The fractions containing the desired product were collected and concentrated under reduced pressure to afford 2-[4-(5-ethylpyrimidin-4-yl)piperazine-1-carbonyl]-6-(2-methoxyethoxy)-1-(2-methoxyethyl)indole as a yellow solid.

Yield 220 mg (95%). ¹H NMR (400 MHz, DMSO) δ 11.41 (s, 1H), 8.57 (s, 1H), 8.30 (s, 1H), 7.49 (d, J=8.8 Hz, 1H), 6.90 (d, J=1.6 Hz, 1H), 6.81 (d, J=1.6 Hz, 1H), 6.73 (dd, J=1.6, 8.8 Hz, 1H), 4.09 (t, J=4.4 Hz, 2H), 3.91 (t, J=4.8 Hz, 4H), 3.69 (t, J=4.4 Hz, 2H), 3.51 (t, J=4.8 Hz, 4H), 3.31 (s, 3H), 2.65 (q, J=7.6 Hz, 2H), 1.22 (t, J=7.6 Hz, 3H). m/z: [ESI⁺] 410 (M+H)⁺.

### Synthesis of 2-chloro-N-(2-fluoro-6-hydroxyphenyl)acetamide

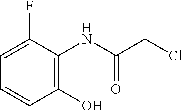 [SMILES: O=C(CCl)Nc1c(O)cccc1F]

Chloroacetyl chloride (0.98 g, 8.68 mmol) was added to a solution of 2-amino-3-fluorophenol (1.00 g, 7.87 mmol) in DCM (20 mL) at room temperature. The resulting solution was stirred for 2 h at room temperature and then concentrated under reduced pressure, to afford 2-chloro-N-(2-fluoro-6-hydroxyphenyl)acetamide as a black solid. The crude intermediate was taken onto the next step without further purification.

Yield 2.00 g (crude). m/z: [ESI⁺] 204, 206 (M+H)⁺.

### Synthesis of 2-(chloromethyl)-4-fluorobenzo[d]oxazole

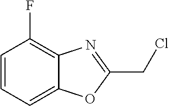 [SMILES: Fc1cccc2oc(CCl)nc12]

A mixture of 2-chloro-N-(2-fluoro-6-hydroxyphenyl)acetamide (2.00 g, 9.82 mmol) in polyphosphoric acid (4.00 g) was stirred for 2 h at 150° C. under a nitrogen atmosphere. The resulting mixture was cooled to room temperature and basified to pH 8 with saturated aqueous NaHCO₃ solution. The mixture was then extracted with ethyl acetate (3×200 mL). The combined organic layers were washed with brine (100 mL) and dried over anhydrous Na₂SO₄. After filtration, the filtrate was concentrated under reduced pressure. The residue was purified by silica gel column chromatography, eluting with 17% ethyl acetate in petroleum ether to afford 2-(chloromethyl)-4-fluorobenzo[d]oxazole as a yellow oil.

Yield 0.50 g (27%). ¹H NMR (400 MHz, DMSO) δ 7.67 (d, J=8.4 Hz, 1H), 7.54-7.46 (m, 1H), 7.31 (dd, J=8.4, 10.4 Hz, 1H), 5.11 (s, 2H). m/z: [ESI⁺] 186, 188 (M+H)⁺.

### Synthesis of 2-chloro-N-(3-fluoro-2-hydroxyphenyl)acetamide

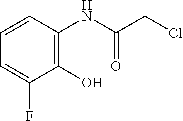 [SMILES: O=C(CCl)Nc1cccc(F)c1O]

Compound 2-chloro-N-(3-fluoro-2-hydroxyphenyl)acetamide was prepared from 2-amino-6-fluorophenol (3.00 g, 23.60 mmol) following a procedure similar to that described for the synthesis of 2-chloro-N-(2-fluoro-6-hydroxyphenyl)acetamide, as a brown solid. The crude intermediate was taken onto the next step without further purification.

Yield 3.00 g (crude). m/z: [ESI⁺] 204, 206 (M+H)⁺.

### Synthesis of 2-(chloromethyl)-7-fluorobenzo[d]oxazole

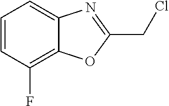 [SMILES: Fc1cccc2nc(CCl)oc12]

Compound 2-(chloromethyl)-7-fluorobenzo[d]oxazole was prepared from 2-chloro-N-(3-fluoro-2-hydroxyphenyl)acetamide (3.00 g, 14.74 mmol) following a procedure similar to that described for the synthesis of 2-(chloromethyl)-4-fluorobenzo[d]oxazole and was isolated as a yellow oil.

Yield 1.13 g (41%). ¹H NMR (400 MHz, DMSO) δ 7.66 (d, J=8.8 Hz, 1H), 7.46-7.38 (m, 2H), 5.13 (s, 2H). m/z: [ESI⁺] 186, 188 (M+H)⁺.

### Synthesis of 2-(chloromethyl)-4-(trifluoromethyl)benzo[d]oxazole

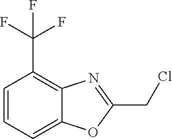 [SMILES: FC(F)(F)c1cccc2oc(CCl)nc12]

A mixture of 2-amino-3-(trifluoromethyl)phenol (1.00 g, 5.65 mmol) and 2-chloro-1,1,1-trimethoxyethane (4.36 g, 28.20 mmol) was stirred for 2 h at 130° C. under a nitrogen atmosphere. The resulting mixture was cooled to room temperature and concentrated onto silica gel. The resulting residue was purified by flash column chromatography, eluting with 17% ethyl acetate in petroleum ether to afford 2-(chloromethyl)-4-(trifluoromethyl)-1,3-benzoxazole as a yellow oil.

Yield 1.00 g (75%). ¹H NMR (400 MHz, DMSO) δ 8.16 (d, J=8.4 Hz, 1H), 7.81 (d, J=7.6 Hz, 1H), 7.67 (dd, J=7.6, 8.4 Hz, 1H), 5.18 (s, 2H). m/z: [ESI⁺] 236, 238 (M+H)⁺.

### Synthesis of 2-(chloromethyl)-6-(trifluoromethyl)benzo[d]oxazole

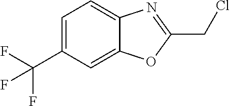 [SMILES: FC(F)(F)c1ccc2nc(CCl)oc2c1]

Compound 2-(chloromethyl)-6-(trifluoromethyl)benzo[d]oxazole was prepared from 2-amino-5-(trifluoromethyl)phenol (1.00 g, 5.64 mmol) following a procedure similar to that described for the synthesis of 2-(chloromethyl)-4-(trifluoromethyl)-1,3-benzoxazole and was isolated as a yellow oil.

Yield 1.00 g (75%). ¹H NMR (400 MHz, DMSO) δ 8.31 (d, J=1.6 Hz, 1H), 8.01 (d, J=8.4 Hz, 1H), 7.78 (dd, J=1.6, 8.4 Hz, 1H), 5.15 (s, 2H). m/z: [ESI⁺] 236, 238 (M+H)⁺.

### Synthesis of 2-(chloromethyl)-7-(trifluoromethyl)benzo[d]oxazole

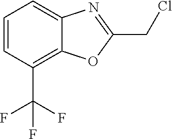 [SMILES: FC(F)(F)c1cccc2nc(CCl)oc12]

Compound 2-(chloromethyl)-7-(trifluoromethyl)benzo[d]oxazole was prepared from 2-amino-6-(trifluoromethyl)phenol (0.50 g, 2.82 mmol) following a procedure similar to that described for the synthesis of 2-(chloromethyl)-4-(trifluoromethyl)-1,3-benzoxazole and was isolated as a yellow solid.

Yield 0.60 g (90%). ¹H NMR (400 MHz, DMSO) δ 8.15 (d, J=8.0 Hz, 1H), 7.84 (d, J=7.6 Hz, 1H), 7.63 (dd, J=1.6, 8.0 Hz, 1H), 5.17 (s, 2H). No Mass signal.

### Synthesis of 6-bromo-2-(chloromethyl)benzo[d]oxazole

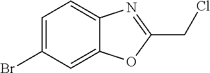 [SMILES: ClCc1nc2ccc(Br)cc2o1]

A mixture of 2-amino-5-bromophenol (5.00 g, 26.59 mmol) and ethyl 2-chloroacetimidate hydrochloride (5.50 g, 34.81 mmol) in DCM (50 mL) was stirred overnight at room temperature under a nitrogen atmosphere. The resulting mixture was quenched with water (100 mL) and extracted with ethyl acetate (2×100 mL). The combined organic layers were washed with brine (50 mL) and dried over anhydrous Na₂SO₄. After filtration, the filtrate was concentrated under reduced pressure. The residue was purified by silica gel column chromatography, eluting with 17% ethyl acetate in petroleum ether to afford 6-bromo-2-(chloromethyl)benzo[d]oxazole as an orange solid.

Yield 5.30 g (81%). ¹H NMR (400 MHz, DMSO) δ 8.15 (d, J=1.6 Hz, 1H), 7.76 (d, J=8.4 Hz, 1H), 7.60 (dd, J=1.6, 8.4 Hz, 1H), 5.08 (s, 2H). m/z: [ESI⁺] 246, 248, 250 (M+H)⁺.

### Synthesis of Benzyl 4-(1-methyl-1H-1,2,4-triazol-3-yl)piperazine-1-carboxylate

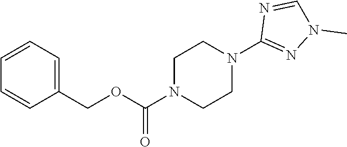 [SMILES: Cn1cnc(N2CCN(C(=O)OCc3ccccc3)CC2)n1]

A solution of 1-methyl-1H-1,2,4-triazol-3-amine (1.00 g, 10.19 mmol), benzyl bis(2-oxoethyl)carbamate (2.88 g, 12.23 mmol), sodium cyanoborohydride (2.24 g, 35.65 mmol) and acetic acid (2.14 g, 35.68 mmol) in MeOH (10 mL) was stirred for 16 h at room temperature under a nitrogen atmosphere. The resulting mixture was concentrated under reduced pressure. The residue was purified by silica gel column chromatography, eluting with 9% MeOH in DCM to afford benzyl 4-(1-methyl-1H-1,2,4-triazol-3-yl)piperazine-1-carboxylate as a yellow oil.

Yield 2.20 g (72%). ¹H NMR (400 MHz, DMSO) δ 8.10 (s, 1H), 7.43-7.27 (m, 5H), 5.10 (s, 2H), 3.68 (s, 3H), 3.53-3.45 (m, 4H), 3.30-3.22 (m, 4H). m/z: [ESI⁺] 302 (M+H)⁺.

### Synthesis of 1-(1-methyl-1H-1,2,4-triazol-3-yl)piperazine

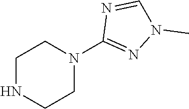

To a solution of benzyl 4-(1-methyl-1,2,4-triazol-3-yl)piperazine-1-carboxylate (272 mg, 0.903 mmol) in methanol (10 mL) was added 10% wt. palladium on carbon (48 mg). Following degassing of the stirred mixture with nitrogen, a hydrogen atmosphere was introduced and the mixture was stirred for 16 h at room temperature. The resulting mixture was filtered through a Celite pad and washed with MeOH (3×10 mL). The filtrate was concentrated under reduced pressure to afford 1-(1-methyl-1,2,4-triazol-3-yl)piperazine as an off-white solid.

Yield 75 mg (50%). ¹H NMR (400 MHz, DMSO) δ 8.05 (s, 1H), 3.67 (s, 3H), 3.25 (brs, 1H), 3.21-3.14 (m, 4H), 2.79-2.72 (m, 4H). m/z: [ESI⁺] 168 (M+H)⁺.

### Synthesis of Tert-Butyl 4-(5-fluoro-6-methylpyrimidin-4-yl)piperazine-1-carboxylate

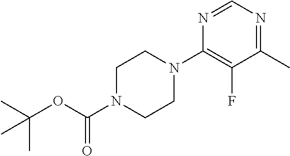 [SMILES: Cc1ncnc(N2CCN(C(=O)OC(C)(C)C)CC2)c1F]

To a solution of 5-fluoro-6-methyl-3H-pyrimidin-4-one (5.00 g, 39.03 mmol) and tert-butyl piperazine-1-carboxylate (10.90 g, 58.52 mmol) in DMF (50 mL) were added DIPEA (10.09 g, 78.06 mmol) and BOP (22.44 g, 50.74 mmol) at room temperature under a nitrogen atmosphere. The resulting solution was stirred for 16 h at 100° C. under a nitrogen atmosphere. The resulting solution was cooled to room temperature, diluted with water (100 mL) and extracted with ethyl acetate (3×200 mL). The combined organic layers were washed with brine (100 mL) and dried over anhydrous Na₂SO₄. After filtration, the filtrate was concentrated under reduced pressure. The residue was purified by silica gel column chromatography, eluting with 66% ethyl acetate in DCM to afford tert-butyl 4-(5-fluoro-6-methylpyrimidin-4-yl)piperazine-1-carboxylate as a light yellow solid.

Yield 770 mg (7%), ¹H NMR (400 MHz, DMSO) δ 8.25 (d, J=2.4 Hz, 1H), 3.69-3.62 (m, 4H), 3.46-3.40 (m, 4H), 2.30 (d, J=3.6 Hz, 3H), 1.42 (s, 9H). m/z: [ESI⁺] 297 (M+H)⁺.

### Synthesis of 5-fluoro-4-methyl-6-(piperazin-1-yl)pyrimidine Hydrochloride

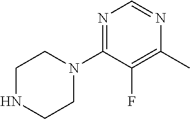

Compound 5-fluoro-4-methyl-6-(piperazin-1-yl)pyrimidine hydrochloride was prepared from tert-butyl 4-(5-fluoro-6-methylpyrimidin-4-yl)piperazine-1-carboxylate (770 mg, 2.598 mmol) following a similar procedure to that described for the synthesis of 2-(piperazin-1-ylmethyl)-1H-indole, and was isolated as a white solid. The intermediate was taken onto the next step without further purification.

Yield 518 mg (86%). ¹H NMR (400 MHz, DMSO) δ 9.75 (br s, 2H), 8.65 (d, J=1.6 Hz, 1H), 4.20-4.09 (m, 4H), 3.26-3.22 (m, 4H), 2.44 (d, J=3.6 Hz, 3H). m/z: [ESI⁺] 197 (M+H)⁺.

### Synthesis of Tert-Butyl 4-(5-chloro-6-methylpyrimidin-4-yl)piperazine-1-carboxylate

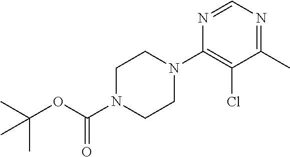 [SMILES: Cc1ncnc(N2CCN(C(=O)OC(C)(C)C)CC2)c1Cl]

To a solution of 4,5-dichloro-6-methylpyrimidine (2.00 g, 12.27 mmol) in dioxane (20 mL) were added tert-butyl piperazine-1-carboxylate (2.74 g, 14.71 mmol) and DIPEA (4.76 g, 36.83 mmol) at room temperature under a nitrogen atmosphere. The resulting mixture was stirred for 16 h at 100° C. The resulting mixture was cooled down to room temperature and diluted with water (100 mL). The resulting mixture was extracted with ethyl acetate (3×100 ml). The combined organic layers were washed with brine (100 mL) and dried over anhydrous Na₂SO₄. After filtration, the filtrate was concentrated under reduced pressure and the residue was purified by silica gel column chromatography, eluted with 20% ethyl acetate in petroleum ether to afford tert-butyl 4-(5-chloro-6-methylpyrimidin-4-yl)piperazine-1-carboxylate as a yellow solid.

Yield 3.40 g (89%). ¹H NMR (400 MHz, DMSO) δ 8.48 (s, 1H), 3.55-3.49 (m, 4H), 3.48-3.42 (m, 4H), 2.46 (s, 3H), 1.42 (s, 9H). m/z: [ESI⁺] 313 (M+H)⁺.

### Synthesis of 5-chloro-4-methyl-6-(piperazin-1-yl)pyrimidine Hydrochloride

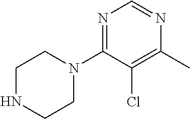

Compound 5-chloro-4-methyl-6-(piperazin-1-yl)pyrimidine hydrochloride was prepared from tert-butyl 4-(5-chloro-6-methylpyrimidin-4-yl)piperazine-1-carboxylate (1.00 g, 3.20 mmol) following a similar procedure to that described for the synthesis of 2-(piperazin-1-ylmethyl)-1H-indole and was isolated as an off-white solid. The intermediate was taken onto the next step without further purification.

Yield 0.70 g (88%). ¹H NMR (400 MHz, DMSO) δ 9.28 (br s, 2H), 8.60 (s, 1H), 3.84-3.78 (m, 4H), 3.24-3.18 (m, 4H), 2.51 (s, 3H). m/z: [ESI⁺] 213, 215 (M+H)⁺.

### Synthesis of Tert-Butyl 5-(5,6-dimethylpyrimidin-4-yl)-2,5-diazabicyclo[2.2.1]heptane-2-carboxylate

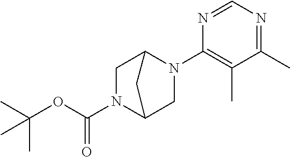 [SMILES: Cc1ncnc(N2CC3CC2CN3C(=O)OC(C)(C)C)c1C]

Compound tert-butyl 5-(5,6-dimethylpyrimidin-4-yl)-2,5-diazabicyclo[2.2.1]heptane-2-carboxylate was prepared from tert-butyl 2,5-diazabicyclo[2.2.1]heptane-2-carboxylate (1.67 g, 8.42 mmol) and 4-chloro-5,6-dimethylpyrimidine (1.00 g, 7.01 mmol) following a similar procedure to that described for the synthesis of tert-butyl 4-(5-chloro-6-methylpyrimidin-4-yl)piperazine-1-carboxylate and was isolated as a yellow solid.

Yield 2.00 g (94%). ¹H NMR (400 MHz, CDCl₃) δ 8.37 (s, 1H), 4.86 (s, 1H), 4.59 (s, 0.6H), 4.47 (s, 0.4H), 3.80 (dd, J=2.0, 9.0 Hz, 1H), 3.65 (d, J=10.4 Hz, 0.4H), 3.58 (d, J=10.4 Hz, 0.6H), 3.48-3.33 (m, 2H), 2.38 (s, 3H), 2.14 (s, 3H), 1.89 (s, 2H), 1.42 (s, 9H). m/z: [ESI⁺] 305 (M+H)⁺.

### Synthesis of 2-(5,6-dimethylpyrimidin-4-yl)-2,5-diazabicyclo[2.2.1]heptane Hydrochloride

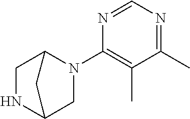

Compound 2-(5,6-dimethylpyrimidin-4-yl)-2,5-diazabicyclo[2.2.1]heptane hydrochloride was prepared from tert-butyl 5-(5,6-dimethylpyrimidin-4-yl)-2,5-diazabicyclo[2.2.1]heptane-2-carboxylate (1.00 g, 3.29 mmol) following a similar procedure to that described for the synthesis of 2-(piperazin-1-ylmethyl)-1H-indole and was isolated as a yellow solid. The intermediate was taken onto the next step without further purification.

Yield 0.70 g (89%). m/z: [ESI⁺] 205 (M+H)⁺.

### Synthesis of Tert-Butyl 4-(5-iodo-6-methylpyrimidin-4-yl)piperazine-1-carboxylate

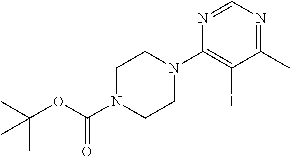

Compound tert-butyl 4-(5-iodo-6-methylpyrimidin-4-yl)piperazine-1-carboxylate was prepared from tert-butyl piperazine-1-carboxylate (3.30 g, 17.72 mmol) and 4-chloro-5-iodo-6-methylpyrimidine (3.00 g, 11.79 mmol) following a similar procedure to that described for the synthesis of tert-butyl 4-(5-chloro-6-methylpyrimidin-4-yl)piperazine-1-carboxylate and was isolated as a yellow solid.

Yield 3.83 g (80%). ¹H NMR (400 MHz, CDCl₃) δ 8.51 (s, 1H), 3.65-3.58 (m, 4H), 3.51-3.41 (m, 4H), 2.71 (s, 3H), 1.50 (s, 9H). m/z: [ESI⁺] 405 (M+H)⁺.

### Synthesis of 5-iodo-4-methyl-6-(piperazin-1-yl)pyrimidine Hydrochloride

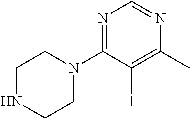

Compound 5-iodo-4-methyl-6-(piperazin-1-yl)pyrimidine hydrochloride was prepared from tert-butyl 4-(5-iodo-6-methylpyrimidin-4-yl)piperazine-1-carboxylate (2.30 g, 5.69 mmol) following a similar procedure to that described for the synthesis of 2-(piperazin-1-ylmethyl)-1H-indole, and was isolated as a white solid. The intermediate was taken onto the next step without further purification.

Yield 1.53 g (79%). ¹H NMR (400 MHz, DMSO) δ 9.34 (s, 2H), 8.71 (s, 1H), 3.92-3.77 (m, 4H), 3.30-3.18 (m, 4H), 2.65 (s, 3H). m/z: [ESI⁺] 305 (M+H)⁺.

### Synthesis of 6-fluoro-2-((4-(5-iodo-6-methylpyrimidin-4-yl)piperazin-1-yl)methyl)benzo[d]oxazole

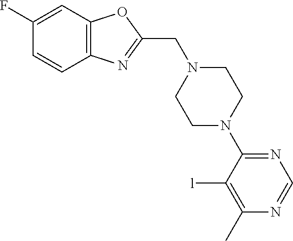

Compound 6-fluoro-2-((4-(5-iodo-6-methylpyrimidin-4-yl)piperazin-1-yl)methyl) benzo[d]oxazole was prepared from 2-(chloromethyl)-6-fluorobenzo[d]oxazole (268 mg, 1.444 mmol) and 5-iodo-4-methyl-6-(piperazin-1-yl)pyrimidine hydrochloride (400 mg, 1.174 mmol) following a similar procedure to that described for the synthesis of 2-[[4-(4-pyridyl)piperazin-1-yl]methyl]-1H-benzimidazole and was isolated as an off-white solid.

Yield 0.20 g (38%). ¹H NMR (400 MHz, DMSO) δ 8.46 (s, 1H), 7.82-7.71 (m, 2H), 7.32-7.22 (m, 1H), 3.95 (s, 2H), 3.47-3.40 (m, 4H), 2.76-2.69 (m, 4H), 2.58 (s, 3H). m/z: [ESI⁺] 454 (M+H)⁺.

### Synthesis of 6-bromo-2-((4-(5,6-dimethylpyrimidin-4-yl)piperazin-1-yl)methyl)benzo[d]oxazole

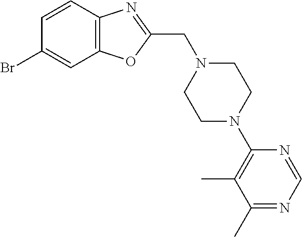 [SMILES: Cc1ncnc(N2CCN(Cc3nc4ccc(Br)cc4o3)CC2)c1C]

Compound 6-bromo-2-((4-(5,6-dimethylpyrimidin-4-yl)piperazin-1-yl)methyl)benzo[d]oxazole was prepared from 6-bromo-2-(chloromethyl)benzo[d]oxazole (2.00 g, 8.11 mmol) and 4,5-dimethyl-6-(piperazin-1-yl)pyrimidine hydrochloride (2.04 g, 8.92 mmol) following a similar procedure to that described for the synthesis of 2-[[4-(4-pyridyl)piperazin-1-yl]methyl]-1H-benzimidazole and was isolated as an off-white solid.

Yield 2.20 g (67%). ¹H NMR (400 MHz, DMSO) δ 8.41 (s, 1H), 7.95 (s, 1H), 7.71 (d, J=8.4 Hz, 1H), 7.55 (dd, J=1.6, 8.4 Hz, 1H), 3.94 (s, 2H), 3.25 (t, J=4.8 Hz, 4H), 2.68 (t, J=4.8 Hz, 4H), 2.31 (s, 3H), 2.09 (s, 3H). m/z: [ESI⁺] 402, 404 (M+H)⁺.

### Synthesis of 2-((4-(5,6-dimethylpyrimidin-4-yl)piperazin-1-yl)methyl)-6-vinylbenzo[d]oxazole

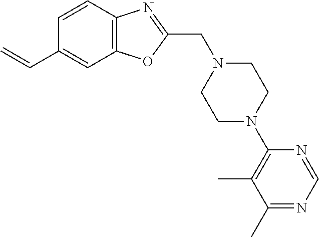 [SMILES: C=Cc1ccc2nc(CN3CCN(c4ncnc(C)c4C)CC3)oc2c1]

Compound 2-((4-(5,6-dimethylpyrimidin-4-yl)piperazin-1-yl)methyl)-6-vinylbenzo[d]oxazole was prepared from 6-bromo-2-((4-(5,6-dimethylpyrimidin-4-yl)piperazin-1-yl)methyl)benzo[d]oxazole (40.00 g, 99.43 mmol) and 2-ethenyl-4,4,5,5-tetramethyl-1,3,2-dioxaborolane (38.29 g, 248.60 mmol) following a similar procedure to that described for the synthesis of tert-butyl 4-(5-ethenylpyrimidin-4-yl)piperazine-1-carboxylate and was isolated as an off-white solid.

Yield 25.00 g (72%). ¹H NMR (400 MHz, DMSO) δ 8.41 (s, 1H), 7.87 (s, 1H), 7.69 (d, J=8.4 Hz, 1H), 7.50 (d, J=8.4 Hz, 1H), 6.85 (dd, J=10.8, 17.6 Hz, 1H), 5.93 (d, J=17.6 Hz, 1H), 5.31 (d, J=10.8 Hz, 1H), 3.93 (s, 2H), 3.30-3.20 (m, 4H), 2.71-2.64 (m, 4H), 2.31 (s, 3H), 2.09 (s, 3H). m/z: [ESI⁺] 350 (M+H)⁺.

### Synthesis of 2-((4-(5,6-dimethylpyrimidin-4-yl)piperazin-1-yl)methyl)benzo[d]oxazole-6-carbaldehyde

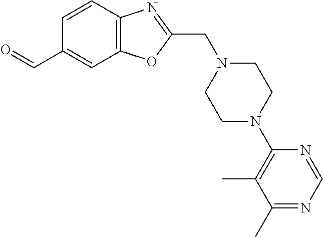 [SMILES: Cc1ncnc(N2CCN(Cc3nc4ccc(C=O)cc4o3)CC2)c1C]

Compound 2-((4-(5,6-dimethylpyrimidin-4-yl)piperazin-1-yl)methyl)benzo[d]oxazole-6-carbaldehyde was prepared from 2-((4-(5,6-dimethylpyrimidin-4-yl)piperazin-1-yl)methyl)-6-vinylbenzo[d]oxazole (25.00 g, 71.54 mmol) following a similar procedure to that described for the synthesis of tert-butyl 4-(5-formylpyrimidin-4-yl)piperazine-1-carboxylate and was isolated as an off-white solid.

Yield 6.30 g (25%). ¹H NMR (400 MHz, CDCl₃) δ 10.11 (s, 1H), 8.54 (s, 1H), 8.08 (d, J=1.6 Hz, 1H), 7.93 (dd, J=1.6, 8.4 Hz, 1H), 7.87 (d, J=8.4 Hz, 1H), 4.01 (s, 2H), 3.45-3.36 (m, 4H), 2.83-2.80 (m, 4H), 2.41 (s, 3H), 2.15 (s, 3H). m/z: [ESI⁺] 352 (M+H)⁺.

### Synthesis of Tert-Butyl 4-(6-methyl-5-vinylpyrimidin-4-yl)piperazine-1-carboxylate

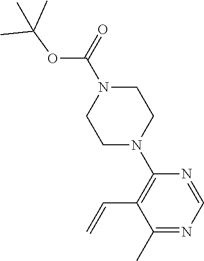 [SMILES: C=Cc1c(C)ncnc1N1CCN(C(=O)OC(C)(C)C)CC1]

Compound tert-butyl 4-(6-methyl-5-vinylpyrimidin-4-yl)piperazine-1-carboxylate was prepared from tert-butyl 4-(5-iodo-6-methylpyrimidin-4-yl)piperazine-1-carboxylate (4.00 g, 9.90 mmol) and potassium vinyltrifluoroborate (4.56 g, 34.04 mmol) following a similar procedure to that described for the synthesis of tert-butyl 4-(5-ethenylpyrimidin-4-yl)piperazine-1-carboxylate and was isolated as an off-white solid.

Yield 1.62 g (54%). ¹H NMR (400 MHz, CDCl₃) δ 8.54 (s, 1H), 6.59 (dd, J=11.6, 18.0 Hz, 1H), 5.61 (dd, J=1.6, 11.6 Hz, 1H), 5.51 (dd, J=1.6, 18.0 Hz, 1H), 3.57-3.43 (m, 8H), 2.49 (s, 3H), 1.49 (s, 9H). m/z: [ESI⁺] 305 (M+H)⁺.

### Synthesis of 4-methyl-6-(piperazin-1-yl)-5-vinylpyrimidine Hydrochloride

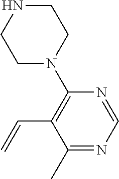

Compound 4-methyl-6-(piperazin-1-yl)-5-vinylpyrimidine hydrochloride was prepared from tert-butyl 4-(6-methyl-5-vinylpyrimidin-4-yl)piperazine-1-carboxylate (1.60 g, 5.26 mmol) following a similar procedure to that described for the synthesis of 2-(piperazin-1-ylmethyl)-1H-indole and was isolated as an off-white solid.

Yield 1.05 g (83%). ¹H NMR (400 MHz, DMSO) δ 9.92 (br s, 2H), 8.80 (s, 1H), 6.71 (dd, J=11.6, 17.8 Hz, 1H), 5.72 (dd, J=1.2, 11.6 Hz, 1H), 5.56 (dd, J=1.2, 17.8 Hz, 1H), 4.03 (t, J=5.2 Hz, 4H), 3.20 (t, J=5.2 Hz, 4H), 2.50 (s, 3H). m/z: [ESI⁺] 205 (M+H)⁺.

### Synthesis of 2-((4-(6-methyl-5-vinylpyrimidin-4-yl)piperazin-1-yl)methyl)benzo[d]oxazole

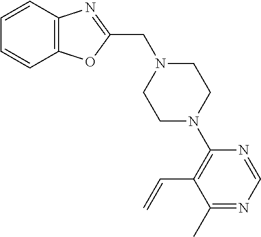 [SMILES: C=Cc1c(C)ncnc1N1CCN(Cc2nc3ccccc3o2)CC1]

Compound 2-((4-(6-methyl-5-vinylpyrimidin-4-yl)piperazin-1-yl)methyl)benzo[d]oxazole was prepared from 2-(chloromethyl)benzo[d]oxazole (0.90 g, 5.37 mmol) and 4-methyl-6-(piperazin-1-yl)-5-vinylpyrimidine hydrochloride (1.00 g, 4.15 mmol) following a similar procedure to that described for the synthesis of 2-[[4-(4-pyridyl)piperazin-1-yl]methyl]-1H-benzimidazole and was isolated as an off-white solid.

Yield 1.07 g (77%). ¹H NMR (400 MHz, CDCl₃) δ 8.51 (s, 1H), 7.79-7.68 (m, 1H), 7.60-7.51 (m, 1H), 7.41-7.31 (m, 2H), 6.56 (dd, J=11.6, 18.0 Hz, 1H), 5.56 (dd, J=1.6, 11.6 Hz, 1H), 5.47 (dd, J=1.6, 18.0 Hz, 1H), 3.93 (s, 2H), 3.66-3.54 (m, 4H), 2.78-2.69 (m, 4H), 2.46 (s, 3H). m/z: [ESI⁺] 336 (M+H)⁺.

### Synthesis of Tert-Butyl 2-(hydroxymethyl)-6-methoxy-1H-indole-1-carboxylate

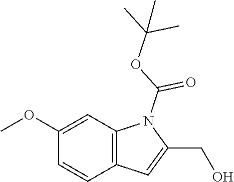 [SMILES: COc1ccc2cc(CO)n(C(=O)OC(C)(C)C)c2c1]

Compound tert-butyl 2-(hydroxymethyl)-6-methoxy-1H-indole-1-carboxylate was prepared from 1-(tert-butyl) 2-methyl 6-methoxy-1H-indole-1,2-dicarboxylate (1.00 g, 3.28 mmol) following a similar procedure to that described for the synthesis of (5-nitro-1H-indol-2-yl)methanol and was isolated as a yellow solid.

Yield 0.80 g (88%). ¹H NMR (400 MHz, DMSO) δ 7.64 (d, J=2.4 Hz, 1H), 7.42 (d, J=8.4 Hz, 1H), 6.85 (dd, J=2.4, 8.4 Hz, 1H), 6.57 (s, 1H), 5.22 (br s, 1H), 4.74 (s, 2H), 3.79 (s, 3H), 1.63 (s, 9H). LCMS signal not observed.

### Synthesis of Tert-Butyl 2-(chloromethyl)-6-methoxy-1H-indole-1-carboxylate

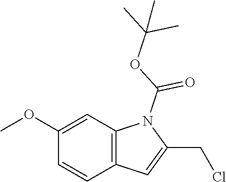 [SMILES: COc1ccc2cc(CCl)n(C(=O)OC(C)(C)C)c2c1]

To a stirred solution of tert-butyl 2-(hydroxymethyl)-6-methoxy-1H-indole-1-carboxylate (200 mg, 0.721 mmol), triethylamine (180 mg, 1.779 mmol) and lithium chloride (310 mg, 7.312 mmol) in THF (6 mL) was added methanesulfonyl chloride (165 mg, 1.440 mmol) dropwise at 0° C. under a nitrogen atmosphere. The resulting solution was stirred overnight at room temperature under a nitrogen atmosphere. The resulting mixture was diluted with water (10 mL) and extracted with diethyl ether (3×10 mL). The combined organic layers were washed with brine (10 mL) and dried over anhydrous Na₂SO₄. After filtration, the filtrate was concentrated under reduced pressure to afford tert-butyl 2-(chloromethyl)-6-methoxy-1H-indole-1-carboxylate as a yellow oil.

Yield 120 mg (crude).

### Synthesis of Tert-Butyl 2-((4-(5-ethylpyrimidin-4-yl)piperazin-1-yl)methyl)-6-methoxy-1H-indole-1-carboxylate

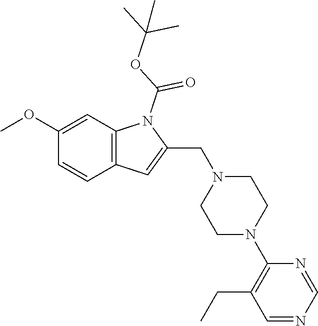 [SMILES: CCc1cncnc1N1CCN(Cc2cc3ccc(OC)cc3n2C(=O)OC(C)(C)C)CC1]

Compound tert-butyl 2-((4-(5-ethylpyrimidin-4-yl)piperazin-1-yl)methyl)-6-methoxy-1H-indole-1-carboxylate was prepared from tert-butyl 2-(chloromethyl)-6-methoxy-1H-indole-1-carboxylate (120 mg, 0.406 mmol) and 5-ethyl-4-(piperazin-1-yl)pyrimidine hydrochloride (115 mg, 0.503 mmol) following a similar procedure to that described for the synthesis of 2-[[4-(4-pyridyl)piperazin-1-yl]methyl]-1H-benzimidazole and was isolated as a yellow oil.

Yield 25 mg (14%). ¹H NMR (400 MHz, CDCl₃) δ 8.62 (s, 1H), 8.23 (s, 1H), 7.69 (d, J=2.4 Hz, 1H), 7.38 (d, J=8.4 Hz, 1H), 6.88 (dd, J=2.4, 8.4 Hz, 1H), 6.51 (s, 1H), 3.99-3.90 (m, 2H), 3.89 (s, 3H), 3.56-3.36 (m, 4H), 2.71-2.64 (m, 4H), 2.63 (q, J=7.2 Hz, 2H), 1.72 (s, 9H), 1.28 (t, J=7.2 Hz, 3H). m/z: [ESI⁺] 452 (M+H)⁺.

### Synthesis of 2-((4-(5-ethylpyrimidin-4-yl)piperazin-1-yl)methyl)-1H-indol-6-ol

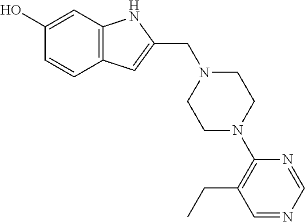 [SMILES: CCc1cncnc1N1CCN(Cc2cc3ccc(O)cc3[nH]2)CC1]

To a solution of tert-butyl 2-((4-(5-ethylpyrimidin-4-yl)piperazin-1-yl)methyl)-6-methoxy-1H-indole-1-carboxylate (25 mg, 0.055 mmol) in DCM (4 mL) was added portionwise boron tribromide (139 mg, 0.555 mmol) −78° C. under a nitrogen atmosphere. The resulting solution was then stirred for 30 min at −78° C. The reaction was quenched by the addition of MeOH (10 mL) at −40° C. The resulting mixture was then concentrated under reduced pressure. The residue was purified by Prep-TLC, eluting with 17% methanol in DCM to afford 2-((4-(5-ethylpyrimidin-4-yl)piperazin-1-yl)methyl)-1H-indol-6-ol as a yellow oil.

Yield 18 mg (96%). ¹H NMR (400 MHz, CD₃OD) δ 8.48 (s, 1H), 8.19 (s, 1H), 7.28 (d, J=8.4 Hz, 1H), 6.77 (d, J=2.0 Hz, 1H), 6.57 (dd, J=2.0, 8.4 Hz, 1H), 6.26 (s, 1H), 3.72 (s, 2H), 3.56 (t, J=5.2 Hz, 4H), 2.73-2.59 (m, 6H), 1.26 (t, J=7.6 Hz, 3H). m/z: [ESI⁺] 338 (M+H)⁺.

### Synthesis of Tert-Butyl 4-(2-((3-hydroxypyridin-4-yl)amino)-2-oxoethyl)piperazine-1-carboxylate

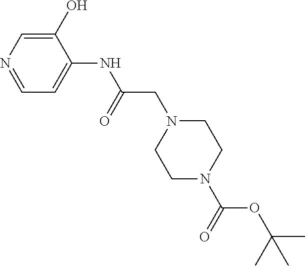 [SMILES: CC(C)(C)OC(=O)N1CCN(CC(=O)Nc2ccncc2O)CC1]

Compound tert-butyl 4-(2-((3-hydroxypyridin-4-yl)amino)-2-oxoethyl)piperazine-1-carboxylate was prepared from 2-(4-(tert-butoxycarbonyl)piperazin-1-yl)acetic acid (3.00 g, 12.28 mmol) and 4-aminopyridin-3-ol (1.49 g, 13.51 mmol) following a similar procedure to that described for the synthesis of (4-(5-ethylpyrimidin-4-yl)piperazin-1-yl)(6-((2-methoxyethoxy)methyl)-1H-indol-2-yl)methanone except HBTU was used and was isolated as an off-white solid.

Yield 2.44 g (59%). ¹H NMR (400 MHz, DMSO) δ 10.54 (br s, 1H), 9.77 (s, 1H), 8.12 (s, 1H), 8.10 (d, J=5.2 Hz, 1H), 7.97 (d, J=5.2 Hz, 1H), 3.42-3.33 (m, 4H), 3.30 (t, J=5.2 Hz, 2H), 3.22 (s, 2H), 2.39 (t, J=5.2 Hz, 2H), 1.41 (s, 9H). m/z: [ESI⁺] 337 (M+H)⁺.

### Synthesis of Tert-Butyl 4-(oxazolo[5,4-c]pyridin-2-ylmethyl)piperazine-1-carboxylate

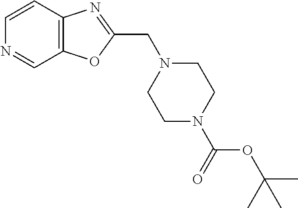 [SMILES: CC(C)(C)OC(=O)N1CCN(Cc2nc3ccncc3o2)CC1]

To a mixture of triphenylphosphine (5.71 g, 21.76 mmol) and triethylamine (5.87 g, 58.03 mmol) in DCM (50 mL) was added hexachloroethane (4.29 g, 18.13 mmol) portionwise at room temperature under a nitrogen atmosphere. To this mixture was added tert-butyl 4-(2-((3-hydroxypyridin-4-yl)amino)-2-oxoethyl)piperazine-1-carboxylate (2.44 g, 7.25 mmol) portionwise at room temperature. The resulting mixture was stirred overnight at room temperature. The reaction mixture was quenched with water (100 mL) and extracted with DCM (3×100 mL). The combined organic layers were washed with brine (200 mL) and dried over anhydrous Na₂SO₄. After filtration, the filtrate was concentrated under reduced pressure. The crude residue was purified by reverse phase flash chromatography under the following conditions: Column: WelFlash™ C18-I, 20-40 nm, 330 g; Eluent A: water (plus 10 mmol/L NH₄HCO₃); Eluent B: acetonitrile; Gradient: 25%-45% B in 25 min; Flow rate: 80 mL/min; Detector: UV 220/254 nm. Desired fractions were collected concentrated under reduced pressure to afford tert-butyl 4-(oxazolo[5,4-c]pyridin-2-ylmethyl)piperazine-1-carboxylate as an off-white solid.

Yield 0.65 g (28%). ¹H NMR (400 MHz, DMSO) δ 9.09 (d, J=1.0 Hz, 1H), 8.55 (d, J=5.2 Hz, 1H), 7.83 (dd, J=1.0, 5.2 Hz, 1H), 3.99 (s, 2H), 3.37-3.32 (m, 4H), 2.56-2.51 (m, 4H), 1.38 (s, 9H). m/z: [ESI⁺] 319 (M+H)⁺.

### Synthesis of 2-(piperazin-1-ylmethyl)oxazolo[5,4-c]pyridine bis(trifluoroacetate)

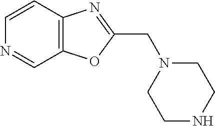 [SMILES: c1cc2nc(CN3CCNCC3)oc2cn1]

Compound 2-(piperazin-1-ylmethyl)oxazolo[5,4-c]pyridine bis(trifluoroacetate) was prepared from tert-butyl 4-(oxazolo[5,4-c]pyridin-2-ylmethyl)piperazine-1-carboxylate (0.65 g, 2.04 mmol) following a similar procedure to that described for the synthesis of 2-(pyridin-4-yl)-2,6-diazaspiro[3.3]heptane, and was isolated as an off-white solid.

Yield 1.80 g (crude). ¹H NMR (400 MHz, DMSO) δ 9.37 (s, 1H), 8.72 (d, J=5.6 Hz, 1H), 8.10 (d, J=5.6 Hz, 1H), 4.19 (s, 2H), 3.19-3.10 (m, 4H), 2.90-2.83 (m, 4H). Trifluoroacetate protons not observed. m/z: [ESI⁺] 219 (M+H)⁺.

### Synthesis of Ethyl 1-(4-benzylpiperazin-1-yl)cyclopropane-1-carboxylate

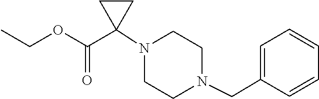 [SMILES: CCOC(=O)C1(N2CCN(Cc3ccccc3)CC2)CC1]

To a mixture of ethyl 1-aminocyclopropane-1-carboxylate hydrochloride (10.00 g, 60.38 mmol) and benzyl bis(2-chloroethyl)amine (18.00 g, 77.54 mmol) in ethanol (133 mL) was added DIPEA (90.00 g, 696.36 mmol) at room temperature. The resulting mixture was stirred for 16 h at 80° C. under a nitrogen atmosphere. The resulting mixture was cooled to room temperature and concentrated under reduced pressure. The residue was purified by silica gel column chromatography, eluting with 50% ethyl acetate in petroleum ether to afford ethyl 1-(4-benzylpiperazin-1-yl)cyclopropane-1-carboxylate as a yellow oil.

Yield 4.70 g (27%). ¹H NMR (400 MHz, DMSO) δ 7.28 (m, 5H), 4.06 (q, J=7.2 Hz, 2H), 3.39 (s, 2H), 2.90-2.80 (m, 4H), 2.32-2.15 (m, 4H), 1.23-1.10 (m, 5H), 0.86 (t, J=4.0 Hz, 2H). m/z: [ESI⁺]289 (M+H)⁺.

### Synthesis of 1-(4-benzylpiperazin-1-yl)cyclopropane-1-carboxylic Acid

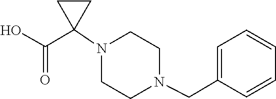 [SMILES: O=C(O)C1(N2CCN(Cc3ccccc3)CC2)CC1]

Compound 1-(4-benzylpiperazin-1-yl)cyclopropane-1-carboxylic acid was prepared from ethyl 1-(4-benzylpiperazin-1-yl)cyclopropane-1-carboxylate (2.00 g, 6.94 mmol) following a similar procedure to that described for the synthesis of 6-[(2-methoxyethoxy)methyl]-1H-indole-2-carboxylic acid, with the reaction carried out at 75° C. and was isolated as a yellow solid.

Yield 623 mg (35%). ¹H NMR (400 MHz, DMSO) δ 12.18 (br s, 1H), 7.41-7.17 (m, 5H), 3.42 (s, 2H), 3.04-2.77 (m, 4H), 2.34-2.10 (m, 4H), 1.12 (q, J=3.6 Hz, 2H), 0.82 (q, J=3.6 Hz, 2H). m/z: [ESI⁺] 261 (M+H)⁺.

### Synthesis of 1-(4-benzylpiperazin-1-yl)-N-(2-hydroxyphenyl)cyclopropane-1-carboxamide

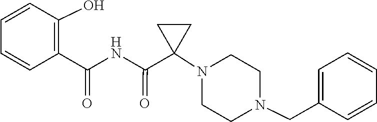 [SMILES: O=C(NC(=O)C1(N2CCN(Cc3ccccc3)CC2)CC1)c1ccccc1O]

Compound 1-(4-benzylpiperazin-1-yl)-N-(2-hydroxyphenyl)cyclopropane-1-carboxamide was prepared from 1-(4-benzylpiperazin-1-yl)cyclopropane-1-carboxylic acid (947 mg, 3.638 mmol) and 2-aminophenol (477 mg, 4.371 mmol) following a similar procedure to that described for the synthesis of (4-(5-ethylpyrimidin-4-yl)piperazin-1-yl)(6-((2-methoxyethoxy)methyl)-1H-indol-2-yl)methanone, and was isolated as an off-white solid.

Yield 706 mg (55%). No HNMR data. m/z: [ESI⁺] 352 (M+H)⁺.

### Synthesis of 2-(1-(4-benzylpiperazin-1-yl)cyclopropyl)benzo[d]oxazole

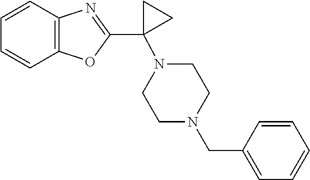 [SMILES: c1ccc(CN2CCN(C3(c4nc5ccccc5o4)CC3)CC2)cc1]

A solution of 1-(4-benzylpiperazin-1-yl)-N-(2-hydroxyphenyl)cyclopropane-1-carboxamide (700 mg, 1.992 mmol) in polyphosphoric acid (5 mL) was stirred for 2 h at 150° C. under a nitrogen atmosphere. The resulting mixture was cooled to room temperature and diluted with water (50 mL). The resulting mixture was extracted with ethyl acetate (3×50 mL) and dried over anhydrous Na₂SO₄. After filtration, the filtrate was concentrated under reduced pressure. The residue was purified by silica gel column chromatography, eluting with 50% ethyl acetate in petroleum ether to afford 2-(1-(4-benzylpiperazin-1-yl)cyclopropyl)benzo[d]oxazole as a yellow oil.

Yield 172 mg (26%). ¹H NMR (400 MHz, DMSO) δ 7.76-7.61 (m, 2H), 7.39-7.17 (m, 7H), 3.44 (s, 2H), 3.04-2.95 (m, 4H), 2.41-2.24 (m, 4H), 1.37-1.33 (m, 2H), 1.16-1.11 (m, 2H). m/z: [ESI⁺] 334 (M+H)⁺.

### Synthesis of 2-(1-(piperazin-1-yl)cyclopropyl)benzo[d]oxazole

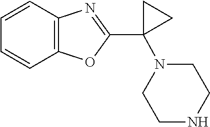 [SMILES: c1ccc2oc(C3(N4CCNCC4)CC3)nc2c1]

To a stirred solution of 2-[1-(4-benzylpiperazin-1-yl)cyclopropyl]-1,3-benzoxazole (705 mg, 2.114 mmol) in methanol (5 mL) was added 10% wt. palladium on carbon (500 mg) at room temperature. The resulting mixture was stirred for overnight at room temperature under a hydrogene atmosphere (1.5 atm). The resulting mixture was filtered and the filtered cake was washed with methanol (3×10 mL). The filtrate was concentrated under reduced pressure. The residue was purified by reverse phase flash chromatography with the following conditions: Column: WelHash™ C18-I, 20-40 um, 120 g; Eluent A: water (plus 10 mmol/L NH₄HCO₃); Eluent B: ACN; Gradient: 13%-33% B in 25 min; How rate: 60 mL/min; Detector: UV 220/254 nm. Desired fractions were collected and concentrated under reduced pressure to afford 2-[1-(piperazin-1-yl)cyclopropyl]-1,3-benzoxazole as a yellow solid.

Yield 23 mg (4%). ¹H NMR (400 MHz, DMSO) δ 7.71-7.60 (m, 2H), 7.39-7.28 (m, 2H), 2.99 (t, J=4.8 Hz, 4H), 2.76 (t, J=4.8 Hz, 4H), 1.36 (q, J=4.4 Hz, 2H), 1.23 (br s, 1H), 1.16 (q, J=4.4 Hz, 2H). m/z: [ESI⁺] 244 (M+H)⁺.

### Synthesis of 2-((4-(2-chloro-5-(trifluoromethyl)pyrimidin-4-yl)piperazin-1-yl)methyl)benzo[d]oxazole

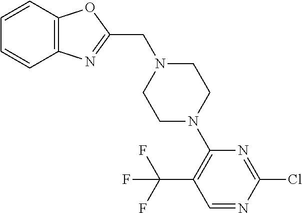 [SMILES: FC(F)(F)c1cnc(Cl)nc1N1CCN(Cc2nc3ccccc3o2)CC1]

Compound 2-((4-(2-chloro-5-(trifluoromethyl)pyrimidin-4-yl)piperazin-1-yl)methyl)benzo[d]oxazole was prepared from 2-(piperazin-1-ylmethyl)benzo[d]oxazole bistrifluoroacetate (500 mg, 1.123 mmol) and 2,4-dichloro-5-(trifluoromethyl)pyrimidine (642 mg, 2.959 mmol) following a similar procedure to that described for the synthesis of tert-butyl 4-(5-chloro-6-methylpyrimidin-4-yl)piperazine-1-carboxylate, and was isolated as a colorless oil.

Yield 100 mg (22%). ¹H NMR (400 MHz, DMSO) δ 8.67 (s, 1H), 7.80-7.66 (m, 2H), 7.45-7.33 (m, 2H), 3.96 (s, 2H), 3.92-3.76 (m, 4H), 2.71-2.58 (m, 4H). m/z: [ESI⁺] 398 (M+H)⁺.

### Synthesis of 2-((4-(5-iodo-6-methylpyrimidin-4-yl)piperazin-1-yl)methyl)benzo[d]oxazole

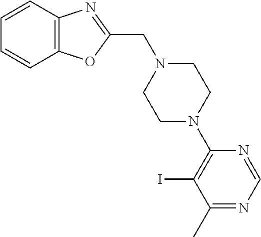 [SMILES: Cc1ncnc(N2CCN(Cc3nc4ccccc4o3)CC2)c1I]

Compound 2-((4-(5-iodo-6-methylpyrimidin-4-yl)piperazin-1-yl)methyl)benzo[d]oxazole was prepared from 2-(piperazin-1-ylmethyl)benzo[d]oxazole bistrifluoroacetate (1.00 g, 2.25 mmol) and 4-chloro-5-iodo-6-methylpyrimidine (1.50 g, 5.91 mmol) following a similar procedure to that described for the synthesis of tert-butyl 4-(5-chloro-6-methylpyrimidin-4-yl)piperazine-1-carboxylate and was isolated as a yellow oil.

Yield 300 mg (31%). ¹H NMR (400 MHz, DMSO) δ 8.46 (s, 1H), 7.79-7.70 (m, 2H), 7.46-7.33 (m, 2H), 3.96 (s, 2H), 3.48-3.39 (m, 4H), 2.77-2.68 (m, 4H), 2.58 (s, 3H). m/z: [ESI⁺] 436 (M+H)⁺.

### Synthesis of Tert-Butyl 4-(6-chloro-5-methoxypyrimidin-4-yl)piperazine-1-carboxylate

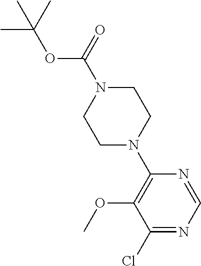 [SMILES: COc1c(Cl)ncnc1N1CCN(C(=O)OC(C)(C)C)CC1]

Compound tert-butyl 4-(6-chloro-5-methoxypyrimidin-4-yl)piperazine-1-carboxylate was prepared from 4,6-dichloro-5-methoxypyrimidine (5.50 g, 30.73 mmol) and tert-butyl piperazine-1-carboxylate (5.78 g, 31.03 mmol) following a similar procedure to that described for the synthesis of tert-butyl 4-(5-chloro-6-methylpyrimidin-4-yl)piperazine-1-carboxylate and was isolated as a yellow oil.

Yield 10.00 g (99%). ¹H NMR (400 MHz, CDCl₃) δ 8.20 (s, 1H), 3.86-3.80 (m, 4H), 3.76 (s, 3H), 3.61-3.49 (m, 4H), 1.50 (s, 9H). m/z: [ESI⁺] 329, 331 (M+H)⁺.

### Synthesis of Tert-Butyl 4-(5-methoxypyrimidin-4-yl)piperazine-1-carboxylate

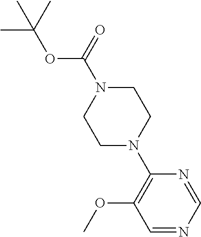 [SMILES: COc1cncnc1N1CCN(C(=O)OC(C)(C)C)CC1]

To a solution of tert-butyl 4-(6-chloro-5-methoxypyrimidin-4-yl)piperazine-1-carboxylate (10.00 g, 30.41 mmol) in methanol (100 mL) was added 10% wt. palladium on carbon (1.62 g) under a nitrogen atmosphere. Following degassing of the stirred mixture with nitrogen, the mixture was stirred for 18 h at room temperature under a hydrogen atmosphere. The resulting mixture was filtered through a Celite pad and washed with MeOH (3×10 mL). The filtrate was concentrated under reduced pressure to afford tert-butyl 4-(5-methoxypyrimidin-4-yl)piperazine-1-carboxylate as a light yellow solid.

Yield 8.00 g (89%). ¹H NMR (400 MHz, CDCl₃) δ 8.41 (s, 1H), 7.99 (s, 1H), 4.18-4.07 (m, 4H), 3.97 (s, 3H), 3.63-3.54 (m, 4H), 1.49 (s, 9H). m/z: [ESI⁺] 295 (M+H)⁺.

### Synthesis of 5-methoxy-4-(piperazin-1-yl)pyrimidine Hydrochloride

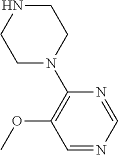

Compound 5-methoxy-4-(piperazin-1-yl)pyrimidine hydrochloride was prepared from tert-butyl 4-(5-methoxypyrimidin-4-yl)piperazine-1-carboxylate (8.00 g, 27.18 mmol) following a similar procedure to that described for the synthesis of 2-(piperazin-1-ylmethyl)-1H-indole and was isolated as a light yellow solid.

Yield 6.00 g (96%). ¹H NMR (300 MHz, CD₃OD) δ 8.61 (s, 1H), 8.15 (s, 1H), 4.50 (t, J=4.8 Hz, 4H), 4.03 (s, 3H), 3.46 (t, J=4.8 Hz, 4H). NH proton not observed. m/z: [ESI⁺] 195 (M+H)⁺.

### Synthesis of (1H-indol-2-yl)(4-(5-methoxypyrimidin-4-yl)piperazin-1-yl)methanone

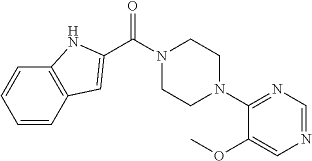 [SMILES: COc1cncnc1N1CCN(C(=O)c2cc3ccccc3[nH]2)CC1]

Compound (1H-indol-2-yl)(4-(5-methoxypyrimidin-4-yl)piperazin-1-yl)methanone was prepared from 1H-indole-2-carboxylic acid (5.97 g, 37.07 mmol) and 5-methoxy-4-(piperazin-1-yl)pyrimidine hydrochloride (6.00 g, 26.01 mmol) following a similar procedure to that described for the synthesis of (4-(5-ethylpyrimidin-4-yl)piperazin-1-yl)(6-((2-methoxyethoxy)methyl)-1H-indol-2-yl)methanone and was isolated as a light yellow solid.

Yield 6.00 g (68%). ¹H NMR (400 MHz, CDCl₃) δ 9.68 (br s, 1H), 8.41 (s, 1H), 8.00 (s, 1H), 7.68 (d, J=8.0 Hz, 1H), 7.47 (d, J=8.4 Hz, 1H), 7.34-7.29 (m, 1H), 7.19-7.14 (m, 1H), 6.84 (s, 1H), 4.12-4.04 (m, 4H), 3.93-3.89 (m, 4H). m/z: [ESI⁺] 338 (M+H)⁺.

### Synthesis of 2-((4-(5-methoxypyrimidin-4-yl)piperazin-1-yl)methyl)-1H-indole

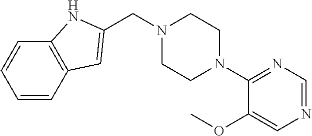 [SMILES: COc1cncnc1N1CCN(Cc2cc3ccccc3[nH]2)CC1]

Compound 2-((4-(5-methoxypyrimidin-4-yl)piperazin-1-yl)methyl)-1H-indole was prepared from (1H-indol-2-yl)(4-(5-methoxypyrimidin-4-yl)piperazin-1-yl)methanone (1.00 g, 2.96 mmol) following a similar procedure to that described for the synthesis of 2-((4-(5-ethylpyrimidin-4-yl)piperazin-1-yl)methyl)-6-((2-methoxyethoxy)methyl)-1H-benzo[d]imidazole and was isolated as a light yellow solid.

Yield 0.67 g (70%). ¹H NMR (400 MHz, CDCl₃) δ 8.58 (br s, 1H), 8.36 (s, 1H), 7.89 (s, 1H), 7.62-7.55 (m, 1H), 7.40-7.35 (m, 1H), 7.21-7.15 (m, 1H), 7.15-7.07 (m, 1H), 6.40 (s, 1H), 3.84 (s, 3H), 3.80 (t, J=5.2 Hz, 4H), 3.72 (s, 2H) 2.60 (t, J=5.2 Hz, 4H). m/z: [ESI⁺] 324 (M+H)⁺.

### Synthesis of 4-(4-((1H-indol-2-yl)methyl)piperazin-1-yl)pyrimidin-5-ol

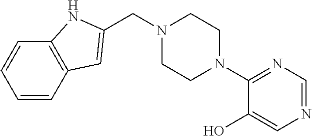 [SMILES: Oc1cncnc1N1CCN(Cc2cc3ccccc3[nH]2)CC1]

Compound 4-(4-((1H-indol-2-yl)methyl)piperazin-1-yl)pyrimidin-5-ol was prepared from 2-((4-(5-methoxypyrimidin-4-yl)piperazin-1-yl)methyl)-1H-indole (0.50 g, 1.55 mmol) following a similar procedure to that described for the synthesis of 2-((4-(5-ethylpyrimidin-4-yl)piperazin-1-yl)methyl)-1H-indol-6-ol and was isolated as a dark yellow solid.

Yield 0.14 g (29%). ¹H NMR (400 MHz, DMSO) δ 11.04 (br s, 1H), 10.00 (br s, 1H), 8.13 (s, 1H), 7.81 (s, 1H), 7.46 (d, J=7.8 Hz, 1H), 7.33 (dd, J=0.8, 7.8 Hz, 1H), 7.07-6.99 (m, 1H), 6.99-6.88 (m, 1H), 6.31 (s, 1H), 3.77-3.67 (m, 6H), 2.58-2.53 (m, 4H). m/z: [ESI⁺] 310 (M+H)⁺.

### Synthetic Details for Compounds of the Invention

### Synthesis of 2-((4-(pyridin-4-yl)piperazin-1-yl)methyl)-6-(trifluoromethyl)-1H-benzo[d]imidazole) (Compound 209)

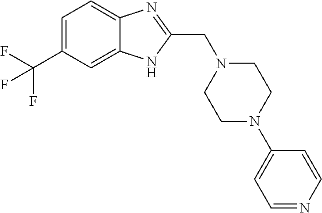 [SMILES: FC(F)(F)c1ccc2nc(CN3CCN(c4ccncc4)CC3)[nH]c2c1]

Compound 2-((4-(pyridin-4-yl)piperazin-1-yl)methyl)-6-(trifluoromethyl)-1H-benzo[d]imidazole was prepared from 1-(pyridin-4-yl)piperazine following a procedure similar to that described for the synthesis of 2-((4-phenylpiperazin-1-yl)methyl)-6-(trifluoromethyl)-1H-benzo[d]imidazole, and was isolated as an off-white solid.

Yield 77 mg (42%). ¹H NMR (400 MHz, DMSO) δ 12.83 (br s, 1H), 8.16 (d, J=6.4 Hz, 2H), 7.89-7.88 (m, 1H), 7.71 (m, 1H), 7.50 (d, J=8.2 Hz, 1H), 6.83 (d, J=6.7 Hz, 2H), 3.86 (s, 2H), 3.36 (t, J=5.4 Hz, 4H), 2.61 (dd, J=5.0, 5.0 Hz, 4H). m/z: [ESI⁺] 362 (M+H)⁺, (C₁₈H₁₈F₃N₅).

### Synthesis of 2-((4-(pyrimidin-4-yl)piperazin-1-yl)methyl)-6-(trifluoromethyl)-1H-benzo[d]imidazole (Compound 203)

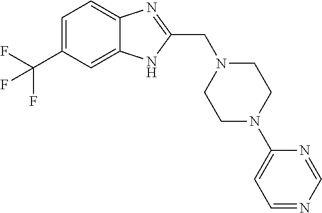 [SMILES: FC(F)(F)c1ccc2nc(CN3CCN(c4ccncn4)CC3)[nH]c2c1]

Compound 2-((4-(pyrimidin-4-yl)piperazin-1-yl)methyl)-6-(trifluoromethyl)-1H-benzo[d]imidazole was prepared from 4-(piperazin-1-yl)pyrimidine following a procedure similar to that described for the synthesis of 2-((4-phenylpiperazin-1-yl)methyl)-6-(trifluoromethyl)-1H-benzo[d]imidazole, and was isolated as an off-white solid.

Yield 112 mg (60%). ¹H NMR (400 MHz, DMSO) δ 12.81 (br s, 1H), 8.50 (s, 1H), 8.19 (d, J=6.1 Hz, 1H), 7.88 (s, 1H), 7.71 (d, J=8.4 Hz, 1H), 7.50 (dd, J=1.4, 8.4 Hz, 1H), 6.85-6.83 (m, 1H), 3.86 (s, 2H), 3.67 (dd, J=4.8, 4.8 Hz, 4H), 2.58 (t, J=5.0 Hz, 4H). m/z: [ESI⁺] 363 (M+H)⁺, (C₁₇H₁₇F₃N₆).

### Synthesis of 2-((4-(5,6-dimethylpyrimidin-4-yl)piperazin-1-yl)methyl)-6-(trifluoromethyl)-1H-benzo[d]imidazole (Compound 201)

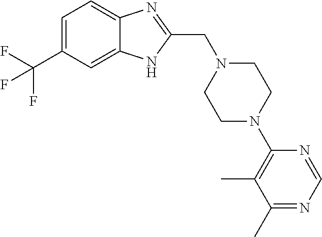 [SMILES: Cc1ncnc(N2CCN(Cc3nc4ccc(C(F)(F)F)cc4[nH]3)CC2)c1C]

To a degassed suspension of 2-(piperazin-1-ylmethyl)-6-(trifluoromethyl)-1H-benzo[d]imidazole (160 mg, 0.563 mmol), 4-chloro-5,6-dimethylpyrimidine (88 mg, 0.619 mmol) and cesium carbonate (367 mg, 1.13 mmol) in anhydrous dioxane (5 mL) was added palladium(II) acetate (13 mg, 0.056 mmol) and RuPhos (53 mg, 0.113 mmol) and the reaction mixture was stirred at 95° C. under a nitrogen atmosphere for 18 hours. After cooling to room temperature, the mixture was diluted with ethyl acetate (30 mL) and filtered through Celite. The filtrate was washed with water (50 mL) and brine (20 mL), dried (MgSO₄), filtered and evaporated. The residue was purified by preparative HPLC to give 2-((4-(5,6-dimethylpyrimidin-4-yl)piperazin-1-yl)methyl)-6-(trifluoromethyl)-1H-benzo[d]imidazole as an off-white solid.

Yield 36 mg (16%). ¹H NMR (400 MHz, DMSO) δ 8.42 (s, 1H), 7.86 (s, 1H), 7.69 (d, J=8.4 Hz, 1H), 7.47 (dd, J=1.4, 8.5 Hz, 1H), 3.85 (s, 2H), 3.27 (t, J=4.6 Hz, 4H), 2.62 (dd, J=4.7, 4.7 Hz, 4H), 2.32 (s, 3H), 2.10 (s, 3H). m/z: [ESI⁺] 391 (M+H)⁺, (C₁₉H₂₁F₃N₆).

### Synthesis of 6-chloro-2-((4-(5-ethylpyrimidin-4-yl)piperazin-1-yl)methyl)-1H-benzo[d]imidazole (Compound 211)

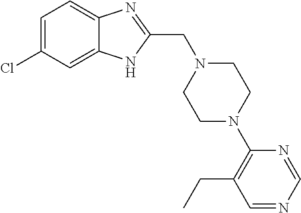 [SMILES: CCc1cncnc1N1CCN(Cc2nc3ccc(Cl)cc3[nH]2)CC1]

Compound 6-chloro-2-((4-(5-ethylpyrimidin-4-yl)piperazin-1-yl)methyl)-1H-benzo[d]imidazole was prepared from 6-chloro-2-(piperazin-1-ylmethyl)-1H-benzo[d]imidazole and 4-chloro-5-ethylpyrimidine following a procedure similar to that described for the synthesis of 2-((4-(5,6-dimethylpyrimidin-4-yl)piperazin-1-yl)methyl)-6-(trifluoromethyl)-1H-benzo[d]imidazole, and was isolated as an off-white solid.

Yield 38 mg (23%). ¹H NMR (400 MHz, DMSO) δ 8.53 (s, 1H), 8.26 (s, 1H), 7.58 (m, 1H), 7.52 (d, J=8.5 Hz, 1H), 7.19 (dd, J=2.1, 8.5 Hz, 1H), 3.80 (s, 2H), 3.45-3.40 (m, 4H), 2.63-2.57 (m, 6H), 1.19 (t, J=7.5 Hz, 3H). m/z: [ESI⁺] 357 (M+H)⁺, (C₁₈H₂₁ClN₆).

### Synthesis of 2-((4-(5,6-dimethylpyrimidin-4-yl)piperazin-1-yl)methyl)-1H-indole (Compound 202)

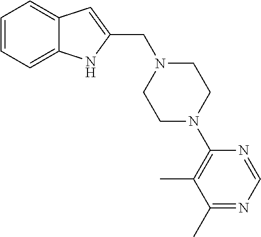 [SMILES: Cc1ncnc(N2CCN(Cc3cc4ccccc4[nH]3)CC2)c1C]

Compound 2-((4-(5,6-dimethylpyrimidin-4-yl)piperazin-1-yl)methyl)-1H-indole was prepared from 2-(piperazin-1-ylmethyl)-1H-indole following a procedure similar to that described for the synthesis of 2-((4-(5,6-dimethylpyrimidin-4-yl)piperazin-1-yl)methyl)-6-(trifluoromethyl)-1H-benzo[d]imidazole, and was isolated as a pink solid.

Yield 52 mg (35%). ¹H NMR (400 MHz, DMSO) δ 11.02 (br s, 1H), 8.41 (s, 1H), 7.44 (d, J=7.8 Hz, 1H), 7.32 (d, J=8.1 Hz, 1H), 7.02 (dd, J=7.5, 7.5 Hz, 1H), 6.94 (dd, J=7.3, 7.3 Hz, 1H), 6.29 (s, 1H), 3.67 (s, 2H), 3.29-3.21 (m, 4H), 2.55 (m, 4H), 2.32 (s, 3H), 2.09 (s, 3H). m/z: [ESI⁺] 322 (M+H)⁺, (C₁₉H₂₃N₅).

### Synthesis of 5-chloro-2-((4-(5,6-dimethylpyrimidin-4-yl)piperazin-1-yl)methyl)-1H-indole (Compound 210)

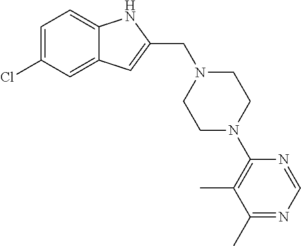 [SMILES: Cc1ncnc(N2CCN(Cc3cc4cc(Cl)ccc4[nH]3)CC2)c1C]

Compound 5-chloro-2-((4-(5,6-dimethylpyrimidin-4-yl)piperazin-1-yl)methyl)-1H-indole was prepared from 5-chloro-2-(piperazin-1-ylmethyl)-1H-indole following a procedure similar to that described for the synthesis of 2-((4-(5,6-dimethylpyrimidin-4-yl)piperazin-1-yl)methyl)-6-(trifluoromethyl)-1H-benzo[d]imidazole, and was isolated as an off-white solid.

Yield 31 mg (27%). ¹H NMR (400 MHz, DMSO) δ 11.29 (br s, 1H), 8.47 (s, 1H), 7.55 (d, J=1.5 Hz, 1H), 7.38 (d, J=8.6 Hz, 1H), 7.08 (dd, J=2.0, 8.6 Hz, 1H), 6.36 (s, 1H), 3.73 (s, 2H), 3.31 (m, 4H), 2.61 (m, 4H), 2.37 (s, 3H), 2.15 (s, 3H). m/z: [ESI⁺] 356 (M+H)⁺, (C₁₉H₂₂ClN₅).

### Synthesis of 2-((4-(pyridin-4-yl)piperazin-1-yl)methyl)-1H-indole (Compound 212)

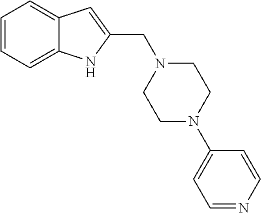 [SMILES: c1ccc2[nH]c(CN3CCN(c4ccncc4)CC3)cc2c1]

To a solution of 1H-indole-2-carbaldehyde (100 mg, 0.689 mmol) and 1-(4-pyridyl)piperazine (124 mg, 0.758 mmol) in anhydrous DCM (5 mL) were added sequentially sodium triacetoxyborohydride (365 mg, 1.72 mmol) and acetic acid (4 μL, 0.069 mmol), and the reaction mixture was stirred at room temperature for 18 hours. The reaction mixture was partitioned between ethyl acetate (40 mL) and a diluted solution of Na₂CO₃ (50%, 30 mL). The organic phase was washed with brine (20 mL), dried (MgSO₄), filtered and evaporated. The residue was purified by preparative HPLC to give 2-((4-(pyridin-4-yl)piperazin-1-yl)methyl)-1H-indole as a white solid.

Yield 48 mg (24%). ¹H NMR (400 MHz, DMSO) δ 11.08 (br s, 1H), 8.20 (m, 2H), 7.50 (d, J=7.8 Hz, 1H), 7.38 (d, J=7.8 Hz, 1H), 7.08 (dd, J=7.1, 7.1 Hz, 1H), 7.00 (dd, J=7.1, 7.1 Hz, 1H), 6.86 (d, J=6.1 Hz, 2H), 6.35 (s, 1H), 3.72 (s, 2H). 8 protons (piperazine) obstructed by solvent/water peak. m/z: [ESI⁺] 293 (M+H)⁺, (C₁₈H₂₀N₄).

### Synthesis of 2-((4-(pyrimidin-4-yl)piperazin-1-yl)methyl)-1H-indole (Compound 213)

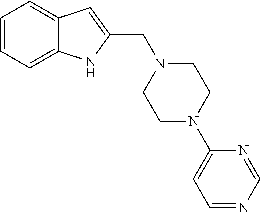 [SMILES: c1ccc2[nH]c(CN3CCN(c4ccncn4)CC3)cc2c1]

Compound 2-((4-(pyrimidin-4-yl)piperazin-1-yl)methyl)-1H-indole was prepared from 4-(piperazin-1-yl)pyrimidine following a procedure similar to that described for the synthesis of 2-((4-(pyridin-4-yl)piperazin-1-yl)methyl)-1H-indole, and was isolated as a white solid.

Yield 127 mg (63%). ¹H NMR (400 MHz, DMSO) δ 11.08 (br s, 1H), 8.53 (s, 1H), 8.22 (d, J=6.3 Hz, 1H), 7.50 (d, J=7.8 Hz, 1H), 7.38 (d, J=7.8 Hz, 1H), 7.08 (dd, J=7.1, 7.1 Hz, 1H), 7.00 (dd, J=7.1, 7.1 Hz, 1H), 6.86 (d, J=6.1 Hz, 1H), 6.35 (s, 1H), 3.72 (s, 2H), 3.67 (t, J=4.2 Hz, 4H), 2.52 (m, 4H). m/z: [ESI⁺] 294 (M+H)⁺, (C₁₇H₁₉N₅).

### Synthesis of 2-[[4-(2-pyridyl)piperazin-1-yl]methyl]-1H-indole (Compound 216)

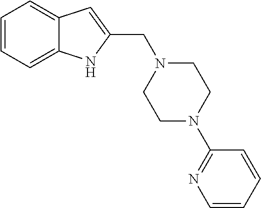 [SMILES: c1ccc(N2CCN(Cc3cc4ccccc4[nH]3)CC2)nc1]

To a solution of indole-2-carboxaldehyde (100 mg, 0.689 mmol) and 1-(pyridin-2-yl)piperazine (124 mg, 0.758 mmol) in anhydrous DCM (3 mL) were added sequentially sodium triacetoxyborohydride (365 mg, 1.72 mmol) and acetic acid (4 μL, 0.069 mmol), and the reaction mixture was stirred at room temperature for 1.5 hours. The reaction mixture was diluted with DCM (10 mL) and washed with a saturated aqueous solution of NaHCO₃ (10 mL) and brine (10 mL). The organic phase was dried (Na₂SO₄), filtered and evaporated. The residue was purified by column chromatography on silica gel (0-10% methanol in DCM) to afford 2-[[4-(2-pyridyl)piperazin-1-yl]methyl]-1H-indole as a brown solid.

Yield 170 mg (84%). ¹H NMR (400 MHz, DMSO) δ 11.05 (s, 1H), 8.11 (dd, J=1.4, 4.8 Hz, 1H), 7.56-7.49 (m, 1H), 7.46 (d, J=7.8 Hz, 1H), 7.34 (d, J=7.8 Hz, 1H), 7.04 (dd, J=7.2, 7.2 Hz, 1H), 6.96 (dd, J=7.2, 7.2 Hz, 1H), 6.81 (d, J=8.7 Hz, 1H), 6.64 (dd, J=5.1, 6.7 Hz, 1H), 6.31 (d, J=1.4 Hz, 1H), 3.67 (s, 2H), 3.52-3.48 (m, 4H), 2.53 (s, 4H). m/z: [ESI⁺] 293 (M+H)⁺, (C₁₈H₂₀N₄).

### Synthesis of 2-[[4-(3-pyridyl)piperazin-1-yl]methyl]-1H-indole (Compound 217)

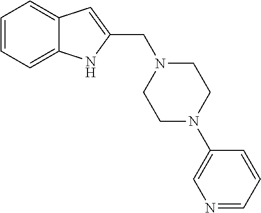 [SMILES: c1cncc(N2CCN(Cc3cc4ccccc4[nH]3)CC2)c1]

Compound 2-[[4-(3-pyridyl)piperazin-1-yl]methyl]-1H-indole was prepared from 1-(pyridin-3-yl)piperazine following a similar procedure to that described for the synthesis of 2-[[4-(2-pyridyl)piperazin-1-yl]methyl]-1H-indole, and was isolated as a white solid.

Yield 65 mg (65%). ¹H NMR (400 MHz, DMSO) δ 11.05 (s, 1H), 8.30 (d, J=2.8 Hz, 1H), 8.00-7.98 (m, 1H), 7.47 (d, J=8.0 Hz, 1H), 7.35-7.30 (m, 2H), 7.23-7.19 (m, 1H), 7.04 (dd, J=7.2, 7.2 Hz, 1H), 6.96 (dd, J=7.2, 7.2 Hz, 1H), 6.32 (d, J=1.4 Hz, 1H), 3.69 (s, 2H), 3.25-3.21 (m, 4H), 2.60-2.54 (m, 4H). m/z: [ESI⁺] 293 (M+H)⁺, (C₁₈H₂₀N₄).

### Synthesis of 2-[[4-(3-pyridylmethyl)piperazin-1-yl]methyl]-1H-indole (Compound 218)

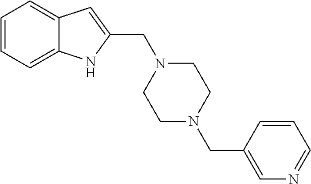 [SMILES: c1cncc(CN2CCN(Cc3cc4ccccc4[nH]3)CC2)c1]

Compound 2-[[4-(3-pyridylmethyl)piperazin-1-yl]methyl]-1H-indole was prepared from 1-(pyridin-3-ylmethyl)piperazine following a similar procedure to that described for the synthesis of 2-[[4-(2-pyridyl)piperazin-1-yl]methyl]-1H-indole, and was isolated as a brown solid.

Yield 70 mg (66%). ¹H NMR (400 MHz, DMSO) δ 10.96 (s, 1H), 8.49-8.45 (m, 2H), 7.71-7.68 (m, 1H), 7.44 (d, J=7.8 Hz, 1H), 7.37-7.31 (m, 2H), 7.02 (dd, J=7.0, 7.0 Hz, 1H), 6.94 (dd, J=7.0, 7.0 Hz, 1H), 6.26 (d, J=1.4 Hz, 1H), 3.60 (s, 2H), 3.51 (s, 2H), 2.42 (br s, 8H). m/z: [ESI⁺] 293 (M+H)⁺, (C₁₉H₂₂N₄).

### Synthesis of 2-[[4-(2-methyl-4-pyridyl)piperazin-1-yl]methyl]-1H-indole (Compound 221)

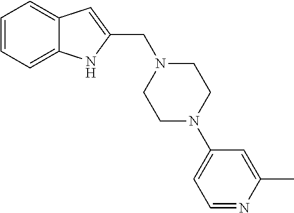 [SMILES: Cc1cc(N2CCN(Cc3cc4ccccc4[nH]3)CC2)ccn1]

To a degassed suspension of 2-(piperazin-1-ylmethyl)-1H-indole (100 mg, 0.464 mmol), 4-bromo-2-methylpyridine (88 mg, 0.511 mmol) and cesium carbonate (303 mg, 0.929 mmol) in anhydrous dioxane (4 mL) was added at room temperature palladium(II) acetate (10 mg, 0.046 mmol) and RuPhos (43 mg, 0.093 mmol). The mixture was sparged for 10 minutes with nitrogen and heated at 95° C. for 2 hours in a sealed tube. After cooling to room temperature, the reaction mixture was partioned between ethyl acetate (20 mL) and water (20 mL). The layers were separated, and the aqueous layer was extracted with ethyl acetate (2×15 mL). The combined organic extracts were washed with brine (10 mL), dried (Na₂SO₄), filtered and concentrated. The residue was purified by preparative HPLC to afford 2-[[4-(2-methyl-4-pyridyl)piperazin-1-yl]methyl]-1H-indole as a yellow solid.

Yield 13 mg (9%). ¹H NMR (400 MHz, DMSO) δ 11.04 (s, 1H), 8.03 (d, J=5.9 Hz, 1H), 7.46 (d, J=8.0 Hz, 1H), 7.34 (d, J=8.0 Hz, 1H), 7.04 (dd, J=7.0, 7.0 Hz, 1H), 6.96 (dd, J=7.0, 7.0 Hz, 1H), 6.69 (d, J=2.5 Hz, 1H), 6.64 (dd, J=2.5, 6.0 Hz, 1H), 6.31 (s, 1H), 3.67 (s, 2H), 3.32-3.29 (m, 4H), 2.56-2.54 (m, 4H), 2.32 (s, 3H). m/z: [ESI⁺] 307 (M+H)⁺, (C₁₉H₂₂N₄).

### Synthesis of 2-[[4-(3-methyl-4-pyridyl)piperazin-1-yl]methyl]-1H-indole (Compound 222)

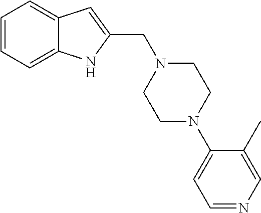 [SMILES: Cc1cnccc1N1CCN(Cc2cc3ccccc3[nH]2)CC1]

Compound 2-[[4-(3-methyl-4-pyridyl)piperazin-1-yl]methyl]-1H-indole was prepared from 1-(3-methylpyridin-4-yl)piperazine following a similar procedure to that described for the synthesis of 2-[[4-(2-pyridyl)piperazin-1-yl]methyl]-1H-indole, and was isolated as an off-white solid.

Yield 25 mg (24%). ¹H NMR (400 MHz, DMSO) δ 11.04 (s, 1H), 8.25-8.20 (m, 2H), 7.47 (d, J=8.0 Hz, 1H), 7.34 (d, J=8.0 Hz, 1H), 7.04 (dd, J=7.2, 7.2 Hz, 1H), 6.96 (dd, J=7.2, 7.2 Hz, 1H), 6.89 (d, J=5.5 Hz, 1H), 6.32 (d, J=1.3 Hz, 1H), 3.71 (s, 2H), 3.03-2.99 (m, 4H), 2.62-2.58 (m, 4H), 2.19 (s, 3H). m/z: [ESI⁺] 307 (M+H)⁺, (C₁₉H₂₂N₄).

### Synthesis of 2-[[4-[3-(trifluoromethyl)-2-pyridyl]piperazin-1-yl]methyl]-1H-indole (Compound 225)

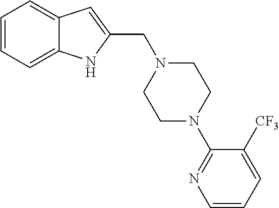 [SMILES: FC(F)(F)c1cccnc1N1CCN(Cc2cc3ccccc3[nH]2)CC1]

Compound 2-[[4-[3-(trifluoromethyl)-2-pyridyl]piperazin-1-yl]methyl]-1H-indole was prepared from 1-(3-(trifluoromethyl)pyridin-2-yl)piperazine following a similar procedure to that described for the synthesis of 2-[[4-(2-pyridyl)piperazin-1-yl]methyl]-1H-indole, and was isolated as an off-white solid.

Yield 82 mg (66%). ¹H NMR (400 MHz, DMSO) δ 11.07 (s, 1H), 8.56 (dd, J=1.3, 4.8 Hz, 1H), 8.10 (dd, J=1.8, 7.8 Hz, 1H), 7.50 (d, J=7.8 Hz, 1H), 7.38 (d, J=7.8 Hz, 1H), 7.22 (dd, J=4.8, 7.1 Hz, 1H), 7.08 (dd, J=6.9, 8.1 Hz, 1H), 7.00 (dd, J=6.9, 8.1 Hz, 1H), 6.35 (d, J=1.3 Hz, 1H), 3.73 (s, 2H), 3.29-3.24 (m, 4H), 2.64-2.60 (m, 4H). m/z: [ESI⁺] 361 (M+H)⁺, (C₁₉H₁₉F₃N₄).

### Synthesis of 2-[[4-(1-methyl-4-piperidyl)piperazin-1-yl]methyl]-1H-indole (Compound 227)

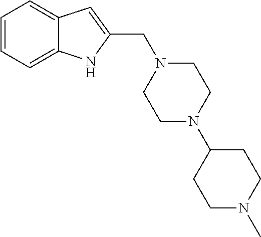 [SMILES: CN1CCC(N2CCN(Cc3cc4ccccc4[nH]3)CC2)CC1]

Compound 2-[[4-(1-methyl-4-piperidyl)piperazin-1-yl]methyl]-1H-indole was prepared from 1-methylpiperidin-4-one and 2-(piperazin-1-ylmethyl)-1H-indole following a similar procedure to that described for the synthesis of 2-[[4-(2-pyridyl)piperazin-1-yl]methyl]-1H-indole, except that it was purified by column chromatography on silica gel (0-20% methanol in DCM, followed by 20% 1 N ammonia/methanol in DCM), and was isolated as an off-white solid.

Yield 44 mg (61%). ¹H NMR (400 MHz, DMSO) δ 10.81 (s, 1H), 7.30 (d, J=7.8 Hz, 1H), 7.18 (d, J=7.1 Hz, 1H), 6.88 (dd, J=1.1, 7.5 Hz, 1H), 6.80 (dd, J=1.1, 7.5 Hz, 1H), 6.12 (d, J=1.3 Hz, 1H), 3.45 (s, 2H), 2.62 (d, J=12.0 Hz, 2H), 2.36-2.32 (m, 4H), 2.27-2.23 (m, 4H), 1.98 (s, 3H), 1.97-1.91 (m, 1H), 1.71-1.63 (m, 2H), 1.55 (d, J=12.0 Hz, 2H), 1.30-1.18 (m, 2H). m/z: [ESI⁺] 313 (M+H)⁺, (C₁₉H₂₈N₄).

### Synthesis of 2-[4-(1H-indol-2-ylmethyl)piperazin-1-yl]thiazole (Compound 229)

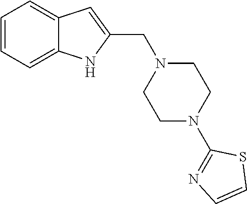 [SMILES: c1ccc2[nH]c(CN3CCN(c4nccs4)CC3)cc2c1]

Compound 2-[4-(1H-indol-2-ylmethyl)piperazin-1-yl]thiazole was prepared from 2-(piperazin-1-yl)thiazole following a similar procedure to that described for the synthesis of 2-[[4-(2-pyridyl)piperazin-1-yl]methyl]-1H-indole, and was isolated as a yellow solid.

Yield 58 mg (57%). ¹H NMR (400 MHz, DMSO) δ 11.05 (s, 1H), 7.46 (d, J=7.8 Hz, 1H), 7.34 (d, J=7.8 Hz, 1H), 7.17 (d, J=3.6 Hz, 1H), 7.04 (dd, J=7.0, 7.0 Hz, 1H), 6.96 (dd, J=7.0, 7.0 Hz, 1H), 6.85 (d, J=3.6 Hz, 1H), 6.31 (d, J=1.3 Hz, 1H), 3.69 (s, 2H), 3.42 (dd, J=5.1, 5.1 Hz, 4H), 2.56 (dd, J=5.1, 5.1 Hz, 4H). m/z: [ESI⁺] 299 (M+H)⁺, (C₁₆H₁₈N₄S).

### Synthesis of 2-[(4-propylpiperazin-171)methyl]-1H-indole (Compound 230)

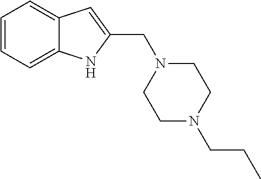 [SMILES: CCCN1CCN(Cc2cc3ccccc3[nH]2)CC1]

Compound 2-[(4-propylpiperazin-1-yl)methyl]-1H-indole was prepared from 1-propylpiperazine following a similar procedure to that described for the synthesis of 2-[[4-(2-pyridyl)piperazin-1-yl]methyl]-1H-indole, and was isolated as a yellow solid.

Yield 38 mg (43%). ¹H NMR (400 MHz, DMSO) δ 10.97 (s, 1H), 7.45 (d, J=8.0 Hz, 1H), 7.32 (d, J=8.0 Hz, 1H), 7.02 (dd, J=7.0, 7.0 Hz, 1H), 6.94 (dd, J=7.0, 7.0 Hz, 1H), 6.27 (s, 1H), 3.59 (s, 2H), 2.45 (br s, 8H), 2.22 (t, J=7.4 Hz, 2H), 1.44-1.40 (m, 2H), 0.85 (t, J=7.4 Hz, 3H). m/z: [ESI⁺] 258 (M+H)⁺, (C₁₆H₂₃N₃).

### Synthesis of [4-(1H-indol-2-ylmethyl)piperazin-1-yl]-(3-pyridyl)methanone (Compound 231)

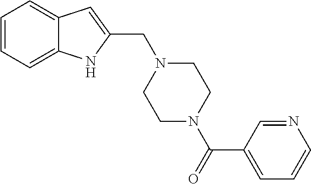 [SMILES: O=C(c1cccnc1)N1CCN(Cc2cc3ccccc3[nH]2)CC1]

Compound [4-(1H-indol-2-ylmethyl)piperazin-1-yl]-(3-pyridyl)methanone was prepared from piperazin-1-yl(pyridin-3-yl)methanone following a similar procedure to that described for the synthesis of 2-[[4-(2-pyridyl)piperazin-1-yl]methyl]-1H-indole, except it was further purified by preparative HPLC, and was isolated as a white solid.

Yield 41 mg (37%). ¹H NMR (400 MHz, DMSO) δ 11.03 (s, 1H), 8.65 (dd, J=1.9, 4.8 Hz, 1H), 8.61 (s, 1H), 7.85-7.81 (m, 1H), 7.50-7.44 (m, 2H), 7.33 (d, J=8.0 Hz, 1H), 7.03 (dd, J=7.2, 7.2 Hz, 1H), 6.95 (dd, J=7.2, 7.2 Hz, 1H), 6.29 (d, J=1.4 Hz, 1H), 3.68 (s, 2H), 3.37-3.37 (br s, 4H), 2.41-2.46 (br s, 4H). m/z: [ESI⁺] 321 (M+H)⁺, (C₁₉H₂₀N₄O).

### Synthesis of Cyclohexyl-[4-(1H-indol-2-ylmethyl)piperazin-1-yl]methanone (Compound 232)

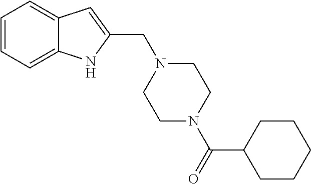 [SMILES: O=C(C1CCCCC1)N1CCN(Cc2cc3ccccc3[nH]2)CC1]

Compound cyclohexyl-[4-(1H-indol-2-ylmethyl)piperazin-1-yl]methanone was prepared from cyclohexyl(piperazin-1-yl)methanone following a similar procedure to that described for the synthesis of 2-[[4-(2-pyridyl)piperazin-1-yl]methyl]-1H-indole, and was isolated as an off-white solid.

Yield 76 mg (68%). ¹H NMR (400 MHz, DMSO) δ 11.01 (s, 1H), 7.45 (d, J=8.0 Hz, 1H), 7.33 (d, J=8.0 Hz, 1H), 7.03 (dd, J=7.0, 7.0 Hz, 1H), 6.95 (dd, J=7.0, 7.0 Hz, 1H), 6.28 (d, J=1.3 Hz, 1H), 3.64 (s, 2H), 3.52-3.44 (m, 4H), 2.58-2.54 (m, 1H), 2.45-2.35 (m, 4H), 1.70-1.58 (m, 5H), 1.40-1.30 (m, 4H) 1.20-1.12 (m, 1H). m/z: [ESI⁺] 326 (M+H)⁺, (C₂₀H₂₇N₃O).

### Synthesis of 2-[(4-cyclopentylpiperazin-1-yl)methyl]-1H-indole (Compound 233)

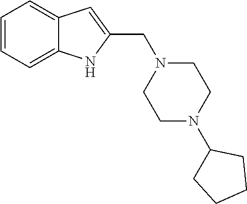 [SMILES: c1ccc2[nH]c(CN3CCN(C4CCCC4)CC3)cc2c1]

Compound 2-[(4-cyclopentylpiperazin-1-yl)methyl]-1H-indole was prepared from 1-cyclopentylpiperazine following a similar procedure to that described for the synthesis of 2-[[4-(2-pyridyl)piperazin-1-yl]methyl]-1H-indole, and was isolated as a white solid.

Yield 70 mg (72%). ¹H NMR (400 MHz, DMSO) δ 10.96 (s, 1H), 7.44 (d, J=8.0 Hz, 1H), 7.32 (d, J=8.0 Hz, 1H), 7.02 (dd, J=7.0, 7.0 Hz, 1H), 6.94 (dd, J=7.0, 7.0 Hz, 1H), 6.26 (d, J=1.3 Hz, 1H), 3.58 (s, 2H), 2.49-2.36 (m, 9H), 1.79-1.71 (m, 2H), 1.63-1.44 (m, 4H), 1.35-1.24 (m, 2H). m/z: [ESI⁺] 284 (M+H)⁺, (C₁₈H₂₅N₃).

### Synthesis of 1-[4-(1H-indol-2-ylmethyl)piperazin-1-yl]isoquinoline (Compound 224)

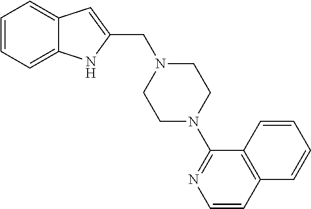 [SMILES: c1ccc2[nH]c(CN3CCN(c4nccc5ccccc45)CC3)cc2c1]

Compound 1-[4-(1H-indol-2-ylmethyl)piperazin-1-yl]isoquinoline was prepared from 1-(piperazin-1-yl)isoquinoline following a similar procedure to that described for the synthesis of 2-[[4-(2-pyridyl)piperazin-1-yl]methyl]-1H-indole, and was isolated as a yellow solid.

Yield 89 mg (76%). ¹H NMR (400 MHz, DMSO) δ 11.08 (s, 1H), 8.11 (d, J=5.6 Hz, 1H), 8.08 (d, J=8.3 Hz, 1H), 7.88 (d, J=8.0 Hz, 1H), 7.70 (dd, J=7.0, 7.0 Hz, 1H), 7.60 (dd, J=7.0, 7.0 Hz, 1H), 7.48 (d, J=7.9 Hz, 1H), 7.39 (d, J=5.6 Hz, 1H), 7.35 (d, J=8.3 Hz, 1H), 7.04 (dd, J=7.2, 7.2 Hz, 1H), 6.96 (dd, J=7.2, 7.2 Hz, 1H), 6.35 (d, J=1.3 Hz, 1H), 3.76 (s, 2H), 3.39-3.35 (m, 4H), 2.75-2.71 (m, 4H). m/z: [ESI⁺] 343 (M+H)⁺, (C₂₂H₂₂N₄).

### Synthesis of 4-[4-(1H-indol-2-ylmethyl)piperazin-1-yl]furo[3,2-c]pyridine (Compound 235)

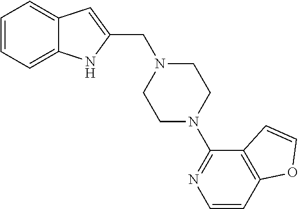 [SMILES: c1ccc2[nH]c(CN3CCN(c4nccc5occc45)CC3)cc2c1]

Compound 4-[4-(1H-indol-2-ylmethyl)piperazin-1-yl]furo[3,2-c]pyridine was prepared from 4-(piperazin-1-yl)furo[3,2-c]pyridine following a similar procedure to that described for the synthesis of 2-[[4-(2-pyridyl)piperazin-1-yl]methyl]-1H-indole, except it was further purified by SCX-2 ion exchange chromatography (2 g, 0.6 mmol/g loading, washed with MeOH and eluted with 1 M ammonia/MeOH), and was isolated as an off-white solid.

Yield 65 mg (57%). ¹H NMR (400 MHz, DMSO) δ 11.06 (s, 1H), 7.97 (d, J=5.8 Hz, 1H), 7.94 (d, J=2.3 Hz, 1H), 7.47 (d, J=8.0 Hz, 1H), 7.34 (d, J=8.0 Hz, 1H), 7.18-7.16 (m, 1H), 7.06-7.02 (m, 2H), 6.96 (dd, J=7.2, 7.2 Hz, 1H), 6.32 (s, 1H), 3.71-3.65 (m, 6H), 2.61-2.59 (m, 4H). m/z: [ESI⁺] 333 (M+H)⁺, (C₂₀H₂₀N₄O).

### Synthesis of 4-[4-(1H-indol-2-ylmethyl)piperazin-1-yl]quinolone (Compound 236)

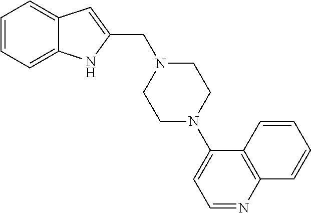 [SMILES: c1ccc2[nH]c(CN3CCN(c4ccnc5ccccc45)CC3)cc2c1]

Compound 4-[4-(1H-indol-2-ylmethyl)piperazin-1-yl]quinolone was prepared from 4-(piperazin-1-yl)quinoline following a similar procedure to that described for the synthesis of 2-[[4-(2-pyridyl)piperazin-1-yl]methyl]-1H-indole, and was isolated as a white solid.

Yield 50 mg (42%). ¹H NMR (400 MHz, DMSO) δ 11.07 (s, 1H), 8.69 (d, J=5.0 Hz, 1H), 8.02 (d, J=8.4 Hz, 1H), 7.95 (d, J=8.4 Hz, 1H), 7.70 (dd, J=7.6, 7.6 Hz, 1H), 7.55 (dd, J=7.6, 7.6 Hz, 1H), 7.48 (d, J=8.0 Hz, 1H), 7.35 (d, J=8.0 Hz, 1H), 7.07-6.94 (m, 3H), 6.36 (s, 1H), 3.78 (s, 2H), 3.23 (br s, 4H), 2.75 (br s, 4H). m/z: [ESI⁺] 343 (M+H)⁺, (C₂₂H₂₂N₄).

### Synthesis of 2-[4-(1H-indol-2-ylmethyl)piperazin-1-yl]quinolone (Compound 237)

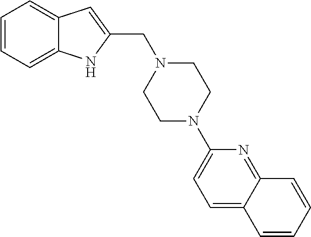 [SMILES: c1ccc2nc(N3CCN(Cc4cc5ccccc5[nH]4)CC3)ccc2c1]

Compound 2-[4-(1H-indol-2-ylmethyl)piperazin-1-yl]quinolone was prepared from 2-(piperazin-1-yl)quinoline following a similar procedure to that described for the synthesis of 2-[[4-(2-pyridyl)piperazin-1-yl]methyl]-1H-indole, and was isolated as an off-white solid.

Yield 73 mg (62%). ¹H NMR (400 MHz, DMSO) δ 11.06 (s, 1H), 8.04 (d, J=9.0 Hz, 1H), 7.70 (d, J=7.5 Hz, 1H), 7.58-7.50 (m, 2H), 7.47 (d, J=7.5 Hz, 1H), 7.35 (d, J=8.0 Hz, 1H), 7.26-7.20 (m, 2H), 7.06-7.02 (dd, J=7.2, 7.2 Hz, 1H), 6.96 (dd, J=7.2, 7.2 Hz, 1H), 6.32 (d, J=1.3 Hz, 1H), 3.74-3.72 (m, 4H), 3.69 (s, 2H), 2.58-2.56 (m, 4H). m/z: [ESI⁺] 343 (M+H)⁺, (C₂₂H₂₂N₄).

### Synthesis of 6-(4-((1H-indol-2-yl)methyl)piperazin-1-yl)-1H-indazole Compound 286)

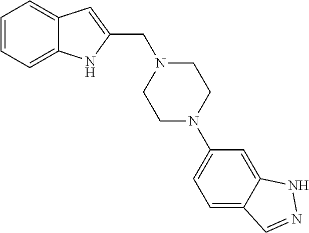 [SMILES: c1ccc2[nH]c(CN3CCN(c4ccc5cn[nH]c5c4)CC3)cc2c1]

Compound 6-(4-((1H-indol-2-yl)methyl)piperazin-1-yl)-1H-indazole was prepared from 1H-indole-2-carbaldehyde and 6-(piperazin-1-yl)-1H-indazole following a similar procedure to that described for the synthesis of 2-[[4-(2-pyridyl)piperazin-1-yl]methyl]-1H-indole, except it was purified by preparative HPLC and was isolated as a white solid.

Yield 24 mg (21%). ¹H NMR (400 MHz, DMSO) δ 12.63 (s, 1H), 11.05 (s, 1H), 7.86 (s, 1H), 7.55 (d, J=8.9 Hz, 1H), 7.47 (d, J=8.0 Hz, 1H), 7.34 (d, J=8.0 Hz, 1H), 7.04 (dd, J=7.0, 7.0 Hz, 1H), 6.98-6.91 (m, 2H), 6.78 (s, 1H), 6.33 (d, J=1.1 Hz, 1H), 3.70 (s, 2H), 3.24-3.18 (m, 4H), 2.63-2.59 (m, 4H). m/z: [ESI⁺] 343 (M+H)⁺, (C₂₀H₂₁N₅).

### Synthesis of 2-[[4-(3-methyl-2-pyridyl)piperazin-1-yl]methyl]-1H-indole (Compound 238)

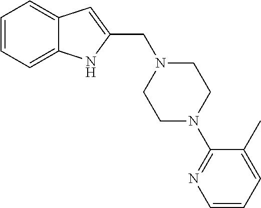 [SMILES: Cc1cccnc1N1CCN(Cc2cc3ccccc3[nH]2)CC1]

Compound 2-[[4-(3-methyl-2-pyridyl)piperazin-1-yl]methyl]-1H-indole was prepared from 1-(3-methylpyridin-2-yl)piperazine following a similar procedure to that described for the synthesis of 2-[[4-(2-pyridyl)piperazin-1-yl]methyl]-1H-indole, and was isolated as an off-white solid.

Yield 55 mg (52%). ¹H NMR (400 MHz, DMSO) δ 11.05 (s, 1H), 8.10 (d, J=4.8 Hz, 1H), 7.49-7.45 (m, 2H), 7.34 (d, J=7.7 Hz, 1H), 7.04 (dd, J=7.0, 7.0 Hz, 1H), 6.97-6.90 (m, 2H), 6.31 (d, J=1.3 Hz, 1H), 3.69 (s, 2H), 3.11-3.07 (m, 4H), 2.61-2.57 (m, 4H), 2.23 (s, 3H). m/z: [ESI⁺] 307 (M+H)⁺, (C₁₉H₂₂N₄).

### Synthesis of 1-[4-(1H-indol-2-ylmethyl)piperazin-1-yl]-3-methyl-butan-1-one (Compound 239)

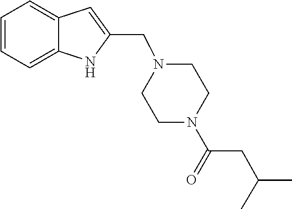 [SMILES: CC(C)CC(=O)N1CCN(Cc2cc3ccccc3[nH]2)CC1]

To a solution of isovaleric acid (28 μL, 0.255 mmol) in anhydrous DMF at room temperature (1 mL) was added DIPEA (0.12 mL, 0.697 mmol), HATU (97 mg, 0.255 mmol) and 2-(piperazin-1-ylmethyl)-1H-indole (50 mg, 0.232 mmol), and the reaction was stirred at room temperature for 1 hour. The reaction mixture was partitioned between ethyl acetate (20 mL) and water (10 mL). The layers were separated, and the organic phase was washed with water (10 mL), water/brine 1:1 (10 mL) and brine (10 mL). The organic layer was dried (Na₂SO₄), filtered and evaporated and the residue was purified by column chromatography on silica gel (0-10% methanol in DCM) to furnish a yellow gum. This material was further purified by preparative HPLC to afford 1-[4-(1H-indol-2-ylmethyl)piperazin-1-yl]-3-methyl-butan-1-one as an off-white solid.

Yield 60 mg (86%). ¹H NMR (400 MHz, DMSO) δ 11.01 (s, 1H), 7.45 (d, J=8.0 Hz, 1H), 7.33 (d, J=8.0 Hz, 1H), 7.04 (dd, J=7.5, 7.5 Hz, 1H), 6.95 (dd, J=7.5, 7.5 Hz, 1H), 6.28 (d, J=1.3 Hz, 1H), 3.64 (s, 2H), 3.49-3.44 (m, 4H), 2.42-2.34 (m, 4H), 2.18 (d, J=7.0 Hz, 2H), 2.01-1.93 (m, 1H), 0.89 (d, J=6.7 Hz, 6H). m/z: [ESI⁺] 300 (M+H)⁺, (C₁₈H₂₅N₃O).

### Synthesis of 2-[(4-phenylpiperazin-1-yl)methyl]-1H-indole (Compound 241)

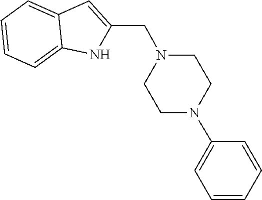 [SMILES: c1ccc(N2CCN(Cc3cc4ccccc4[nH]3)CC2)cc1]

Compound 2-[(4-phenylpiperazin-1-yl)methyl]-1H-indole was prepared from 1-phenylpiperazine following a similar procedure to that described for the synthesis of 2-[[4-(2-pyridyl)piperazin-1-yl]methyl]-1H-indole, and was isolated as a yellow solid.

Yield 93 mg (81%). ¹H NMR (400 MHz, DMSO) δ 11.04 (s, 1H), 7.47 (d, J=8.0 Hz, 1H), 7.35 (d, J=8.0 Hz, 1H), 7.21 (dd, J=8.2, 8.2 Hz, 2H), 7.04 (dd, J=7.1, 7.1 Hz, 1H), 6.98-6. (m, 3H), 6.78 (dd, J=7.1, 7.1 Hz, 1H), 6.32 (s, 1H), 3.68 (s, 2H), 3.19-3.13 (m, 4H), 2.59-2.55 (m, 4H). m/z: [ESI⁺] 292 (M+H)⁺, (C₁₉H₂₁N₃).

### Synthesis of 4-[4-(1H-indol-2-ylmethyl)piperazin-1-yl]-1H-pyrrolo[2,3-b]pyridine (Compound 242)

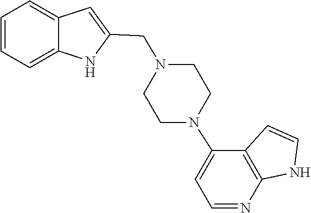 [SMILES: c1ccc2[nH]c(CN3CCN(c4ccnc5[nH]ccc45)CC3)cc2c1]

To a solution of 4-(4-((1H-indol-2-yl)methyl)piperazin-1-yl)-1-((2-(trimethylsilyl)ethoxy)-methyl)-1H-pyrrolo[2,3-b]pyridine (142 mg, 0.308 mmol) in anhydrous THF (3 mL) was added at room temperature TBAF (1 M THF, 0.92 mL, 0.92 mmol) and ethylenediamine (62 μL, 0.92 mmol), and the reaction mixture was heated at 80° C. for 18 hours. After cooling to room temperature, the mixture was partitioned between ethyl acetate (20 mL) and water (20 mL). The layers were separated, and the organic phase was washed with water (2×15 mL) and brine (10 mL), dried (Na₂SO₄), filtered and evaporated. The residue was purified by column chromatography on silica gel (0-8% methanol in DCM) to afford 4-[4-(1H-indol-2-ylmethyl)piperazin-1-yl]-1H-pyrrolo[2,3-b]pyridine as a yellow solid.

Yield 52 mg (51%). ¹H NMR (400 MHz, DMSO) δ 11.40 (s, 1H), 11.05 (s, 1H), 7.94 (d, J=5.5 Hz, 1H), 7.45 (d, J=7.9 Hz, 1H), 7.34 (d, J=7.9 Hz, 1H), 7.23-7.21 (m, 1H), 7.04 (dd, J=7.6, 7.6 Hz, 1H), 6.95 (dd, J=7.2, 7.2 Hz, 1H), 6.47-6.44 (m, 1H), 6.41 (d, J=5.5 Hz, 1H), 6.32 (d, J=1.3 Hz, 1H), 3.71 (s, 2H), 3.45-3.40 (m, 4H), 2.65-2.61 (m, 4H). m/z: [ESI⁺] 332 (M+H)⁺, (C₂₀H₂₁N₅).

### Synthesis of 2-[[4-(5-methylpyrimidin-4-yl)piperazin-1-yl]methyl]-1H-indole Formate (Compound 266)

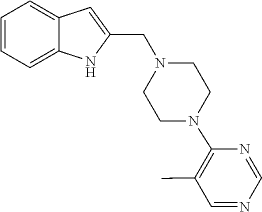 [SMILES: Cc1cncnc1N1CCN(Cc2cc3ccccc3[nH]2)CC1]

Compound 2-[[4-(5-methylpyrimidin-4-yl)piperazin-1-yl]methyl]-1H-indole formate was prepared from 5-methyl-4-(piperazin-1-yl)pyrimidine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by preparative HPLC, and was isolated as an off-white solid.

Yield 66 mg (63%). ¹H NMR (400 MHz, DMSO) δ 11.05 (s, 1H), 8.51 (s, 1H), 8.18-8.16 (m, 2H), 7.46 (d, J=7.8 Hz, 1H), 7.34 (d, J=7.8 Hz, 1H), 7.04 (dd, J=7.3, 7.3 Hz, 1H), 6.95 (dd, J=7.3, 7.3 Hz, 1H), 6.31 (s, 1H), 3.68 (s, 2H), 3.50-3.40 (m, 4H), 2.58-2.53 (m, 4H), 2.19 (s, 3H). m/z: [ESI⁺] 308 (M+H)⁺, (C₁₈H₂₁N₅).

### Synthesis of 2-[(4-pyridazin-4-ylpiperazin-1-yl)methyl]-1H-indole Formate (Compound 279)

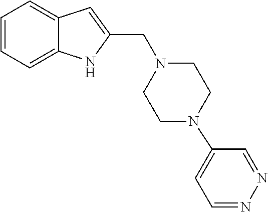 [SMILES: c1ccc2[nH]c(CN3CCN(c4ccnnc4)CC3)cc2c1]

To a degassed suspension of 2-(piperazin-1-ylmethyl)-1H-indole (100 mg, 0.464 mmol), 4-bromopyridazine (81 mg, 0.511 mmol) and cesium carbonate (303 mg, 0.929 mmol) in anhydrous dioxane (4 mL) was added at room temperature palladium(II) acetate (10 mg, 0.046 mmol) and RuPhos (43 mg, 0.093 mmol). The mixture was sparged for 5 minutes with nitrogen and heated at 95° C. for 5 hours in a sealed tube. The reaction was re-charged with 4-bromopyridazine (81 mg, 0.511 mmol), palladium(II) acetate (10 mg, 0.046 mmol) and RuPhos (43 mg, 0.093 mmol) and continued to heat at 95° C. for 18 hours. After cooling to room temperature, the reaction mixture was partitioned between ethyl acetate (20 mL) and water (20 mL). The layers were separated, and the aqueous layer was extracted with ethyl acetate (2×15 mL). The combined organic extracts were washed with brine (10 mL), dried (Na₂SO₄), filtered and concentrated. The residue was purified by column chromatography on silica gel (0-10% methanol in DCM) followed by preparative HPLC to afford 2-[(4-pyridazin-4-ylpiperazin-1-yl)methyl]-1H-indole formate as an orange solid.

Yield 44 mg (28%). ¹H NMR (400 MHz, DMSO) δ 11.05 (s, 1H), 8.97 (d, J=3.0 Hz, 1H), 8.63 (d, J=6.7 Hz, 1H), 8.17 (s, 1H), 7.47 (d, J=7.9 Hz, 1H), 7.34 (d, J=7.9 Hz, 1H), 7.04 (dd, J=7.6, 7.6 Hz, 1H), 6.98-6.92 (m, 2H), 6.31 (d, J=1.3 Hz, 1H), 3.69 (s, 2H), 3.46-2.42 (m, 4H), 2.57-2.53 (m, 4H). m/z: [ESI⁺] 294 (M+H)⁺, (C₁₇H₁₉N₅).

### Synthesis of 2-[(4-pyrimidin-5-ylpiperazin-1-yl)methyl]-1H-indole Formate (Compound 278)

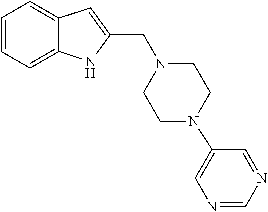 [SMILES: c1ccc2[nH]c(CN3CCN(c4cncnc4)CC3)cc2c1]

To a degassed suspension of 2-(piperazin-1-ylmethyl)-1H-indole (100 mg, 0.464 mmol), 5-bromopyrimidine (81 mg, 0.511 mmol) and cesium carbonate (303 mg, 0.929 mmol) in anhydrous dioxane (4 mL) was added at room temperature palladium(II) acetate (10 mg, 0.046 mmol) and RuPhos (43 mg, 0.093 mmol). The mixture was sparged for 5 minutes with nitrogen and heated at 95° C. for 18 hours in a sealed tube. After cooling to room temperature, the reaction mixture was partitioned between ethyl acetate (20 mL) and water (20 mL). The layers were separated, and the aqueous layer was extracted with ethyl acetate (2×15 mL). The combined organic extracts were washed with brine (10 mL), dried (Na₂SO₄), filtered and concentrated. The residue was purified by column chromatography on silica gel (0-10% methanol in DCM) followed by preparative HPLC to afford 2-[(4-pyrimidin-5-ylpiperazin-1-yl)methyl]-1H-indole formate as an off-white solid.

Yield 47 mg (35%). ¹H NMR (400 MHz, DMSO) δ 11.05 (s, 1H), 8.59 (s, 1H), 8.49 (s, 2H), 8.17 (s, 0.6H), 7.47 (d, J=8.0 Hz, 1H), 7.34 (d, J=8.0 Hz, 1H), 7.04 (dd, J=7.5, 7.5 Hz, 1H), 6.96 (dd, J=7.5, 7.5 Hz, 1H), 6.32 (d, J=1.3 Hz, 1H), 3.69 (s, 2H), 3.32-3.28 (m, 4H), 2.60-2.56 (m, 4H). m/z: [ESI⁺] 294 (M+H)⁺, (C₁₇H₁₉N₅).

### Synthesis of 5-phenyl-2-[[4-(4-pyridyl)piperazin-1-yl]methyl]-1H-indole (Compound 219)

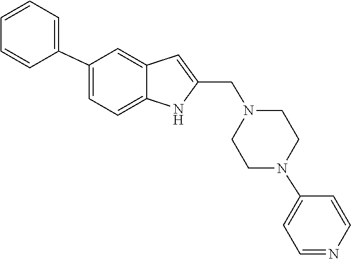 [SMILES: c1ccc(-c2ccc3[nH]c(CN4CCN(c5ccncc5)CC4)cc3c2)cc1]

To a degassed suspension of 5-bromo-2-[[4-(4-pyridyl)piperazin-1-yl]methyl]-1H-indole (50 mg, 0.135 mmol), phenylboronic acid pinacol ester (33 mg, 162 mmol) and cesium carbonate (132 mg, 0.404 mmol) in dioxane (2 mL) and water (0.5 mL) was added at room temperature [1,1′-bis(diphenyl-phosphino)ferrocene]dichloropalladium(II) complex with dichloromethane (11 mg, 0.014 mmol). The mixture was sparged for 5 minutes with nitrogen and heated at 100° C. for 2 hours in a sealed tube. After cooling to room temperature, the reaction mixture was partitioned between ethyl acetate (20 mL) and water (20 mL). The layers were separated, and the aqueous phase was extracted with ethyl acetate (2×15 mL). The combined organic extracts were washed with brine (10 mL), dried (Na₂SO₄), filtered and concentrated. The residue was purified by column chromatography on silica gel (0-20% methanol in DCM) to afford 5-phenyl-2-[[4-(4-pyridyl)piperazin-1-yl]methyl]-1H-indole as a grey solid.

Yield 19 mg (38%). ¹H NMR (400 MHz, DMSO) δ 11.14 (s, 1H), 8.16 (d, J=6.5 Hz, 2H), 7.75 (s, 1H), 7.66 (d, J=7.4 Hz, 2H), 7.47-7.40 (m, 3H), 7.36 (d, J=8.4 Hz, 1H), 7.30 (m, 1H), 6.82 (d, J=5.1 Hz, 2H), 6.39 (d, J=1.3 Hz, 1H), 3.70 (s, 2H), 3.37-3.34 (m, 4H), 2.60-2.53 (m, 4H). m/z: [ESI⁺] 369 (M+H)⁺, (C₂₄H₂₄N₄).

### Synthesis of 5-(3-pyridyl)-2-[[4-(4-pyridyl)piperazin-1-yl]methyl]-1H-indole (Compound 220)

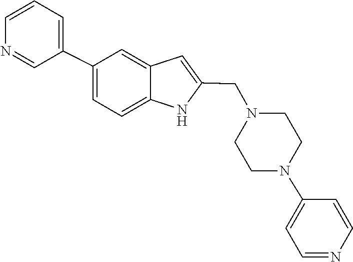 [SMILES: c1cncc(-c2ccc3[nH]c(CN4CCN(c5ccncc5)CC4)cc3c2)c1]

Compound 5-(3-pyridyl)-2-[[4-(4-pyridyl)piperazin-1-yl]methyl]-1H-indole was prepared from 5-bromo-2-[[4-(4-pyridyl)piperazin-1-yl]methyl]-1H-indole following a similar procedure to that described for the synthesis of 5-phenyl-2-[[4-(4-pyridyl)piperazin-1-yl]methyl]-1H-indole, and was isolated as a brown solid.

Yield 24 mg (48%). ¹H NMR (400 MHz, DMSO) δ 11.22 (s, 1H), 8.90 (d, J=1.9 Hz, 1H), 8.51 (d, J=4.7 Hz, 1H), 8.17 (br s, 2H), 8.08-8.04 (m, 1H), 7.82 (d, J=1.5 Hz, 1H), 7.48-7.39 (m, 3H), 6.83 (d, J=5.4 Hz, 2H), 6.42 (s, 1H), 3.71 (s, 2H), 3.40-3.34 (m, 4H), 2.60-2.53 (m, 4H). m/z: [ESI⁺] 370 (M+H)⁺, (C₂₃H₂₃N₅).

### Synthesis of 5-methoxy-2-[[4-(4-pyridyl)piperazin-1-yl]methyl]-1H-indole (Compound 223)

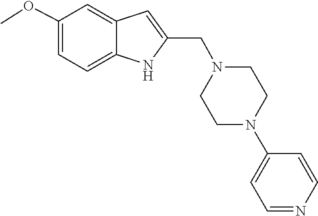 [SMILES: COc1ccc2[nH]c(CN3CCN(c4ccncc4)CC3)cc2c1]

Compound 5-methoxy-2-[[4-(4-pyridyl)piperazin-1-yl]methyl]-1H-indole was prepared from 1-(pyridin-4-yl)piperazine following a similar procedure to that described for the synthesis of 2-[[4-(2-pyridyl)piperazin-1-yl]methyl]-1H-indole, and was isolated as a white solid.

Yield 51 mg (56%). ¹H NMR (400 MHz, DMSO) δ 10.86 (s, 1H), 8.16 (d, J=6.3 Hz, 2H), 7.21 (d, J=8.7 Hz, 1H), 6.97 (d, J=2.5 Hz, 1H), 6.81 (d, J=5.1 Hz, 2H), 6.69 (d, J=8.7 Hz, 1H), 6.23 (d, J=1.4 Hz, 1H), 3.74 (s, 3H), 3.65 (s, 2H), 3.34-3.30 (m, 4H), 2.55-2.51 (m, 4H). m/z: [ESI⁺] 323 (M+H)⁺, (C₁₉H₂₂N₄O).

### Synthesis of 2-[[4-(4-pyridyl)piperazin-1-yl]methyl]-5-(trifluoromethyl)-1H-indole (Compound 226)

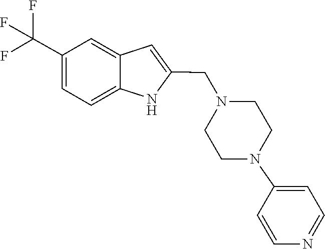 [SMILES: FC(F)(F)c1ccc2[nH]c(CN3CCN(c4ccncc4)CC3)cc2c1]

Compound 2-[[4-(4-pyridyl)piperazin-1-yl]methyl]-5-(trifluoromethyl)-1H-indole was prepared from 5-(trifluoromethyl)-1H-indole-2-carbaldehyde and 1-(pyridin-4-yl)piperazine following a similar procedure to that described for the synthesis of 2-[[4-(2-pyridyl)piperazin-1-yl]methyl]-1H-indole, and was isolated as an off-white solid.

Yield 81 mg (69%). ¹H NMR (400 MHz, DMSO) δ 11.45 (s, 1H), 8.08 (d, J=4.3 Hz, 2H), 7.79 (s, 1H), 7.44 (d, J=8.6 Hz, 1H), 7.26 (d, J=8.6 Hz, 1H), 6.73 (d, J=6.1 Hz, 2H), 6.42 (d, J=1.3 Hz, 1H), 3.65 (s, 2H), 3.31-3.25 (m, 4H), 2.50-2.45 (m, 4H). m/z: [ESI⁺] 361 (M+H)⁺, (C₁₉H₁₉F₃N₄).

### Synthesis of 6-methoxy-2-[[4-(4-pyridyl)piperazin-1-yl]methyl]-1H-indole (Compound 228)

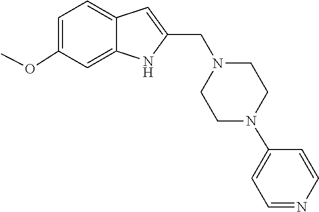 [SMILES: COc1ccc2cc(CN3CCN(c4ccncc4)CC3)[nH]c2c1]

Compound 6-methoxy-2-[[4-(4-pyridyl)piperazin-1-yl]methyl]-1H-indole was prepared from 6-methoxy-1H-indole-2-carbaldehyde and 1-(pyridin-4-yl)piperazine following a similar procedure to that described for the synthesis of 2-[[4-(2-pyridyl)piperazin-1-yl]methyl]-1H-indole, and was isolated as a brown solid.

Yield 91 mg (38%). ¹H NMR (400 MHz, DMSO) δ 10.89 (s, 1H), 8.20 (d, J=6.1 Hz, 2H), 7.37 (d, J=8.6 Hz, 1H), 6.88 (d, J=2.3 Hz, 1H), 6.85 (d, J=6.6 Hz, 2H), 6.66 (dd, J=2.3, 8.6 Hz, 1H), 6.26 (d, J=1.3 Hz, 1H), 3.80 (s, 3H), 3.67 (s, 2H). 8 protons obscured by solvent peaks. m/z: [ESI⁺] 323 (M+H)⁺, (C₁₉H₂₂N₄O).

### Synthesis of 2-[[4-(4-pyridyl)piperazin-1-yl]methyl]-6-(trifluoromethyl)-1H-indole (Compound 255)

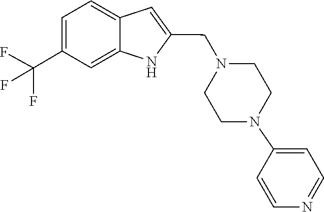 [SMILES: FC(F)(F)c1ccc2cc(CN3CCN(c4ccncc4)CC3)[nH]c2c1]

Compound 2-[[4-(4-pyridyl)piperazin-1-yl]methyl]-6-(trifluoromethyl)-1H-indole was prepared from 6-(trifluoromethyl)-1H-indole-2-carbaldehyde and 1-(pyridin-4-yl)piperazine following a similar procedure to that described for the synthesis of 2-[[4-(2-pyridyl)piperazin-1-yl]methyl]-1H-indole, and was isolated as a brown solid.

Yield 85 mg (72%). ¹H NMR (400 MHz, DMSO) δ 11.53 (s, 1H), 8.16 (d, J=6.5 Hz, 2H), 7.68 (d, J=8.0 Hz, 1H), 7.66 (s, 1H), 7.26 (d, J=8.0 Hz, 1H), 6.82 (d, J=5.0 Hz, 2H), 6.48 (d, J=1.4 Hz, 1H), 3.74 (s, 2H), 3.38-3.33 (m, 4H), 2.58-2.53 (m, 4H). m/z: [ESI⁺] 361 (M+H)⁺, (C₁₉H₁₉F₃N₄).

### Synthesis of 6-chloro-2-[[4-(4-pyridyl)piperazin-1-yl]methyl]-1H-indole (Compound 240)

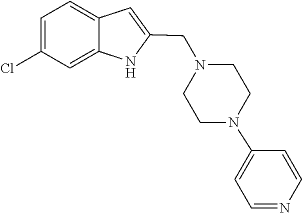 [SMILES: Clc1ccc2cc(CN3CCN(c4ccncc4)CC3)[nH]c2c1]

Compound 6-chloro-2-[[4-(4-pyridyl)piperazin-1-yl]methyl]-1H-indole was prepared from 6-chloro-1H-indole-2-carbaldehyde and 1-(pyridin-4-yl)piperazine following a similar procedure to that described for the synthesis of 2-[[4-(2-pyridyl)piperazin-1-yl]methyl]-1H-indole, and was isolated as an off-white solid.

Yield 40 mg (44%). ¹H NMR (400 MHz, DMSO) δ 11.25 (s, 1H), 8.20 (d, J=6.3 Hz, 2H), 7.51 (d, J=8.0 Hz, 1H), 7.40 (d, J=2.0 Hz, 1H), 7.02 (dd, J=2.0, 8.3 Hz, 1H), 6.85 (d, J=6.3 Hz, 2H), 6.39 (d, J=1.3 Hz, 1H), 3.71 (s, 2H), 3.39-3.33 (m, 4H), 2.60-2.57 (m, 4H). m/z: [ESI⁺] 327 (M+H)⁺, (C₁₈H₁₉ClN₄).

### Synthesis of 4-chloro-2-[[4-(4-pyridyl)piperazin-1-yl]methyl]-1H-indole (Compound 243)

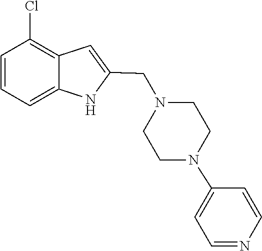 [SMILES: Clc1cccc2[nH]c(CN3CCN(c4ccncc4)CC3)cc12]

Compound 4-chloro-2-[[4-(4-pyridyl)piperazin-1-yl]methyl]-1H-indole was prepared from 4-chloro-1H-indole-2-carbaldehyde and 1-(pyridin-4-yl)piperazine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH), and was isolated as an off-white solid.

Yield 83 mg (91%). ¹H NMR (400 MHz, DMSO) δ 11.45 (s, 1H), 8.16 (d, J=6.0 Hz, 2H), 7.34-7.32 (m, 1H), 7.06-7.03 (m, 2H), 6.82 (d, J=6.0 Hz, 2H), 6.37 (d, J=1.4 Hz, 1H), 3.70 (s, 2H), 3.37-3.33 (m, 4H), 2.58-2.52 (m, 4H). m/z: [ESI⁺] 327 (M+H)⁺, (C₁₈H₁₉ClN₄).

### Synthesis of 5-chloro-2-[[4-(4-pyridyl)piperazin-1-yl]methyl]-1H-indole Formate (Compound 245)

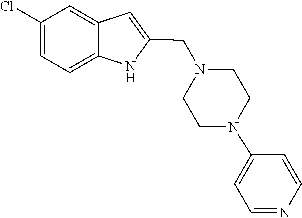 [SMILES: Clc1ccc2[nH]c(CN3CCN(c4ccncc4)CC3)cc2c1]

Compound 5-chloro-2-[[4-(4-pyridyl)piperazin-1-yl]methyl]-1H-indole was prepared from 5-chloro-1H-indole-2-carbaldehyde and 1-(pyridin-4-yl)piperazine following a similar procedure to that described for the synthesis of 2-[[4-(2-pyridyl)piperazin-1-yl]methyl]-1H-indole, except it was purified by preparative HPLC to afford 5-chloro-2-[[4-(4-pyridyl)piperazin-1-yl]methyl]-1H-indole formate as an off-white solid.

Yield 32 mg (36%). ¹H NMR (400 MHz, DMSO) δ 11.27 (s, 1H), 8.18 (s, 1H), 8.16 (d, J=6.5 Hz, 2H), 7.51 (d, J=2.1 Hz, 1H), 7.34 (d, J=8.7 Hz, 1H), 7.04 (dd, J=2.1, 8.7 Hz, 1H), 6.83 (d, J=6.5 Hz, 2H), 6.32 (d, J=1.3 Hz, 1H), 3.68 (s, 2H), 3.38-3.32 (m, 4H). 4 protons obscured by solvent peak. m/z: [ESI⁺] 327 (M+H)⁺, (C₁₈H₁₉ClN₄).

### Synthesis of 7-chloro-2-[[4-(4-pyridyl)piperazin-1-yl]methyl]-1H-indole (Compound 247)

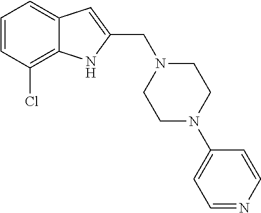 [SMILES: Clc1cccc2cc(CN3CCN(c4ccncc4)CC3)[nH]c12]

Compound 7-chloro-2-[[4-(4-pyridyl)piperazin-1-yl]methyl]-1H-indole was prepared from 7-chloro-1H-indole-2-carbaldehyde and 1-(pyridin-4-yl)piperazine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH), and was isolated as an off-white solid.

Yield 63 mg (69%). ¹H NMR (400 MHz, DMSO) δ 11.35 (s, 1H), 8.16 (dd, J=1.4, 5.0 Hz, 2H), 7.47 (d, J=7.8 Hz, 1H), 7.13 (d, J=7.8 Hz, 1H), 6.98 (dd, J=7.8, 7.8 Hz, 1H), 6.81 (dd, J=1.5, 5.0 Hz, 2H), 6.44 (d, J=1.5 Hz, 1H), 3.72 (s, 2H), 3.38-3.34 (m, 4H), 2.60-2.53 (m, 4H). m/z: [ESI⁺] 327 (M+H)⁺, (C₁₈H₁₉ClN₄).

### Synthesis of 3-chloro-2-[[4-(4-pyridyl)piperazin-1-yl]methyl]-1H-indole (Compound 248)

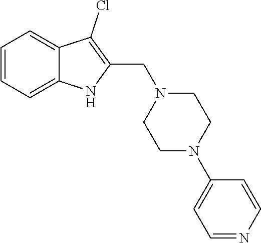 [SMILES: Clc1c(CN2CCN(c3ccncc3)CC2)[nH]c2ccccc12]

Compound 3-chloro-2-[[4-(4-pyridyl)piperazin-1-yl]methyl]-1H-indole was prepared from 3-chloro-1H-indole-2-carbaldehyde and 1-(pyridin-4-yl)piperazine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH), and was isolated as an off-white solid.

Yield 52 mg (57%). ¹H NMR (400 MHz, DMSO) δ 11.45 (s, 1H), 8.16 (dd, J=1.4, 5.0 Hz, 2H), 7.43 (d, J=8.0 Hz, 1H), 7.40 (d, J=8.0 Hz, 1H), 7.15 (dd, J=7.9, 7.9 Hz, 1H), 7.10 (dd, J=7.9, 7.9 Hz, 1H), 6.81 (dd, J=1.4, 5.0 Hz, 2H), 3.74 (s, 2H), 3.37-3.33 (m, 4H), 2.59-2.54 (m, 4H). m/z: [ESI⁺] 327 (M+H)⁺, (C₁₈H₁₉ClN₄).

### Synthesis of 2-[[4-(4-pyridyl)piperazin-1-yl]methyl]-1H-indol-5-amine (Compound 246)

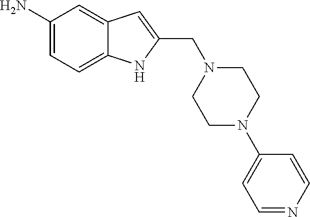 [SMILES: Nc1ccc2[nH]c(CN3CCN(c4ccncc4)CC3)cc2c1]

To a suspension of 5-nitro-2-((4-(pyridin-4-yl)piperazin-1-yl)methyl)-1H-indole (636 mg, 1.89 mmol) in ethanol (24 mL) and water (6 mL) were added at room temperature iron (632 mg, 11.3 mmol) and ammonium chloride (61 mg, 1.13 mmol) and the mixture was heated to 90° C. and stirred for 18 hours. After cooling to room temperature, the reaction mixture was filtered and partitioned between ethyl acetate (50 mL) and saturated aqueous solution of NaHCO₃ (50 mL). The layers were separated, and the organic phase was washed with water (50 mL), brine (50 mL), dried (Na₂SO₄), filtered and concentrated to afford 2-[[4-(4-pyridyl)piperazin-1-yl]methyl]-1H-indol-5-amine as a beige solid.

Yield 275 mg (47%). ¹H NMR (400 MHz, DMSO) δ 10.52 (s, 1H), 8.15 (dd, J=1.5, 5.0 Hz, 2H), 7.02 (d, J=8.5 Hz, 1H), 6.81 (dd, J=1.5, 5.0 Hz, 2H), 6.62 (d, J=2.0 Hz, 1H), 6.44 (dd, J=2.0, 8.5 Hz, 1H), 6.02 (d, J=1.4 Hz, 1H), 4.38 (br s, 2H), 3.59 (s, 2H), 3.33-3.30 (m, 4H), 2.52-2.48 (m, 4H). m/z: [ESI⁺] 308 (M+H)⁺, (C₁₈H₂₁N₅).

### Synthesis of N-[2-[[4-(4-pyridyl)piperazin-1-yl-methyl]-1H-indol-5-yl]benzamide (Compound 249)

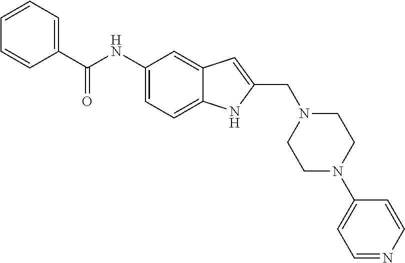 [SMILES: O=C(Nc1ccc2[nH]c(CN3CCN(c4ccncc4)CC3)cc2c1)c1ccccc1]

To a solution of benzoic acid (13 mg, 0.11 mmol) in anhydrous DMF (1 mL) were added at room temperature HATU (41 mg, 0.11 mmol), DIPEA (51 μL, 0.29 mmol) and 2-[[4-(4-pyridyl)piperazin-1-yl]methyl]-1H-indol-5-amine (30 mg, 0.10 mmol) and the mixture was stirred at room temperature for 1 hour. The reaction mixture was partitioned between ethyl acetate (20 mL) and water (10 mL). The layers were separated, and the organic phase was washed with water (10 mL), water/brine 1:1 (10 mL) and brine (10 mL). The organic layer was dried (Na₂SO₄), filtered and evaporated and the residue was purified by column chromatography on silica gel (0-10% 1 M ammonia/methanol in DCM) to furnish N-[2-[[4-(4-pyridyl)piperazin-1-yl]methyl]-1H-indol-5-yl]benzamide as a pink solid.

Yield 15 mg (38%). ¹H NMR (400 MHz, DMSO) δ 11.03 (s, 1H), 10.06 (s, 1H), 8.16 (dd, J=1.5, 5.1 Hz, 2H), 8.00 (d, J=7.0 Hz, 2H), 7.91 (s, 1H), 7.59-7.51 (m, 3H), 7.36 (dd, J=1.9, 8.7 Hz, 1H), 7.28 (d, J=7.5 Hz, 1H), 6.83 (dd, J=1.5, 5.1 Hz, 2H), 6.31 (d, J=1.4 Hz, 1H), 3.68 (s, 2H), 3.40-3.34 (m, 4H), 2.60-2.53 (m, 4H). m/z: [ESI⁺] 412 (M+H)⁺, (C₂₅H₂₅N₅O).

### Synthesis of N-phenyl-2-[[4-(4-pyridyl)piperazin-1-yl]methyl]-1H-indole-5-carboxamide (Compound 250)

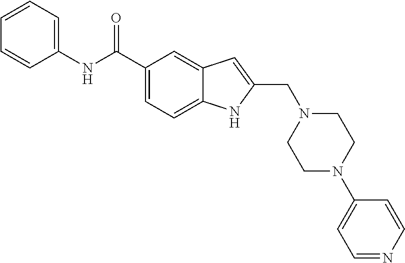 [SMILES: O=C(Nc1ccccc1)c1ccc2[nH]c(CN3CCN(c4ccncc4)CC3)cc2c1]

To a solution of 2-((4-(pyridin-4-yl)piperazin-1-yl)methyl)-1H-indole-5-carboxylic acid (32 mg, 0.10 mmol) in anhydrous DMF (1 mL) were added at room temperature aniline (10 μL, 0.11 mmol), HATU (40 mg, 0.11 mmol) and DIPEA (50 μL, 0.29 mmol) and the mixture was stirred at this temperature for 1 hour and then at 60° C. for 1 hour. After cooling to room temperature, the reaction mixture was partitioned between ethyl acetate (20 mL) and water (10 mL). The layers were separated, and the organic phase was washed with water (10 mL), water/brine 1:1 (10 mL) and brine (10 mL). The organic layer was dried (Na₂SO₄), filtered and evaporated and the residue purified by column chromatography on silica gel (0-10% 1 M ammonia/methanol in DCM) to furnish N-phenyl-2-[[4-(4-pyridyl)piperazin-1-yl]methyl]-1H-indole-5-carboxamide as a white solid.

Yield 20 mg (51%). ¹H NMR (400 MHz, DMSO) δ 11.39 (s, 1H), 10.09 (s, 1H), 8.20 (d, J=1.6 Hz, 1H), 8.17 (dd, J=1.3, 5.2 Hz, 2H), 7.82 (d, J=8.7 Hz, 2H), 7.72 (d, J=8.5 Hz, 1H), 7.45 (d, J=8.5 Hz, 1H), 7.35 (dd, J=8.0, 8.0 Hz, 2H), 7.08 (dd, J=7.4, 7.4 Hz, 1H), 6.85 (dd, J=1.3, 5.2 Hz, 2H), 6.49 (d, J=1.1 Hz, 1H), 3.72 (s, 2H), 3.40-3.35 (m, 4H), 2.60-2.53 (m, 4H). m/z: [ESI⁺] 412 (M+H)⁺, (C₂₅H₂₅N₅O).

### Synthesis of N-[2-[[4-(4-pyridyl)piperazin-1-yl]methyl]-1H-indol-5-yl]methane-sulfonamide (Compound 251)

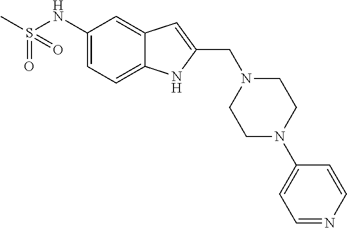 [SMILES: CS(=O)(=O)Nc1ccc2[nH]c(CN3CCN(c4ccncc4)CC3)cc2c1]

To a suspension of 2-[[4-(4-pyridyl)piperazin-1-yl]methyl]-1H-indol-5-amine (30 mg, 0.10 mmol) and triethylamine (20 μL, 0.15 mmol) in anhydrous DCM (1 mL) at 0° C. was added a solution of mesyl chloride (9 μL, 0.11 mmol) in anhydrous DCM (0.3 mL) and the mixture was allowed to warm to room temperature and stirred for 30 min. The reaction mixture was partitioned between ethyl acetate (20 mL) and water (10 mL). The layers were separated, and the organic phase was washed with brine (10 mL), dried (Na₂SO₄), filtered and concentrated. The residue was purified by column chromatography on silica gel (0-10% 1 M ammonia/methanol in DCM) followed by preparative HPLC to furnish N-[2-[[4-(4-pyridyl)piperazin-1-yl]methyl]-1H-indol-5-yl]methanesulfonamide as a white solid.

Yield 7 mg (18%). ¹H NMR (400 MHz, DMSO) δ 11.10 (s, 1H), 9.22 (s, 1H), 8.16 (dd, J=1.4, 5.2 Hz, 2H), 7.35 (s, 1H), 7.28 (d, J=7.5 Hz, 1H), 6.95 (d, J=7.5 Hz, 1H), 6.81 (dd, J=1.4, 5.2 Hz, 2H), 6.31 (d, J=1.3 Hz, 1H), 3.67 (s, 2H), 3.36-3.30 (m, 4H) 2.86 (s, 3H), 2.56-2.51 (m, 4H). m/z: [ESI⁺] 412 (M+H)⁺, (C₁₉H₂₃N₅O₂S).

### Synthesis of N-[2-[[4-(4-pyridyl)piperazin-1-yl]methyl]-1H-indol-5-yl]acetamide (Compound 258)

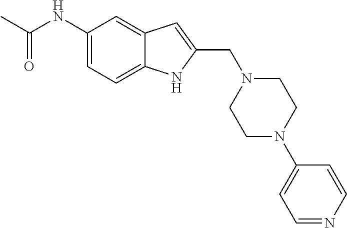 [SMILES: CC(=O)Nc1ccc2[nH]c(CN3CCN(c4ccncc4)CC3)cc2c1]

Compound N-[2-[[44-(4-pyridyl)piperazin-1-yl]methyl]-1H-indol-5-yl]acetamide was prepared from 2-[[4-(4-pyridyl)piperazin-1-yl]methyl]-1H-indol-5-amine and acetic acid following a similar procedure to that described for the synthesis of N-[2-[[4-(4-pyridyl)piperazin-1-yl]methyl]-1H-indol-5-yl]benzamide, except it was purified by preparative HPLC to furnish N-[2-[[4-(4-pyridyl)piperazin-1-yl]methyl]-1H-indol-5-yl]acetamide as a white solid.

Yield 11 mg (24%). ¹H NMR (400 MHz, DMSO) δ 10.93 (s, 1H), 9.68 (s, 1H), 8.16 (dd, J=1.3, 5.2 Hz, 2H), 7.78 (d, J=2.0 Hz, 1H), 7.23 (d, 8.7 Hz, 1H), 7.13 (dd, J=2.0, 8.7 Hz, 1H), 6.83 (dd, J=1.3, 5.2 Hz, 2H), 6.26 (d, J=1.3 Hz, 1H), 3.65 (s, 2H), 3.41-3.34 (m, 4H), 2.56-2.51 (m, 4H), 2.03 (s, 3H). m/z: [ESI⁺] 350 (M+H)⁺, (C₂₀H₂₃N₅O).

### Synthesis of 5-methyl-2-[[4-(4-pyridyl)piperazin-1-yl]methyl]-1H-indole (Compound 252)

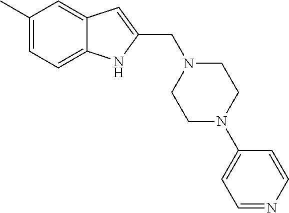 [SMILES: Cc1ccc2[nH]c(CN3CCN(c4ccncc4)CC3)cc2c1]

Compound 5-methyl-2-[[4-(4-pyridyl)piperazin-1-yl]methyl]-1H-indole was prepared from 5-methyl-1H-indole-2-carbaldehyde and 1-(pyridin-4-yl)piperazine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH), and was isolated as a white solid.

Yield 118 mg (82%). ¹H NMR (400 MHz, DMSO) δ 10.88 (s, 1H), 8.16 (d, J=5.6 Hz, 2H), 7.23 (d, J=7.0 Hz, 1H), 7.21 (s, 1H), 6.86 (dd, J=1.4, 7.0 Hz, 1H), 6.81 (dd, J=1.4, 5.0 Hz, 2H), 6.21 (d, J=1.3 Hz, 1H), 3.64 (s, 2H), 3.33-3.30 (m, 4H), 2.56-2.51 (m, 4H), 2.36 (s, 3H). m/z: [ESI⁺] 307 (M+H)⁺, (C₁₉H₂₂N₄).

### Synthesis of 2-[[4-(4-pyridyl)piperazin-1-yl]methyl]-1H-indole-5-carbonitrile (Compound 256)

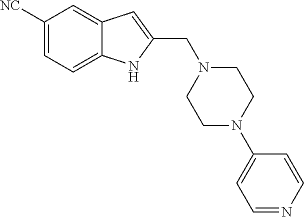 [SMILES: N#Cc1ccc2[nH]c(CN3CCN(c4ccncc4)CC3)cc2c1]

Compound 2-[[4-(4-pyridyl)piperazin-1-yl]methyl]-1H-indole-5-carbonitrile was prepared from 2-formyl-1H-indole-5-carbonitrile and 1-(pyridin-4-yl)piperazine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH), and was isolated as a white solid.

Yield 635 mg (57%). ¹H NMR (400 MHz, DMSO) δ 11.69 (s, 1H), 8.16 (dd, J=1.2, 5.0 Hz, 2H), 8.01 (s, 1H), 7.50 (d, J=7.5 Hz, 1H), 7.40 (dd, J=1.6, 7.5 Hz, 1H), 6.81 (dd, J=1.2, 5.0 Hz, 2H), 6.49 (d, J=1.1 Hz, 1H), 3.72 (s, 2H), 3.36-3.31 (m, 4H), 2.58-2.52 (m, 4H). m/z: [ESI⁺] 318 (M+H)⁺, (C₁₉H₁₉N₅)/

### Synthesis of 5-(2-methoxyethoxy)-2-[[4-(4-pyridyl)piperazin-1-yl]methyl]-1H-indole (Compound 260) and 5-(2-methoxyethoxy)-1-(2-methoxyethyl)-2-((4-(pyridin-4-yl)piperazin-1-yl)methyl)-1H-indole (Compound 261)

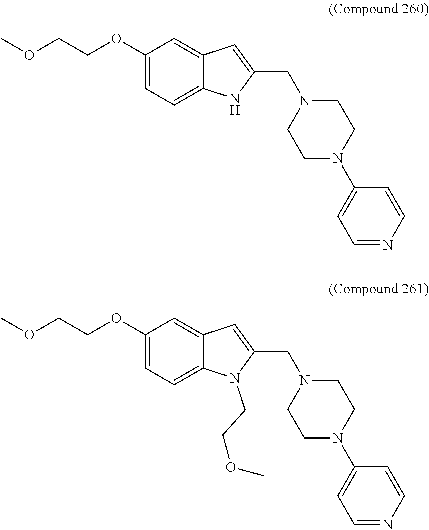 [SMILES: COCCOc1ccc2[nH]c(CN3CCN(c4ccncc4)CC3)cc2c1 ; COCCOc1ccc2c(c1)cc(CN1CCN(c3ccncc3)CC1)n2CCOC]

Compounds 5-(2-methoxyethoxy)-2-[[4-(4-pyridyl)piperazin-1-yl]methyl]-1H-indole and 5-(2-methoxyethoxy)-1-(2-methoxyethyl)-2-((4-(pyridin-4-yl)piperazin-1-yl)methyl)-1H-indole were prepared from a mixture of 5-(2-methoxyethoxy)-1H-indole-2-carbaldehyde and 5-(2-methoxyethoxy)-1-(2-methoxyethyl)-1H-indole-2-carbaldehyde and 1-(pyridin-4-yl)piperazine following a similar procedure to that described for the synthesis of 2-[[4-(2-pyridyl)piperazin-1-yl]methyl]-1H-indole, except they were purified by SCX-2 ion exchange chromatography (2 g, 0.6 mmol/g loading, washed with MeOH and eluted with 1 M ammonia/MeOH), followed by column chromatography on silica gel (0-10% methanol in DCM) and were isolated as white and beige solids respectively.

### 5-(2-Methoxyethoxy)-2-[[4-(4-pyridyl)piperazin-1-yl]methyl]-1H-indole (Compound 260)

Yield 58 mg (30%, over 3 steps). ¹H NMR (400 MHz, DMSO) δ 10.88 (s, 1H), 8.16 (dd, J=1.6, 5.0 Hz, 2H), 7.22 (d, J=8.7 Hz, 1H), 6.98 (d, J=2.4 Hz, 1H), 6.81 (dd, J=1.6, 5.0 Hz, 2H), 6.69 (dd, J=2.4, 8.7 Hz, 1H), 6.22 (d, J=1.4 Hz, 1H), 4.08-4.04 (m, 2H), 3.69-3.65 (m, 2H), 3.64 (s, 2H), 3.36-3.30 (m, 7H), 2.55-2.51 (m, 4H). m/z: [ESI⁺] 367 (M+H)⁺, (C₂₁H₂₆N₄O₂).

### 5-(2-Methoxyethoxy)-1-(2-methoxyethyl)-2-((4-(pyridin-4-yl)piperazin-1-yl)methyl)-1H-indole (Compound 261)

Yield 5 mg. ¹H NMR (400 MHz, DMSO) δ 8.16 (dd, J=1.5, 5.0 Hz, 2H), 7.34 (d, J=8.8 Hz, 1H), 7.01 (d, J=2.4 Hz, 1H), 6.81 (dd, J=1.5, 5.0 Hz, 2H), 6.77 (dd, J=2.4, 8.8 Hz, 1H), 6.28 (s, 1H), 4.40 (t, J=5.7 Hz, 2H), 4.09-4.06 (m, 2H), 3.68 (s, 2H), 3.67-3.61 (m, 4H), 3.34 (s, 3H), 3.33-3.28 (m, 4H), 3.22 (s, 3H). 4 protons obscured by solvent peak. m/z: [ESI⁺] 425 (M+H)⁺, (C₂₄H₃₂N₄O₃).

### Synthesis of 2-[[4-(4-pyridyl)piperazin-1-yl]methyl]-1H-indole-3-carbonitrile (Compound 280)

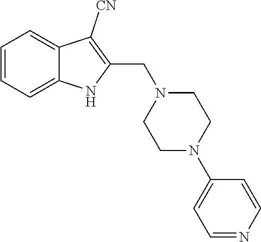 [SMILES: N#Cc1c(CN2CCN(c3ccncc3)CC2)[nH]c2ccccc12]

To a solution of 1-(diethoxymethyl)-2-((4-(pyridin-4-yl)piperazin-1-yl)methyl)-1H-indole-3-carbonitrile (180 mg, 0.43 mmol) in dioxane (2 mL) was added at room temperature a solution of HCl in dioxane solution (4 M, 5 mL, 20 mmol) and the mixture was stirred at room temperature for 1 hour. The reaction mixture was concentrated and purified by SCX-2 ion exchange chromatography (2 g, 0.6 mmol/g loading, washed with MeOH and eluted with 1 M ammonia/MeOH), followed by column chromatography on silica gel (010% 1 M ammonia/methanol in DCM) to afford 2-[[4-(4-pyridyl)piperazin-1-yl]methyl]-1H-indole-3-carbonitrile as a yellow solid.

Yield 20 mg (15%). ¹H NMR (400 MHz, DMSO) δ 12.27 (s, 1H), 8.17 (d, J=6.1 Hz, 2H), 7.60 (d, J=7.8 Hz, 1H), 7.50 (d, J=7.8 Hz, 1H), 7.30-7.20 (m, 2H), 6.84 (d, J=6.1 Hz, 2H), 3.87 (s, 2H), 3.42-3.34 (m, 4H), 2.65-2.55 (m, 4H). m/z: [ESI⁺] 318 (M+H)⁺, (C₁₉H₁₉N₅).

### Synthesis of (1H-indol-2-yl)(4-(pyridin-4-yl)piperazin-1-yl)methanone (Compound 244)

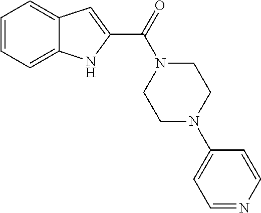 [SMILES: O=C(c1cc2ccccc2[nH]1)N1CCN(c2ccncc2)CC1]

To a solution of 1H-indole-2-carboxylic acid (50 mg, 0.31 mmol), HATU (130 mg, 0.34 mmol) and DIPEA (160 μL, 0.93 mmol) in DMF (1 mL) was added at room temperature 1-(pyridin-4-yl)piperazine (56 mg, 0.34 mmol) and the mixture was stirred at this temperature for 2 hours. The reaction mixture was partitioned between ethyl acetate (20 mL) and water (10 mL). The layers were separated, and the organic phase was washed with water (10 mL), water/brine 1:1 (10 mL) and brine (10 mL). The organic layer was dried (Na₂SO₄), filtered and evaporated and the residue purified by column chromatography on silica gel (0-10% methanol in DCM) to afford (1H-indol-2-yl)(4-(pyridin-4-yl)piperazin-1-yl)methanone as a white solid.

Yield 33 mg (35%). ¹H NMR (400 MHz, DMSO) δ 11.63 (s, 1H), 8.21 (dd, J=1.5, 5.0 Hz, 2H), 7.66 (d, J=8.2 Hz, 1H), 7.45 (d, J=8.2 Hz, 1H), 7.24 (dd, J=7.9 Hz, 2.0 Hz, 1H), 7.08 (dd, J=7.9 Hz, 2.0 Hz, 1H), 6.89-6.85 (m, 3H), 3.92 (br s, 4H), 3.54-3.49 (m, 4H). m/z: [ESI⁺] 307 (M+H)⁺, (C₁₈H₁₈N₄O).

### Synthesis of 2-[[4-(4-pyridyl)-1-piperidyl]methyl]-1H-indole (Compound 269)

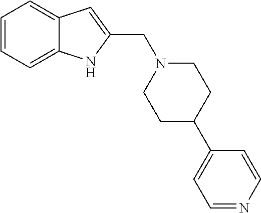 [SMILES: c1ccc2[nH]c(CN3CCC(c4ccncc4)CC3)cc2c1]

Compound 2-[[4-(4-pyridyl)-1-piperidyl]methyl]-1H-indole was prepared from 1H-indole-2-carbaldehyde and 4-(piperidin-4-yl)pyridine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH), and was isolated as an off white solid

Yield 92 mg (92%). ¹H NMR (400 MHz, DMSO) δ 10.98 (s, 1H), 8.47 (dd, J=1.6, 4.4 Hz, 2H), 7.45 (d, J=7.5 Hz, 1H), 7.35 (d, J=7.5 Hz, 1H), 7.27 (dd, J=1.6, 4.4 Hz, 2H), 7.03 (dd, J=7.5, 7.5 Hz, 1H), 6.95 (dd, J=7.5, 7.5 Hz, 1H), 6.29 (d, J=1.3 Hz, 1H), 3.66 (s, 2H), 2.99 (d, J=11.5 Hz, 2H), 2.60-2.53 (m, 1H), 2.15-2.06 (m, 2H), 1.82-1.75 (m, 2H), 1.73-1.62 (m, 2H). m/z: [ESI⁺] 292 (M+H)⁺, (C₁₉H₂₁N₃).

### Synthesis of 1-(1H-indol-2-ylmethyl)-4-(4-pyridyl)piperidin-4-ol (Compound 267)

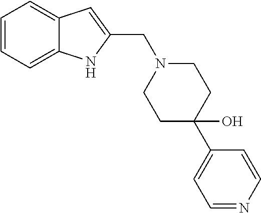 [SMILES: OC1(c2ccncc2)CCN(Cc2cc3ccccc3[nH]2)CC1]

Compound 1-(1H-indol-2-ylmethyl)-4-(4-pyridyl)piperidin-4-ol was prepared from 1H-indole-2-carbaldehyde and 4-(pyridin-4-yl)piperidin-4-ol following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by preparative HPLC and was isolated as a white solid.

Yield 27 mg (25%). ¹H NMR (400 MHz, DMSO) δ 10.98 (s, 1H), 8.51 (dd, J=1.6, 4.4 Hz, 2H), 7.47-7.45 (m, 3H), 7.32 (d, J=8.0 Hz, 1H), 7.03 (dd, J=7.2, 7.2 Hz, 1H), 6.95 (dd, J=7.2, 7.2 Hz, 1H), 6.30 (d, J=1.3 Hz, 1H), 5.07 (s, 1H), 3.68 (s, 2H), 2.71 (d, J=10.7 Hz, 2H), 2.50-2.43 (m, 2H), 1.95 (dt, J=4.2, 12.0 Hz, 2H), 1.59 (d, J=12.0 Hz, 2H). m/z: [ESI⁺] 308 (M+H)⁺, (C₁₉H₂₁N₃O).

### Synthesis of 2-[[4-(4-pyridyloxy)-1-piperidyl]methyl]-1H-indole (Compound 268)

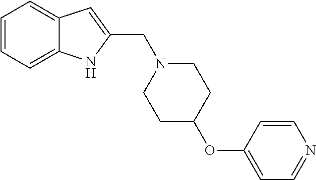 [SMILES: c1ccc2[nH]c(CN3CCC(Oc4ccncc4)CC3)cc2c1]

Compound 2-[[4-(4-pyridyloxy)-1-piperidyl]methyl]-1H-indole was prepared from 1H-indole-2-carbaldehyde and 4-(piperidin-4-yloxy)pyridine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by preparative HPLC and was isolated as a white solid.

Yield 59 mg (56%). ¹H NMR (400 MHz, DMSO) δ 11.00 (s, 1H), 8.35 (dd, J=1.4, 4.9 Hz, 2H), 7.45 (d, J=8.0 Hz, 1H), 7.33 (d, J=8.0 Hz, 1H), 7.03 (dd, J=7.5, 7.5 Hz, 1H), 6.99-6.95 (m, 3H), 6.28 (d, J=1.4 Hz, 1H), 4.60-4.52 (m, 1H), 3.65 (s, 2H), 2.77-2.68 (m, 2H), 2.37-2.28 (m, 2H), 2.01-1.97 (m, 2H), 1.73-1.62 (m, 2H). m/z: [ESI⁺] 308 (M+H)⁺, (C₁₉H₂₁N₃O).

### Synthesis of 2-[[2-(4-pyridyl)-2,6-diazaspiro[3.3]heptan-6-yl]methyl]-1H-indole (Compound 271)

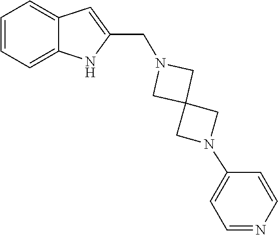 [SMILES: c1ccc2[nH]c(CN3CC4(C3)CN(c3ccncc3)C4)cc2c1]

Compound 2-[[2-(4-pyridyl)-2,6-diazaspiro[3.3]heptan-6-yl]methyl]-1H-indole was prepared from 1H-indole-2-carbaldehyde and 2-(pyridin-4-yl)-2,6-diazaspiro[3.3]heptane following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by preparative HPLC and was isolated as an off-white solid.

Yield 45 mg (43%). ¹H NMR (400 MHz, DMSO) δ 11.00 (s, 1H), 8.11 (dd, J=1.6, 4.8 Hz, 2H), 7.44 (d, J=8.0 Hz, 1H), 7.30 (d, J=8.0 Hz, 1H), 7.02 (dd, J=7.5, 7.5 Hz, 1H), 6.94 (dd, J=7.5, 7.5 Hz, 1H), 6.34 (dd, J=1.6, 4.8 Hz, 2H), 6.25 (s, 1H), 3.98 (s, 4H), 3.66 (s, 2H), 3.36 (s, 4H). m/z: [ESI⁺] 305 (M+H)⁺, (C₁₉H₂₀N₄).

### Synthesis of 2-[[4-(4-pyridyl)-1,4-diazepan-1-yl]methyl]-1H-indole (Compound 273)

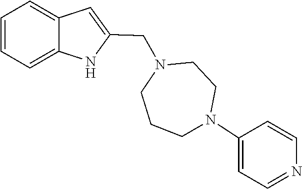 [SMILES: c1ccc2[nH]c(CN3CCCN(c4ccncc4)CC3)cc2c1]

Compound 2-[[4-(4-pyridyl)-1,4-diazepan-1-yl]methyl]-1H-indole was prepared from 1H-indole-2-carbaldehyde and 1-(pyridin-4-yl)-1,4-diazepane dihydrochloride following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by preparative HPLC and was isolated as an off-white solid.

Yield 46 mg (43%). ¹H NMR (400 MHz, DMSO) δ 10.94 (s, 1H), 8.08 (dd, J=1.6, 5.0 Hz, 2H), 7.44 (d, J=8.0 Hz, 1H), 7.32 (d, J=8.0 Hz, 1H), 7.02 (dd, J=7.6, 7.6 Hz, 1H), 6.94 (dd, J=7.6, 7.6 Hz, 1H), 6.62 (dd, J=1.6, 5.0 Hz, 2H), 6.26 (d, J=1.1 Hz, 1H), 3.77 (s, 2H), 3.57-3.49 (m, 4H), 2.75-2.71 (m, 2H), 2.63-2.55 (m, 2H), 1.93-1.85 (m, 2H). m/z: [ESI⁺] 307 (M+H)⁺, (C₁₉H₂₂N₄).

### Synthesis of 7-(1H-indol-2-ylmethyl)-2-(4-pyridyl)-2,7-diazaspiro[4.4]nonane (Compound 272)

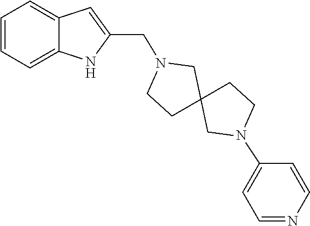 [SMILES: c1ccc2[nH]c(CN3CCC4(CCN(c5ccncc5)C4)C3)cc2c1]

Compound 7-(1H-indol-2-ylmethyl)-2-(4-pyridyl)-2,7-diazaspiro[4.4]nonane was prepared from 1H-indole-2-carbaldehyde and 2-(pyridin-4-yl)-2,7-diazaspiro[4.4]nonane following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by column chromatography on silica gel (0-10% 1 M ammonia/methanol in DCM) and was isolated as a red solid.

Yield 93 mg (81%). ¹H NMR (400 MHz, DMSO) δ 10.99 (s, 1H), 8.07 (dd, J=1.5, 4.9 Hz, 2H), 7.44 (d, J=8.0 Hz, 1H), 7.31 (d, J=8.0 Hz, 1H), 7.02 (dd, J=7.5, 7.5 Hz, 1H), 6.94 (dd, J=7.5, 7.5 Hz, 1H), 6.42 (dd, J=1.5, 4.9 Hz, 2H), 6.27 (d, J=1.3 Hz, 1H), 3.74 (s, 2H), 3.33-3.16 (m, 4H), 2.75-2.53 (m, 4H), 2.09-1.95 (m, 2H), 1.80 (t, J=7.0 Hz, 2H). m/z: [ESI⁺] 333 (M+H)⁺, (C₂₁H₂₄N₄).

### Synthesis of 2-[[(3S,5R)-3,5-dimethyl-4-(4-pyridyl)piperazin-1-yl]methyl]-1H-indole (Compound 276)

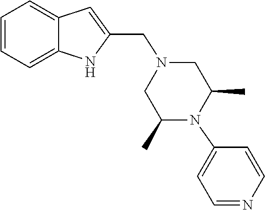 [SMILES: C[C@@H]1CN(Cc2cc3ccccc3[nH]2)C[C@H](C)N1c1ccncc1]

Compound 2-[[(3S,5R)-3,5-dimethyl-4-(4-pyridyl)piperazin-1-yl]methyl]-1H-indole was prepared from 1H-indole-2-carbaldehyde and (2R,6S)-2,6-dimethyl-1-(pyridin-4-yl)piperazine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by preparative HPLC and was isolated as an off-white solid.

Yield 3 mg (8%, over 2 steps). ¹H NMR (400 MHz, DMSO) δ 10.99 (s, 1H), 8.13 (d, J=6.4 Hz, 2H), 7.47 (d, J=8.0 Hz, 1H), 7.37 (d, J=8.0 Hz, 1H), 7.04 (dd, J=7.3, 7.3 Hz, 1H), 6.96 (dd, J=7.3, 7.3 Hz, 1H), 6.67 (d, J=6.4 Hz, 2H), 6.34 (d, J=1.1 Hz, 1H), 4.06-4.00 (m, 2H), 3.70 (s, 2H), 2.81 (d, J=11.0 Hz, 2H), 2.23 (dd, J=4.1, 11.0 Hz, 2H), 1.24 (d, J=6.5 Hz, 6H). m/z: [ESI⁺] 321 (M+H)⁺, (C₂₀H₂₄N₄).

### Synthesis of 2-[[(3aS,6aR)-5-(4-pyridyl)-1,3,3a,4,6,6a-hexahydropyrrolo[3,4-c]pyrrol-2-yl]methyl]-1H-indole (Compound 285)

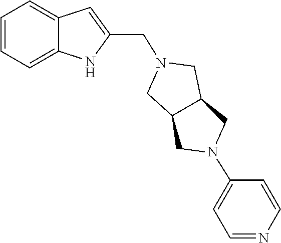 [SMILES: c1ccc2[nH]c(CN3C[C@@H]4CN(c5ccncc5)C[C@@H]4C3)cc2c1]

Compound 2-[[(3aS,6aR)-5-(4-pyridyl)-1,3,3a,4,6,6a-hexahydropyrrolo[3,4-c]pyrrol-2-yl]methyl]-1H-indole was prepared from 1H-indole-2-carbaldehyde and (3aR,6aS)-2-(pyridin-4-yl) octahydropyrrolo[3,4-c]pyrrole following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by column chromatography on silica gel (0-10% 1N ammonia/methanol in DCM) and was isolated as a pink solid.

Yield 70 mg (64%). ¹H NMR (400 MHz, DMSO) δ 10.98 (s, 1H), 8.11 (dd, J=1.5, 5.0 Hz, 2H), 7.44 (d, J=8.0 Hz, 1H), 7.28 (d, J=8.0 Hz, 1H), 7.02 (dd, J=7.5, 7.5 Hz, 1H), 6.93 (dd, J=7.5, 7.5 Hz, 1H), 6.50 (dd, J=1.5, 5.0 Hz, 2H), 6.25 (d, J=1.3 Hz, 1H), 3.71 (s, 2H), 3.53-3.46 (m, 2H), 3.18 (dd, J=10.2, 3.8 Hz, 1H), 2.97-2.93 (m, 2H), 2.68-2.62 (m, 2H), 2.56-2.51 (m, 2H). m/z: [ESI⁺] 319 (M+H)⁺, (C₂₀H₂₂N₄).

### Synthesis of 2-[[4-(4-pyridyl)piperazin-1-yl]methyl]-1H-benzimidazole (Compound 253)

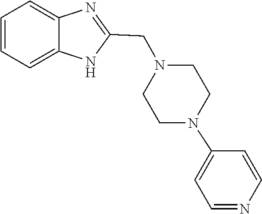 [SMILES: c1ccc2[nH]c(CN3CCN(c4ccncc4)CC3)nc2c1]

To a solution of 2-(chloromethyl)-1H-benzo[d]imidazole (91 mg, 0.55 mmol) and potassium carbonate (377 mg, 2.73 mmol) in acetonitrile (1.5 mL) was added at room temperature 1-(pyridin-4-yl)piperazine (98 mg, 0.60 mmol) and the mixture was stirred at this temperature for 18 hours. The reaction mixture was partitioned between ethyl acetate (20 mL) and brine (10 mL). The layers were separated, and the organic phase was dried (Na₂SO₄), filtered and evaporated. The residue was purified by SCX-2 ion exchange chromatography (2 g, 0.6 mmol/g loading, washed with MeOH and eluted with 1 M ammonia/MeOH), followed by column chromatography on silica gel (0-10% 1N ammonia/methanol in DCM) and preparative HPLC to afford 2-[[4-(4-pyridyl)piperazin-1-yl]methyl]-1H-benzimidazole as an off-white solid.

Yield 20 mg (13%). ¹H NMR (400 MHz, DMSO) δ 12.34 (s, 1H), 8.16 (dd, J=1.5, 5.0 Hz, 2H), 7.56 (d, J=6.7 Hz, 1H), 7.46 (d, J=6.7 Hz, 1H), 7.16 (br s, 2H), 6.82 (dd, J=1.5, 5.0 Hz, 2H), 3.80 (s, 2H), 3.39-3.28 (m, 4H), 2.62-2.58 (m, 4H). m/z: [ESI⁺] 294 (M+H)⁺, (C₁₇H₁₉N₅).

### Synthesis of 2-[[4-(5,6-dimethylpyrimidin-4-yl)piperazin-1-yl]methyl]-1H-benzimidazole (Compound 257)

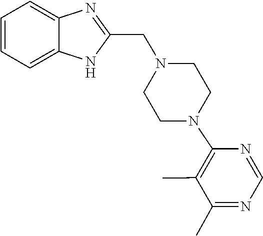 [SMILES: Cc1ncnc(N2CCN(Cc3nc4ccccc4[nH]3)CC2)c1C]

Compound 2-[[4-(5,6-dimethylpyrimidin-4-yl)piperazin-1-yl]methyl]-1H-benzimidazole was prepared from 2-(chloromethyl)-1H-benzo[d]imidazole and 4,5-dimethyl-6-(piperazin-1-yl)pyrimidine hydrochloride following a similar procedure to that described for the synthesis of 2-[[4-(4-pyridyl)piperazin-1-yl]methyl]-1H-benzimidazole, and it was isolated as an off-white solid.

Yield 15 mg (15%). ¹H NMR (400 MHz, DMSO) δ 12.34 (s, 1H), 8.44 (s, 1H), 7.56 (d, J=7.2 Hz, 1H), 7.46 (d, J=7.2 Hz, 1H), 7.20-7.14 (m, 2H), 3.79 (s, 2H), 3.28 (br s, 4H), 2.61 (br s, 4H), 2.33 (s, 3H), 2.12 (s, 3H). m/z: [ESI⁺] 294 (M+H)⁺, (C₁₈H₂₂N₆).

### Synthesis of 2-[[4-(5,6-dimethylpyrimidin-4-yl)piperazin-1-yl]methyl]-1,3-benzothiazole (Compound 254)

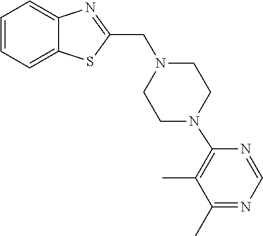 [SMILES: Cc1ncnc(N2CCN(Cc3nc4ccccc4s3)CC2)c1C]

Compound 2-[[4-(5,6-dimethylpyrimidin-4-yl)piperazin-1-yl]methyl]-1,3-benzothiazole was prepared from 2-(chloromethyl)benzo[d]thiazole and 4,5-dimethyl-6-(piperazin-1-yl)pyrimidine hydrochloride following a similar procedure to that described for the synthesis of 2-[[4-(4-pyridyl)piperazin-1-yl]methyl]-1H-benzimidazole, except it was only purified by SCX-2 ion exchange chromatography and it was isolated as a pale orange solid.

Yield 45 mg (42%). ¹H NMR (400 MHz, DMSO) δ 8.45 (s, 1H), 8.09 (d, J=7.5 Hz, 1H), 7.96 (d, J=7.5 Hz, 1H), 7.50 (dd, J=7.6, 7.6 Hz, 1H), 7.43 (dd, J=7.6, 7.6 Hz, 1H), 4.04 (s, 2H), 3.33-3.28 (m, 4H), 2.75-2.70 (m, 4H), 2.35 (s, 3H), 2.13 (s, 3H). m/z: [ESI⁺] 340 (M+H)⁺, (C₁₈H₂₁N₅S).

### Synthesis of 2-[[4-(5,6-dimethylpyrimidin-4-yl)piperazin-1-yl]methyl]-1,3-benzoxazole (Compound 259)

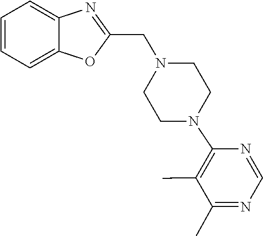 [SMILES: Cc1ncnc(N2CCN(Cc3nc4ccccc4o3)CC2)c1C]

Compound 2-[[4-(5,6-dimethylpyrimidin-4-yl)piperazin-1-yl]methyl]-1,3-benzoxazole was prepared from 2-(chloromethyl)benzo[d]oxazole and 4,5-dimethyl-6-(piperazin-1-yl)pyrimidine hydrochloride following a similar procedure to that described for the synthesis of 2-[[4-(4-pyridyl)piperazin-1-yl]methyl]-1H-benzimidazole, except it was purified by preparative HPLC twice and it was isolated as an off-white solid.

Yield 11 mg (11%). ¹H NMR (400 MHz, DMSO) δ 8.43 (s, 1H), 7.75 (dd, J=6.2, 6.2 Hz, 2H), 7.45-7.36 (m, 2H), 3.96 (s, 2H), 3.30-3.23 (m, 4H), 2.73-2.68 (m, 4H), 2.33 (s, 3H), 2.10 (s, 3H). m/z: [ESI⁺] 324 (M+H)⁺, (C₁₈H₂N₅O).

### Synthesis of 2-[[4-(4-pyridyl)piperazin-1-yl]methyl]-1,3-benzoxazole Formate (Compound 282)

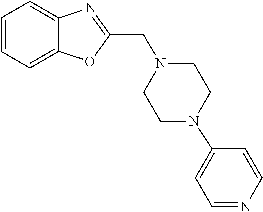 [SMILES: c1ccc2oc(CN3CCN(c4ccncc4)CC3)nc2c1]

Compound 2-[[4-(4-pyridyl)piperazin-1-yl]methyl]-1,3-benzoxazole formate was prepared from 2-(chloromethyl)benzo[d]oxazole and 1-(pyridin-4-yl)piperazine following a similar procedure to that described for the synthesis of 2-[[4-(4-pyridyl)piperazin-1-yl]methyl]-1H-benzimidazole and it was isolated as a white solid.

Yield 30 mg (34%). ¹H NMR (400 MHz, DMSO) δ 8.19 (s, 1H), 8.16 (dd, J=1.5, 5.1 Hz, 2H), 7.77-7.73 (m, 2H), 7.45-7.36 (m, 2H), 6.83 (dd, J=1.5, 5.1 Hz, 2H), 3.95 (s, 2H), (dd, J=5.0, 5.0 Hz, 4H), 2.68 (dd, J=5.0, 5.0 Hz, 4H). m/z: [ESI⁺] 295 (M+H)⁺, (C₁₇H₁₈N₄O).

### Synthesis of 2-[(4-pyrimidin-4-ylpiperazin-1-yl)methyl]-, 3-benzoxazole Hemiformate (Compound 281)

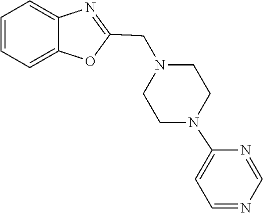 [SMILES: c1ccc2oc(CN3CCN(c4ccncn4)CC3)nc2c1]

Compound 2-[(4-pyrimidin-4-ylpiperazin-1-yl)methyl]-1,3-benzoxazole hemiformate was prepared from 2-(chloromethyl)benzo[d]oxazole and 4-(piperazin-1-yl)pyrimidine following a similar procedure to that described for the synthesis of 2-[[4-(4-pyridyl)piperazin-1-yl]methyl]-1H-benzimidazole and it was isolated as an off-white solid.

Yield 32 mg (34%). ¹H NMR (400 MHz, DMSO) δ 8.49 (s, 1H), 8.18 (d, J=2.8 Hz, 1H), 8.17 (s, 0.5H), 7.77-7.73 (m, 2H), 7.44-7.36 (m, 2H), 6.83 (dd, J=1.2, 6.3 Hz, 1H), 3.96 (s, 2H), 3.69-3.63 (m, 4H), 2.66-2.62 (m, 4H). m/z: [ESI⁺] 296 (M+H)⁺, (C₁₆H₁₇N₅O).

### Synthesis of 3-((4-(pyridin-4-yl)piperazin-1-yl)methyl)-1H-indole bis 2,2,2-trifluoroacetate (Compound 277)

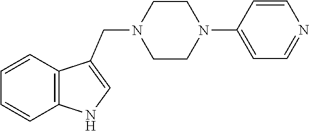 [SMILES: c1ccc2c(CN3CCN(c4ccncc4)CC3)c[nH]c2c1]

Compound 3-((4-(pyridin-4-yl)piperazin-1-yl)methyl)-1H-indole bis 2,2,2-trifluoroacetate was prepared from 1H-indole-3-carbaldehyde and 1-(pyridin-4-yl)piperazine following a similar procedure to that described for the synthesis of 2-[[4-(2-pyridyl)piperazin-1-yl]methyl]-1H-indole, except it was not purified by SCX-2 ion exchange chromatography but was purified by preparative HPLC twice and was isolated as a red gum.

Yield 60 mg (37%). ¹H NMR (400 MHz, DMSO) δ 13.75 (br s, 1H), 11.58 (s, 1H), 10.18 (br s, 1H), 8.38 (d, J=7.1 Hz, 2H), 7.80 (d, J=7.5 Hz, 1H), 7.60 (d, J=2.6 Hz, 1H), 7.49 (d, J=7.5 Hz, 1H), 7.23 (d, J=7.1 Hz, 2H), 7.19-7.10 (m, 2H), 4.60 (s, 2H), 4.45-4.39 (m, 2H), 3.60-3.56 (m, 2H), 3.51-3.35 (m, 2H), 3.29-3.17 (m, 2H). m/z: [ESI⁺] 293 (M+H)⁺, (C₁₈H₂₀N₄).

### Synthesis of 4-[[4-(4-pyridyl)piperazin-1-yl]methyl]-1H-indole (Compound 263)

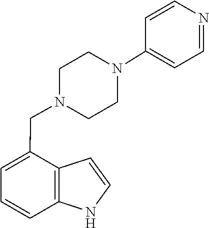 [SMILES: c1cc(CN2CCN(c3ccncc3)CC2)c2cc[nH]c2c1]

Compound 4-[[4-(4-pyridyl)piperazin-1-yl]methyl]-1H-indole was prepared from 1H-indole-4-carbaldehyde and 1-(pyridin-4-yl)piperazine following a similar procedure to that described for the synthesis of 2-[[4-(2-pyridyl)piperazin-1-yl]methyl]-1H-indole, except it was only purified by SCX-2 ion exchange chromatography (2 g, 0.6 mmol/g loading, washed with MeOH and eluted with 1 M ammonia/MeOH), and was isolated as an off-white solid.

Yield 75 mg (74%). ¹H NMR (400 MHz, DMSO) δ 11.09 (s, 1H), 8.15 (dd, J=1.6, 5.0 Hz, 2H), 7.33-7.30 (m, 2H), 7.06 (dd, J=7.6, 2.7 Hz, 1H), 6.97 (d, J=5.0 Hz, 1H), 6.80 (dd, J=1.6, 5.0 Hz, 2H), 6.63-6.61 (m, 1H), 3.77 (s, 2H), 3.29-3.23 (m, 4H), 2.57-2.51 (m, 4H). m/z: [ESI⁺] 293 (M+H)⁺, (C₁₈H₂₀N₄).

### Synthesis of 5-[[4-(4-pyridyl)piperazin-1-yl]methyl]-1H-indole (Compound 264)

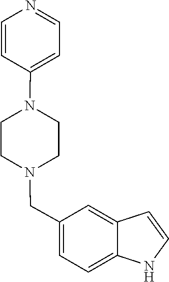 [SMILES: c1cc(N2CCN(Cc3ccc4[nH]ccc4c3)CC2)ccn1]

Compound 5-[[4-(4-pyridyl)piperazin-1-yl]methyl]-1H-indole was prepared from 1H-indole-5-carbaldehyde and 1-(pyridin-4-yl)piperazine following a similar procedure to that described for the synthesis of 2-[[4-(2-pyridyl)piperazin-1-yl]methyl]-1H-indole, except it was only purified by SCX-2 ion exchange chromatography (2 g, 0.6 mmol/g loading, washed with MeOH and eluted with 1 M ammonia/MeOH), and was isolated as an off-white solid.

Yield 72 mg (71%). ¹H NMR (400 MHz, DMSO) δ 11.04 (s, 1H), 8.15 (dd, J=1.6, 5.1 Hz, 2H), 7.46 (s, 1H), 7.37-7.31 (m, 2H), 7.08 (d, J=7.9 Hz, 1H), 6.80 (dd, J=1.6, 5.1 Hz, 2H), 6.41-6.38 (m, 1H), 3.58 (s, 2H), 3.30 (dd, J=5.0, 5.0 Hz, 4H), 2.48 (dd, J=5.0, 5.0 Hz, 4H). m/z: [ESI⁺] 293 (M+H)⁺, (C₁₈H₂₀N₄).

### Synthesis of 6-[[4-(4-pyridyl)piperazin-1-yl]methyl]-1H-indole (Compound 265)

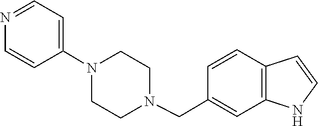 [SMILES: c1cc(N2CCN(Cc3ccc4cc[nH]c4c3)CC2)ccn1]

Compound 6-[[4-(4-pyridyl)piperazin-1-yl]methyl]-1H-indole was prepared from 1H-indole-6-carbaldehyde and 1-(pyridin-4-yl)piperazine following a similar procedure to that described for the synthesis of 2-[[4-(2-pyridyl)piperazin-1-yl]methyl]-1H-indole, except it was only purified by SCX-2 ion exchange chromatography (2 g, 0.6 mmol/g loading, washed with MeOH and eluted with 1 M ammonia/MeOH), and was isolated as a white solid.

Yield 75 mg (74%). ¹H NMR (400 MHz, DMSO) δ 11.01 (s, 1H), 8.15 (dd, J=1.4, 5.0 Hz, 2H), 7.48 (d, J=8.0 Hz, 1H), 7.35 (s, 1H), 7.32 (t, J=2.7 Hz, 1H), 6.98 (d, J=8.0 Hz, 1H), 6.80 (dd, J=1.4, 5.0 Hz, 2H), 6.41-6.39 (m, 1H), 3.60 (s, 2H), 3.33-3.28 (m, 4H), 2.51-2.47 (m, 4H). m/z: [ESI⁺] 293 (M+H)⁺, (C₁₈H₂₀N₄).

### Synthesis of 7-[[4-(4-pyridyl)piperazin-1-yl]methyl]-1H-indole (Compound 262)

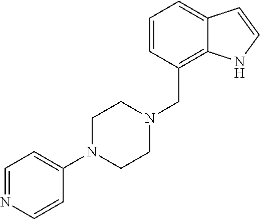 [SMILES: c1cc(CN2CCN(c3ccncc3)CC2)c2[nH]ccc2c1]

Compound 7-[[4-(4-pyridyl)piperazin-1-yl]methyl]-1H-indole was prepared from 1H-indole-7-carbaldehyde and 1-(pyridin-4-yl)piperazine following a similar procedure to that described for the synthesis of 2-[[4-(2-pyridyl)piperazin-1-yl]methyl]-1H-indole, except it was only purified by SCX-2 ion exchange chromatography (2 g, 0.6 mmol/g loading, washed with MeOH and eluted with 1 M ammonia/MeOH), and was isolated as a white solid.

Yield 76 mg (75%). ¹H NMR (400 MHz, DMSO) δ 10.85 (s, 1H), 8.15 (dd, J=1.5, 5.0 Hz, 2H), 7.48 (d, J=7.2 Hz, 1H), 7.33 (dd, J=1.9, 3.1 Hz, 1H), 7.02-6.94 (m, 2H), 6.81 (dd, J=1.5, 5.0 Hz, 2H), 6.45 (dd, J=1.9, 3.1 Hz, 1H), 3.80 (s, 2H), 3.38-3.31 (m, 4H), 2.58-2.52 (m, 4H). m/z: [ESI⁺] 293 (M+H)⁺, (C₁₈H₂₀N₄).

### Synthesis of 2-((4-(pyridin-4-yl)piperazin-1-yl)methyl)pyrazolo[1,5-a]pyridine 2,2,2-trifluoroacetate (Compound 283)

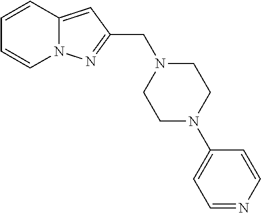 [SMILES: c1ccn2nc(CN3CCN(c4ccncc4)CC3)cc2c1]

Compound 2-((4-(pyridin-4-yl)piperazin-1-yl)methyl)pyrazolo[1,5-a]pyridine 2,2,2-trifluoroacetate was prepared from pyrazolo[1,5-a]pyridine-2-carbaldehyde and 1-(pyridin-4-yl)piperazine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by preparative HPLC and was isolated as an off-white solid.

Yield 113 mg (81%). ¹H NMR (400 MHz, DMSO) δ 13.90 (br s, 1H), 8.72 (dd, J=1.0, 7.0 Hz, 1H), 8.38 (d, J=7.5 Hz, 2H), 7.78 (dd, J=1.0, 7.0 Hz, 1H), 7.31 (dd, J=7.0, 7.0 Hz, 1H), 7.27 (d, J=7.5 Hz, 2H), 7.00 (dd, J=7.0, 7.0 Hz, 1H), 6.78 (s, 1H), 4.53 (br s, 2H), 3.96 (br s, 4H). 4 protons obstructed by solvent peak. m/z: [ESI⁺] 294 (M+H)⁺, (C₁₇H₁₉N₅).

### Synthesis of 2-[[4-(4-pyridyl)piperazin-1-yl]methyl]-1H-pyrrolo[2,3-b]pyridine (Compound 275)

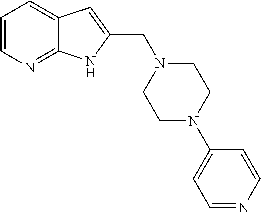 [SMILES: c1cnc2[nH]c(CN3CCN(c4ccncc4)CC3)cc2c1]

Compound 2-[[4-(4-pyridyl)piperazin-1-yl]methyl]-1H-pyrrolo[2,3-b]pyridine was prepared from 1H-pyrrolo[2,3-b]pyridine-2-carbaldehyde and 1-(pyridin-4-yl)piperazine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 N ammonia/MeOH) followed by preparative HPLC and column chromatography on silica gel (0-10% 1 M ammonia/methanol in DCM) and was isolated as a white solid.

Yield 12 mg (12%). ¹H NMR (400 MHz, DMSO) δ 11.60 (s, 1H), 8.17-8.14 (m, 3H), 7.87 (dd, J=0.9, 7.8 Hz, 1H), 7.02 (dd, J=4.6, 7.8 Hz, 1H), 6.85-6.79 (m, 2H), 6.34 (d, J=1.9 Hz, 1H), 3.70 (s, 2H), 3.38-3.34 (m, 4H), 2.58-2.53 (m, 4H). m/z: [ESI⁺] 294 (M+H)⁺, (C₁₇H₁₉N₅).

### Synthesis of 2-[[4-(4-pyridyl)piperazin-1-yl]methyl]-1H-pyrrolo[2,3-c]pyridine (Compound 270)

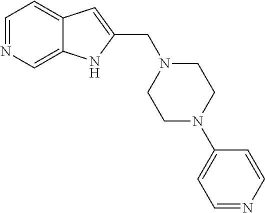 [SMILES: c1cc(N2CCN(Cc3cc4ccncc4[nH]3)CC2)ccn1]

Compound 2-[[4-(4-pyridyl)piperazin-1-yl]methyl]-1H-pyrrolo[2,3-c]pyridine was prepared from 1H-pyrrolo[2,3-c]pyridine-2-carbaldehyde and 1-(pyridin-4-yl)piperazine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by preparative HPLC and was isolated as a white solid.

Yield 8 mg (8%). ¹H NMR (400 MHz, DMSO) δ 11.57 (s, 1H), 8.67 (s, 1H), 8.16 (d, J=4.8 Hz, 2H), 8.05 (d, J=5.0 Hz, 1H), 7.45 (d, J=5.0 Hz, 1H), 6.81 (d, J=4.8 Hz, 2H), 6.40 (s, 1H), 3.75 (s, 2H), 2.58-2.53 (m, 4H). 4 protons obscured by water peak. m/z: [ESI⁺] 294 (M+H)⁺, (C₁₇H₁₉N₅).

### Synthesis of 2-[[4-(4-pyridyl)piperazin-1-yl]methyl]-1H-pyrrolo[3,2-b]pyridine (Compound 284)

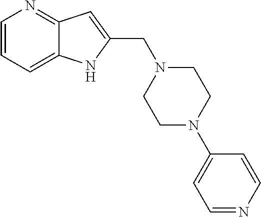 [SMILES: c1cnc2cc(CN3CCN(c4ccncc4)CC3)[nH]c2c1]

Compound 2-[[4-(4-pyridyl)piperazin-1-yl]methyl]-1H-pyrrolo[3,2-b]pyridine was prepared from 1H-pyrrolo[3,2-b]pyridine-2-carbaldehyde and 1-(pyridin-4-yl)piperazine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by column chromatography on silica gel (0-10% 1 M ammonia/methanol in DCM) and was isolated as a white solid.

Yield 30 mg (30%). ¹H NMR (400 MHz, DMSO) δ 11.29 (s, 1H), 8.27 (dd, J=1.5, 4.6 Hz, 1H), 8.16 (dd, J=1.6, 5.0 Hz, 2H), 7.68 (dt, J=1.2, 8.1 Hz, 1H), 7.05 (dd, J=4.6, 8.1 Hz, 1H), 6.82 (dd, J=1.6, 5.0 Hz, 2H), 6.45 (d, J=1.2 Hz, 1H), 3.74 (s, 2H), 3.39-3.33 (m, 4H), 2.59-2.53 (m, 4H). m/z: [ESI⁺] 294 (M+H)⁺, (C₁₇H₁₉N₅).

### Synthesis of 2-[[4-(4-pyridyl)piperazin-1-yl]methyl]imidazo[1,2-a]pyridine (Compound 274)

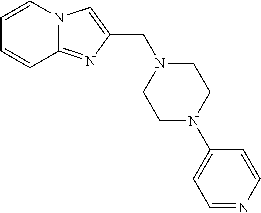 [SMILES: c1ccn2cc(CN3CCN(c4ccncc4)CC3)nc2c1]

Compound 2-[[4-(4-pyridyl)piperazin-1-yl]methyl]imidazo[1,2-a]pyridine was prepared from imidazo[1,2-a]pyridine-2-carbaldehyde and 1-(pyridin-4-yl)piperazine following a similar procedure to that described for the synthesis of 2-[[4-(2-pyridyl)piperazin-1-yl]methyl]-1H-indole, except it was purified by SCX-2 ion exchange chromatography (2 g, 0.6 mmol/g loading, washed with MeOH and eluted with 1 M ammonia/MeOH) followed by column chromatography on silica gel (0-10% 1 M ammonia/methanol in DCM) and was isolated as a brown solid.

Yield 45 mg (45%). ¹H NMR (400 MHz, DMSO) δ 8.50 (dt, J=1.2, 6.5 Hz, 1H), 8.15 (dd, J=1.6, 5.0 Hz, 2H), 7.85 (s, 1H), 7.49 (dd, J=1.2, 9.2 Hz, 1H), 7.21 (dd, J=8.0, 9.2 Hz, 1H), 6.89 (dd, J=6.5, 8.0 Hz, 1H), 6.81 (dd, J=1.6, 5.0 Hz, 2H), 3.67 (s, 2H), 3.34-3.28 (m, 4H), 2.63-2.56 (m, 4H). m/z: [ESI⁺]294 (M+H)⁺, (C₁₇H₁₉N₅).

### Synthesis of 2-((4-(5-((2-methoxyethoxy)methyl)pyrimidin-4-yl)piperazin-1-yl)methyl)-1H-indole (Compound 287)

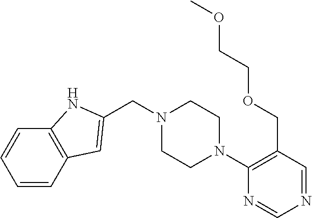 [SMILES: COCCOCc1cncnc1N1CCN(Cc2cc3ccccc3[nH]2)CC1]

To a solution of 5-[(2-methoxyethoxy)methyl]-4-(piperazin-1-yl)pyrimidine (100 mg, 0.396 mmol) in DCM (2 mL) were added 1H-indole-2-carbaldehyde (63 mg, 0.434 mmol), sodium triacetoxyborohydride (168 mg, 0.793 mmol) and acetic acid (0.01 mL, 0.173 mmol) at room temperature under a nitrogen atmosphere. The resulting mixture was stirred for 1 h at room temperature and then concentrated under reduced pressure. The residue was purified by reverse phase flash chromatography with the following conditions: Column: Spherical C18, 20-40 um, 330 g; Mobile Phase A: water (plus 10 mM NH₄HCO₃); Mobile Phase B: ACN; How rate: 80 mL/min; Gradient: 40% B-60% B in 20 min; Detector: UV 254/220 nm. The fractions containing the desired product were collected and concentrated under reduced pressure to afford 2-[(4-[5-[(2-methoxyethoxy)methyl]pyrimidin-4-yl]piperazin-1-yl)methyl]-1H-indole as a light yellow sticky oil.

Yield 107 mg (71%). ¹H NMR (400 MHz, DMSO) δJ=1.2, 7.6 Hz, 1H), 7.32 (dd, J=0.8, 8.0 Hz, 1H), 7.02 (dd, J=0.8, 7.8 Hz, 1H), 6.96 (dd, J=0.8, 7.8 Hz, 1H), 6.29 (s, 1H), 4.38 (s, 2H), 3.67 (s, 2H), 3.64 (t, J=4.4 Hz, 4H), 3.55 (t, J=5.6 Hz, 2H), 11.03 (br s, 1H), 8.51 (s, 1H), 8.23 (s, 1H), 7.45 (dd, 3.46 (t, J=5.6 Hz, 2H), 3.32 (t, J=4.4 Hz, 4H), 3.21 (s, 3H). m/z: [ESI⁺] 382 (M+H)⁺, (C₂₁H₂₇N₅O₂).

### Synthesis of 2-((4-(1-methyl-1H-1,2,3-triazol-4-yl)piperazin-1-yl)methyl)-1H-indole (Compound 288)

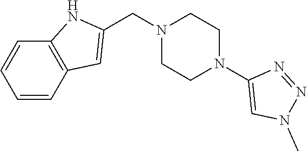 [SMILES: Cn1cc(N2CCN(Cc3cc4ccccc4[nH]3)CC2)nn1]

To a solution of 1-(1-methyl-1,2,3-triazol-4-yl)piperazine (25 mg, 0.149 mmol) in DCM (2 mL) were added 1H-indole-2-carbaldehyde (24 mg, 0.165 mmol), sodium triacetoxyborohydride (63 mg, 0.297 mmol) and acetic acid (0.01 mL, 0.173 mmol) at room temperature under an argon atmosphere. The resulting mixture was stirred for 2 h at room temperature. The resulting mixture was concentrated under reduced pressure and the residue was purified by Prep-HPLC with the following conditions (Column: XBridge Prep OBD C18 column, 30×150 mm 5 um; Mobile Phase A: water (10 mmol/L NH₄HCO₃), Mobile Phase B: ACN; How rate: 60 mL/min; Gradient: 17% B to 44% B in 8 min; Detector: UV 254/220 nm. The fractions containing the desired product were collected, concentrated and lyophilized to afford 2-[[4-(1-methyl-1,2,3-triazol-4-yl)piperazin-1-yl]methyl]-1H-indole as a brown solid.

Yield 5.5 mg (12%). ¹H NMR (400 MHz, DMSO) δ 11.02 (br s, 1H), 7.45 (d, J=8.0 Hz, 1H), 7.41 (s, 1H), 7.32 (d, J=7.6 Hz, 1H), 7.03 (dd, J=1.2, 7.2 Hz, 1H), 6.96 (dd, J=0.8, 7.2 Hz, 1H), 6.30 (s, 1H), 3.92 (s, 3H), 3.66 (s, 2H), 3.10 (t, J=6.4 Hz, 4H), 2.57 (t, J=6.4 Hz, 4H). m/z: [ESI⁺] 297 (M+H)⁺, (C₁₆H₂₀N₆).

### Synthesis of 2-[[4-(5-ethylpyrimidin-4-yl)piperazin-1-yl]methyl]-5-[(2-methoxyethoxy)methyl]-3H-1,3-benzodiazole (Compound 289)

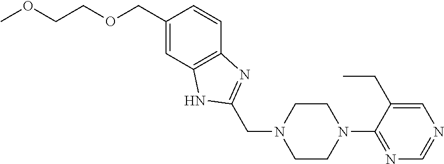 [SMILES: CCc1cncnc1N1CCN(Cc2nc3ccc(COCCOC)cc3[nH]2)CC1]

A solution of 2-[[4-(5-ethylpyrimidin-4-yl)piperazin-1-yl]methyl]-6-[(2-methoxyethoxy)methyl]-1-[[2-(trimethylsilyl)ethoxy]methyl]-1,3-benzodiazole (0.23 g, 0.43 mmol) in tetrahydrofuran (15 mL) was treated with TBAF (0.67 g, 2.56 mmol) for 3 h at 60° C. under a nitrogen atmosphere. After cooling down to room temperature, the resulting mixture was concentrated under reduced pressure. The residue was purified by reverse phase flash chromatography with the following conditions: Column: Spherical C18, 20-40 μm, 330 g; Mobile Phase A: water (plus 10 mM NH₄HCO₃); Mobile Phase B: ACN; Flow rate: 80 mL/min; Gradient: 25% B-45% B in 20 min; Detector: UV 254/220 nm. The fractions containing the desired product were collected, concentrated under reduced pressure and lyophilized to afford 2-((4-(5-ethylpyrimidin-4-yl)piperazin-1-yl)methyl)-6-((2-methoxyethoxy)methyl)-1H-benzo[d]imidazole as a brown sticky oil.

Yield 79 mg (45%). ¹H NMR (400 MHz, DMSO) δ 12.34 (br s, 1H), 8.53 (s, 1H), 8.25 (s, 1H), 7.52 (d, J=8.0 Hz, 1H), 7.42 (d, J=8.0 Hz, 1H), 7.18-7.06 (m, 1H), 4.56 (d, J=6.4 Hz, 2H), 3.79 (s, 2H), 3.60-3.52 (m, 2H), 3.52-3.45 (m, 2H), 3.45-3.38 (m, 4H), 3.26 (s, 3H), 2.64-2.54 (m, 6H), 1.19 (t, J=7.5 Hz, 3H). m/z: [ESI⁺] 411 (M+H)⁺, (C₂₂H₃₀N₆O₂).

### Synthesis of 2-[[4-(5-ethylpyrimidin-4-yl)piperazin-1-yl]methyl]-5-(2-methoxyethoxy)-3H-1,3-benzodiazole (Compound 290)

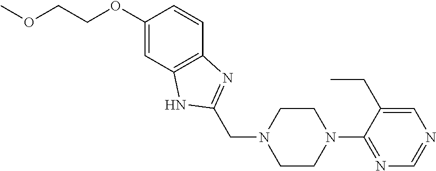 [SMILES: CCc1cncnc1N1CCN(Cc2nc3ccc(OCCOC)cc3[nH]2)CC1]

To a stirred solution of [4-(5-ethylpyrimidin-4-yl)piperazin-1-yl]acetic acid (0.30 g, 1.20 mmol) in DCM (30 mL) were added triethylamine (0.50 mL, 3.597 mmol), HATU (0.68 g, 1.79 mmol) and 4-(2-methoxyethoxy)benzene-1,2-diamine (0.44 g, 2.41 mmol) at room temperature. After stirring for additional 4 h, the reaction was quenched with water (50 mL) and extracted with DCM (3×30 mL). The combined organic layers were concentrated under reduced pressure to afford the crude product as a red oil (0.24 g). Half of the above material was dissolved into acetic acid (10 mL) at room temperature. The resulting mixture was stirred for 16 h at 40° C. under an air atmosphere. After cooling down to room temperature, the resulting mixture was concentrated under reduced pressure. The residue was purified by Prep-HPLC with the following conditions: Column: YMC-DIPEAActus Triart C18, 30×250 mm, 5 μm; Mobile Phase A: water (plus 10 mM NH₄HCO₃); Mobile Phase B: ACN; How rate: 30 mL/min; Gradient: 30% B-50% B in 8 min; Detector: UV 254/220 nm. The fractions containing the desired product were collected, concentrated under reduced pressure and lyophilized to afford 2-[[4-(5-ethylpyrimidin-4-yl)piperazin-1-yl]methyl]-5-(2-methoxyethoxy)-3H-1,3-benzodiazole as a colorless sticky oil.

Yield 15 mg (7% over two steps). ¹H NMR (400 MHz, DMSO) δ 12.18 (br s, 1H), 8.52 (s, 1H), 8.24 (s, 1H), 7.42 (d, J=8.8 Hz, 0.6H), 7.32 (d, J=8.8 Hz, 0.4H), 7.09 (d, J=2.4 Hz, 0.4H), 6.94 (d, J=2.4 Hz, 0.6H), 6.82-6.75 (m, 1H), 4.10-4.0 (m, 2H), 3.74 (d, J=6.4 Hz, 2H), 3.69-3.66 (m, 2H), 3.43-3.37 (m, 4H), 3.23 (s, 3H), 2.63-2.53 (m, 6H), 1.19 (t, J=7.5 Hz, 3H) (tautomers). m/z: [ESI⁺] 397 (M+H)⁺, (C₂₁H₂₈N₆O₂).

### Synthesis of 2-[[4-(5-ethylpyrimidin-4-yl)piperazin-1-yl]methyl]-6-[(2-methoxyethoxy)methyl]-1H-indole (Compound 291)

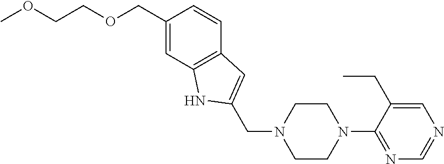 [SMILES: CCc1cncnc1N1CCN(Cc2cc3ccc(COCCOC)cc3[nH]2)CC1]

To a solution of 2-[4-(5-ethylpyrimidin-4-yl)piperazine-1-carbonyl]-6-[(2-methoxyethoxy)methyl]-1H-indole (200 mg, 0.472 mmol) in THF (5 mL) was added lithium aluminum hydride (54 mg, 1.423 mmol) at room temperature under a nitrogen atmosphere. The resulting mixture was stirred for 2 h at 70° C. under a nitrogen atmosphere. After cooling down to room temperature, the resulting mixture was quenched with water (10 mL). The resulting mixture was extracted with ethyl acetate (3×20 mL). The combined organic extracts were dried over anhydrous Na₂SO₄. After filtration, the filtrate was concentrated under reduced pressure. The residue was purified by reverse phase flash chromatography with the following conditions: Column: Spherical C18, 20-40 m, 120 g; Mobile Phase A: water (plus 10 mM NH₄HCO₃); Mobile Phase B: ACN; How rate: 60 mL/min; Gradient: 45% B-65% B in 20 min; Detector: UV 254/220 nm. The fractions containing the desired product were collected and concentrated under reduced pressure to afford 2-[[4-(5-ethylpyrimidin-4-yl)piperazin-1-yl]methyl]-6-[(2-methoxyethoxy)methyl]-1H-indole as a brown oil:

Yield 35 mg (18%). ¹H NMR (400 MHz, DMSO) δ 11.03 (d, J=2.2 Hz, 1H), 8.52 (s, 1H), 8.24 (s, 1H), 7.41 (d, J=8.0 Hz, 1H), 7.29 (s, 1H), 6.92 (dd, J=0.8, 8.0 Hz, 1H), 6.29 (d, J=0.8 Hz, 1H), 4.53 (s, 2H), 3.67 (s, 2H), 3.54-3.52 (m, 2H), 3.49-3.47 (m, 2H), 3.42-3.36 (m, 4H), 3.26 (s, 3H), 2.63-2.52 (m, 6H), 1.18 (t, J=7.5 Hz, 3H).

¹H NMR (400 MHz, CDCl₃) δ 8.64 (br s, 1H), 8.63 (s, 1H), 8.25 (s, 1H), 7.54 (d, J=8.0 Hz, 1H), 7.39 (s, 1H), 7.10 (dd, J=1.2, 8.0 Hz, 1H), 6.39 (s, 1H), 4.70 (s, 2H), 3.79 (s, 2H), 3.65 (t, J=4.4 Hz, 2H), 3.62 (t, J=4.4 Hz, 2H), 3.42-3.36 (m, 4H), 3.26 (s, 3H), 2.67-2.59 (m, 4H), 2.52 (q, J=7.6 Hz, 2H), 1.18 (t, J=7.6 Hz, 3H). m/z: [ESI⁺] 410 (M+H)⁺, (C₂₃H₃₁N₅O₂).

### Synthesis of 2-[[4-(5-ethylpyrimidin-4-yl)piperazin-1-yl]methyl]-6-(2-methoxyethoxy)-1H-indole (Compound 292)

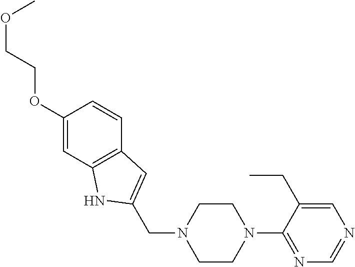 [SMILES: CCc1cncnc1N1CCN(Cc2cc3ccc(OCCOC)cc3[nH]2)CC1]

To a solution of (4-(5-ethylpyrimidin-4-yl)piperazin-1-yl)(6-(2-methoxyethoxy)-1H-indol-2-yl)methanone (220 mg, 0.537 mmol) in THF (15 mL) was added lithium aluminum hydride (62 mg, 1.633 mmol) at 0° C. under an argon atmosphere. The resulting mixture was stirred for 4 h at room temperature. The reaction was quenched with water (4 mL) at 0° C. The resulting mixture was filtered and the collected filtered cake was washed with ethyl acetate (3×20 mL). The combined washings and filtrate were concentrated under reduced pressure. The residue was purified by Prep-HPLC with the following conditions: Column: YMC-Actus Triart C18, 30×250 mm, 5 μm; Mobile Phase A: water (plus 5 mM NH₄HCO₃); Mobile Phase B: ACN; How rate: 30 mL/min; Gradient: 50% B to 80% B in 8 min; Detector: UV 254/220 nm. The fractions containing the desired product were collected, concentrated under reduced pressure and lyophilized to afford 2-[[4-(5-ethylpyrimidin-4-yl)piperazin-1-yl]methyl]-6-(2-methoxyethoxy)-1H-indole as a yellow oil.

Yield 11 mg (6%). ¹H NMR (400 MHz, DMSO) δ 10.84 (s, 1H), 8.52 (s, 1H), 8.24 (s, 1H), 7.31 (d, J=8.4 Hz, 1H), 6.84 (d, J=2.0 Hz, 1H), 6.62 (dd, J=2.4, 8.4 Hz, 1H), 6.21 (s, 1H), 4.10 (t, J=4.8 Hz, 2H), 3.68 (t, J=4.8 Hz, 2H), 3.62 (s, 2H), 3.42-3.38 (m, 4H), 3.31 (s, 3H), 2.59 (t, J=7.6 Hz, 2H), 2.56-2.51 (m, 4H), 1.18 (t, J=7.6 Hz, 3H). m/z: [ESI⁺] 396 (M+H)⁺, (C₂₂H₂₉N₅O₂).

### Synthesis of 2-((4-(5,6-dimethylpyrimidin-4-yl)piperazin-1-yl)methyl)-4-fluorobenzo[d]oxazole (Compound 293)

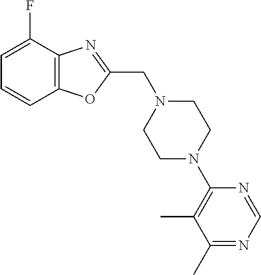 [SMILES: Cc1ncnc(N2CCN(Cc3nc4c(F)cccc4o3)CC2)c1C]

Compound 2-((4-(5,6-dimethylpyrimidin-4-yl)piperazin-1-yl)methyl)-4-fluorobenzo[d]oxazole was prepared from 2-(chloromethyl)-4-fluorobenzo[d]oxazole (0.40 g, 2.16 mmol) and 4,5-dimethyl-6-(piperazin-1-yl)pyrimidine hydrochloride (0.55 g, 2.40 mmol) following a procedure similar to that described for the synthesis of 2-[[4-(4-pyridyl)piperazin-1-yl]methyl]-1H-benzimidazole, and was isolated as a light yellow solid.

Yield 0.22 g (30%). ¹H NMR (400 MHz, DMSO) δ 8.42 (s, 1H), 7.63 (dd, J=0.8, 8.0 Hz, 1H), 7.48-7.39 (m, 1H), 7.31-7.21 (m, 1H), 3.97 (s, 2H), 3.28-3.24 (m, 4H), 2.72-2.68 (m, 4H), 2.32 (s, 3H), 2.10 (s, 3H). ¹⁹F NMR (376 MHz, DMSO) δ −126.22. m/z: [ESI⁺] 342 (M+H)⁺, (C₁₈H₂₀FN₅O).

### Synthesis of 2-((4-(5,6-dimethylpyrimidin-4-yl)piperazin-1-yl)methyl)-5-fluorobenzo[d]oxazole (Compound 297)

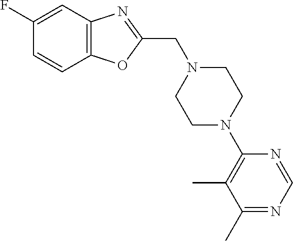 [SMILES: Cc1ncnc(N2CCN(Cc3nc4cc(F)ccc4o3)CC2)c1C]

Compound 2-((4-(5,6-dimethylpyrimidin-4-yl)piperazin-1-yl)methyl)-5-fluorobenzo[d]oxazole was prepared from 2-(chloromethyl)-5-fluorobenzo[d]oxazole (0.60 g, 3.23 mmol) and 4,5-dimethyl-6-(piperazin-1-yl)pyrimidine hydrochloride (0.80 g, 3.50 mmol) following a procedure similar to that described for the synthesis of 2-[[4-(4-pyridyl)piperazin-1-yl]methyl]-1H-benzimidazole, and was isolated as a yellow solid.

Yield 0.28 g (25%). ¹H NMR (400 MHz, DMSO) δ 8.42 (s, 1H), 7.78 (dd, J=4.4, 8.8 Hz, 1H), 7.64 (dd, J=2.8, 8.8 Hz, 1H), 7.32-7.22 (m, 1H), 3.95 (s, 2H), 3.26 (t, J=4.8 Hz, 4H), 2.69 (t, J=4.8 Hz, 4H), 2.32 (s, 3H), 2.10 (s, 3H). ¹⁹F NMR (376 MHz, DMSO) δ −118.10. m/z: [ESI⁺] 342 (M+H)⁺, (C₁₈H₂₀FN₅O).

### Synthesis of 2-((4-(5,6-dimethylpyrimidin-4-yl)piperazin-1-yl)methyl)-6-fluorobenzo[d]oxazole (Compound 301)

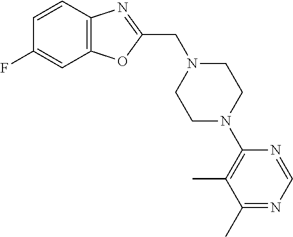 [SMILES: Cc1ncnc(N2CCN(Cc3nc4ccc(F)cc4o3)CC2)c1C]

Compound 2-((4-(5,6-dimethylpyrimidin-4-yl)piperazin-1-yl)methyl)-6-fluorobenzo[d]oxazole was prepared from 2-(chloromethyl)-6-fluorobenzo[d]oxazole (0.60 g, 3.23 mmol) and 4,5-dimethyl-6-(piperazin-1-yl)pyrimidine hydrochloride (0.80 g, 3.50 mmol) following a procedure similar to that described for the synthesis of 2-[[4-(4-pyridyl)piperazin-1-yl]methyl]-1H-benzimidazole, and was isolated as a light yellow solid.

Yield 0.52 g (47%). ¹H NMR (400 MHz, DMSO) δ 8.41 (s, 1H), 7.81-7.70 (m, 2H), 7.30-7.19 (m, 1H), 3.93 (s, 2H), 3.26 (t, J=4.8 Hz, 4H), 2.68 (t, J=4.8 Hz, 4H), 2.32 (s, 3H), 2.09 (s, 3H). ¹⁹F NMR (376 MHz, DMSO) δ −115.61. m/z: [ESI⁺] 342 (M+H)⁺, (C₁₈H₂₀FN₅O).

### Synthesis of 2-((4-(5,6-dimethylpyrimidin-4-yl)piperazin-1-yl)methyl)-7-fluorobenzo[d]oxazole (Compound 305)

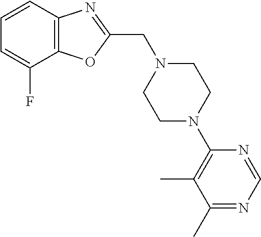 [SMILES: Cc1ncnc(N2CCN(Cc3nc4cccc(F)c4o3)CC2)c1C]

Compound 2-((4-(5,6-dimethylpyrimidin-4-yl)piperazin-1-yl)methyl)-7-fluorobenzo[d]oxazole was prepared from 2-(chloromethyl)-7-fluorobenzo[d]oxazole (0.60 g, 3.23 mmol) and 4,5-dimethyl-6-(piperazin-1-yl)pyrimidine hydrochloride (0.80 g, 3.50 mmol) following a procedure similar to that described for the synthesis of 2-[[4-(4-pyridyl)piperazin-1-yl]methyl]-1H-benzimidazole, and was isolated as a yellow solid.

Yield 0.58 g (53%). ¹H NMR (400 MHz, DMSO) δ 8.42 (s, 1H), 7.62 (dd, J=1.2, 7.6 Hz, 1H), 7.45-7.27 (m, 2H), 3.99 (s, 2H), 3.27 (t, J=4.8 Hz, 4H), 2.71 (t, J=4.8 Hz, 4H), 2.32 (s, 3H), 2.10 (s, 3H). ¹⁹F NMR (376 MHz, DMSO) δ −134.55. m/z: [ESI⁺] 342 (M+H)⁺, (C₁₈H₂₀FN₅O).

### Synthesis of 2-((4-(5,6-dimethylpyrimidin-4-yl)piperazin-1-yl)methyl)-4-(trifluoromethyl)benzo[d]oxazole (Compound 295)

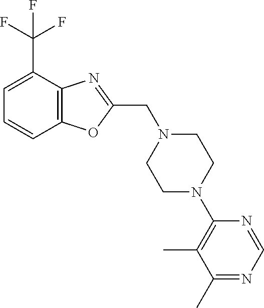 [SMILES: Cc1ncnc(N2CCN(Cc3nc4c(C(F)(F)F)cccc4o3)CC2)c1C]

Compound 2-((4-(5,6-dimethylpyrimidin-4-yl)piperazin-1-yl)methyl)-4-(trifluoromethyl) benzo[d]oxazole was prepared from 2-(chloromethyl)-4-(trifluoromethyl)benzo[d]oxazole (400 mg, 1.698 mmol) and 4,5-dimethyl-6-(piperazin-1-yl)pyrimidine hydrochloride (427 mg, 1.867 mmol) following a procedure similar to that described for the synthesis of 2-[[4-(4-pyridyl)piperazin-1-yl]methyl]-1H-benzimidazole, and was isolated as a yellow oil.

Yield 40 mg (6%). ¹H NMR (400 MHz, DMSO) δ 8.42 (s, 1H), 8.11 (dd, J=0.8, 8.0 Hz, 1H), 7.75 (dd, J=0.8, 8.0 Hz, 1H), 7.64-7.54 (m, 1H), 4.02 (s, 2H), 3.27 (t, J=4.8 Hz, 4H), 2.70 (t, J=4.8 Hz, 4H), 2.32 (s, 3H), 2.10 (s, 3H). ¹⁹F NMR (376 MHz, DMSO) δ −59.40. m/z: [ESI⁺] 392 (M+H)⁺, (C₁₉H₂₀F₃N₅O).

### Synthesis of 2-((4-(5,6-dimethylpyrimidin-4-yl)piperazin-1-yl)methyl)-5-(trifluoromethyl)benzo[d]oxazole (Compound 299)

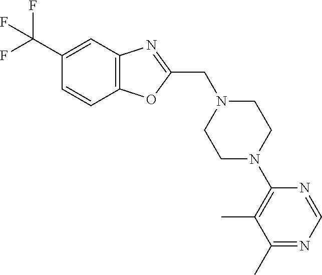 [SMILES: Cc1ncnc(N2CCN(Cc3nc4cc(C(F)(F)F)ccc4o3)CC2)c1C]

Compound 2-((4-(5,6-dimethylpyrimidin-4-yl)piperazin-1-yl)methyl)-5-(trifluoromethyl) benzo[d]oxazole was prepared from 2-(chloromethyl)-5-(trifluoromethyl)benzo[d]oxazole (0.40 g, 1.70 mmol) and 4,5-dimethyl-6-(piperazin-1-yl)pyrimidine (0.43 g, 1.88 mmol) hydrochloride following a procedure similar to that described for the synthesis of 2-[[4-(4-pyridyl)piperazin-1-yl]methyl]-1H-benzimidazole, and was isolated as an orange solid.

Yield 0.22 g (33%). ¹H NMR (400 MHz, DMSO) δ 8.42 (s, 1H), 8.24-8.15 (m, 1H), 8.00 (d, J=8.4 Hz, 1H), 7.79 (dd, J=2.0, 8.4 Hz, 1H), 4.01 (s, 2H), 3.27 (t, J=4.8 Hz, 4H), 2.71 (t, J=4.8 Hz, 4H), 2.33 (s, 3H), 2.10 (s, 3H). ¹⁹F NMR (376 MHz, DMSO) δ −59.35. m/z: [ESI⁺] 392 (M+H)⁺, (C₁₉H₂₀F₃N₅O).

### Synthesis of 2-((4-(5,6-dimethylpyrimidin-4-yl)piperazin-1-yl)methyl)-6-(trifluoromethyl)benzo[d]oxazole (Compound 303)

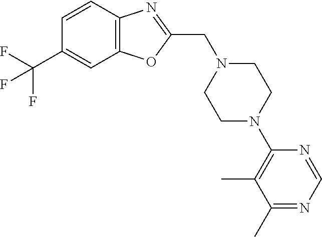 [SMILES: Cc1ncnc(N2CCN(Cc3nc4ccc(C(F)(F)F)cc4o3)CC2)c1C]

Compound 2-((4-(5,6-dimethylpyrimidin-4-yl)piperazin-1-yl)methyl)-6-(trifluoromethyl) benzo[d]oxazole was prepared from 2-(chloromethyl)-6-(trifluoromethyl)benzo[d]oxazole (400 mg, 1.698 mmol) and 4,5-dimethyl-6-(piperazin-1-yl)pyrimidine hydrochloride (427 mg, 1.867 mmol) following a procedure similar to that described for the synthesis of 2-[[4-(4-pyridyl)piperazin-1-yl]methyl]-1H-benzimidazole, and was isolated as an orange solid.

Yield 166 mg (25%). ¹H NMR (400 MHz, DMSO) δ 8.42 (s, 1H), 8.31-8.23 (m, 1H), 7.98 (d, J=8.4 Hz, 1H), 7.75 (dd, J=1.6, 8.4 Hz, 1H), 4.02 (s, 2H), 3.29-3.23 (t, J=4.8 Hz, 4H), 2.71 (t, J=4.8 Hz, 4H), 2.32 (s, 3H), 2.10 (s, 3H). ¹⁹F NMR (376 MHz, DMSO) δ −59.40. m/z: [ESI⁺] 392 (M+H)⁺, (C₁₉H₂₀F₃N₅O).

### Synthesis of 2-((4-(5,6-dimethylpyrimidin-4-yl)piperazin-1-yl)methyl)-7-(trifluoromethyl)benzo[d]oxazole (Compound 307)

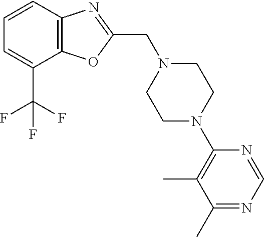 [SMILES: Cc1ncnc(N2CCN(Cc3nc4cccc(C(F)(F)F)c4o3)CC2)c1C]

Compound 2-((4-(5,6-dimethylpyrimidin-4-yl)piperazin-1-yl)methyl)-7-(trifluoromethyl) benzo[d]oxazole was prepared from 2-(chloromethyl)-7-(trifluoromethyl)benzo[d]oxazole (250 mg, 1.061 mmol) and 4,5-dimethyl-6-(piperazin-1-yl)pyrimidine hydrochloride (266 mg, 1.163 mmol) following a procedure similar to that described for the synthesis of 2-[[4-(4-pyridyl)piperazin-1-yl]methyl]-1H-benzimidazole, and was isolated as a light yellow oil.

Yield 50 mg (12%). ¹H NMR (400 MHz, DMSO) δ 8.42 (s, 1H), 8.11 (dd, J=0.8, 8.0 Hz, 1H), 7.78 (dd, J=0.8, 7.8 Hz, 1H), 7.59 (dd, J=7.8, 8.0 Hz, 1H), 4.04 (s, 2H), 3.27 (t, J=4.8 Hz, 4H), 2.73 (t, J=4.8 Hz, 4H), 2.32 (s, 3H), 2.10 (s, 3H). ¹⁹F NMR (376 MHz, DMSO) δ −59.49. m/z: [ESI⁺] 392 (M+H)⁺, (C₁₉H₂₀F₃N₅O).

### Synthesis of 6-chloro-2-((4-(5,6-dimethylpyrimidin-4-yl)piperazin-1-yl)methyl)benzo[d]oxazole (Compound 302)

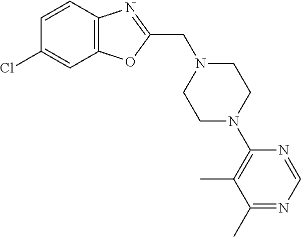 [SMILES: Cc1ncnc(N2CCN(Cc3nc4ccc(Cl)cc4o3)CC2)c1C]

Compound 6-chloro-2-((4-(5,6-dimethylpyrimidin-4-yl)piperazin-1-yl)methyl)benzo[d]oxazole was prepared from 6-chloro-2-(chloromethyl)benzo[d]oxazole (486 mg, 2.405 mmol) and 4,5-dimethyl-6-(piperazin-1-yl)pyrimidine hydrochloride (500 mg, 2.186 mmol) following a procedure similar to that described for the synthesis of 2-[[4-(4-pyridyl)piperazin-1-yl]methyl]-1H-benzimidazole, and was isolated as an orange oil.

Yield 0.40 g (51%). ¹H NMR (400 MHz, DMSO) δ 8.42 (s, 1H), 7.98 (d, J=2.0 Hz, 1H), 7.77 (d, J=8.4 Hz, 1H), 7.44 (dd, J=2.0, 8.4 Hz, 1H), 3.95 (s, 2H), 3.25 (t, J=4.8 Hz, 4H), 2.68 (t, J=4.8 Hz, 4H), 2.32 (s, 3H), 2.09 (s, 3H). m/z: [ESI⁺] 358, 360 (M+H)⁺, (C₁₈H₂₀ClN₅O).

### Synthesis of 2-((4-(5-chloro-6-methylpyrimidin-4-yl)piperazin-1-yl)methyl)-6-fluorobenzo[d]oxazole (Compound 327)

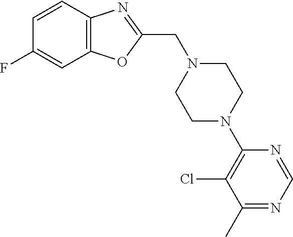 [SMILES: Cc1ncnc(N2CCN(Cc3nc4ccc(F)cc4o3)CC2)c1Cl]

Compound 2-((4-(5-chloro-6-methylpyrimidin-4-yl)piperazin-1-yl)methyl)-6-fluorobenzo [d]oxazole was prepared from 2-(chloromethyl)-6-fluorobenzo[d]oxazole (384 mg, 2.069 mmol) and 5-chloro-4-methyl-6-(piperazin-1-yl)pyrimidine hydrochloride (400 mg, 1.606 mmol) following a procedure similar to that described for the synthesis of 2-[[4-(4-pyridyl)piperazin-1-yl]methyl]-1H-benzimidazole, and was isolated as an off-white solid.

Yield 30 mg (5%). ¹H NMR (400 MHz, DMSO) δ 8.46 (s, 1H), 7.82-7.71 (m, 2H), 7.31-7.21 (m, 1H), 3.94 (s, 2H), 3.58 (t, J=4.8 Hz, 4H), 2.69 (t, J=4.8 Hz, 4H), 2.44 (s, 3H). m/z: [ESI⁺] 362, 364 (M+H)⁺, (C₁₇H₁₇ClFN₅O).

### Synthesis of 2-((5-(5,6-dimethylpyrimidin-4-yl)-2,5-diazabicyclo[2.2.1]heptan-2-yl)methyl)-6-fluorobenzo[d]oxazole (Compound 334)

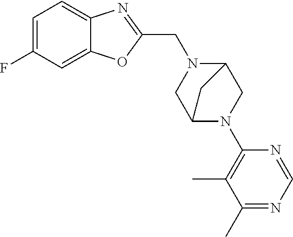 [SMILES: Cc1ncnc(N2CC3CC2CN3Cc2nc3ccc(F)cc3o2)c1C]

Compound 2-((5-(5,6-dimethylpyrimidin-4-yl)-2,5-diazabicyclo[2.2.1]heptan-2-yl)methyl)-6-fluorobenzo[d]oxazole was prepared from 2-(chloromethyl)-6-fluorobenzo[d]oxazole (200 mg, 1.078 mmol) and 2-(5,6-dimethylpyrimidin-4-yl)-2,5-diazabicyclo[2.2.1]heptane hydrochloride (389 mg, 1.616 mmol) following a procedure similar to that described for the synthesis of 2-[[4-(4-pyridyl)piperazin-1-yl]methyl]-1H-benzimidazole, and was isolated as a dark yellow oil.

Yield 105 mg (28%). ¹H NMR (400 MHz, DMSO) δ 8.23 (s, 1H), 7.78-7.61 (m, 2H), 7.32-7.17 (m, 1H), 4.66 (s, 1H), 3.99 (s, 2H), 3.75-3.62 (m, 2H), 3.56-3.48 (m, 1H), 3.01-2.87 (m, 2H), 2.29 (s, 3H), 2.11 (s, 3H), 1.87 (dd, J=2.4, 9.6 Hz, 1H), 1.75 (d, J=9.6 Hz, 1H). ¹⁹F NMR (376 MHz, DMSO) δ −115.79. m/z: [ESI⁺] 354 (M+H)⁺, (C₁₉H₂₀FN₅O).

### Synthesis of 2-((4-(5-fluoro-6-methylpyrimidin-4-yl)piperazin-1-yl)methyl)benzo[d]oxazole (Compound 309)

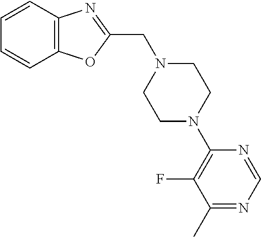 [SMILES: Cc1ncnc(N2CCN(Cc3nc4ccccc4o3)CC2)c1F]

Compound 2-((4-(5-fluoro-6-methylpyrimidin-4-yl)piperazin-1-yl)methyl)benzo[d]oxazole was prepared from 2-(chloromethyl)benzo[d]oxazole (235 mg, 1.402 mmol) and 5-fluoro-4-methyl-6-(piperazin-1-yl)pyrimidine hydrochloride (250 mg, 1.074 mmol) following a procedure similar to that described for the synthesis of 2-[[4-(4-pyridyl)piperazin-1-yl]methyl]-1H-benzimidazole, and was isolated as an off-white solid.

Yield 88 mg (25%). ¹H NMR (400 MHz, DMSO) δ 8.22 (d, J=2.4 Hz, 1H), 7.76-7.71 (m, 2H), 7.44-7.31 (m, 2H), 3.93 (s, 2H), 3.70 (t, J=4.8 Hz, 4H), 2.65 (t, J=4.8 Hz, 4H), 2.28 (d, J=3.6 Hz, 3H). ¹⁹F NMR (376 MHz, DMSO) δ −144.04. m/z: [ESI⁺] 328 (M+H)⁺, (C₁₇H₁₈FN₅O).

### Synthesis of 2-((4-(1-methyl-1H-1,2,4-triazol-3-yl)piperazin-1-yl)methyl)benzo[d]oxazole (Compound 326)

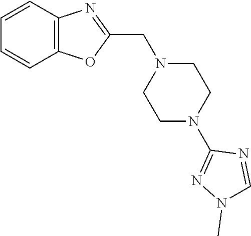 [SMILES: Cn1cnc(N2CCN(Cc3nc4ccccc4o3)CC2)n1]

Compound 2-((4-(1-methyl-1H-1,2,4-triazol-3-yl)piperazin-1-yl)methyl)benzo[d]oxazole was prepared from 2-(chloromethyl)benzo[d]oxazole (83 mg, 0.495 mmol) and 1-(1-methyl-1H-1,2,4-triazol-3-yl)piperazine (75 mg, 0.449 mmol) following a procedure similar to that described for the synthesis of 2-[[4-(4-pyridyl)piperazin-1-yl]methyl]-1H-benzimidazole, and was isolated as a light yellow solid.

Yield 10 mg (7%). ¹H NMR (400 MHz, DMSO) δ 8.06 (d, J=0.8 Hz, 1H), 7.79-7.70 (m, 2H), 7.46-7.34 (m, 2H), 3.91 (s, 2H), 3.67 (s, 3H), 3.29 (t, J=4.8 Hz, 4H), 2.62 (t, J=4.8 Hz, 4H). m/z: [ESI⁺]299 (M+H)⁺, (C₁₅H₁₈N₆O).

### Synthesis of 2-((4-(1-methyl-1H-pyrazol-3-yl)piperazin-1-yl)methyl)benzo[d]oxazole (Compound 324)

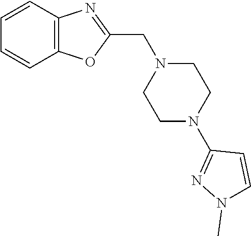 [SMILES: Cn1ccc(N2CCN(Cc3nc4ccccc4o3)CC2)n1]

Compound 2-((4-(1-methyl-1H-pyrazol-3-yl)piperazin-1-yl)methyl)benzo[d]oxazole was prepared from 2-(chloromethyl)benzo[d]oxazole (200 mg, 1.203 mmol) and 1-(1-methyl-1H-pyrazol-3-yl)piperazine (222 mg, 1.335 mmol) following a procedure similar to that described for the synthesis of 2-[[4-(4-pyridyl)piperazin-1-yl]methyl]-1H-benzimidazole, and was isolated as an off-white solid.

Yield 30 mg (8%). ¹H NMR (400 MHz, DMSO) δ 7.78-7.70 (m, 2H), 7.46-7.34 (m, 3H), 5.64 (d, J=2.4 Hz, 1H), 3.90 (s, 2H), 3.64 (s, 3H), 3.08 (t, J=4.8 Hz, 4H), 2.63 (t, J=4.8 Hz, 4H). m/z: [ESI⁺]298 (M+H)⁺, (C₁₆H₁₉N₅O).

### Synthesis of 2-((4-(5-chloro-6-methylpyrimidin-4-yl)piperazin-1-yl)methyl)benzo[d]oxazole (Compound 310)

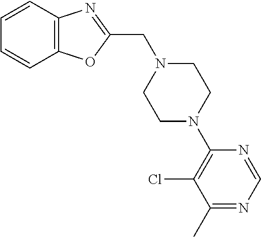 [SMILES: Cc1ncnc(N2CCN(Cc3nc4ccccc4o3)CC2)c1Cl]

Compound 2-((4-(5-chloro-6-methylpyrimidin-4-yl)piperazin-1-yl)methyl)benzo[d]oxazole was prepared from 2-(piperazin-1-ylmethyl)benzo[d]oxazole bis(trifluoroacetate) (100 mg, 0.225 mmol) and 4,5-dichloro-6-methylpyrimidine (96 mg, 0.589 mmol) following a procedure similar to that described for the synthesis of tert-butyl 4-(5-chloro-6-methylpyrimidin-4-yl)piperazine-1-carboxylate, except DMF was used as solvent, and was isolated as an off-white solid.

Yield 32 mg (41%). ¹H NMR (400 MHz, DMSO) δ 8.45 (s, 1H), 7.77-7.69 (m, 2H), 7.45-7.32 (m, 2H), 3.95 (s, 2H), 3.58 (t, J=4.8 Hz, 4H), 2.70 (t, J=4.8 Hz, 4H), 2.44 (s, 3H). m/z: [ESI⁺] 344, 346 (M+H)⁺, (C₁₇H₁₈ClN₅O).

### Synthesis of 2-((4-(5-chloropyrimidin-4-yl)piperazin-1-yl)methyl)benzo[d]oxazole (Compound 314)

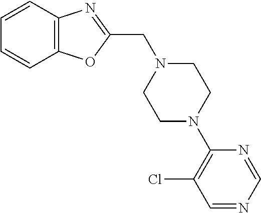 [SMILES: Clc1cncnc1N1CCN(Cc2nc3ccccc3o2)CC1]

Compound 2-((4-(5-chloropyrimidin-4-yl)piperazin-1-yl)methyl)benzo[d]oxazole was prepared from 2-(piperazin-1-ylmethyl)benzo[d]oxazole bis(trifluoroacetate) (600 mg, 1.347 mmol) and 4,5-dichloropyrimidine (527 mg, 3.538 mmol) following a procedure similar to that described for the synthesis of tert-butyl 4-(5-chloro-6-methylpyrimidin-4-yl)piperazine-1-carboxylate, except DMF was used as solvent, and was isolated as a yellow oil.

Yield 7 mg (2%). ¹H NMR (300 MHz, DMSO) δ 8.56 (s, 1H), 8.39 (s, 1H), 7.79-7.62 (m, 2H), 7.46-7.32 (m, 2H), 3.95 (s, 2H), 3.68 (t, J=4.8 Hz, 4H), 2.69 (t, J=4.8 Hz, 4H). m/z: [ESI⁺] 330, 332 (M+H)⁺, (C₁₆H₁₆ClN₅O).

### Synthesis of 2-((4-(5,6-dimethylpyrimidin-4-yl)piperazin-1-yl)methyl)oxazolo[5,4-c]pyridine (Compound 330)

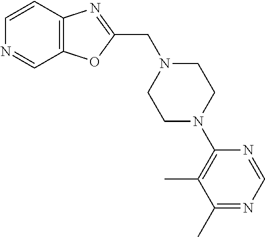 [SMILES: Cc1ncnc(N2CCN(Cc3nc4ccncc4o3)CC2)c1C]

Compound 2-((4-(5,6-dimethylpyrimidin-4-yl)piperazin-1-yl)methyl)oxazolo[5,4-c]pyridine was prepared from 2-(piperazin-1-ylmethyl)oxazolo[5,4-c]pyridine bistrifluoroacetate (500 mg, 1.120 mmol) and 4-chloro-5,6-dimethylpyrimidine (490 mg, 3.436 mmol) following a procedure similar to that described for the synthesis of tert-butyl 4-(5-chloro-6-methylpyrimidin-4-yl)piperazine-1-carboxylate, except DMF was used as solvent, and was isolated as a yellow solid.

Yield 50 mg (14%). ¹H NMR (400 MHz, DMSO) δ 9.10 (d, J=1.2 Hz, 1H), 8.55 (d, J=5.4 Hz, 1H), 8.42 (s, 1H), 7.84 (dd, J=1.2, 5.4 Hz, 1H), 4.04 (s, 2H), 3.27 (t, J=4.8 Hz, 4H), 2.74-2.70 (m, 4H), 2.32 (s, 3H), 2.09 (s, 3H). m/z: [ESI⁺] 325 (M+H)⁺, (C₁₇H₂₀N₆O).

### Synthesis of 2-(1-(4-(5,6-dimethylpyrimidin-4-yl)piperazin-1-yl)cyclopropyl)benzo[d]oxazole (Compound 321)

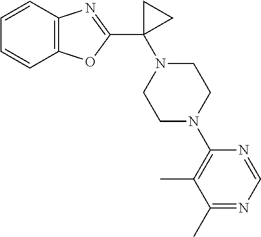 [SMILES: Cc1ncnc(N2CCN(C3(c4nc5ccccc5o4)CC3)CC2)c1C]

Compound 2-(1-(4-(5,6-dimethylpyrimidin-4-yl)piperazin-1-yl)cyclopropyl)benzo[d]oxazole was prepared from 2-(1-(piperazin-1-yl)cyclopropyl)benzo[d]oxazole (23 mg, 0.095 mmol) and 4-chloro-5,6-dimethylpyrimidine (20 mg, 0.140 mmol) following a procedure similar to that described for the synthesis of tert-butyl 4-(5-chloro-6-methylpyrimidin-4-yl)piperazine-1-carboxylate, except DMF was used as solvent, and was isolated as an off white solid.

Yield 5 mg (15%). ¹H NMR (400 MHz, DMSO) δ 8.42 (s, 1H), 7.72-7.68 (m, 1H), 7.68-7.64 (m, 1H), 7.38-7.28 (m, 2H), 3.22 (s, 4H), 3.13 (t, J=4.8 Hz, 4H), 2.33 (s, 3H), 2.13 (s, 3H), 1.41 (q, J=4.4 Hz, 2H), 1.23 (q, J=4.4 Hz, 2H). m/z: [ESI⁺] 350 (M+H)⁺, (C₂₀H₂₃N₅O).

### Synthesis of 4-(4-((6-fluorobenzo[d]oxazol-2-yl)methyl)piperazin-1-yl)-6-methylpyrimidine-5-carbonitrile (Compound 329)

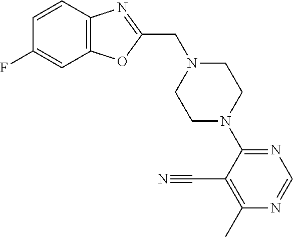 [SMILES: Cc1ncnc(N2CCN(Cc3nc4ccc(F)cc4o3)CC2)c1C#N]

To a mixture of 6-fluoro-2-((4-(5-iodo-6-methylpyrimidin-4-yl)piperazin-1-yl)methyl)benzo[d]oxazole (200 mg, 0.441 mmol) and zinc cyanide (104 mg, 0.886 mmol) in DMF (5 mL) was added tetrakis(triphenylphosphine)-palladium(0) (51 mg, 0.044 mmol). The reaction mixture was purged with nitrogen gas and subjected to microwave irradiation at 120° C. for 3 h. The resulting mixture was cooled to room temperature and purified by reverse phase flash chromatography with the following conditions Column: WelHash™ C18-I, 20-40 um, 120 g; Eluent A: water (plus 10 mmol/L NH₄HCO₃); Eluent B: acetonitrile; Gradient: 45%-65% B in 20 min; How rate: 60 mL/min; Detector: UV 220/254 nm. Desired fractions were collected and concentrated under reduced pressure to afford 4-(4-((6-fluorobenzo[d]oxazol-2-yl)methyl)piperazin-1-yl)-6-methylpyrimidine-5-carbonitrile as an off-white solid.

Yield 36 mg (23%). ¹H NMR (400 MHz, DMSO) δ 8.55 (s, 1H), 7.81-7.71 (m, 2H), 7.29-7.23 (m, 1H), 3.95 (s, 2H), 3.93 (t, J=4.8 Hz, 4H), 2.69 (t, J=4.8 Hz, 4H), 2.49 (s, 3H). ¹⁹F NMR (376 MHz, DMSO) δ −115.58. m/z: [ESI⁺] 353 (M+H)⁺, (C₁₈H₁₇FN₆O).

### Synthesis of 2-((4-(6-methyl-5-(trifluoromethyl)pyrimidin-4-yl)piperazin-1-yl)methyl)benzo[d]oxazole (Compound 311)

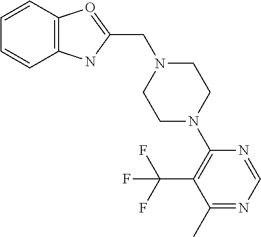

A mixture of copper(I) iodide (315 mg, 1.654 mmol) and potassium fluoride (80 mg, 1.377 mmol) was stirred for 2 h at 150° C. under vacuum. After cooling to room temperature, a nitrogen degassed solution of trifluoromethyltrimethylsilane (196 mg, 1.378 mmol) and 2-((4-(5-iodo-6-methylpyrimidin-4-yl)piperazin-1-yl)methyl)benzo[d]oxazole (200 mg, 0.459 mmol) in NMP (4 mL) was added to the mixture. The resulting mixture was then stirred under a nitrogen atmosphere for 16 h at room temperature. The resulting solution was purified by reverse phase flash chromatography with the following conditions: Column: WelHash™ C18-I 20-40 um, 120 g; Eluent A: water (plus 10 mmol/L NH₄HCO₃); Eluent B: acetonitrile; Gradient: 30%-50% B in 25 min; How rate: 60 mL/min; Detector: UV 220/254 nm. Desired fractions were collected and concentrated under reduced pressure to afford 2-((4-(6-methyl-5-(trifluoromethyl)pyrimidin-4-yl)piperazin-1-yl)methyl)benzo[d]oxazole as a yellow oil.

Yield 10 mg (6%). ¹H NMR (400 MHz, DMSO) δ 8.49 (s, 1H), 7.78-7.65 (m, 2H), 7.44-7.30 (m, 2H), 3.93 (s, 2H), 3.54 (t, J=4.8 Hz, 4H), 2.64 (t, J=4.8 Hz, 4H), 2.45 (s, 3H). ¹⁹F NMR (376 MHz, DMSO) δ −54.74. m/z: [ESI⁺] 378 (M+H)⁺, (C₁₈H₁₈F₃N₅O).

### Synthesis of 6-(difluoromethyl)-2-((4-(5,6-dimethylpyrimidin-4-yl)piperazin-1-yl)methyl)benzo[d]oxazole (Compound 304)

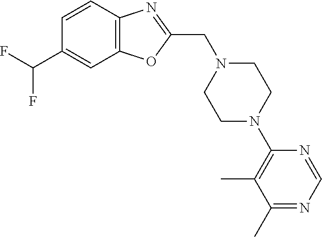 [SMILES: Cc1ncnc(N2CCN(Cc3nc4ccc(C(F)F)cc4o3)CC2)c1C]

A solution of 2-((4-(5,6-dimethylpyrimidin-4-yl)piperazin-1-yl)methyl)benzo[d]oxazole-6-carbaldehyde (400 mg, 1.138 mmol) and DAST (917 mg, 5.689 mmol) in DCM (8 mL), was stirred for 16 h at room temperature under a nitrogen atmosphere. The resulting solution was concentrated under reduced pressure. The residue was purified by reverse phase flash chromatography with the following conditions: Column: WelHash™ C18-I, 20-40 um, 120 g; Eluent A: water (plus 10 mmol/L NH₄HCO₃); Eluent B: acetonitrile; Gradient: 25%-45% B in 25 min; How rate: 60 mL/min; Detector: UV 220/254 nm; desired fractions were collected and concentrated under reduced pressure to afford 6-(difluoromethyl)-2-((4-(5,6-dimethylpyrimidin-4-yl)piperazin-1-yl)methyl)benzo[d]oxazole as a red oil.

Yield 100 mg (24%). ¹H NMR (400 MHz, DMSO) δ 8.42 (s, 1H), 8.01 (s, 1H), 7.89 (d, J=8.0 Hz, 1H), 7.60 (d, J=8.0 Hz, 1H), 7.17 (t, J=55.6 Hz, 1H), 3.98 (s, 2H), 3.26 (d, J=4.8 Hz, 4H), 2.70 (t, J=4.8 Hz, 4H), 2.32 (s, 3H), 2.09 (s, 3H). ¹⁹F NMR (376 MHz, DMSO) δ −107.15. m/z: [ESI⁺] 374 (M+H)⁺, (C₁₉H₂₁F₂N₅O).

### Synthesis of 4-(4-(benzo[d]oxazol-2-ylmethyl)piperazin-1-yl)-6-methylpyrimidine-5-carbaldehyde (Compound 336)

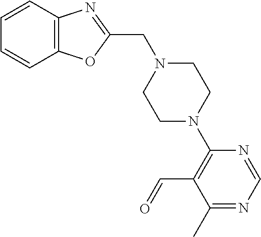 [SMILES: Cc1ncnc(N2CCN(Cc3nc4ccccc4o3)CC2)c1C=O]

Compound 4-(4-(benzo[d]oxazol-2-ylmethyl)piperazin-1-yl)-6-methylpyrimidine-5-carbaldehyde was prepared from 2-((4-(6-methyl-5-vinylpyrimidin-4-yl)piperazin-1-yl)methyl)benzo[d]oxazole (500 mg, 1.491 mmol) following a similar procedure to that described for the synthesis of tert-butyl 4-(5-formylpyrimidin-4-yl)piperazine-1-carboxylate, and was isolated as a yellow solid.

Yield 89 mg (18%). ¹H NMR (400 MHz, DMSO) δ 10.03 (s, 1H), 8.46 (s, 1H), 7.78-7.67 (m, 2H), 7.45-7.31 (m, 2H), 3.94 (s, 2H), 3.60 (t, J=4.8 Hz, 4H), 2.66 (t, J=4.8 Hz, 4H), 2.59 (s, 3H). m/z: [ESI⁺] 338 (M+H)⁺, (C₁₈H₁₉N₅O₂).

### Synthesis of 2-((4-(5-(difluoromethyl)-6-methylpyrimidin-4-yl)piperazin-1-yl)methyl)benzo[d]oxazole (Compound 312)

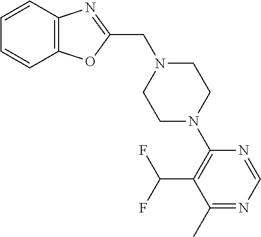 [SMILES: Cc1ncnc(N2CCN(Cc3nc4ccccc4o3)CC2)c1C(F)F]

To a stirred solution of 4-[4-(1,3-benzoxazol-2-ylmethyl)piperazin-1-yl]-6-methylpyrimidine-5-carbaldehyde (195 mg, 0.578 mmol) in DCM (4 mL) was added DAST (559 mg, 3.468 mmol) at room temperature under a nitrogene atmosphere. The resulting mixture was stirred for overnight at room temperature. The resulting mixture was concentrated under reduced pressure. The residue was purified by reverse phase flash chromatography with the following conditions: column: WelFlash™ C18-I, 20-40 um, 120 g; Eluent A: water (plus 10 mmol/L NH₄HCO₃); Eluent B: ACN; Gradient: 25%-45% B in 25 min; How rate: 60 mL/min; Detector: UV 220/254 nm. Desired fractions were collected and concentrated under reduced pressure to afford 2-([4-[5-(difluoromethyl)-6-methylpyrimidin-4-yl]piperazin-1-yl]methyl)-1,3-benzoxazole as a yellow oil.

Yield 77 mg (37%). ¹H NMR (400 MHz, DMSO) δ 8.54 (s, 1H), 7.78-7.66 (m, 2H), 7.45-7.31 (m, 2H), 7.03 (t, J=52.8 Hz, 1H), 3.93 (s, 2H), 3.48 (t, J=4.8 Hz, 4H), 2.69 (t, J=4.8 Hz, 4H), 2.47 (s, 3H). ¹⁹F NMR (376 MHz, CDCl₃) δ −112.04. m/z: [ESI⁺] 360 (M+H)⁺, (C₁₈H₁₉F₂N₅O).

### Synthesis of 2-((4-(5-(trifluoromethyl)pyrimidin-4-yl)piperazin-1-yl)methyl)benzo[d]oxazole (Compound 315)

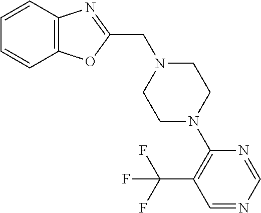 [SMILES: FC(F)(F)c1cncnc1N1CCN(Cc2nc3ccccc3o2)CC1]

To a stirred solution of 2-((4-(2-chloro-5-(trifluoromethyl)pyrimidin-4-yl)piperazin-1-yl)methyl)benzo[d]oxazole (225 mg, 0.566 mmol) in ethanol (45 mL) was added 10% wt. palladium on carbon (1.00 g) at room temperature. Following nitrogen degassing, the mixture was stirred for 16 h at room temperature under a hydrogen atmosphere. The resulting mixture was filtered through Celite and washed with ethanol (4×20 mL). The washings were combined and concentrated under reduced pressure. The residue was purified by reverse phase flash chromatography with the following conditions: Column: WelHash™ C18-I, 20-40 um, 120 g; Eluent A: water (plus 10 mmol/L NH₄HCO₃); Eluent B: acetonitrile; Gradient: 35%-55% B in 25 min; How rate: 60 mL/min; Detector: UV 220/254 nm. Desired fractions were collected and concentrated under reduced pressure to afford 2-((4-(5-(trifluoromethyl)pyrimidin-4-yl)piperazin-1-yl)methyl)benzo[d]oxazole as an off-white solid.

Yield 10 mg (5%). ¹H NMR (400 MHz, DMSO₆) δ 8.72 (s, 1H), 8.68 (s, 1H), 7.79-7.65 (m, 2H), 7.46-7.30 (m, 2H), 3.94 (s, 2H), 3.65 (t, J=4.8 Hz, 4H), 2.67 (t, J=4.8 Hz, 4H). ¹⁹F NMR (376 MHz, DMSO) δ −56.29. m/z: [ESI⁺] 364 (M+H)⁺, (C₁₇H₁₆F₃N₅O).

### Synthesis of 2-((4-(5-(2-(3-(but-3-yn-1-yl)-3H-diazirin-3-yl)ethoxy)pyrimidin-4-yl)piperazin-1-yl)methyl)-1H-indole (Compound 337)

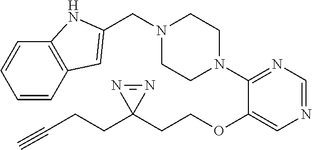 [SMILES: C#CCCC1(CCOc2cncnc2N2CCN(Cc3cc4ccccc4[nH]3)CC2)N=N1]

A mixture of 3-(but-3-yn-1-yl)-3-(2-iodoethyl)-3H-diazirine (150 mg, 0.605 mmol), 4-(4-((1H-indol-2-yl)methyl)piperazin-1-yl)pyrimidin-5-ol (220 mg, 0.711 mmol) and cesium carbonate (590 mg, 1.811 mmol) in DMF (10 mL) was stirred at 80° C., under a nitrogen atmosphere, for 16 h. The resulting mixture was cooled to room temperature and purified by reverse phase flash chromatography with the following conditions: column, C18 silica gel; mobile phase, acetonitrile in water (plus 5 mmol/L NH₄HCO₃), 50% to 65% gradient in 20 min; detector, UV 220/254 nm. Desired fractions were collected and concentrated under reduced pressure to afford to afford 2-((4-(5-(2-(3-(but-3-yn-1-yl)-3H-diazirin-3-yl)ethoxy)pyrimidin-4-yl)piperazin-1-yl)methyl)-1H-indole as a light yellow solid.

Yield 40 mg (15%). ¹H NMR (400 MHz, DMSO) δ 11.03 (br s, 1H), 8.24 (s, 1H), 7.99 (s, 1H), 7.45 (d, J=7.8 Hz, 1H), 7.33 (dd, J=1.2, 8.0 Hz, 1H), 7.05-6.99 (m, 1H), 6.97-6.90 (m, 1H), 6.30 (s, 1H), 3.90 (t, J=5.6 Hz, 2H), 3.75 (t, J=5.2 Hz, 4H), 3.67 (s, 2H), 2.80 (t, J=2.8 Hz, 1H), 2.56 (t, J=5.2 Hz, 4H), 2.00 (dt, J=2.8, 7.2 Hz, 2H), 1.89 (t, J=5.6 Hz, 2H), 1.65 (t, J=7.2 Hz, 2H). m/z: [ESI⁺] 430 (M+H)⁺, (C₂₄H₂₇N₇O).

### Synthesis of 6-(2-(3-(but-3-yn-1-yl)-3H-diazirin-3-yl)ethoxy)-2-((4-(5-ethylpyrimidin-4-yl)piperazin-1-yl)methyl)-1H-indole (Compound 338)

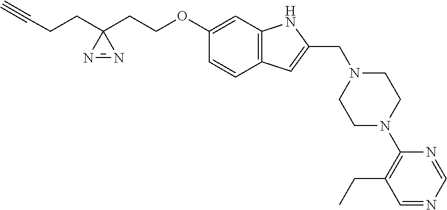 [SMILES: C#CCCC1(CCOc2ccc3cc(CN4CCN(c5ncncc5CC)CC4)[nH]c3c2)N=N1]

Compound 6-(2-(3-(but-3-yn-1-yl)-3H-diazirin-3-yl)ethoxy)-2-((4-(5-ethylpyrimidin-4-yl)piperazin-1-yl)methyl)-1H-indole was prepared from 2-((4-(5-ethylpyrimidin-4-yl)piperazin-1-yl)methyl)-1H-indol-6-ol (50 mg, 0.148 mmol) and 3-(but-3-yn-1-yl)-3-(2-iodoethyl)-3H-diazirine (44 mg, 0.177 mmol) following a procedure similar to that described for the synthesis of 2-((4-(5-(2-(3-(but-3-yn-1-yl)-3H-diazirin-3-yl)ethoxy)pyrimidin-4-yl)piperazin-1-yl)methyl)-1H-indole, and was isolated as a brown oil.

Yield 15 mg (22%). ¹H NMR (400 MHz, DMSO) δ 10.85 (br s, 1H), 8.52 (s, 1H), 8.24 (s, 1H), 7.32 (d, J=8.4 Hz, 1H), 6.82 (d, J=2.0 Hz, 1H), 6.61 (dd, J=2.0, 8.4 Hz, 1H), 6.21 (s, 1H), 3.79 (t, J=6.0 Hz, 2H), 3.62 (s, 2H), 3.40-3.30 (m, 4H), 2.84 (t, J=2.0 Hz, 1H), 2.62-2.50 (m, 6H), 2.06 (t, J=7.2 Hz, 2H), 1.88 (t, J=6.0 Hz, 2H), 1.68 (t, J=7.2 Hz, 2H), 1.18 (t, J=7.6 Hz, 3H). m/z: [ESI⁺] 458 (M+H)⁺, (C₂₆H₃₁N₇O).

### Example 2

### Biological Activity of Compounds of the Invention

The biological activity results of all compounds of the invention is summarized in Table 2.

TABLE 2
Cellular EC₅₀ values of compounds of the invention
in the WI-38 collagen 1 inhibition assay.

| | COL1 Efficacy (LogEC50) |
|---|---|
| | −: inactive +: >−5 |
| Compound No. | ++: −5 to −6 +++: <−6 |
| 200 | − |
| 201 | +++ |
| 202 | +++ |
| 203 | ++ |
| 204 | ++ |
| 205 | ++ |
| 207 | ++ |
| 208 | ++ |
| 209 | + |
| 210 | +++ |
| 211 | +++ |
| 212 | +++ |
| 213 | +++ |
| 214 | +++ |
| 215 | − |
| 216 | + |
| 217 | + |
| 218 | + |
| 219 | − |
| 220 | − |
| 221 | − |
| 222 | ++ |
| 223 | − |
| 224 | + |
| 225 | + |
| 226 | + |
| 227 | − |
| 228 | + |
| 229 | ++ |
| 230 | + |
| 231 | − |
| 232 | ++ |
| 233 | + |
| 234 | + |
| 235 | + |
| 236 | ++ |
| 237 | ++ |
| 238 | ++ |
| 239 | ++ |
| 240 | + |
| 241 | ++ |
| 242 | + |
| 243 | − |
| 244 | + |
| 245 | − |
| 246 | ++ |
| 247 | +++ |
| 248 | +++ |
| 249 | +++ |
| 250 | + |
| 251 | + |
| 252 | ++ |
| 253 | ++ |
| 254 | +++ |
| 255 | +++ |
| 256 | +++ |
| 257 | ++ |
| 258 | ++ |
| 259 | +++ |
| 260 | ++ |
| 261 | − |
| 262 | − |
| 263 | − |
| 264 | − |
| 265 | ++ |
| 266 | + |
| 267 | − |
| 268 | ++ |
| 269 | − |
| 270 | − |
| 271 | − |
| 272 | − |
| 273 | − |
| 274 | − |
| 275 | − |
| 276 | − |
| 277 | − |
| 278 | − |
| 279 | − |
| 280 | − |
| 281 | + |
| 282 | + |
| 283 | − |
| 284 | − |
| 285 | − |
| 286 | − |
| 287 | − |
| 288 | ++ |
| 289 | +++ |
| 290 | ++ |
| 291 | +++ |
| 292 | ++ |
| 293 | ++ |
| 295 | +++ |
| 297 | ++ |
| 299 | +++ |
| 301 | +++ |
| 302 | +++ |
| 303 | +++ |
| 304 | +++ |
| 305 | ++ |
| 307 | ++ |
| 309 | ++ |
| 310 | ++ |
| 311 | + |
| 313 | + |
| 314 | ++ |
| 315 | + |
| 321 | ++ |
| 322 | + |
| 323 | − |
| 324 | + |
| 325 | − |
| 326 | + |
| 327 | ++ |
| 328 | − |
| 329 | ++ |
| 330 | ++ |
| 331 | − |
| 332 | − |
| 333 | − |
| 334 | +++ |
| 336 | ++ |
| 337 | ++ |
| 338 | +++ |
| 339 | − |
| 340 | − |

### Example 3

### Experimental Methods

### High Content Screen for the Identification of Collagen I Modulators

Compound effect on translation of Collagen I in WI38, human lung fibroblast cell line was conducted using specific PSM assay using tRNAgly and tRNApro isoacceptors, as described above. A library of diverse small molecules, 90,000 compounds, was used at a final concentration of 30 uM. Image and data analyses were conducted using Anima's proprietary algorithms. False positive and toxic compounds were eliminated. A total of 3,600 compounds were identified as hits, compounds which increased or decreased the FRET signal generated by ribosomes during collagen I translation.

Positive hits were re-screened in the specific PSM assay, using tRNAPro and tRNAGly, and counter-screened to eliminate general translation inhibitors in bulk tRNA PSM assay and in a metabolic labeling assay [Click-IT™, L-Azidohomoalanine (AHA)]; collagen-specific regulators were assays using anti-Collagen I immunofluorescence; all assays were run on activated WI38 cells. Hits were scored using Anima's proprietary algorithms, and 360 compounds which selectively inhibited specific PSM assay and reduced collagen I as detected by immunofluorescence were selected as confirmed hits. These compounds were purchased as powder to confirm activity. Re-purchased hits were tested in the specific PSM assay (tRNApro-tRNAgly) and anti-Collagen I immunofluorescence, and in counter assays to eliminate global translation modulators: (1) bulk tRNA and metabolic labeling using Click-IT™ AHA (L-Azidohomoalanine).

### Cell Culture

WI-38 cells (ATCC® CCL-75™) were maintained in MEM EAGLE (NEAA) W. GLUTAMIN (Biological Industries, Cat. 06-1040-15-1A) containing 10% fetal bovine serum (FBS) and 1% Penicillin-Streptomycin Solution. To synchronize the cells (cell cycle synchronization) prior to induction of collagen synthesis, the cells were starved using DMEM-low glucose supplemented with 0.25% FBS for two hours and then without FBS for 24 hours. To induce collagen synthesis, the cells were treated with a collagen induction cocktail for the indicated time. Compounds were added with induction.

Primary human pulmonary fibroblasts (HPF, PromoCell C-12360) were maintained in fibroblast growth medium 2 (PromoCell C-23020) according to manufacture instruction. Collagen synthesis was inducted using the same cocktail as for the WI-38 cells.

Primary human dermal fibroblasts (HDF) (PromoCell C-12302) were maintained in PromoCell's proprietary Fibroblast Growth Medium 2 (ready-to-use, Cat. C-23020). For collagen synthesis induction, cells were seeded on experimental plates for 24 hours followed by addition of collagen induction cocktail. Tested compounds were added together with induction.

Specific tRNA (tRNA Isoacceptor) Isolation and Labeling

The specific tRNAproline (AGG) and tRNAglycine (GCC) were isolated for from baker's yeast (Roche) using biotinylated oligos complimentary to sequences encompassing the D-loop and anti-codon. The biotinylated oligos were mixed with total yeast tRNA and heated up to 82° C. for 10 minutes, followed by addition of TMA buffer (20 mM Tris, pH 7.6, 1.8M tetramethylammonium chloride, 0.2 mM EDTA). The mixture was incubated at 68° C. for 10 minutes, and annealed by slow cooling to 37° C. tRNA:DNA oligo mixture then was incubated with streptavidin linked agarose beads at room temperature for 30 min while shaking. Unbound tRNA and tRNA:DNA complexes were removed by centrifugation and beads washed with 10 mM Tris-HCl (pH 7.6). The target tRNA was eluted from the resin by incubation at 45° C. or 55° C. for 7 minutes followed by centrifugation and collection of the supernatant to clean tubes.

The purity of the isolated tRNA isoacceptors was confirmed using fluorescent polarization assay. Purified tRNA was annealed to a complementary oligo tagged at the 3′-end with Cy3. The annealed purified tRNA isoacceptor FP signal was compared to the signal derived from annealing of a tRNA isoacceptor oligo annealed to the same Cy3-oligo. Samples with more than 80% purity were selected for labeling.

The dihydrouridines of the target tRNAs or total yeast tRNA were labeled as described in U.S. Pat. No. 8,785,119. Labeled tRNAs are purified by reverse phase HPLC. Labeled tRNA is eluted with an ethanol gradient.

### Protein Synthesis Monitoring (PSM) Assays

Cy3 and Cy5 Labeled tRNA, bulk or specific, are transfected with 0.4 μl HiPerFect (Qiagen) per 384 well. First, HiPerFect is mixed with DMEM and incubated for 5 minutes; next, 8 nanograms Cy3-labeled tRNAPro and 8 ng Cy5-labeled tRNAGly (or 8 ng each Cy3 and Cy5-labelled bulk tRNA are diluted in 1×PBS and then added to the HiPerFect:DMEM cocktail and incubated at room temperature for 20 minutes. The transfection mixture is dispersed automatically into 384-well black plates. Cells are then seeded at 3,500 cells per well in DMEM-10% FBS-1% pencillin-Streptomycin-1% L-Glutamine. Plates are incubated at 37° C., 5% CO₂ overnight. Twenty-four hours after transfection collagen production is stimulated with collagen induction cocktail, and then compounds are added at a final concentration of 30 uM. After an additional 24 hours incubation, cells are fixed with 4% paraformaldehyde and images are captured with Operetta microscope (Perkin Elmer) using ×20 high NA objective lens.

### Metabolic Labeling Assay

Synchronized WI-38 cells are seeded at 3,500 cells per well in DMEM-10% FBS-1% pencillin-Streptomycin-1% L-Glutamine. Plates are incubated at 37° C., 5% CO₂ overnight. The collagen production is stimulated with collagen induction cocktail, and then compounds are added at a final concentration of 30 uM. After 20 hours of incubation, the growth medium is aspirated, and cell washed twice with HBSS. Metabolic labeling medium DMEM (-Cys-Met)-10% dialyzed FBS-1% pencillin-Streptomycin-1% L-Glutamine was added to the cells for 30 minutes. Then medium was replaced by metabolic labeling medium containing 25 μM L-Azidohomoalanine (AHA, ThermoFisher) and incubated for 4 hours at 37° C., 5% CO₂. Cells are washed by HBSS at 37° C. for 15 minutes before fixing with 4% paraformaldehyde. Cells are washed twice with 3% BSA in PBS before permeabilization with 0.5% Triton X-100 in PBS for 20 minutes. The AHA staining with Alexa Fluor™ 555 alkyne is performed according to the manufacture instruction. Images are captured with Operetta microscope (Perkin Elmer) using ×20 high NA objective lens.

### Collagen-I Immunofluorescence Assay

Cells in 96-well or 384-well plates were fixed for 20 min in 4% paraformaldehyde (PFA, ENCO, Cat. sc-281692). Following two washes with 1×PBS, cells were treated with hydrogen peroxide (Acros, Cat: 7722-84-1) for 10 minutes and then washed twice with 1×PBS. Cells were then incubated over-night at 4° C. with Anti-Collagen I (Sigma-Aldrich, Cat: C 2456) antibody and washed three times with 1×PBS. Cell were then incubated with a suitable secondary fluorescently-tagged antibody and nuclei stained with DAPI, for 1 hour, and then washed 3 times with 1×PBS.

Cell images were taken with Operetta (Perkin Elmer, USA), a wide-field fluorescence microscope at 20× magnification. After acquisition, the images were transferred to Columbus software (Perkin-Elmer) for image analysis. In Columbus, cells were identified by their nucleus, using the “Find Nuceli” module and cytoplasm was detected based on the secondary antibody channel. Subsequently, the fluorescent signal was enumerated in the identified cell region. Data was exported to a data analysis and visualization software, Tibco Spotfire, USA.

### Fluorescent In Situ Hybridization (FISH) Assay

WI-38 cells were grown in 384-wells plates (Perkin Elmer, Cat. 6057300) and fixed for 20 min in 4% paraformaldehyde (PFA, ENCO, Cat. sc-281692), and left overnight in 70% ethanol at 4° C. The next day, the cells were washed with 1×PBS and then incubated for 10 min in 10% formamide in 10% saline-sodium citrate (SSC). Fluorescently-labeled DNA probes that target the COL1A1 (Cy5, Biosearch Technologies, Cat. SMF-1063-5) and GAPDH (Cy3, Biosearch Technologies, Cat. VSMF-2150-5) mRNAs were hybridized overnight at 37° C. in a dark chamber in 10% formamide. The next day, cells were washed twice with 10% formamide for 30 min. Next, nuclei were counterstained with DAPI (SIGMA, Cat. 5MG-D9542) and then washed twice with 1×PBS. FISH experiments were performed according to the probes manufacturer's protocol for adherent cells.

Following RNA FISH experiments, images of cells were taken with Operetta (Perkin Elmer, USA), a wide-field fluorescence microscope at 20× magnification. After acquisition, the images were transferred to Columbus software for image analysis. In Columbus, cells were identified by their nucleus, using the “Find Nuceli” module, cytoplasm was detected based on the FISH-channel, and single mRNAs in the cytoplasm and transcription sites in the nucleus were detected using “Find Spots” module. Subsequently, fluorescent signals were collected for each channel in the identified regions, nucleus, cytoplasm and spots. Data was exported to a data analysis and visualization software, Tibco Spotfire, USA.

1-77. (canceled)

78. A compound represented by the structure of formula III: 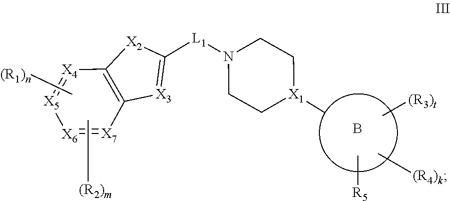
by the structure of formula V(a): 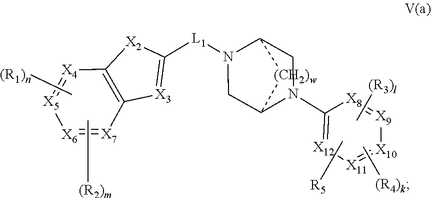
by the structure of formula VI: 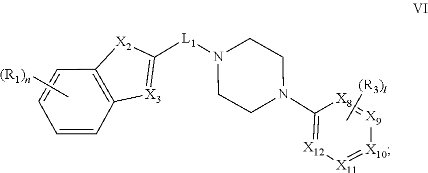
or by the structure of formula VII: 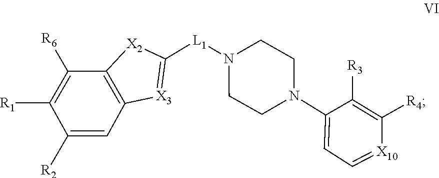
wherein
B ring is a single or fused aromatic or heteroaromatic ring system (e.g., phenyl, pyrimidine, 2-3- or 4-pyridine, pyridazine or pyrazine, thiazole, pyrrole, triazole, imidazole, indazole);
X₁ is N or C(R) (e.g., C—H, C—OH);
L₁ is CH₂, CHR, or C(R)₂
R₁, R₂ and R₆ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R (e.g., NHCO-Ph; NHCO—CH₃), NHC(O)—R₁₀ (e.g., NHCO—CH₃) NHCO—N(R₁₀)(R₁₁), —C(O)Ph, C(O)O—R₁₀, R₈—C(O)—R₁₀, C(O)H, C(O)—R⁺, C₁-C₅ linear or branched C(O)-haloalkyl, —C(O)NH₂, C(O)NHR (e.g., C(O)NH-Ph), C(O)N(R₁₀)(R₁₁), SO₂R, SO₂N(R₁₀)(R₁₁), NHSO₂(R₁₀) (e.g., NHSO₂CH₃), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyridine), substituted or unsubstituted aryl (e.g., phenyl), substituted or unsubstituted benzyl;
or R₂ and R₁ are joined together to form a 5 or 6 membered substituted or unsubstituted, aliphatic or aromatic, carbocyclic (e.g., benzene) or heterocyclic (e.g., pyridine) ring;
R₃, R₄ and R₅ are each independently H, F, Cl, Br, I, OH, SH, R₈—OH, R₈—SH, —R₈—O—R₁₀ (e.g., CH₂—CH₂—O—CH₃, CH₂—O—CH₂—CH₂—O—CH₃), R₈—(C₃-C₈ cycloalkyl), R₈—(C₃-C₈ heterocyclic ring), O—R₂₀, CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear, branched or cyclic haloalkyl (e.g., CHF₂), C₁-C₅ linear, branched or cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring (e.g., pyrazole, thiazole), substituted or unsubstituted aryl (e.g., phenyl), substituted or unsubstituted benzyl;
or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;
R₂₀ is represented by the following structure: 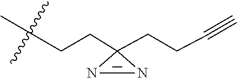 [SMILES: *CCC1(CCC#C)N=N1]
X₂ is NH, S, O, N—R (e.g., N—CH₂—CH₂—O—CH₃);
X₃ is N, C(R) (e.g., CH, C—CH₃, C—Cl, C—CN);
X₄, X₅, X₆, and X₇ are each independently C or N;
X₈, X₉, X₁₀, X₁₁, and X₁₂ are each independently C or N;
R is H, OH, F, Cl, Br, I, CN, CF₃, NO₂, C₁-C₅ linear or branched, substituted or unsubstituted alkyl (e.g., methyl, ethyl), C₁-C₅ linear or branched alkoxy, —R₈—O—R₁₀ (e.g., CH₂—CH₂—O—CH₃), C₁-C₅ linear or branched haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), substituted or unsubstituted aryl (e.g., phenyl), substituted or unsubstituted heteroaryl (e.g., pyridine (2, 3, and 4-pyridine); or
two geminal R substitutions are joined together to form a 3-6 membered substituted or unsubstituted, aliphatic (e.g., cyclopropyl, cyclopentene) or aromatic, carbocyclic (e.g., benzene) or heterocyclic (e.g., thiophene, furane, pyrrol, pyrazole) ring;
R₈ is [CH₂]ₚ
wherein p is between 1 and 10 (e.g., 2);
R₉ is [CH]_{q}, [C]_{q}
wherein q is between 2 and 10;
R₁₀ and Rᵤ are each independently H, substituted or unsubstituted, C₁-C₅ linear or branched alkyl (e.g., methyl, ethyl, CH₂—CH₂—O—CH₃), C₁-C₅ linear or branched alkoxy (e.g., O—CH₃), C(O)R, or S(O)₂R;
or R₁₀ and Rᵤ are joined to form a substituted or unsubstituted C₃-C₈ heterocyclic ring (e.g., piperazine, piperidine),
wherein substitutions include: F, Cl, Br, I, OH, SH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof;
m, n, l and k are each independently an integer between 0 and 4 (e.g., 0, 1 or 2);
w is 0, 1 or 2; wherein if w=0, the bridge on the ring is absent;
wherein if X₃ is N, then X₂ is not NH;
or by the structure of formula IX: 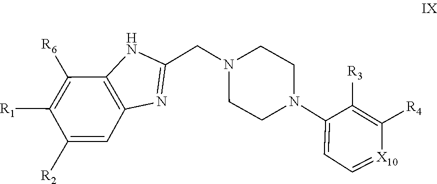
wherein
R₁ is Cl, —R₈—O—R₁₀ (e.g., CH₂—CH₂—O—CH₃, CH₂—O—CH₂—CH₂—O—CH₃), —O—R₈—O—R₁₀ (e.g., 0-CH₂—CH₂—O—CH₃), O—R₂₀, or CF₃;
R₂ is H, Cl, —R₈—O—R₁₀ (e.g., CH₂—CH₂—O—CH₃, CH₂—O—CH₂—CH₂—O—CH₃), —O—R₈—O—R₁₀ (e.g., O—CH₂—CH₂—O—CH₃), O—R₂₀, or CF₃;
R₆ is H;
R₃ and R₄ are each independently H, O—R₂₀, C₁-C₅ linear or branched, substituted or unsubstituted alkyl (e.g., methyl, ethyl), substituted or unsubstituted C₃-C₈ heterocyclic ring (e.g., imidazole), (wherein substitutions include: F, Cl, Br, I, C₁-C₅ linear or branched alkyl, OH, alkoxy, CF₃, aryl, phenyl, heteroaryl, C₃-C₈ cycloalkyl, halophenyl, (benzyloxy)phenyl, CN, NO₂ or any combination thereof);
or R₃ and R₄ are joined together to form a 5 or 6 membered substituted or unsubstituted, aliphatic (e.g., cyclopentene) or aromatic, carbocyclic (e.g., benzene) or heterocyclic (e.g., thiophene, furane, pyrrol, pyrazole) ring;
R₂₀ is represented by the following structure: 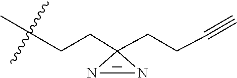 [SMILES: *CCC1(CCC#C)N=N1]
X₁₀ and X₁₂ are each independently C or N;
wherein if R₃ is ethyl, then R₁ or R₂ is not CF₃;
or by the structure of the following compounds:

| Compound | |
|---|---|
| Number | Compound Structure |
| 218 | 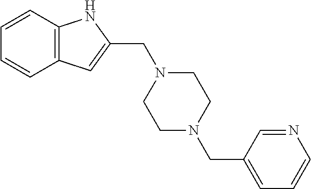 [SMILES: c1cncc(CN2CCN(Cc3cc4ccccc4[nH]3)CC2)c1] |
| 227 | 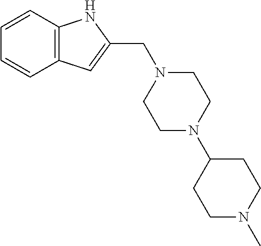 [SMILES: CN1CCC(N2CCN(Cc3cc4ccccc4[nH]3)CC2)CC1] |
| 229 | 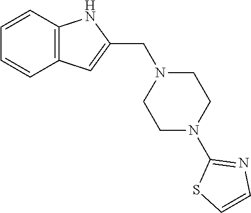 [SMILES: c1ccc2[nH]c(CN3CCN(c4nccs4)CC3)cc2c1] |
| 230 | 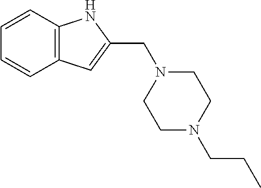 [SMILES: CCCN1CCN(Cc2cc3ccccc3[nH]2)CC1] |
| 232 | 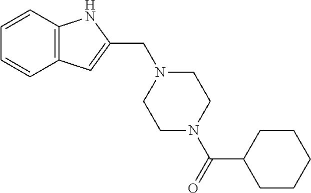 [SMILES: O=C(C1CCCCC1)N1CCN(Cc2cc3ccccc3[nH]2)CC1] |
| 233 | 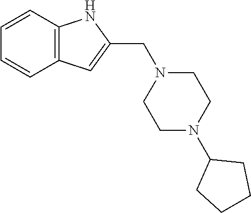 [SMILES: c1ccc2[nH]c(CN3CCN(C4CCCC4)CC3)cc2c1] |
| 239 | 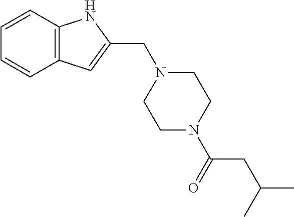 [SMILES: CC(C)CC(=O)N1CCN(Cc2cc3ccccc3[nH]2)CC1] |
| 244 | 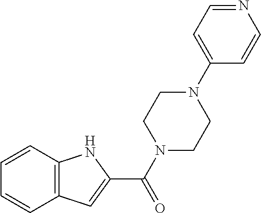 [SMILES: O=C(c1cc2ccccc2[nH]1)N1CCN(c2ccncc2)CC1] |
| 257 | 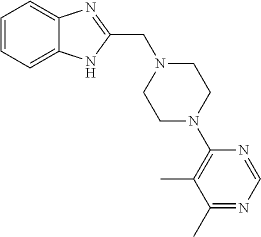 [SMILES: Cc1ncnc(N2CCN(Cc3nc4ccccc4[nH]3)CC2)c1C] |
| 262 | 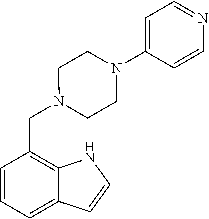 [SMILES: c1cc(CN2CCN(c3ccncc3)CC2)c2[nH]ccc2c1] |
| 263 | 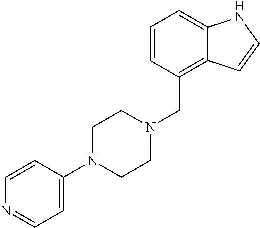 [SMILES: c1cc(CN2CCN(c3ccncc3)CC2)c2cc[nH]c2c1] |
| 264 | 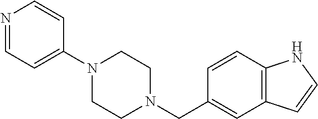 [SMILES: c1cc(N2CCN(Cc3ccc4[nH]ccc4c3)CC2)ccn1] |
| 265 | 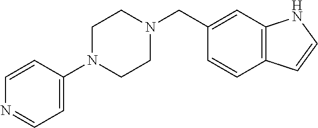 [SMILES: c1cc(N2CCN(Cc3ccc4cc[nH]c4c3)CC2)ccn1] |
| 267 | 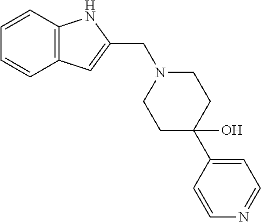 [SMILES: OC1(c2ccncc2)CCN(Cc2cc3ccccc3[nH]2)CC1] |
| 268 | 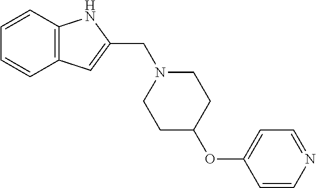 [SMILES: c1ccc2[nH]c(CN3CCC(Oc4ccncc4)CC3)cc2c1] |
| 271 | 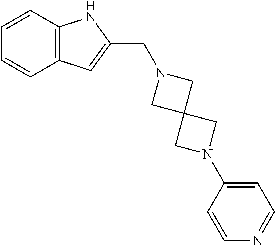 [SMILES: c1ccc2[nH]c(CN3CC4(C3)CN(c3ccncc3)C4)cc2c1] |
| 272 | 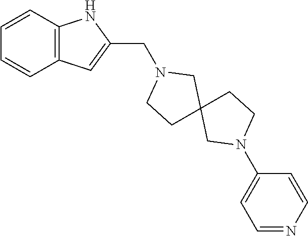 [SMILES: c1ccc2[nH]c(CN3CCC4(CCN(c5ccncc5)C4)C3)cc2c1] |
| 273 | 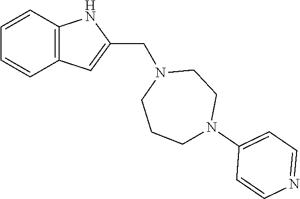 [SMILES: c1ccc2[nH]c(CN3CCCN(c4ccncc4)CC3)cc2c1] |
| 274 | 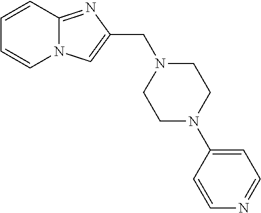 [SMILES: c1ccn2cc(CN3CCN(c4ccncc4)CC3)nc2c1] |
| 276 | 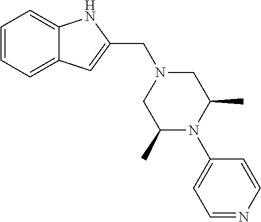 [SMILES: C[C@@H]1CN(Cc2cc3ccccc3[nH]2)C[C@H](C)N1c1ccncc1] |
| 277 | 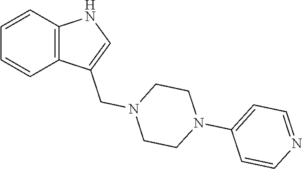 [SMILES: c1ccc2c(CN3CCN(c4ccncc4)CC3)c[nH]c2c1] |
| 283 | 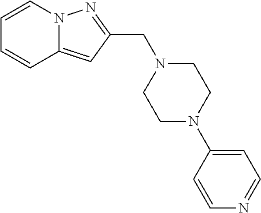 [SMILES: c1ccn2nc(CN3CCN(c4ccncc4)CC3)cc2c1] |
| 285 | 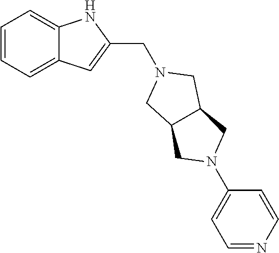 [SMILES: c1ccc2[nH]c(CN3C[C@@H]4CN(c5ccncc5)C[C@@H]4C3)cc2c1] |
| 288 | 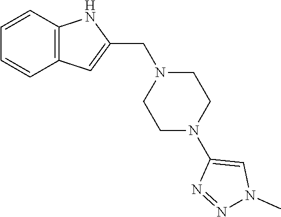 [SMILES: Cn1cc(N2CCN(Cc3cc4ccccc4[nH]3)CC2)nn1] |
| 322 | 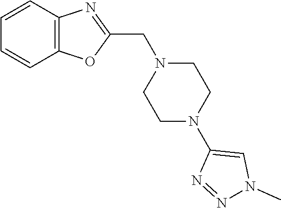 [SMILES: Cn1cc(N2CCN(Cc3nc4ccccc4o3)CC2)nn1] |
| 323 | 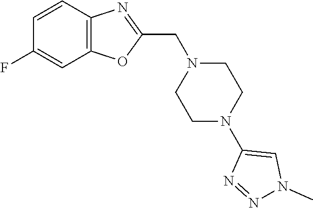 [SMILES: Cn1cc(N2CCN(Cc3nc4ccc(F)cc4o3)CC2)nn1] |
| 324 | 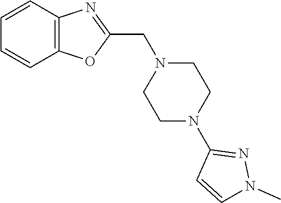 [SMILES: Cn1ccc(N2CCN(Cc3nc4ccccc4o3)CC2)n1] |
| 325 | 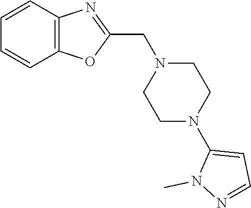 [SMILES: Cn1nccc1N1CCN(Cc2nc3ccccc3o2)CC1] |
| 326 | 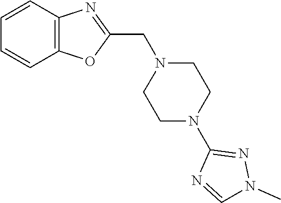 [SMILES: Cn1cnc(N2CCN(Cc3nc4ccccc4o3)CC2)n1] |
| 328 | 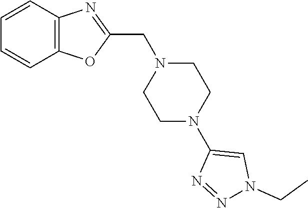 [SMILES: CCn1cc(N2CCN(Cc3nc4ccccc4o3)CC2)nn1] |
| 331 | 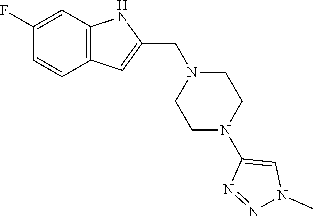 [SMILES: Cn1cc(N2CCN(Cc3cc4ccc(F)cc4[nH]3)CC2)nn1] |
| 332 | 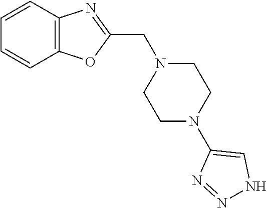 [SMILES: c1ccc2oc(CN3CCN(c4c[nH]nn4)CC3)nc2c1] |
| 339 | 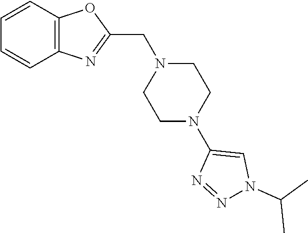 [SMILES: CC(C)n1cc(N2CCN(Cc3nc4ccccc4o3)CC2)nn1] |
| 340 | 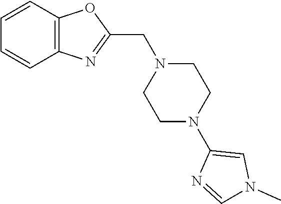 [SMILES: Cn1cnc(N2CCN(Cc3nc4ccccc4o3)CC2)c1] |

or its pharmaceutically acceptable salt, stereoisomer, tautomer, hydrate, N-oxide, reverse amide analog, prodrug, isotopic variant (e.g., deuterated analog), reverse amide, pharmaceutical product or any combination thereof.

79. The compound according to claim 78,
wherein at least one of X₈, X₉, X₁₀, X₁₁, and X₁₂ is N;
wherein X₆ is C or N;
wherein R₃ is H, Cl, F, OH, CF₃, CHF₂, CN, C(O)H, O—R₂₀, CH₃, C₂H₅, —R₈—O—R₁₀, CH₂—O—CH₂—CH₂—O—CH₃, —O—R₈—O—R₁₀, O—CH₂—CH₂—O—CH₃, NH₂, C₁-C₅ linear or branched, substituted or unsubstituted alkyl or methyl.
wherein R₁ is H, Cl, F, OH, CN, NH₂, NHR, N(R)₂, —C(O)NH₂, C(O)NHR, C(O)N(R₁₀)(R₁₁), C₁-C₅ linear or branched, substituted or unsubstituted alkyl, C₁-C₅ linear, branched or cyclic haloalkyl, CHF₂, C₁-C₅ linear, branched or cyclic alkoxy, methoxy, C₁-C₅ linear or branched haloalkoxy, substituted or unsubstituted C₃-C₈ cycloalkyl, substituted or unsubstituted C₃-C₈ heterocyclic ring, NH(CO)-Ph, —R₈—O—R₁₀, CH₂—O—CH₂—CH₂—O—CH₃, —O—R₈—O—R₁₀, O—CH₂—CH₂—O—CH₃, O—R₂₀ or CF₃;
wherein X₂ is NH or O and X₃ is N, CH or C—Cl;
wherein L₁ is CH₂ and/or w is 1 or 0;
wherein 1 is 1 or 2;
or any combination thereof.

80. The compound of claim 78, wherein the heterocyclic ring of R₁ is: piperazine, piperazin-2-one, piperidine, morpholine, triazole, oxadiazole, tetrazole, imidazole, pyrazole, pyrrolidine, pyrrolidin-2-one, oxetane, azetidine, diazepane, 4,7-diazaspiro[2.5]octane or 3-azabicyclo[3.1.0]hexane.

81. The compound of claim 78, wherein at least one of X₁₀ and X₁₂ is N.

82. The compound of claim 78, selected from the following:

| Com- | |
|---|---|
| pound | |
| Number | Compound Structure |
| 202 | 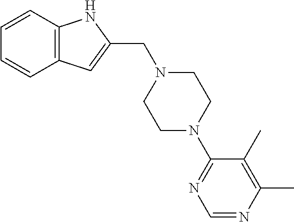 [SMILES: Cc1ncnc(N2CCN(Cc3cc4ccccc4[nH]3)CC2)c1C] |
| 204 | 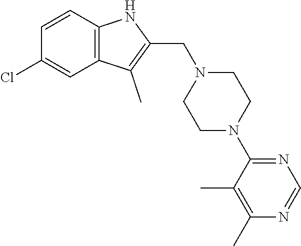 [SMILES: Cc1ncnc(N2CCN(Cc3[nH]c4ccc(Cl)cc4c3C)CC2)c1C] |
| 210 | 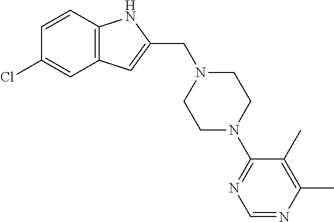 [SMILES: Cc1ncnc(N2CCN(Cc3cc4cc(Cl)ccc4[nH]3)CC2)c1C] |
| 212 | 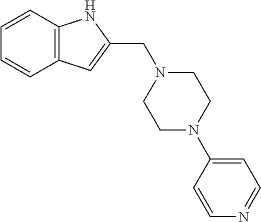 [SMILES: c1ccc2[nH]c(CN3CCN(c4ccncc4)CC3)cc2c1] |
| 213 | 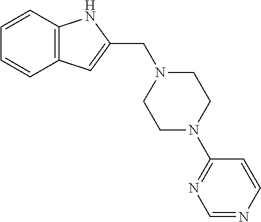 [SMILES: c1ccc2[nH]c(CN3CCN(c4ccncn4)CC3)cc2c1] |
| 215 | 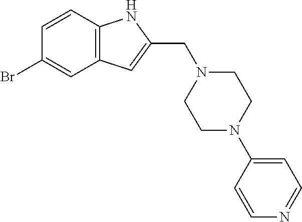 [SMILES: Brc1ccc2[nH]c(CN3CCN(c4ccncc4)CC3)cc2c1] |
| 216 | 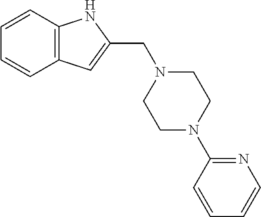 [SMILES: c1ccc(N2CCN(Cc3cc4ccccc4[nH]3)CC2)nc1] |
| 217 | 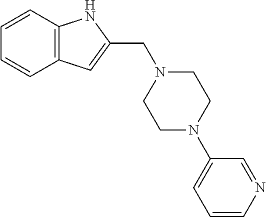 [SMILES: c1cncc(N2CCN(Cc3cc4ccccc4[nH]3)CC2)c1] |
| 219 | 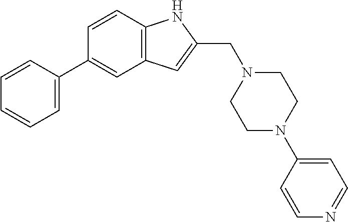 [SMILES: c1ccc(-c2ccc3[nH]c(CN4CCN(c5ccncc5)CC4)cc3c2)cc1] |
| 220 | 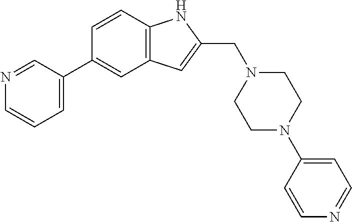 [SMILES: c1cncc(-c2ccc3[nH]c(CN4CCN(c5ccncc5)CC4)cc3c2)c1] |
| 221 | 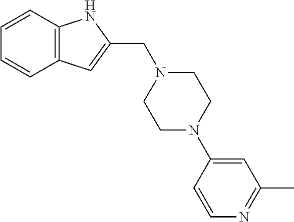 [SMILES: Cc1cc(N2CCN(Cc3cc4ccccc4[nH]3)CC2)ccn1] |
| 222 | 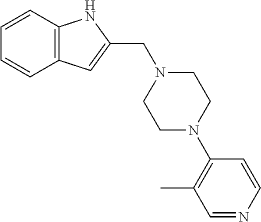 [SMILES: Cc1cnccc1N1CCN(Cc2cc3ccccc3[nH]2)CC1] |
| 223 | 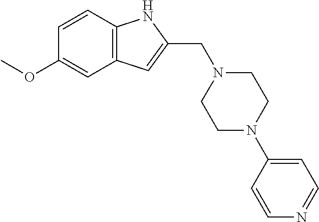 [SMILES: COc1ccc2[nH]c(CN3CCN(c4ccncc4)CC3)cc2c1] |
| 224 | 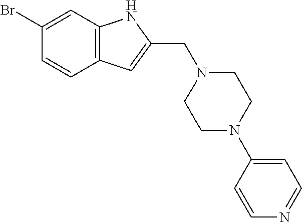 [SMILES: Brc1ccc2cc(CN3CCN(c4ccncc4)CC3)[nH]c2c1] |
| 225 | 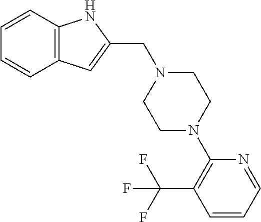 [SMILES: FC(F)(F)c1cccnc1N1CCN(Cc2cc3ccccc3[nH]2)CC1] |
| 226 | 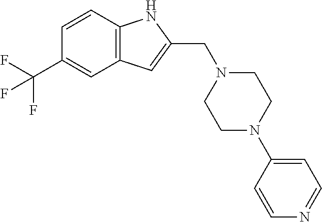 [SMILES: FC(F)(F)c1ccc2[nH]c(CN3CCN(c4ccncc4)CC3)cc2c1] |
| 228 | 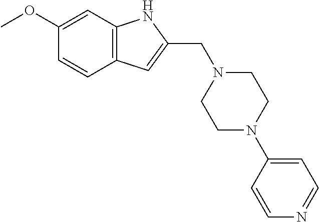 [SMILES: COc1ccc2cc(CN3CCN(c4ccncc4)CC3)[nH]c2c1] |
| 234 | 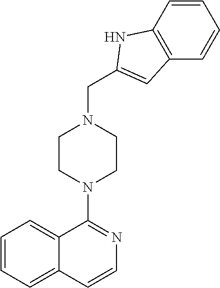 [SMILES: c1ccc2[nH]c(CN3CCN(c4nccc5ccccc45)CC3)cc2c1] |
| 235 | 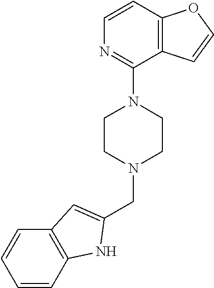 [SMILES: c1ccc2[nH]c(CN3CCN(c4nccc5occc45)CC3)cc2c1] |
| 236 | 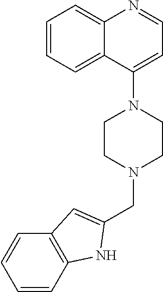 [SMILES: c1ccc2[nH]c(CN3CCN(c4ccnc5ccccc45)CC3)cc2c1] |
| 237 | 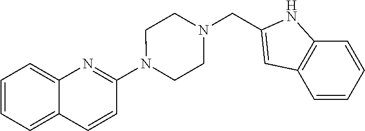 [SMILES: c1ccc2nc(N3CCN(Cc4cc5ccccc5[nH]4)CC3)ccc2c1] |
| 238 | 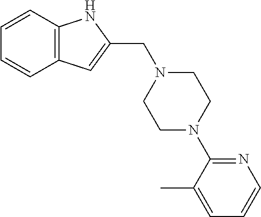 [SMILES: Cc1cccnc1N1CCN(Cc2cc3ccccc3[nH]2)CC1] |
| 240 | 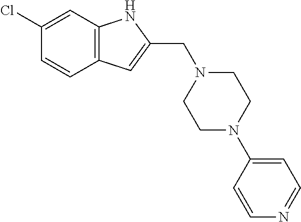 [SMILES: Clc1ccc2cc(CN3CCN(c4ccncc4)CC3)[nH]c2c1] |
| 241 | 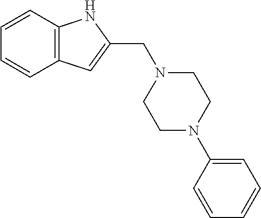 [SMILES: c1ccc(N2CCN(Cc3cc4ccccc4[nH]3)CC2)cc1] |
| 242 | 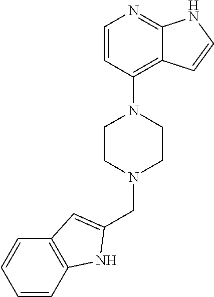 [SMILES: c1ccc2[nH]c(CN3CCN(c4ccnc5[nH]ccc45)CC3)cc2c1] |
| 243 | 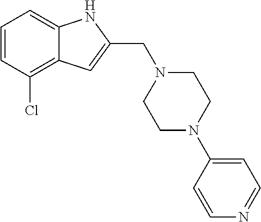 [SMILES: Clc1cccc2[nH]c(CN3CCN(c4ccncc4)CC3)cc12] |
| 245 | 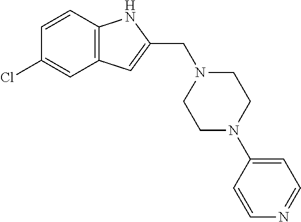 [SMILES: Clc1ccc2[nH]c(CN3CCN(c4ccncc4)CC3)cc2c1] |
| 246 | 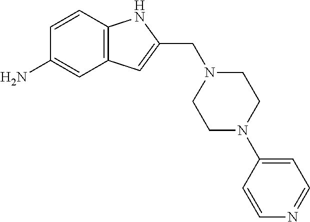 [SMILES: Nc1ccc2[nH]c(CN3CCN(c4ccncc4)CC3)cc2c1] |
| 247 | 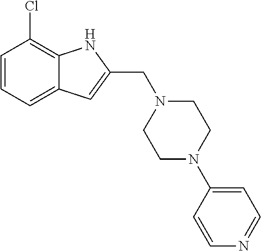 [SMILES: Clc1cccc2cc(CN3CCN(c4ccncc4)CC3)[nH]c12] |
| 248 | 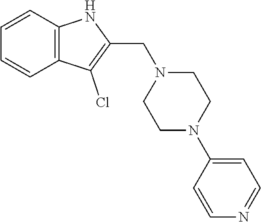 [SMILES: Clc1c(CN2CCN(c3ccncc3)CC2)[nH]c2ccccc12] |
| 249 | 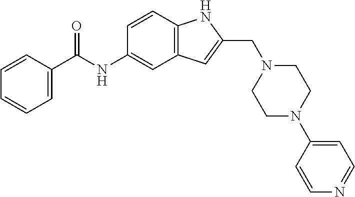 [SMILES: O=C(Nc1ccc2[nH]c(CN3CCN(c4ccncc4)CC3)cc2c1)c1ccccc1] |
| 250 | 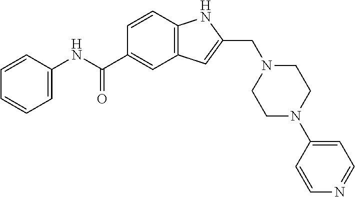 [SMILES: O=C(Nc1ccccc1)c1ccc2[nH]c(CN3CCN(c4ccncc4)CC3)cc2c1] |
| 251 | 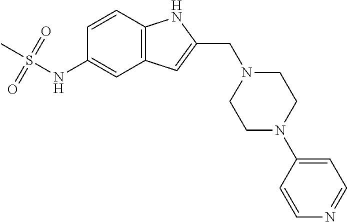 [SMILES: CS(=O)(=O)Nc1ccc2[nH]c(CN3CCN(c4ccncc4)CC3)cc2c1] |
| 252 | 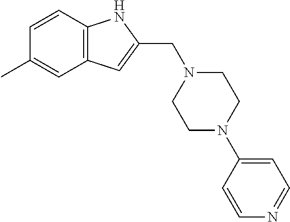 [SMILES: Cc1ccc2[nH]c(CN3CCN(c4ccncc4)CC3)cc2c1] |
| 254 | 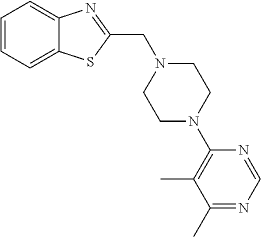 [SMILES: Cc1ncnc(N2CCN(Cc3nc4ccccc4s3)CC2)c1C] |
| 255 | 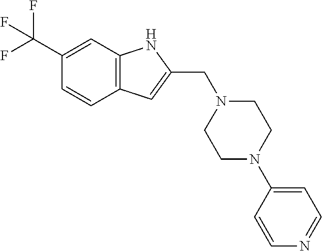 [SMILES: FC(F)(F)c1ccc2cc(CN3CCN(c4ccncc4)CC3)[nH]c2c1] |
| 256 | 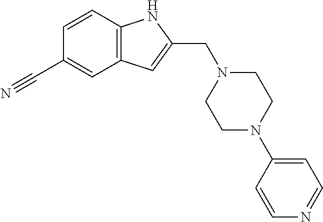 [SMILES: N#Cc1ccc2[nH]c(CN3CCN(c4ccncc4)CC3)cc2c1] |
| 258 | 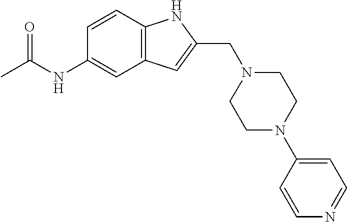 [SMILES: CC(=O)Nc1ccc2[nH]c(CN3CCN(c4ccncc4)CC3)cc2c1] |
| 259 | 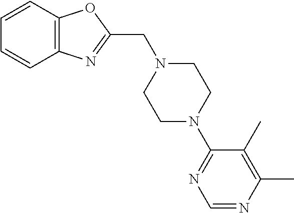 [SMILES: Cc1ncnc(N2CCN(Cc3nc4ccccc4o3)CC2)c1C] |
| 260 | 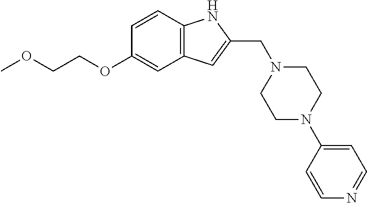 [SMILES: COCCOc1ccc2[nH]c(CN3CCN(c4ccncc4)CC3)cc2c1] |
| 261 | 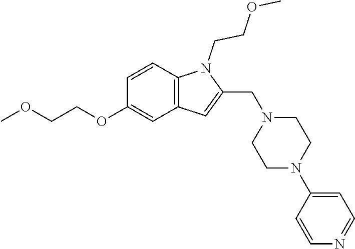 [SMILES: COCCOc1ccc2c(c1)cc(CN1CCN(c3ccncc3)CC1)n2CCOC] |
| 266 | 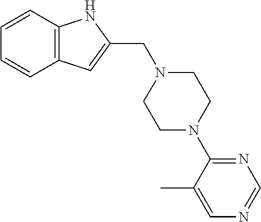 [SMILES: Cc1cncnc1N1CCN(Cc2cc3ccccc3[nH]2)CC1] |
| 269 | 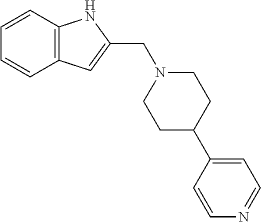 [SMILES: c1ccc2[nH]c(CN3CCC(c4ccncc4)CC3)cc2c1] |
| 270 | 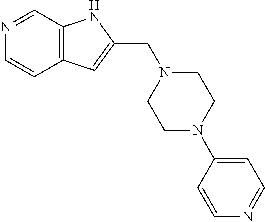 [SMILES: c1cc(N2CCN(Cc3cc4ccncc4[nH]3)CC2)ccn1] |
| 275 | 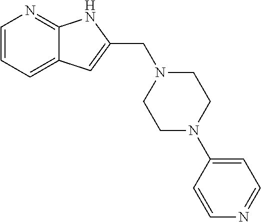 [SMILES: c1cnc2[nH]c(CN3CCN(c4ccncc4)CC3)cc2c1] |
| 278 | 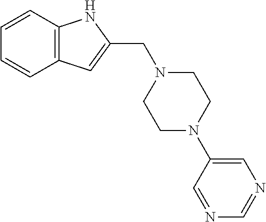 [SMILES: c1ccc2[nH]c(CN3CCN(c4cncnc4)CC3)cc2c1] |
| 279 | 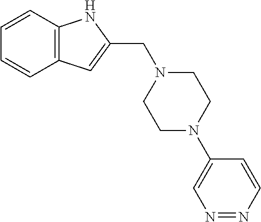 [SMILES: c1ccc2[nH]c(CN3CCN(c4ccnnc4)CC3)cc2c1] |
| 280 | 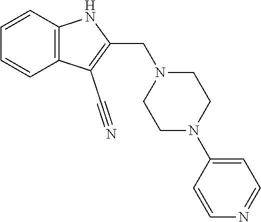 [SMILES: N#Cc1c(CN2CCN(c3ccncc3)CC2)[nH]c2ccccc12] |
| 281 | 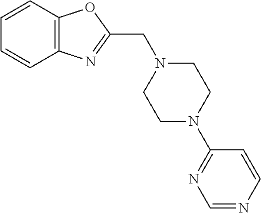 [SMILES: c1ccc2oc(CN3CCN(c4ccncn4)CC3)nc2c1] |
| 282 | 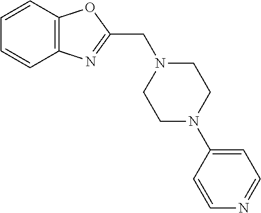 [SMILES: c1ccc2oc(CN3CCN(c4ccncc4)CC3)nc2c1] |
| 284 | 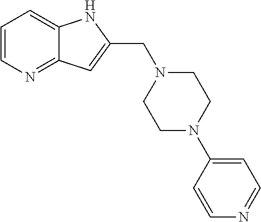 [SMILES: c1cnc2cc(CN3CCN(c4ccncc4)CC3)[nH]c2c1] |
| 286 | 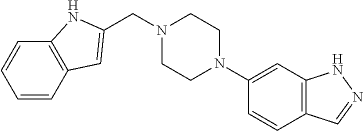 [SMILES: c1ccc2[nH]c(CN3CCN(c4ccc5cn[nH]c5c4)CC3)cc2c1] |
| 287 | 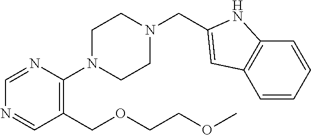 [SMILES: COCCOCc1cncnc1N1CCN(Cc2cc3ccccc3[nH]2)CC1] |
| 291 | 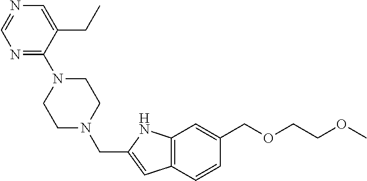 [SMILES: CCc1cncnc1N1CCN(Cc2cc3ccc(COCCOC)cc3[nH]2)CC1] |
| 292 | 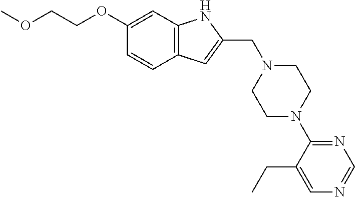 [SMILES: CCc1cncnc1N1CCN(Cc2cc3ccc(OCCOC)cc3[nH]2)CC1] |
| 293 | 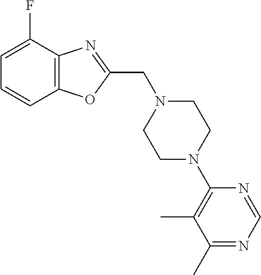 [SMILES: Cc1ncnc(N2CCN(Cc3nc4c(F)cccc4o3)CC2)c1C] |
| 294 | 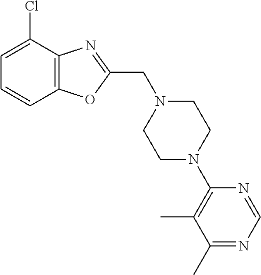 [SMILES: Cc1ncnc(N2CCN(Cc3nc4c(Cl)cccc4o3)CC2)c1C] |
| 295 | 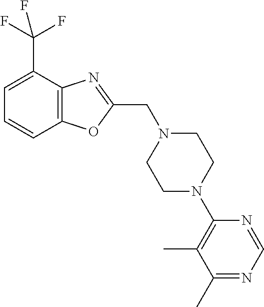 [SMILES: Cc1ncnc(N2CCN(Cc3nc4c(C(F)(F)F)cccc4o3)CC2)c1C] |
| 296 | 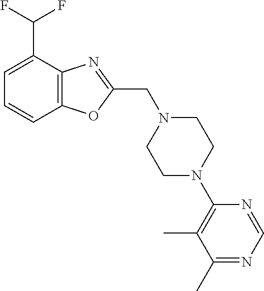 [SMILES: Cc1ncnc(N2CCN(Cc3nc4c(C(F)F)cccc4o3)CC2)c1C] |
| 297 | 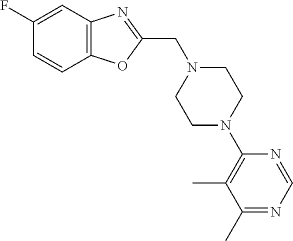 [SMILES: Cc1ncnc(N2CCN(Cc3nc4cc(F)ccc4o3)CC2)c1C] |
| 298 | 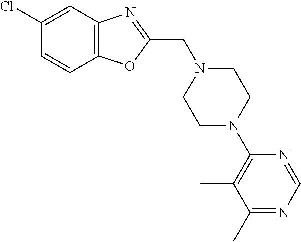 [SMILES: Cc1ncnc(N2CCN(Cc3nc4cc(Cl)ccc4o3)CC2)c1C] |
| 299 | 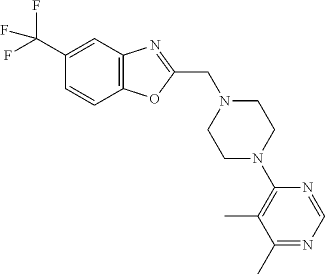 [SMILES: Cc1ncnc(N2CCN(Cc3nc4cc(C(F)(F)F)ccc4o3)CC2)c1C] |
| 300 | 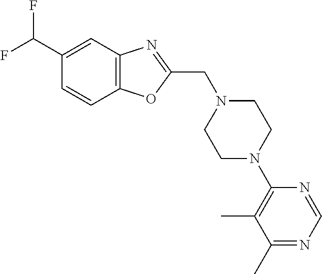 [SMILES: Cc1ncnc(N2CCN(Cc3nc4cc(C(F)F)ccc4o3)CC2)c1C] |
| 301 | 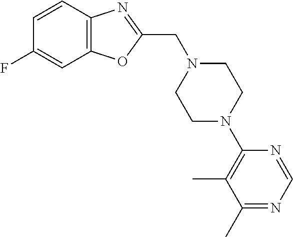 [SMILES: Cc1ncnc(N2CCN(Cc3nc4ccc(F)cc4o3)CC2)c1C] |
| 302 | 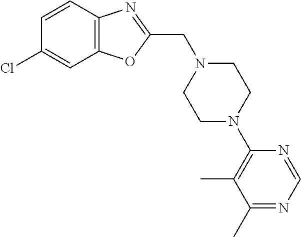 [SMILES: Cc1ncnc(N2CCN(Cc3nc4ccc(Cl)cc4o3)CC2)c1C] |
| 303 | 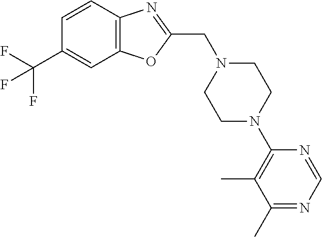 [SMILES: Cc1ncnc(N2CCN(Cc3nc4ccc(C(F)(F)F)cc4o3)CC2)c1C] |
| 304 | 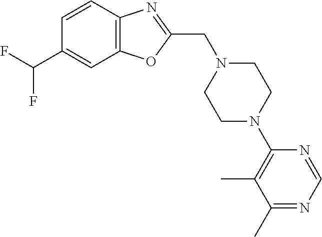 [SMILES: Cc1ncnc(N2CCN(Cc3nc4ccc(C(F)F)cc4o3)CC2)c1C] |
| 305 | 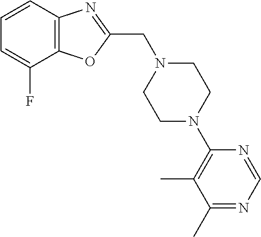 [SMILES: Cc1ncnc(N2CCN(Cc3nc4cccc(F)c4o3)CC2)c1C] |
| 306 | 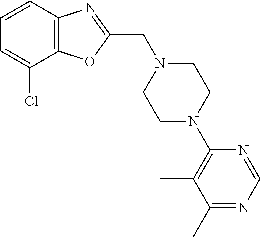 [SMILES: Cc1ncnc(N2CCN(Cc3nc4cccc(Cl)c4o3)CC2)c1C] |
| 307 | 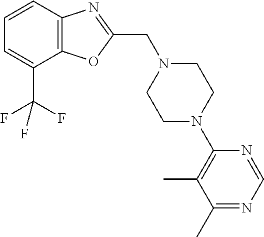 [SMILES: Cc1ncnc(N2CCN(Cc3nc4cccc(C(F)(F)F)c4o3)CC2)c1C] |
| 308 | 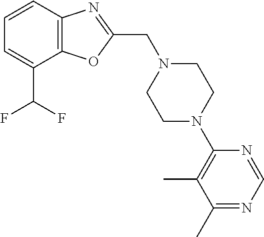 [SMILES: Cc1ncnc(N2CCN(Cc3nc4cccc(C(F)F)c4o3)CC2)c1C] |
| 309 | 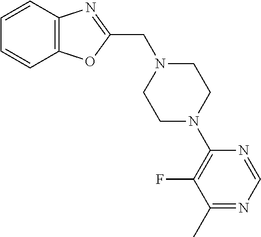 [SMILES: Cc1ncnc(N2CCN(Cc3nc4ccccc4o3)CC2)c1F] |
| 310 | 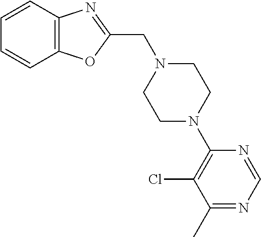 [SMILES: Cc1ncnc(N2CCN(Cc3nc4ccccc4o3)CC2)c1Cl] |
| 311 | 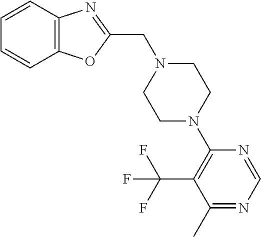 [SMILES: Cc1ncnc(N2CCN(Cc3nc4ccccc4o3)CC2)c1C(F)(F)F] |
| 312 | 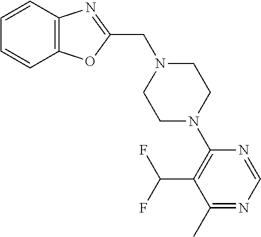 [SMILES: Cc1ncnc(N2CCN(Cc3nc4ccccc4o3)CC2)c1C(F)F] |
| 313 | 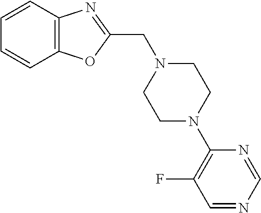 [SMILES: Fc1cncnc1N1CCN(Cc2nc3ccccc3o2)CC1] |
| 314 | 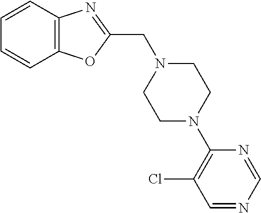 [SMILES: Clc1cncnc1N1CCN(Cc2nc3ccccc3o2)CC1] |
| 315 | 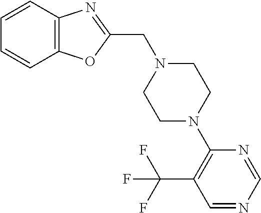 [SMILES: FC(F)(F)c1cncnc1N1CCN(Cc2nc3ccccc3o2)CC1] |
| 316 | 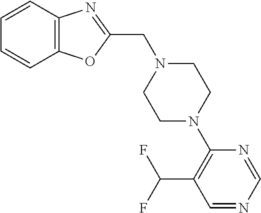 [SMILES: FC(F)c1cncnc1N1CCN(Cc2nc3ccccc3o2)CC1] |
| 317 | 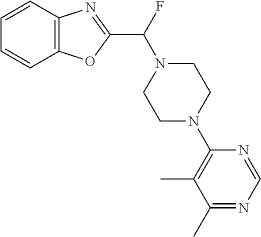 [SMILES: Cc1ncnc(N2CCN(C(F)c3nc4ccccc4o3)CC2)c1C] |
| 318 | 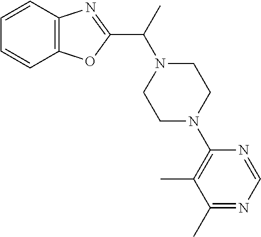 [SMILES: Cc1ncnc(N2CCN(C(C)c3nc4ccccc4o3)CC2)c1C] |
| 320 | 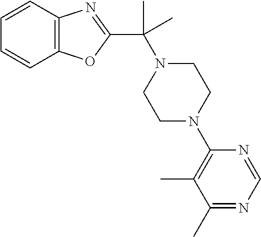 [SMILES: Cc1ncnc(N2CCN(C(C)(C)c3nc4ccccc4o3)CC2)c1C] |
| 321 | 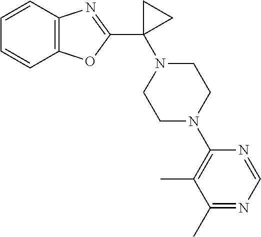 [SMILES: Cc1ncnc(N2CCN(C3(c4nc5ccccc5o4)CC3)CC2)c1C] |
| 327 | 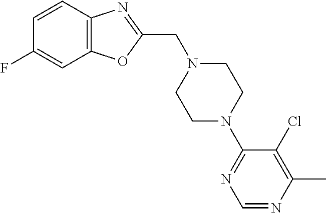 [SMILES: Cc1ncnc(N2CCN(Cc3nc4ccc(F)cc4o3)CC2)c1Cl] |
| 329 | 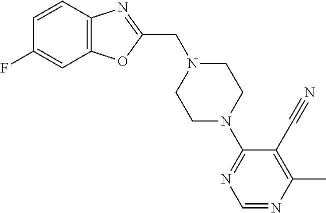 [SMILES: Cc1ncnc(N2CCN(Cc3nc4ccc(F)cc4o3)CC2)c1C#N] |
| 330 | 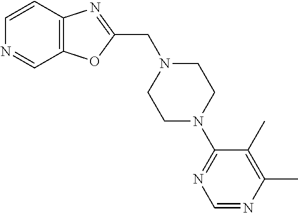 [SMILES: Cc1ncnc(N2CCN(Cc3nc4ccncc4o3)CC2)c1C] |
| 333 | 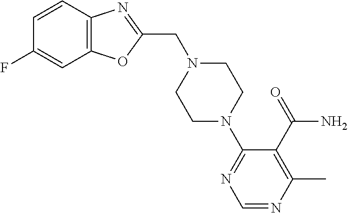 [SMILES: Cc1ncnc(N2CCN(Cc3nc4ccc(F)cc4o3)CC2)c1C(N)=O] |
| 334 | 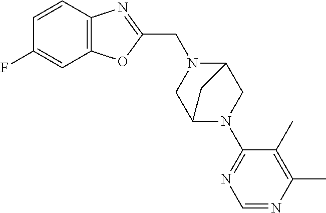 [SMILES: Cc1ncnc(N2CC3CC2CN3Cc2nc3ccc(F)cc3o2)c1C] |
| 336 | 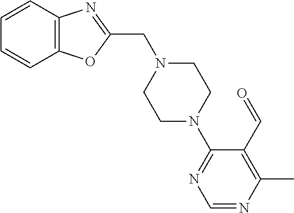 [SMILES: Cc1ncnc(N2CCN(Cc3nc4ccccc4o3)CC2)c1C=O] |
| 337 | 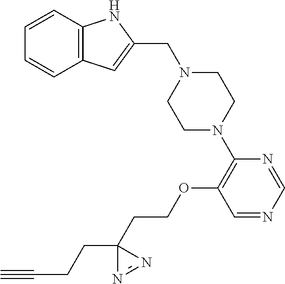 [SMILES: C#CCCC1(CCOc2cncnc2N2CCN(Cc3cc4ccccc4[nH]3)CC2)N=N1] |
| 338 | 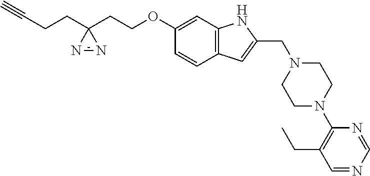 [SMILES: C#CCCC1(CCOc2ccc3cc(CN4CCN(c5ncncc5CC)CC4)[nH]c3c2)N=N1] |

or selected from the following

| Compound | |
|---|---|
| Number | Compound Structure |
| 200 | 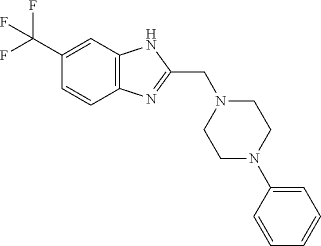 [SMILES: FC(F)(F)c1ccc2nc(CN3CCN(c4ccccc4)CC3)[nH]c2c1] |
| 201 | 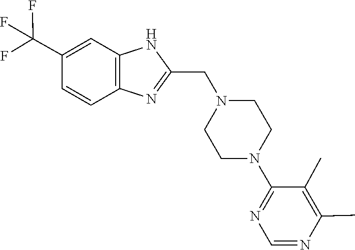 [SMILES: Cc1ncnc(N2CCN(Cc3nc4ccc(C(F)(F)F)cc4[nH]3)CC2)c1C] |
| 203 | 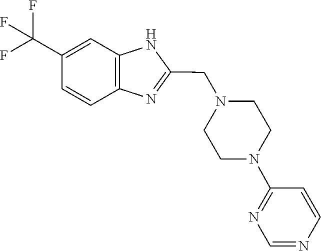 [SMILES: FC(F)(F)c1ccc2nc(CN3CCN(c4ccncn4)CC3)[nH]c2c1] |
| 209 | 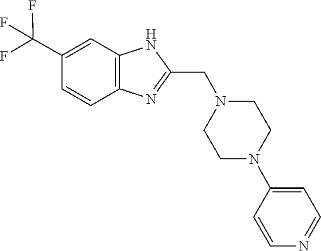 [SMILES: FC(F)(F)c1ccc2nc(CN3CCN(c4ccncc4)CC3)[nH]c2c1] |
| 211 | 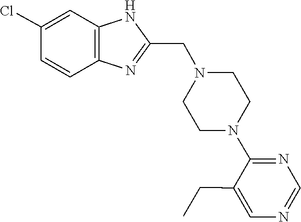 [SMILES: CCc1cncnc1N1CCN(Cc2nc3ccc(Cl)cc3[nH]2)CC1] |
| 289 | 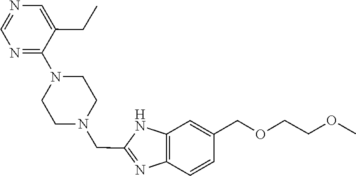 [SMILES: CCc1cncnc1N1CCN(Cc2nc3ccc(COCCOC)cc3[nH]2)CC1] |
| 290 | 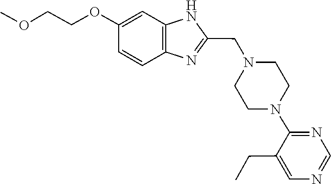 [SMILES: CCc1cncnc1N1CCN(Cc2nc3ccc(OCCOC)cc3[nH]2)CC1] |

83. The compound according to claim 78, wherein the compound is a collagen translation inhibitor.

84. A pharmaceutical composition comprising a compound according to claim 1 and a pharmaceutically acceptable carrier.

85. A method of treating, suppressing, reducing the severity, reducing the risk of developing or inhibiting fibrosis in a subject, comprising administering a compound according to claim 1, to a subject suffering from fibrosis under conditions effective to treat, suppress, reduce the severity, reduce the risk of developing, or inhibit fibrosis in said subject.

86. The method of claim 85,
wherein said fibrosis is a systemic fibrotic disease;
wherein said fibrosis is an organ-specific fibrotic disease;
wherein said fibrosis is primary or secondary fibrosis;
wherein said fibrosis is a result of systemic sclerosis, graft-versus host disease (GVHD), pulmonary fibrosis, autoimmune disorder, tissue injury, inflammation, oxidative stress or any combination thereof;
wherein the fibrosis is hepatic fibrosis, lung fibrosis or dermal fibrosis;
or any combination thereof.

87. The method of claim 86,
wherein said systemic fibrotic disease is systemic sclerosis, multifocal fibrosclerosis (IgG4-associated fibrosis), nephrogenic systemic fibrosis, sclerodermatous graft vs. host disease, or any combination thereof;
wherein said organ-specific fibrotic disease is lung fibrosis, cardiac fibrosis, kidney fibrosis, pulmonary fibrosis, liver and portal vein fibrosis, radiation-induced fibrosis, bladder fibrosis, intestinal fibrosis, peritoneal sclerosis, diffuse fasciitis, wound healing, scaring, or any combination thereof;
wherein the dermal fibrosis is scleroderma;
wherein the dermal fibrosis is a result of a localized or generalized morphea, keloids, hypertrophic scars, familial cutaneous collagenoma, connective tissue nevi of the collagen type, or any combination thereof;
wherein the hepatic fibrosis is a result of hepatic scarring or chronic liver injury;
or any combination thereof.

88. The method of claim 87,
wherein said lung fibrosis is idiopathic pulmonary fibrosis (IPF);
wherein said cardiac fibrosis is hypertension-associated cardiac fibrosis, Post-myocardial infarction, Chagas disease-induced myocardial fibrosis or any combination thereof;
wherein said kidney fibrosis is diabetic and hypertensive nephropathy, urinary tract obstruction-induced kidney fibrosis, inflammatory/autoimmune-induced kidney fibrosis, aristolochic acid nephropathy, polycystic kidney disease, or any combination thereof;
wherein said pulmonary fibrosis is idiopathic pulmonary fibrosis, silica-induced pneumoconiosis (silicosis), asbestos-induced pulmonary fibrosis (asbestosis), chemotherapeutic agent-induced pulmonary fibrosis, or any combination there;
wherein said liver and portal vein fibrosis is alcoholic and nonalcoholic liver fibrosis, hepatitis C-induced liver fibrosis, primary biliary cirrhosis, parasite-induced liver fibrosis (schistosomiasis), or any combination thereof;
wherein said diffuse fasciitis is localized scleroderma, keloids, dupuytren's disease, peyronie's disease, myelofibrosis, oral submucous fibrosis, or any combination thereof;
wherein the chronic liver injury results from alcoholism, malnutrition, hemochromatosis, exposure to poisons, toxins or drugs;
or any combination thereof.

89. The method of claim 85, wherein said subject has a liver cirrhosis.

90. A method of treating, suppressing, reducing the severity, reducing the risk of developing or inhibiting a disease or condition in a subject, comprising administering a compound according to claim 1 to a subject suffering from a disease or condition under conditions effective to treat, suppress, reduce the severity, reduce the risk of developing, or inhibit the disease or condition in said subject, wherein the disease or condition is one or more selected from: lung fibrosis, idiopathic pulmonary fibrosis (IPF), hepato-fibrotic disorder, cirrhosis, alcoholic steatohepatitis (ASH), non-alcoholic steatohepatitis (NASH), alcoholic fatty liver disease (AFLD), non alcoholic fatty liver disease (NAFLD), an autoimmune disease or disorder.

91. The method of claim 90,
wherein the lung fibrosis is idiopathic pulmonary fibrosis (IPF);
wherein the hepato-fibrotic disorder is a portal hypertension, cirrhosis, congenital hepatic fibrosis or any combination thereof;
wherein the cirrhosis is a result of hepatitis or alcoholism;
or any combination thereof.

92. A method of treating, suppressing, reducing the severity, reducing the risk of developing or inhibiting a disease or condition in a subject, comprising administering a compound represented by the following structures:

| Compound | |
|---|---|
| Number | Compound Structure |
| 205 | 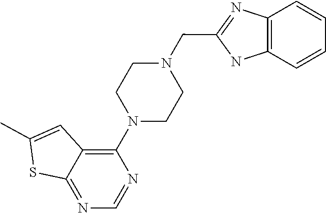 |
| 207 | 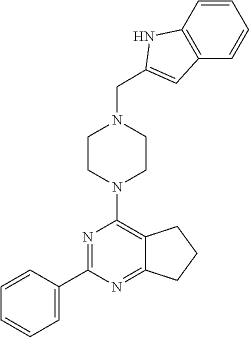 [SMILES: c1ccc(-c2nc3c(c(N4CCN(Cc5cc6ccccc6[nH]5)CC4)n2)CCC3)cc1] |
| 208 | 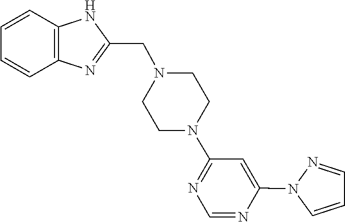 [SMILES: c1ccc2[nH]c(CN3CCN(c4cc(-n5cccn5)ncn4)CC3)nc2c1] |
| 231 | 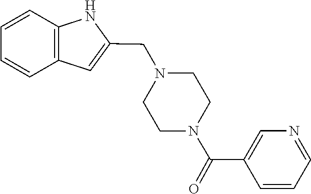 [SMILES: O=C(c1cccnc1)N1CCN(Cc2cc3ccccc3[nH]2)CC1] |
| 253 | 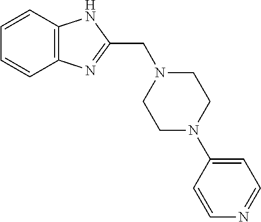 [SMILES: c1ccc2[nH]c(CN3CCN(c4ccncc4)CC3)nc2c1] |

to a subject suffering rom a disease or condition under conditions effective to treat, suppress, reduce the severity, reduce the risk of developing, or inhibit the disease or condition in said subject, wherein the disease or condition is one or more selected from:
lung fibrosis, idiopathic pulmonary fibrosis (IPF), hepato-fibrotic disorder, cirrhosis, alcoholic steatohepatitis (ASH), non-alcoholic steatohepatitis (NASH), alcoholic fatty liver disease (AFLD), non alcoholic fatty liver disease (NAFLD), an autoimmune disease or disorder.

93. The method of claim 92,
wherein the lung fibrosis is idiopathic pulmonary fibrosis (IPF);
wherein the hepato-fibrotic disorder is a portal hypertension, cirrhosis, congenital hepatic fibrosis or any combination thereof;
wherein the cirrhosis is a result of hepatitis or alcoholism;
or any combination thereof.
